(19) United States
(12) Patent Application Publication
(10) Pub. No.: US 2009/0220951 A1
(43) Pub. Date: Sep. 3, 2009

(54) Methods and compositions for classifying bacillus bacteria

(76) Inventors: Katherine Wheeler, Oakland, CA (US); Terrance J. Leighton, Lafayette, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/006,768

(22) Filed: Jan. 4, 2008

Related U.S. Application Data
(60) Provisional application No. 60/878,748, filed on Jan. 5, 2007.

(51) Int. Cl. C12Q 1/68; C07H 21/04

(52) U.S. Cl. 435/6; 536/24.33

(57) ABSTRACT

A method of classifying a Bacillus bacterium is provided. In certain embodiments, the method includes proteotyping a Bacillus bacterium by analyzing a nucleic acid encoding an SspE protein of the Bacillus bacterium.

### CROSS-REFERENCE

This patent application claims the benefit of U.S. provisional patent application Ser. No. 60/878,784, filed on Jan. 5, 2007, which application is incorporated by reference herein.

### STATEMENT REGARDING FEDERALLY SPONSORED RESEARCH

Research was funded by the United States Government under Grant No. DAAD 19-03-C-051 awarded by DARPA. The United States Government may have certain rights in this application.

### BACKGROUND

Unambiguous and precise genetic classification of microorganisms is of pivotal importance to the establishment of strain novelty and utility, associations with existing groups of known commercial importance, association with groups of known biosafety and GRAS classifications, and enabling rapid screening of new isolates for commercial potential by positioning within groups of established economical importance.

This disclosure provides a methodology to reliably and unambiguously identify and stratify members of the Bacillus genus that are or could be used commercially in industrial enzyme, probiotic, biopolymer, biomolecule production, crop protection and other industries.

### SUMMARY OF THE INVENTION

In one embodiment, a method of classifying a Bacillus bacterium is provided. The method may include analyzing a nucleic acid encoding an SspE protein of the Bacillus bacterium to determine an SspE proteotype; and classifying the Bacillus bacterium on the basis of the SspE proteotype. The method may also include further classifying the Bacillus bacterium on the basis of its sspE genotype, and/or by multi-locus sequence typing (MLST) classifiers.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 shows a ClUSTALW multi-sequence alignment of SspE amino acid sequences from the B. thuringiensis group. The SspE sequence for B. cereus strain T was selected as the holotype and used as the reference sequence for the other proteotypes. It has been designated proteotype “A” in FIGS. 1-3 and Tables 1-3. In this figure, the reference sequence “A” is indicated in bold blue type, and amino acid positions (numbering at the bottom of the figure) are in reference to this sequence. Thus, the two amino acid residue inserts found in B. anthracis and some B. mycoides strains are found between residues 54 and 55 of the reference sequence “A.” Amino acid alterations or deletions with respect to reference sequence “A” are highlighted in bold type and their corresponding positions in the holotype reference protein sequence are indicated by bold orange font. The symbol “-” represents missing amino acid residues or deletions with respect to the holotype sequence. From top to bottom: SEQ ID NOS:1-21.

FIG. 2 shows a maximum parsimony phylogenetic tree of Bt group full-length sspE DNA sequences generated by PAUP v.4.0b10 with 100 bootstrap replicates. Genotype labeling corresponds to that used in Tables 1-3. Numbers at the branch nodes indicate bootstrap confidence values as a percentage from 100 replicates. Primary or major claims are indicated with arrows. B. mycoides-related strains are labeled as points of reference. Blue, red and green branch color-coding of clusters corresponds to color coding of clusters in the MLST tree in FIG. 3 and to classifiers and sspE genotypes and strains in Tables 1-3.

FIG. 3 shows a maximum likelihood phylogenetic tree of Bt group concatenated MLST allele sequences (glpF, gmk, ilvD, pta, purH, pycA and tpiA) generated by PHYML with 500 bootstrap replicates. Numbers at the branch nodes indicate bootstrap confidence values as a percentage from 500 replicates. Phylogenetic positions of Bc group strains from this study are indicated by classifiers (see Table 1). Commercially relevant insecticidal Bt strains are indicated with arrows. B. anthracis and B. mycoides-related strains are labeled as points of reference. Blue, red and green branch color-coding of clusters corresponds to color coding of clusters in the sspE tree in FIG. 2 and to classifiers and sspE genotypes and strains in Tables 1-3.

FIG. 4 shows a ClUSTALW multi-sequence alignment of SspE amino acid sequences from the B. subtilis group. The SspE sequence for B. subtilis strain W23 was selected as the holotype and used as the reference sequence for the other proteotypes. It has been designated proteotype “12” in FIGS. 4-6 and Tables 5 and 6. In this figure, the reference sequence “12” is indicated in bold blue type, and amino acid positions (numbering at the bottom of the figure) are in reference to this sequence. Amino acid alterations or deletions with respect to reference sequence “12” are highlighted in bold type and their corresponding numbered positions in the protein sequence are indicated by bold orange font. Numbers in the left column corresponding to SspE proteotypes 1-11 are indicated in bold type since one or more commercially valuable isolates cluster in this proteotype. SspE proteotype numbering assignments remain consistent between this figure and Tables 5 and 6 as well as the B. subtilis group phylogenetic trees in FIGS. 5 and 6. SspE sequences of B. licheniformis (proteotype “6”)-related strains, including B. sonorensis (proteotype “7”) and isolates important in enzyme production (proteotype “11”) and plant protection/biofungicide (proteotypes “8-10”) have a 28 amino acid deletion with respect to the W23 holotype sequence, corresponding to holotype amino acid residue positions 48-75 (inclusive) in FIG. 4. Though the precise position of this sequence gap may be relative and is dependent on the ClUSTALW alignment parameters, we determined that a deletion positioned at residues 48-75 (inclusive) was the most plausible location based upon evolutionary characteristics and motifs found in the sspE gene. The symbol “-” represents missing amino acid residues or deletions with respect to the holotype sequence. From top to bottom: SEQ ID NOS:22-39.

FIG. 5 shows a maximum parsimony phylogenetic tree of Bs group full-length sspE DNA sequences generated by PAUP v.4.0b10 with 1000 bootstrap replicates. Genotype labeling corresponds to that in Tables 5 and 6. Numbers at the branch nodes indicate bootstrap confidence values as a percentage from 1000 replicates. Commercially relevant clusters are indicated. B. atrophaeus, B. vallismortis and B. subtilis-related strains are labeled as points of reference. Violet, coral, gold, dark teal, gray, leaf green and aqua branch color-coding of clusters corresponds to color coding of clusters in the MLST tree in FIG. 6 and to classifiers, sspE genotypes and strains in Tables 5-7.

FIG. 6 shows a maximum likelihood phylogenetic tree of Bs group concatenated MLST allele sequences (glpF, ilvD, pta, purH, pycA, rpoD and tpiA) generated by PHYML with 1000 bootstrap replicates. Numbers at the branch nodes indicate bootstrap confidence values as a percentage from 1000 replicates. Phylogenetic positions of Bs group strains are indicated by classifiers (see Table 5). Commercially relevant clusters are identified. B. atrophaeus, B. vallismortis and B. subtilis-related strains are labeled as points of reference. Violet, coral, gold, dark teal, gray, leaf green and aqua branch color-coding of clusters corresponds to color coding of clusters in the sspE tree in FIG. 5 and to classifiers, sspE genotypes and strains in Tables 5-7.

FIG. 7 ClUSTALW multi-sequence alignment of 54-56 residue SspE amino acid sequences from the B. subtilis group. The SspE sequence for Bacillus species proteotype 8 (biofungicide strain GB03, etc.) was selected as the holotype and used as the reference sequence for the other proteotypes. It has been designated proteotype “8” in FIGS. 4-9 and Tables 5-8. In this figure, the reference sequence “8” is indicated in bold blue type, and amino acid positions (numbering at the bottom of the figure) are in reference to this sequence. Amino acid alterations or deletions with respect to reference sequence “8” are highlighted in bold type and their corresponding numbered positions in the protein sequence are indicated by bold orange font. Numbers in the left column corresponding to SspE proteotypes 19-21 are indicated in bold brown type and represent SspE translated protein sequences from five bona fide Bacillus pumilus isolates. SspE proteotype numbering assignments remain consistent between this figure and Tables 5-8 as well as the B. subtilis group phylogenetic trees in FIGS. 5 and 6. The symbol “-” represents missing amino acid residues or deletions with respect to the holotype sequence. It is unclear whether one or both N-terminal methionine residues are actually incorporated into the B. pumilus SspE protein. From top to bottom: SEQ ID NOS:40-48.

FIGS. 8 and 9 show maximum parsimony phylogenetic trees of Bs group full-length SspE translated amino acid (FIG. 8) and nucleotide (FIG. 9) sequences generated by PAUP v.4.0b10 with 1000 bootstrap replicates. Genotype labeling corresponds to that in Tables 5-8. Numbers at the branch nodes indicate bootstrap confidence values as a percentage from 1000 replicates. Commercially relevant clusters are indicated. B. licheniformis, B. sonorensis and B. pumilus-related strains are labeled as points of reference. Leaf green, aqua and brown branch color-coding of clusters corresponds to color coding of clusters in the FIGS. 5 and 6 and to classifiers and sspE genotypes and strains in Tables 5-8.

FIG. 10 is a table showing classification of Bacillus thuringiensis group isolates by SspE proteotype, sspE genotype and MLST classifiers.

FIG. 11 is a table showing classification of Bacillus subtilis group isolates by SspE proteotype, sspE genotype and MLST classifiers.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

As used herein, the terms “determining,” “measuring,” and “assessing,” and “assaying” are used interchangeably and include both quantitative and qualitative determinations.

The term “nucleic acid” as used herein means a polymer composed of nucleotides, e.g., deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “ribonucleic acid” and “RNA” as used herein refer to a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length. Oligonucleotides may be made synthetically or by copying a template (e.g., an SspE gene template) using a polymerase.

The term “polynucleotide” as used herein refers to a single or double stranded polymer composed of nucleotide monomers, of generally greater than 100 nucleotides in length.

The term “stringent conditions” refers to conditions under which a primer will hybridize preferentially to, or specifically bind to, its complementary binding partner, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90%4 as stringent as the above specific stringent conditions. Other stringent hybridization conditions are known in the art and may also be employed, as appropriate.

Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G=U or U=G base pairs. Thus, two sequences need not have perfect homology to be “complementary” under the invention, and in most situations two sequences are sufficiently complementary when at least about 85% (preferably at least about 90%, and most preferably at least about 95%) of the nucleotides share base pair organization over a defined length of the molecule.

As used herein, a “biological sample” refers to a sample of tissue or fluid isolated from a subject, which in the context of the invention generally refers to samples suspected of containing nucleic acid and/or cellular particles of human B. anthracis, which samples, after optional processing, can be analyzed in an in vitro assay. Typical samples of interest include, but are not necessarily limited to, respiratory secretions (e.g., samples obtained from fluids or tissue of nasal passages, lung, and the like), blood, plasma, serum, blood cells, fecal matter, urine, tears, saliva, milk, organs, biopsies, and secretions of the intestinal and respiratory tracts. Samples also include samples of in vitro cell culture constituents including but not limited to conditioned media resulting from the growth of cells and tissues in culture medium, e.g., recombinant cells, and cell components.

The term “assessing” includes any form of measurement, and includes determining if an element is present or not. The terms “determining”, “measuring”, “evaluating”, “assessing” and “assaying” are used interchangeably and includes quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

The term “Bacillus bacterium” refers to any species in the genus Bacillus, including Bacillus thuringiensis group bacteria and Bacillus subtilis group bacteria. A Bacillus bacterium may be present as a Bacillus isolate (e.g., an isolated bacterium cultured in vitro), or may be present in a sample that contains other bacteria, for example.

The term “Bacillus thuringiensis group” refers to a group of Bacillus bacteria that is phylogenetically related to Bacillus thuringiensis and phylogenetically distinct from Bacillus subtilis group bacteria. The Bacillus thuringiensis group includes, but is not limited to, the following species: Bacillus thuringiensis (Bt), Bacillus anthracis (Ba), Bacillus cereus (Bc), Bacillus mycoides (Bm), Bacillus pseudomycoides (Bp), Bacillus weihenstephanensis (Bw), including subspecies thereof, including serovars kurstaki, israelensis, aizawai/pacificus and thuringiensis. In certain cases, a Bacillus bacterium may be classified as a Bacillus thuringiensis group bacterium using the SspE-based methods described below.

The term “Bacillus subtilis group” refers to a group of Bacillus bacteria that is phylogenetically related to Bacillus subtilis, and phylogenetically distinct from Bacillus thuringiensis group bacteria. The Bacillus subtilis group includes, but is not limited to, the following species: Bacillus subtilis (Bs), Bacillus licheniformis (Bl), Bacillus amyloliquefaciens, Bacillus vallismortis (By), Bacillus pumilus (Bpum), and Bacillus atrophaeus (Bat), including subspecies thereof. In certain cases, a Bacillus bacterium may be classified as a Bacillus subtilis group bacterium using the SspE-based methods described below.

The term “classifying” in the context of classifying a Bacillus bacterium, refers to assigning a Bacillus bacterium to a pre-defined category, such as a genus, species, or sub-species. In certain embodiments, a Bacillus bacterium is classified when it is assigned to a genus and a species (e.g., named using genus-species nomenclature such as “Bacillus licheniformis”). In other embodiments, a Bacillus bacterium is classified when it is assigned to a genus, a species and a sub-species, (e.g., named using genus-species-subspecies nomenclature such as “Bacillus subtilis strain 168). In certain embodiments, the term “classifying” specifically excludes classifying a bacterium as a B. anthracis solely on the basis of a 6 bp deletion or insertion at nucleotides 177-182 of the sspE gene of Kim et al (FEMS Immunol. Med. Microbiol. 2005 43:301-10) in the sspE gene, although this deletion may be used in the methods described herein in combination with other markers.

The term “sspE” refers to a gene encoding a small, acid-soluble spore protein that is found in the genome of Bacillus bacteria. The nucleotide sequences of several Bacillus bacterium sspE genes and the amino acid sequences of the SspE proteins encoded by those genes have been deposited in NCBI's GenBank database, or are set forth herein in the sequence listing. The nucleotide sequence of the genome of B. subtilis is known (see, e.g., Kunst et al, Nature 1997 390:249-56), and the SspE proteins of various Bacillus bacterium are described in Mason et al (J. Bacteriol. 1988 170:239-44), Mason et al (Nucleic Acids Res. 1988 16:6567-83), Cucchi et al (Curr. Microbiol. 1995 31:228-33) and Kim et al (FEMS Immunol. Med. Microbiol. 2005 43:301-10).

The term “SspE proteotype”, in the context of an SspE proteotype of a Bacillus bacterium, indicates the type of SspE protein encoded by that Bacillus bacterium. Different SspE proteotypes differ in amino acid sequence, and, in certain cases, length. As will be described in greater detail below, different SspE proteotypes allow different Bacillus bacterium to be classified. Also as will be described in greater detail below, an SspE proteotype may be determined by analysis of the sspE gene of a Bacillus bacterium.

The term “sspE genotype”, in the context of an sspE genotype of a Bacillus bacterium, indicates the type of sspE gene encoded by that Bacillus bacterium. Different sspE genotypes differ in nucleic acid sequence, and, as will be described in greater detail below, different sspE genotypes allow different Bacillus bacterium having the same SspE proteotype to be further classified. As will be described in greater detail below, an sspE genotype may be determined by analysis of the sspE gene of a Bacillus bacterium.

The term “SspE classifying amino acid signature” refers to a minimal set of contiguous and/or non-contiguous amino acids of an SspE protein that identifies the SspE protein as being of a particular SspE proteotype. An SspE classifying amino acid signature indicates the identify of the classifying amino acids residues at particular positions in an SspE protein, as well as any classifying insertions or deletions within an SspE protein, relative to another SspE protein. A complete list of SspE classifying amino acid signatures is set forth later in this disclosure. The SspE classifying amino acid signature for B. anthracis is not solely based on identification of a deletion or insertion of the amino acids encoded by nucleotides 177-182 of the sspE gene of Kim et al (FEMS Immunol. Med. Microbiol. 2005 43:301-10).

The term “oligonucleotide primer” is an oligonucleotide that can prime nucleic acid synthesis when hybridized to a longer nucleic acid in the presence of a DNA polymerase and nucleotides.

The term “a set of SspE classifying primers” refers to a set of oligonucleotide primers that are designed to detect an SspE classifying amino acid signature. In certain cases, a set of oligonucleotide primers, when employed in a polymerase chain reaction using a Bacillus species genome as a template, amplify products that are diagnostic of the SspE classifying amino acid signature. In particular embodiments, the sizes of the products indicate the SspE classifying amino acid signature. In other embodiments, the presence or absence of particular products may indicate the SspE classifying amino acid signature. Each product may be amplified by a primer pair, where a set of SspE classifying primers comprises a plurality of primer pairs.

The term “translating”, in the context of translating a sequence of nucleotides, refers to the decoding of a sequence of nucleotides into a sequence of amino acids using the genetic code. Translation of a sequence of nucleotides may be done on paper or by a computer (i.e., in silico), for example.

The term “analyzing”, in the context of analyzing a nucleic acid includes sequencing the nucleic acid and analyzing the nucleotide sequence of the nucleic acid on paper or in silico, as well as physically analyzing the nucleic acid to see if it can act as a template for an enzymatic reaction, e.g., primer extension or by hybridization. A nucleic acid may be copied, e.g., amplified, prior to its analysis.

Other definitions of terms may appear below

### DETAILED DESCRIPTION

Before examples of the instant method is described in such detail it is to be understood that method is not limited to particular variations set forth and may, of course, vary. Various changes may be made to the method described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As noted above, certain of the above methods are sspE-based methods, where sspE is a gene that encodes the spore structural protein SspE, a gamma-type SASP (small, acid-soluble protein). SspE is thought to function as a storage protein that provides amino acids required for protein synthesis during early spore germination. This gene is believed to have arisen and evolved solely within the Bacillus genus. As will be described in greater detail below, sspE gene sequences have been used to reliably reconstruct the natural genetic history of over 380 Bacillus isolates. Certain of the SspE-based methods decrease the time and expense required for discovery of new strains of commercial interest by providing rapid identification assays for isolates that are members of commercially important clades. Certain of the SspE-based methods also allow the accurate and decisive phylogenetic positioning of new isolates for which patent protection or GRAS status is sought.

A method of classifying a Bacillus bacterium is provided. In certain embodiments, the method may include: analyzing a nucleic acid encoding an SspE protein of the Bacillus bacterium to determine an SspE proteotype; and classifying the Bacillus bacterium on the basis of that SspE proteotype. The method may further include analyzing the SspE-encoding nucleic acid to determine an sspE genotype that allows the Bacillus bacterium to be further classified. The method may include sequencing the nucleic acid to provide a nucleic acid sequence and, in certain embodiments, analyzing that nucleic acid sequence. An sspE genotype may be determined by analysis of the nucleic acid alone. In certain embodiments, an SspE proteotype may be determined by analysis of the nucleic acid alone (by examining the nucleotide sequence of the nucleic acid to determine whether the nucleic acid contains codons encoding an amino acid signature, or by use of oligonucleotide primers that specifically detect the codons for a classifying SspE amino acid signature, e.g., by use oligonucleotide primers that prime nucleic acid synthesis if particular amino acids are encoded by the nucleic acid, for example). In other embodiments, an SspE proteotype may be determined by analysis of the amino acid sequence of the SspE polypeptide encoded by the SspE nucleic acid. As such, the nucleic acid may be translated as part of its analysis. In particular embodiments, an SspE proteotype may be determined by identifying a classifying amino acid signature in the amino acid sequence of the SspE polypeptide encoded by the nucleic acid. In other embodiments, an SspE proteotype may be determined by comparing the amino acid sequence to a plurality of other Bacillus SspE amino acid sequences to determine which of the plurality is most similar thereto.

For example, in one embodiment, a test SspE amino acid sequence produced by translation of the sspE gene of a test Bacillus bacterium sequence may be compared to the SspE sequences of the sequence listing using any convenient method, e.g., BLAST, ALIGN or ClUSTALW (Altschul, J. Mol. Biol. 1990 215:403-410; Henikoff, Proc. Natl. Acad. Sci. USA 1989 89:10915; Karin, Proc. Natl. Acad. Sci USA 1993 90:5873; and Higgins et al., Gene 1988 73:237-244) using default parameters, to identify the sequence that is most similar to the test sequence and thereby identify the SspE proteotype and/or genotype to which the test Bacillus bacterium belongs. Bacillus thuringiensis group SspE polypeptide sequences are set forth in the sequence listing as the sequences under the header “Examples of SspE Amino Acid Sequences Used For Classification Bt group” (SEQ ID NOS:49-69) and B. subtilis group SspE polypeptide sequences are set forth as in the sequence listing as the sequences under the header “Examples of SspE Amino Acid Sequences Used For Classification B. subtilis group” (SEQ ID NOS:111-131).

The test Bacillus bacterium may be further classified by comparison of the nucleotide sequence of its sspE gene to the nucleotide sequences of other sspE genes, to identify the sequence to which it is most similar, and thereby identify a Bacillus thuringiensis group SspE subgroup to which the test Bacillus bacterium belongs. In certain cases, once an SspE proteotype has been identified, the test Bacillus bacterium may be further classified by comparing its sspE nucleotide sequence to the sspE nucleotide sequences of that SspE proteotype. Bacillus thuringiensis group sspE polynucleotide sequences are set forth in the sequence listing as the sequences under the header “Examples of sspE Nucleic Acid Sequences Used For Classification—Bt group” (SEQ ID NOS:70-110) and B. subtilis group sspE polynucleotide sequences are set forth as in the sequence listing as the sequences under the header “Examples of sspE Nucleic Acid Sequences Used For Classification of B. subtilis group” (SEQ ID NOS:112-155).

In other embodiments and as noted above, a test Bacillus bacterium may be classified by identifying an SspE classifying amino acid signature, where such signatures are listed in the Table 9, entitled “Bacillus thuringiensis group signatures and genotype” and Table 10, entitled “Bacillus subtilis group signatures and genotype”.

TABLE 9
Bacillus thuringiensis group signatures and genotype:

| | SspE | | |
|---|---|---|---|
| SspE | size | | |
| Identifier | (AA)^{d} | Protein Characteristics | Genotype Characteristics |
| A1 | 93 | 29S, 73S, 87Q | 147A |
| A2 | 93 | 29S, 73S, 87Q | 147G |
| B1 | 93 | 87K | Proteotype B has (at least) one genotype |
| C1 | 93 | 29S, 73A, 87Q | Proteotype C has (at least) one genotype |
| D1 | 93 | 29A, 73S, 87Q | Proteotype D has (at least) one genotype |
| E1 | 93 | 73A, 80Q | 30G, 42T, 102G, 114A, 123C, 126A, 138G, 147A, 174C, 180T |
| E2 | 93 | 73A, 80Q | 30G, 42T, 102G, 114A, 123C, 126G, 138A, 147A, 174C, 180T |
| E3 | 93 | 73A, 80Q | 30A, 42T, 102G, 114A, 123C, 126A, 138A, 147A, 174C, 180T |
| E4 | 93 | 73A, 80Q | 30G, 42C, 102G, 114A, 123T, 126A, 138A, 147A, 174C, 180T |
| E5 | 93 | 73A, 80Q | 30A, 42T, 102G, 114A, 123C, 126A, 138A, 147G, 174T, 180C |
| E6 | 93 | 73A, 80Q | 30G, 42T, 102G, 114G, 123C, 126A, 138G, 147A, 174T, 180T |
| E7 | 93 | 73A, 80Q | 30G, 42T, 102A, 114A, 123C, 126A, 138G, 147A, 174T, 180T |
| E8 | 93 | 73A, 80Q | 30G, 42T, 102G, 114A, 123C, 126A, 138G, 147A, 174T, 180T |
| E9 | 93 | 73A, 80Q | 30G, 42T, 102G, 114A, 123C, 126A, 138A, 147A, 174C, 180T |
| E10 | 93 | 73A, 80Q | 30A, 42T, 102G, 114A, 123C, 126A, 138A, 147G, 174T, 180C |
| E11 | 93 | 73A, 80Q | 30A, 42T, 102G, 114A, 123C, 126A, 138A, 147G, 174T, 180C |
| F1 | 93 | 29A, 33N, 73A | 12G, 81G, 87G, 180C |
| F2 | 93 | 29A, 33N, 73A | 12A, 81G, 87G, 180T |
| F3 | 93 | 29A, 33N, 73A | 12A, 81A, 87A, 180T |
| F4 | 93 | 29A, 33N, 73A | 12A, 81A, 87T, 180T |
| G1 | 93 | 29A, 33N, 55K, 73A | Proteotype G has (at least) one genotype |
| H1 | 93 | 29A, 33N, 73A, 93E | 48C, 87G, 180T, 210T, 237A, 240A |
| H2 | 93 | 29A, 33N, 73A, 93E | 48C, 87G, 180T, 210C, 237A, 240A |
| H3 | 93 | 29A, 33N, 73A, 93E | 48C, 87A, 180T, 210C, 237T, 240G |
| H4 | 93 | 29A, 33N, 73A, 93E | 48T, 87A, 180T, 210C, 237T, 240G |
| H5 | 93 | 29A, 33N, 73A, 93E | 48C, 87G, 180C, 210C, 237T, 240G |
| I1 | 93 | 25C, 29A, 33N, 73A, 93E | Proteotype I has (at least) one genotype |
| J1 | 93 | 29A, 33N, 51T, 73A, 93E | Proteotype J has (at least) one genotype |
| K1 | 93 | 29A, 33N, 73A, 80Q | 48T, 57T, 69C, 84T, 108G, 123C, 138A, 147A, 174C, 189T, |
| | | | 195T, 210C |
| K2 | 93 | 29A, 33N, 73A, 80Q | 48C, 57C, 69T, 84T, 108A, 123C, 138T, 147G, 174T, 189A, |
| | | | 195T, 210C |
| K3 | 93 | 29A, 33N, 73A, 80Q | 48C, 57T, 69T, 84C, 108A, 123T, 138A, 147A, 174C, 189A, |
| | | | 195C, 210T |
| L1 | 93 | 29A, 33N, 34A, 73A, 80Q | Proteotype L has (at least) one genotype |
| M1 | 93 | 29A, 33N, 73A, 80Q, 93E | Proteotype M has (at least) one genotype |
| N1 | 92 | 29A, 33N, 73A, 80Q | Proteotype N has (at least) one genotype |
| O1 | 95 | 29A, 33N, 54S, 55I, 59T, 75A, 82Q | Proteotype O has (at least) one genotype |
| P1 | 95 | 29A, 33N, 54S, 55V, 59T, 75A, 82Q | Proteotype P has (at least) one genotype |
| Q1 | 93 | 29A, 33N, 47Q, 53A, 73A, 80Q, 93E | Proteotype Q has (at least) one genotype |
| R1 | 93 | 29A, 33N, 47Q, 53A, 73A, 80Q, | Proteotype R has (at least) one genotype |
| | | 84T, 93E | |
| S1 | 92 | 2N, 28A, 32N, 37Q, 38K, 39Q, 71Q, | Proteotype S has (at least) one genotype |
| | | 72A, 79Q, 83N, 84K | |
| T1 | 95 | 7G, 29A, 33N, 40Q, 53S, 54G, 55V, | Proteotype T has (at least) one genotype |
| | | 70D, 74Q, 75A, 78Q, 82Q, 87K, 93T | |
| U1 | 95 | 7A, 29A, 33N, 40Q, 53S, 54G, 55V | Proteotype U has (at least) one genotype |
| | | 70D, 74Q, 75A, 78Q, 82Q, 87K, 93T | |

TABLE 10
Bacillus subtilis group signatures and genotype

| | SspE | | |
|---|---|---|---|
| SspE | size | | |
| Identifier | (AA)^{d} | Protein Characteristics | Genotype Characteristics |
| 1 | 85 | 7S, 43K, 67A | Genotype 1a: 18C, 234A |
| 1 | 85 | 7S, 43K, 67A | Genotype 1b: 18C, 234A |
| 2 | 84 | 54G, 66A | Proteotype 2 has one genotype |
| 3 | 85 | 66N, 67T | Proteotype 3 has one genotype |
| 4 | 85 | 66D, 67T | Proteotype 4 has one genotype |
| 5 | 85 | 66N, 67T | Proteotype 5 has one genotype |
| 6 | 54 | 41Q, 49K | Proteotype 6 has one genotype |
| 7 | 54 | 41K, 49N | Proteotype 7 has one genotype |
| 8 | 56 | 2A, 10D, 11V, 15K, 16R, 23S, 37D, 38A | Proteotype 8 has one genotype |
| 9 | 56 | 2A, 10D, 11V, 15K, 16R, 23S, 37D, 38V | Proteotype 9 has one genotype |
| 10 | 56 | 2A, 10D, 11V, 15K, 16K, 23S, 37D, 38A | Proteotype 10 has one genotype |
| 11 | 56 | 2E, 10D, 11V, 15K, 16K, 23S, 37D, 38A | Proteotype 11 has one genotype |
| 12 | 85 | 7F, 43R, 67V | Proteotype 12 has one genotype |
| 13 | 85 | 7F, 43R, 67A | Proteotype 13 has one genotype |
| 14 | 84 | 54G, 66V | Proteotype 14 has one genotype |
| 15 | 84 | 54S, 66V | Proteotype 15 has one genotype |
| 16 | 84 | 4Q, 16K, 38V, 65N | Proteotype 16 has one genotype |
| 17 | 84 | 4Q, 16N, 38v, 65N | Proteotype 17 has one genotype |
| 18 | 82 | 22S, 37V, 64A | Proteotype 18 has one genotype |
| 19 | 55 | 1M, 2M, 3D, 4Q, 7N, 21S, 27F, 37V, 39Q, 41K, 43Y, 46K | Genotype 19a: 33A, 99T |
| 19 | 55 | 1M, 2M, 3D, 4Q, 7N, 21S, 27F, 37V, 39Q, 41K, 43Y, 46K | Genotype 19b: 33G, 99T |
| 19 | 55 | 1M, 2M, 3D, 4Q, 7N, 21S, 27F, 37V, 39Q, 41K, 43Y, 46K | Genotype 19c: 33A, 99C |
| 20 | 55 | 1M, 2M, 3D, 4Q, 7N, 21S, 27Y, 37V, 39Q, 41K, 43Y, 46K | Proteotype 20 has one genotype |
| 21 | 55 | 1M, 2M, 3D, 4Q, 7N, 21A, 27Y, 37A, 39H, 41K, 43Y, 46K | Proteotype 21 has one genotype |

As will be described in greater detail below, the subject methods may be employed alone or in conjunction with MLST (multi-locus sequence typing) to classify a Bacillus bacterium. MLST methods for classifying Bacillus thuringiensis group bacteria are known. For example, the methods of Priest et al. (J Bacteriol, 2004, 186: 7959-7970), Baker et al., 2004; Hanage et al., 2005; Maiden et al., 1998; McGregor et al., 2005; Priest et al., 2004; or Spratt, 1999 (citations provided later in this disclosure), may be employed. MLST methods for classifying Bacillus subtilis group bacteria are described in greater detail below. In one embodiment, a Bacillus subtilis group bacterium may be further classified by determining the nucleotide sequence of the glpF, ilvD, pta, purH, pycA, rpoD and tpiA genes of that bacterium. The nucleotide sequence of each of the genes employed in this MLST method is set forth in the sequence listing under the header “MLST Allele Sequences” (SEQ ID NOS: 156-386).

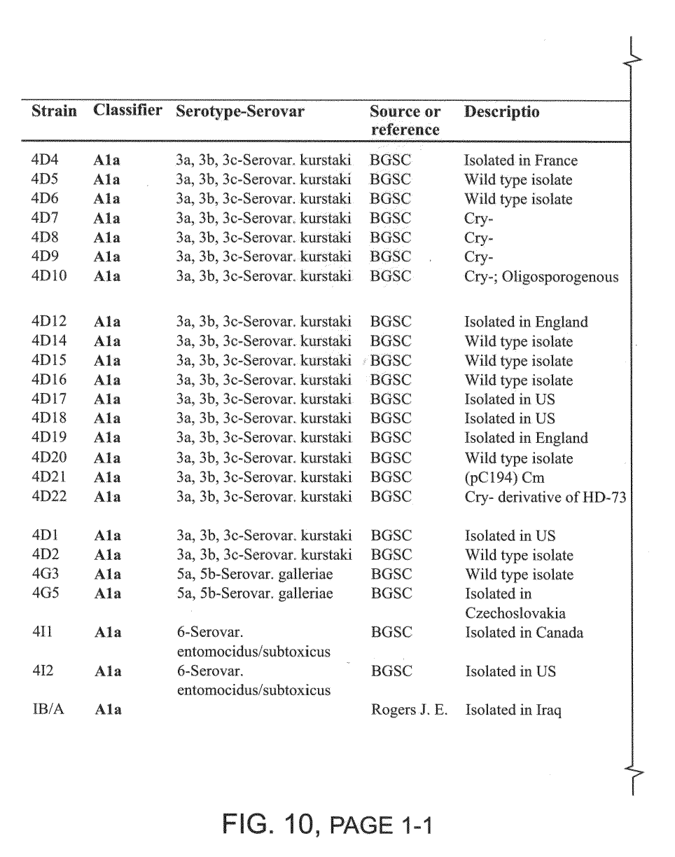

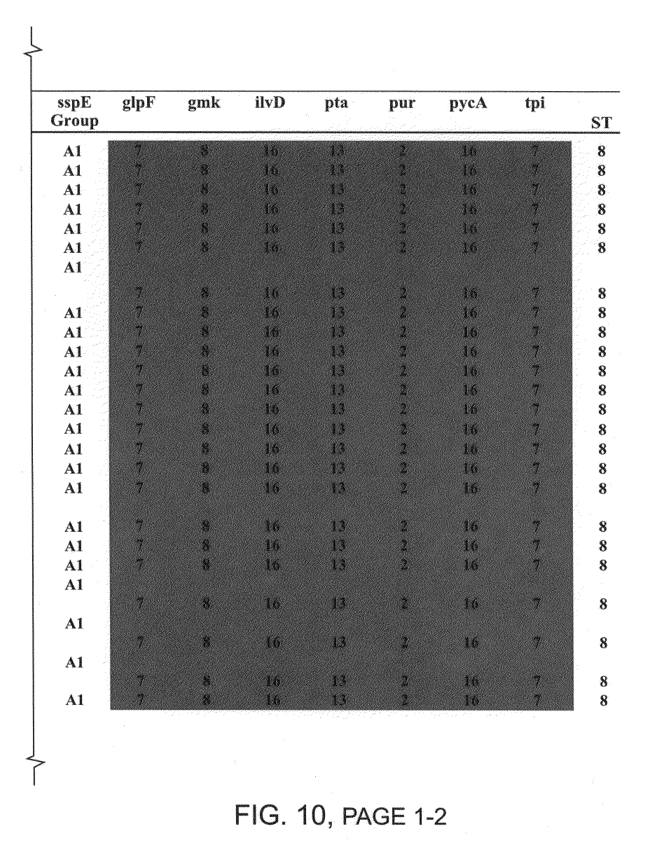

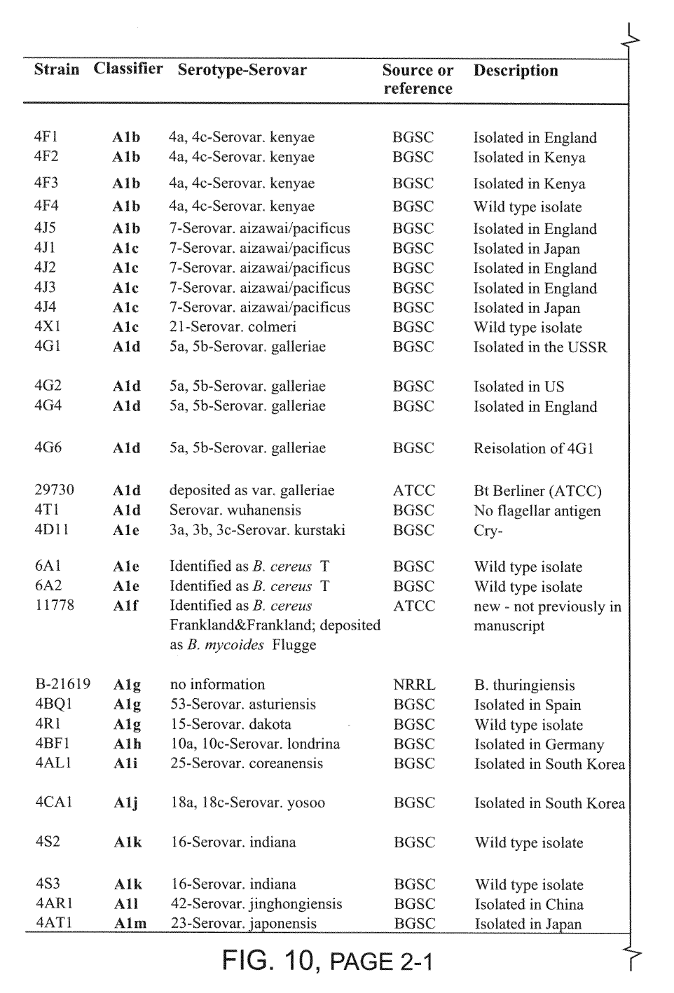

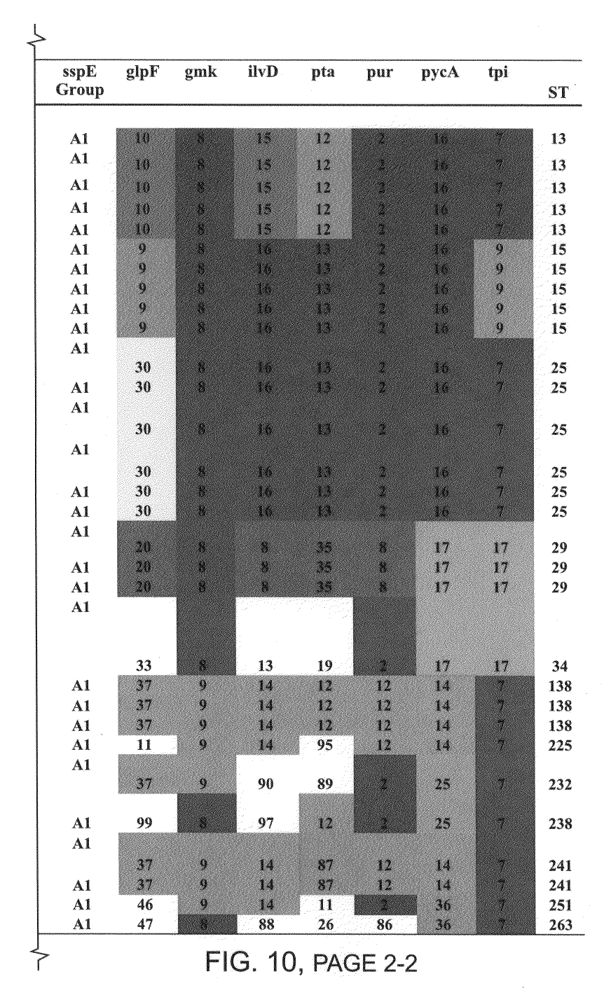

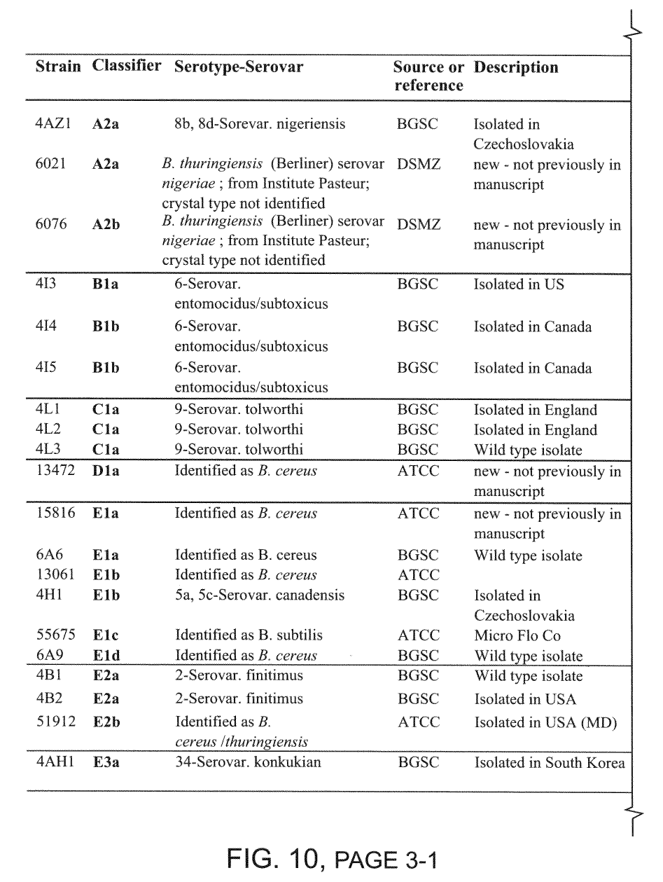

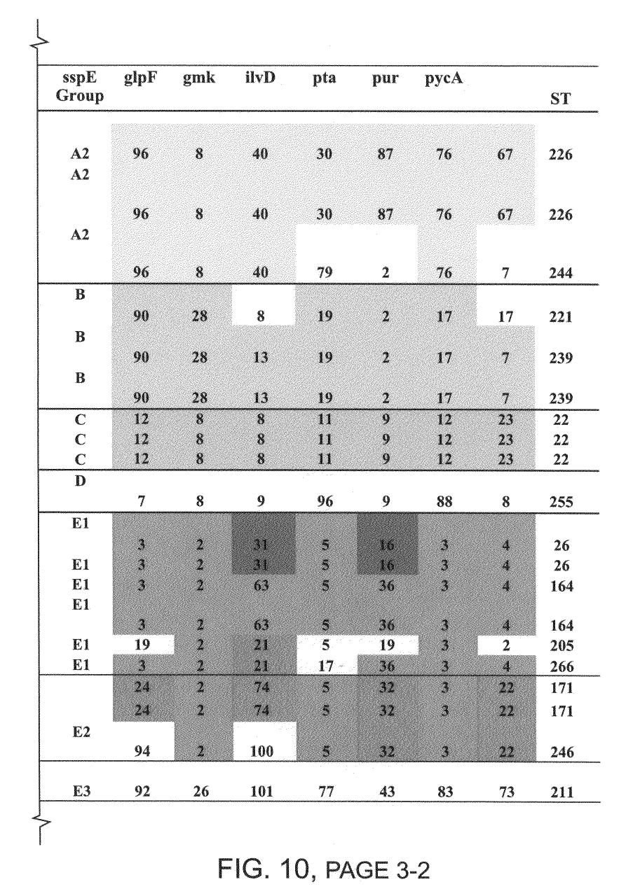

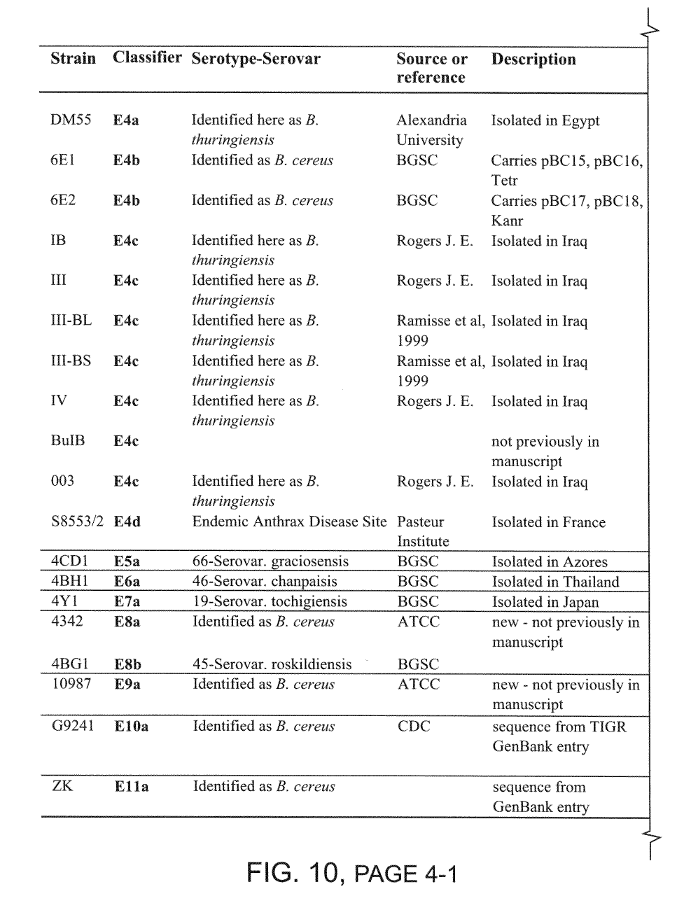

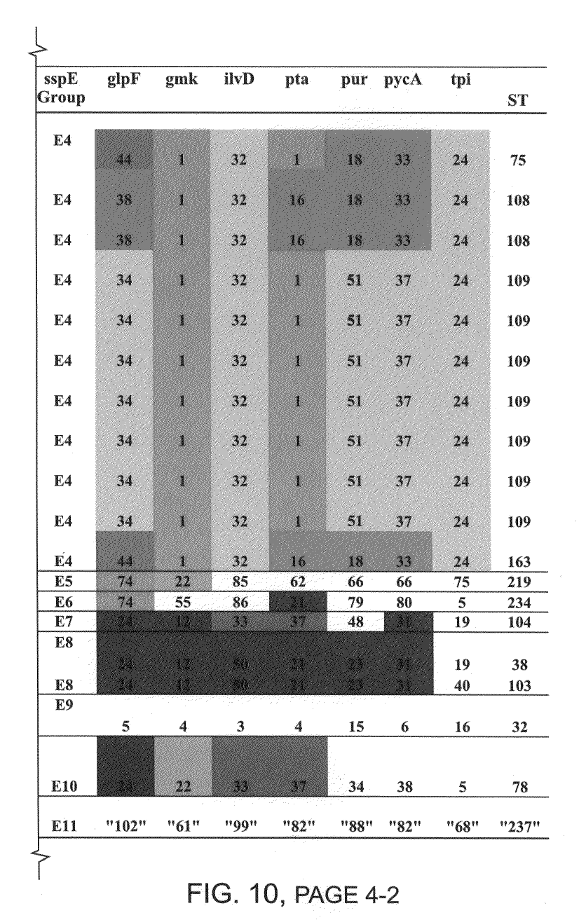

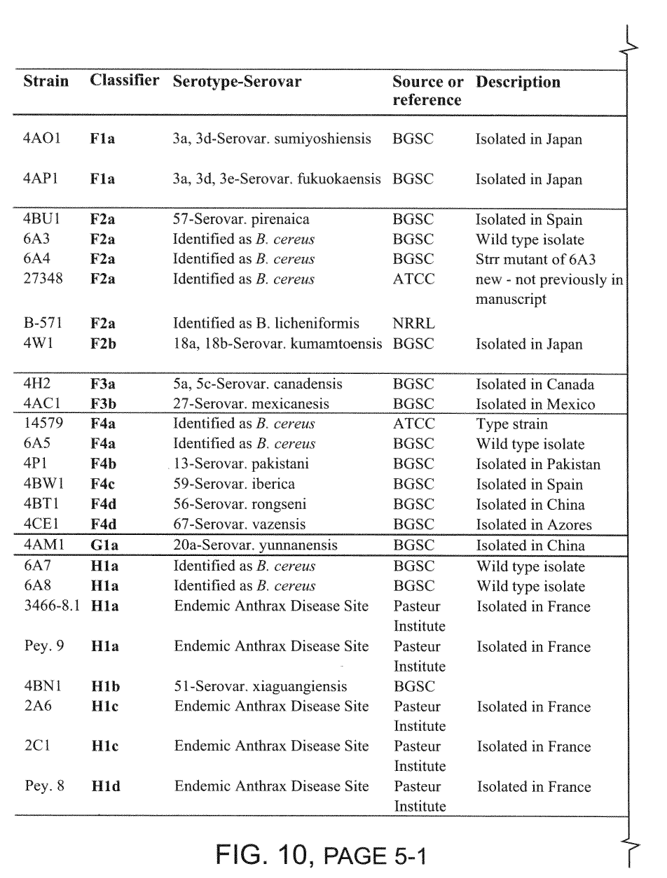

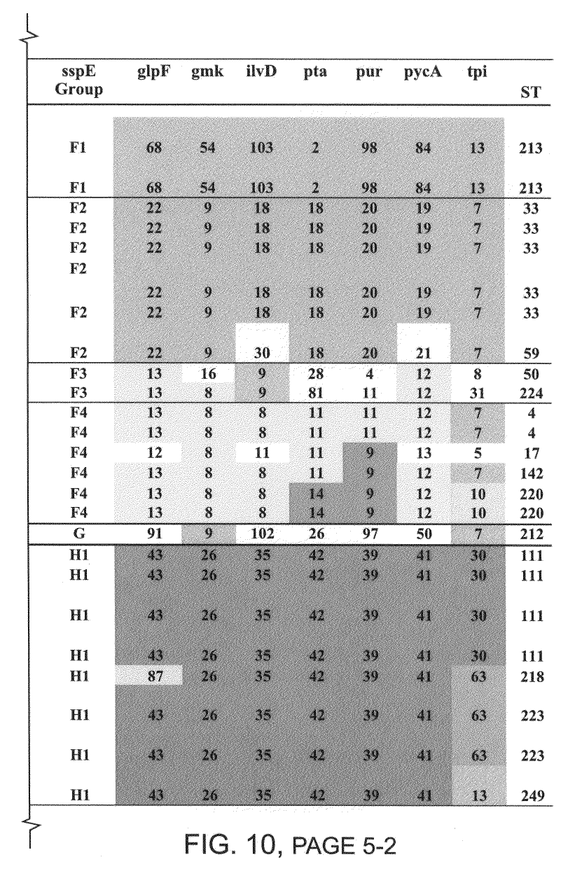

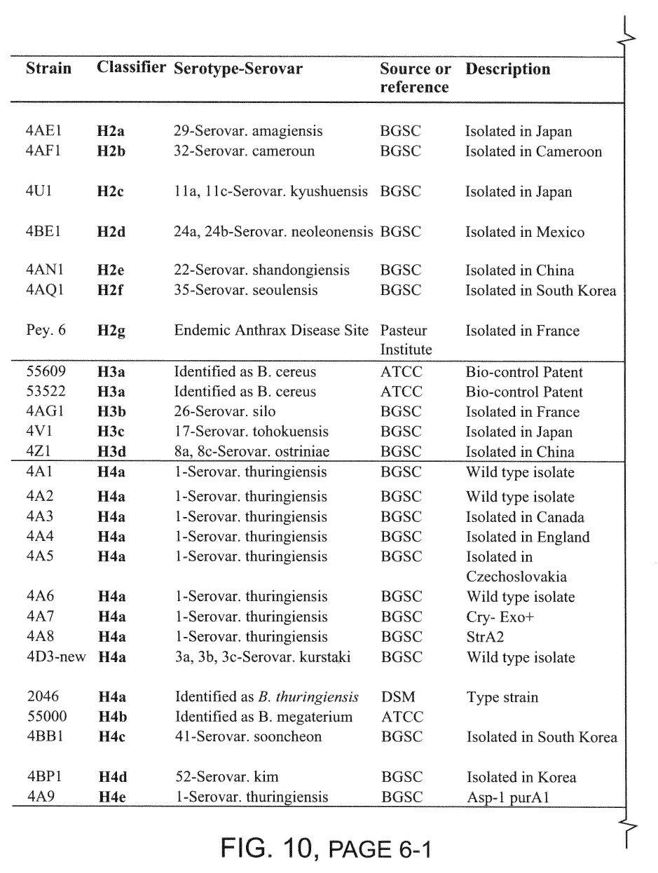

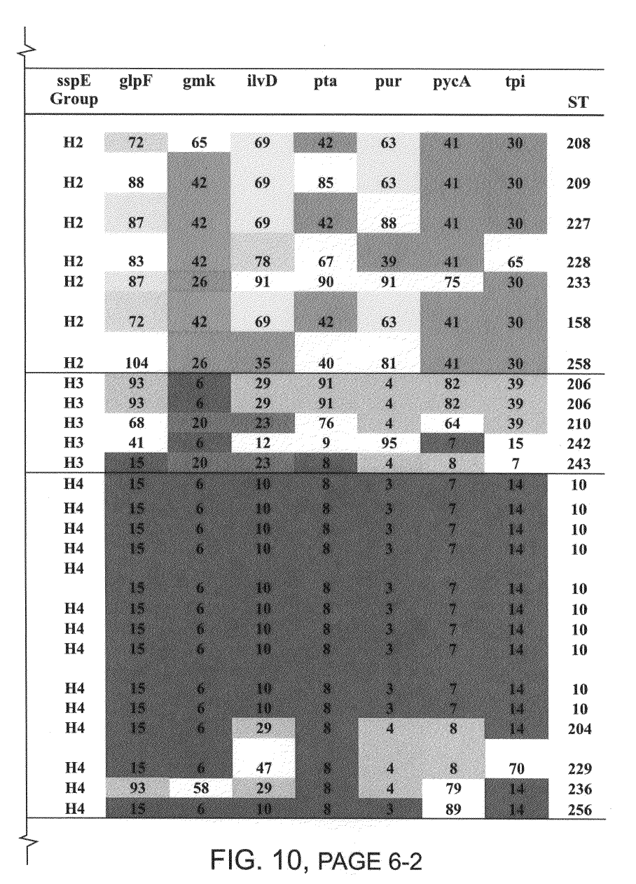

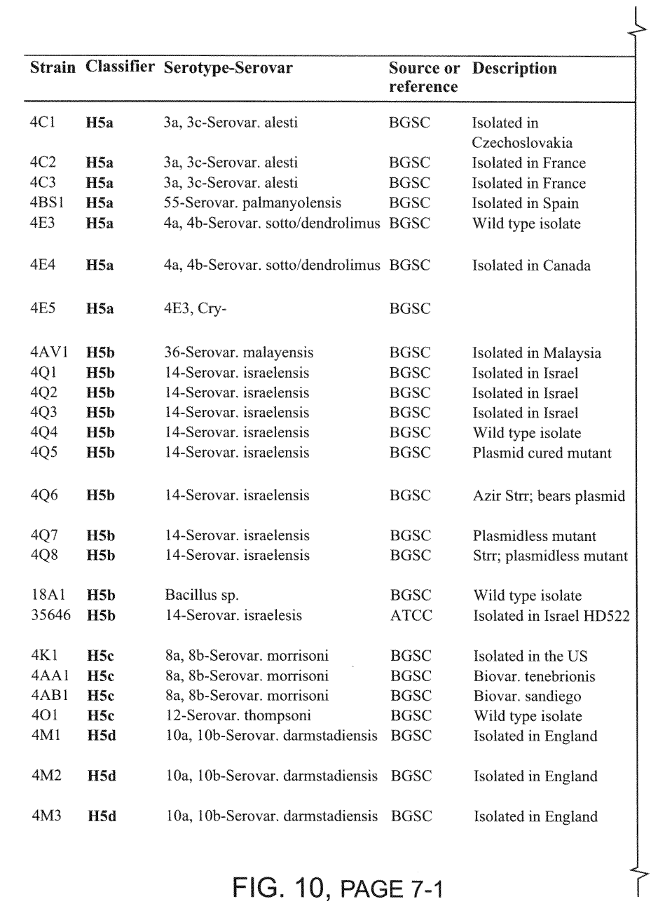

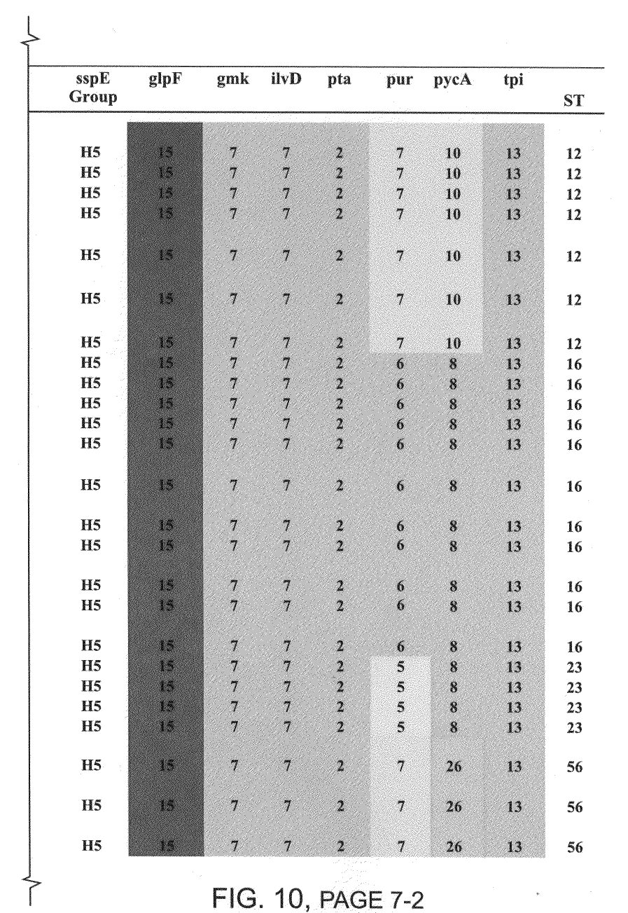

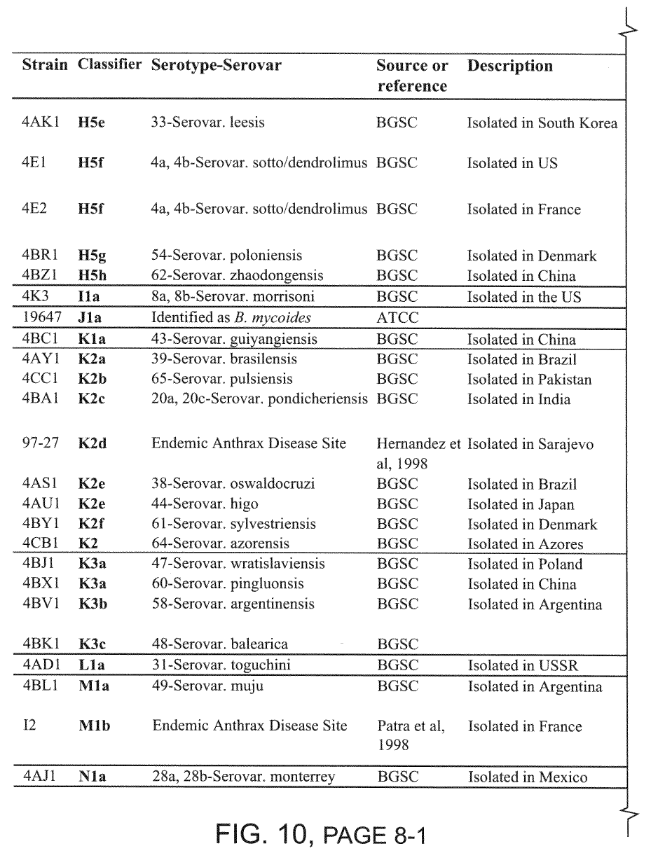

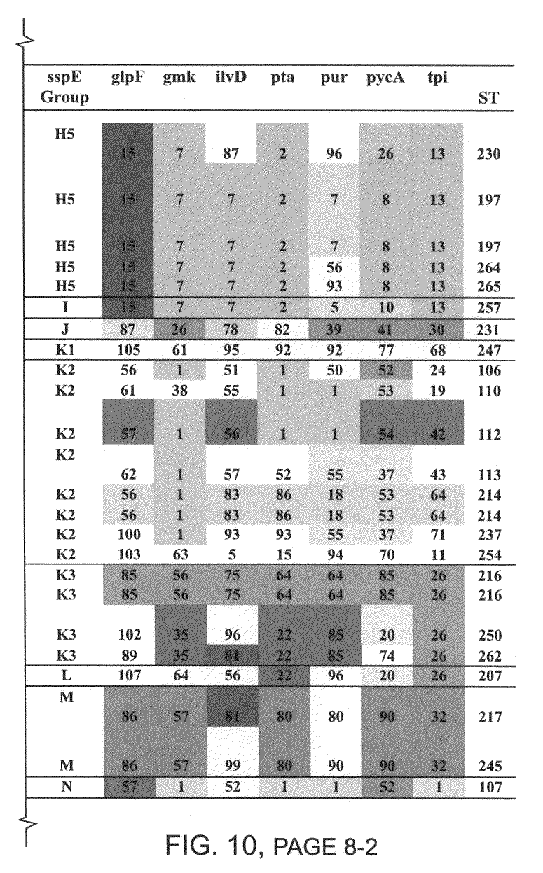

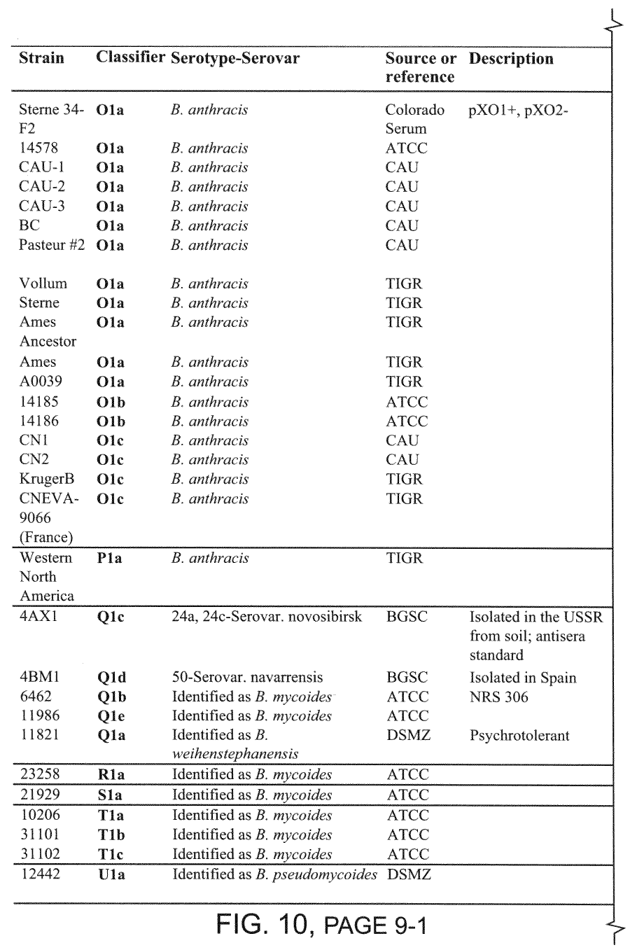

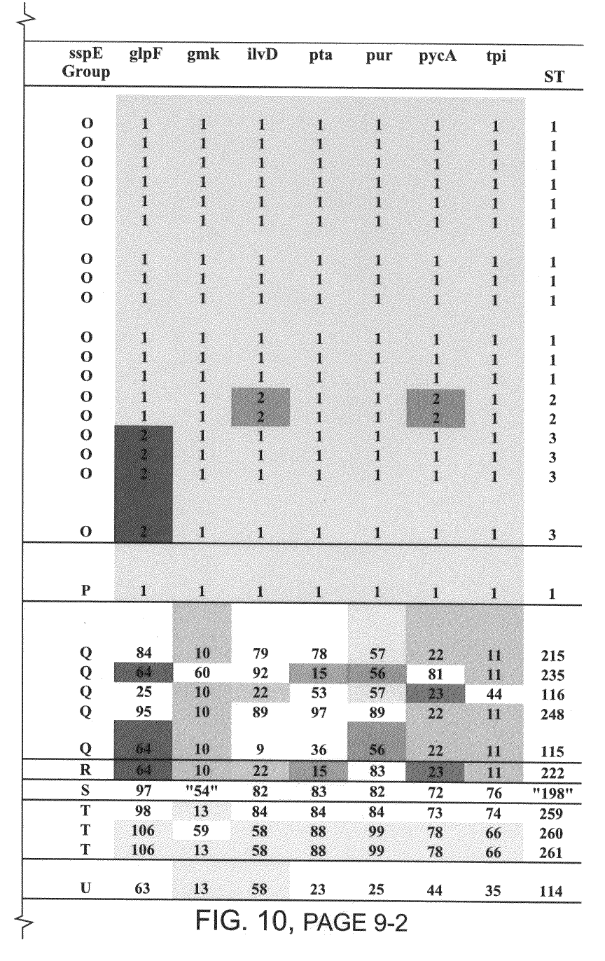

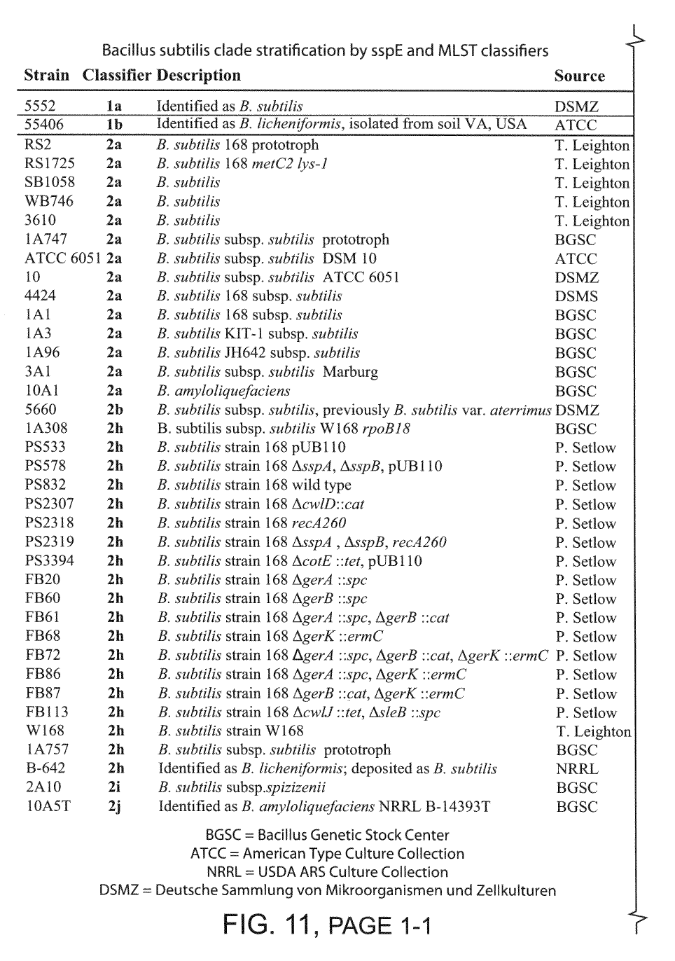

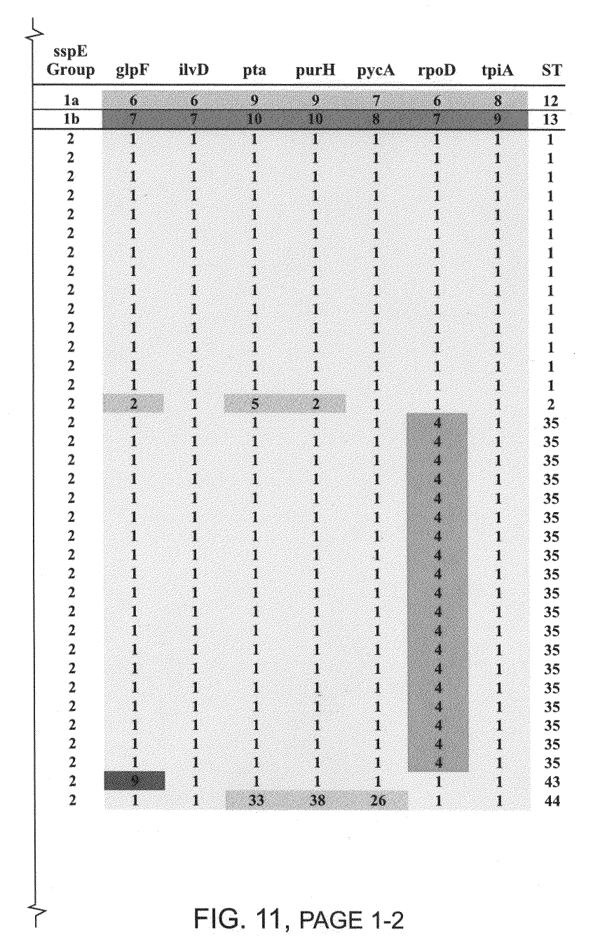

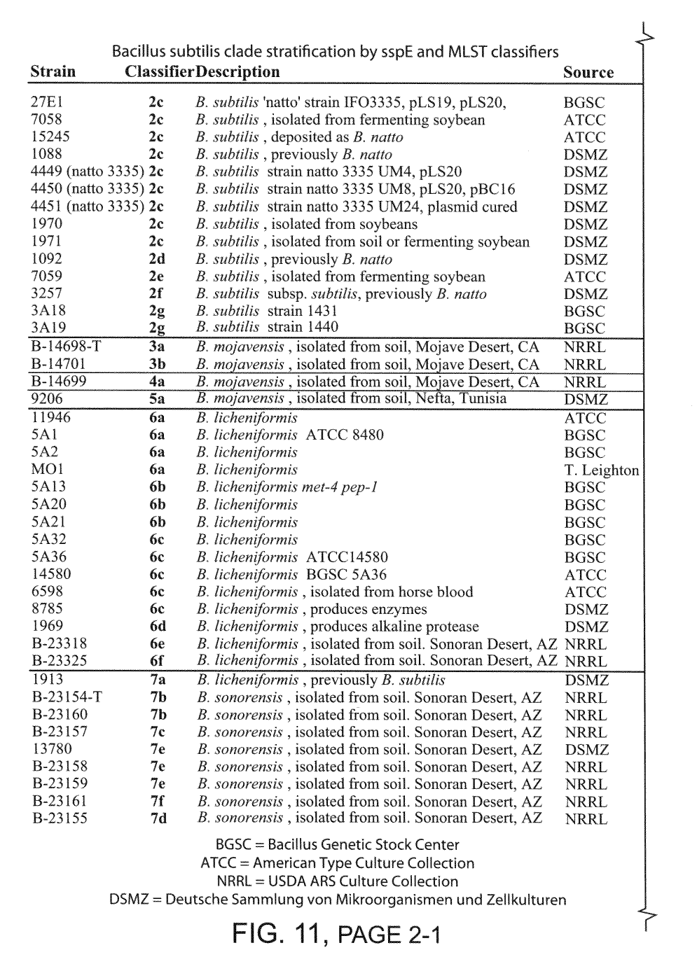

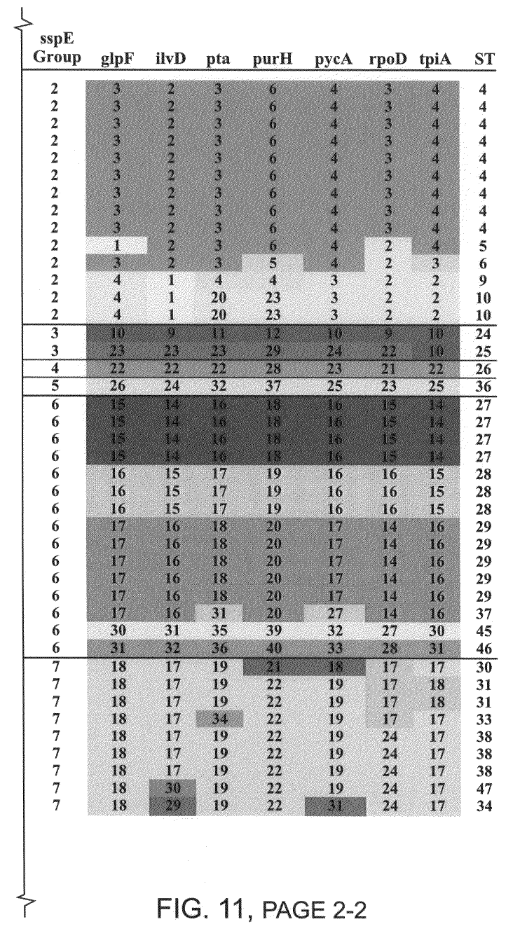

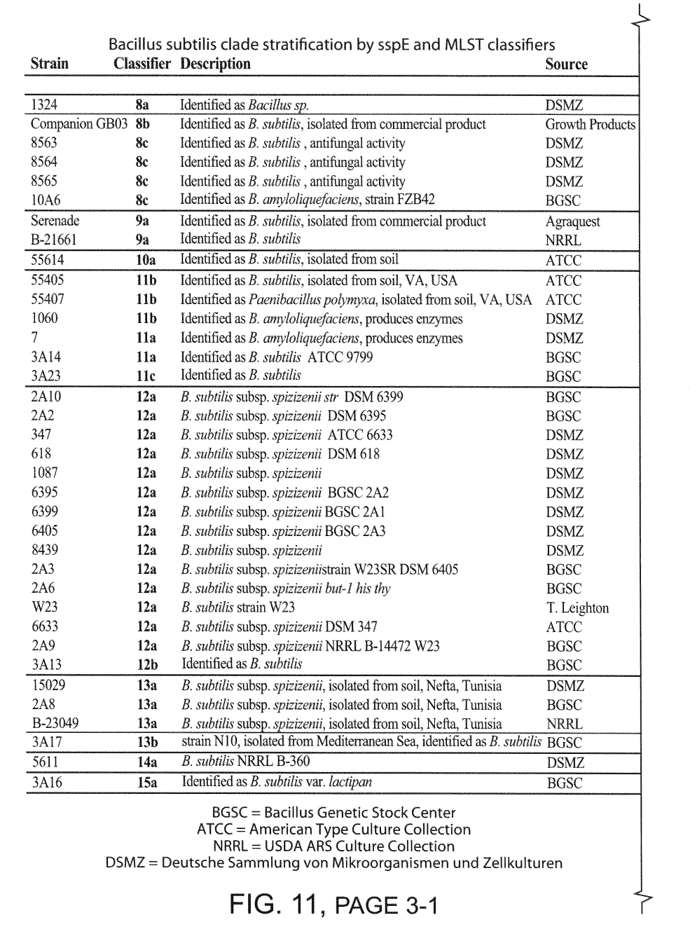

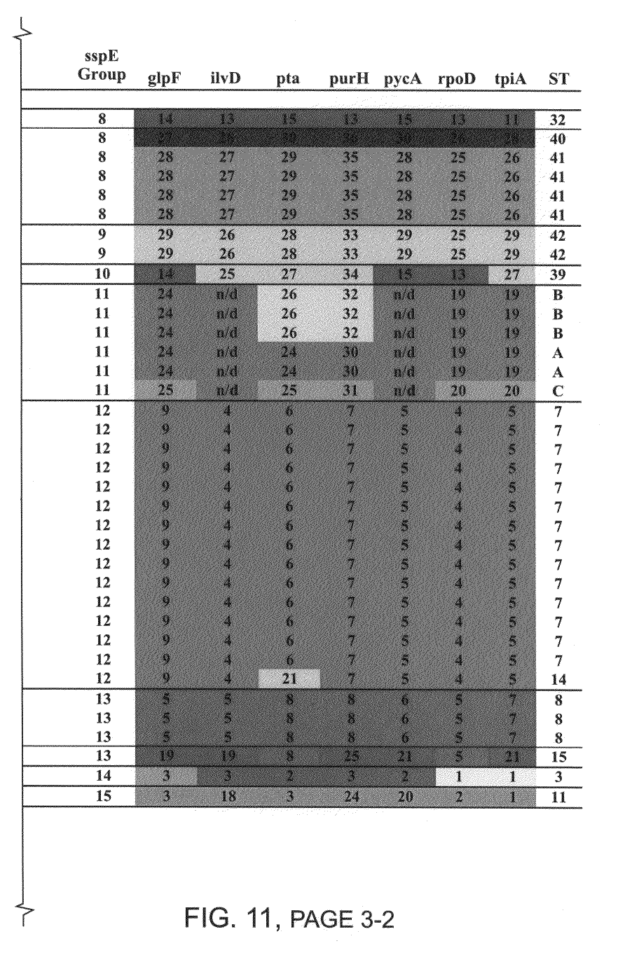

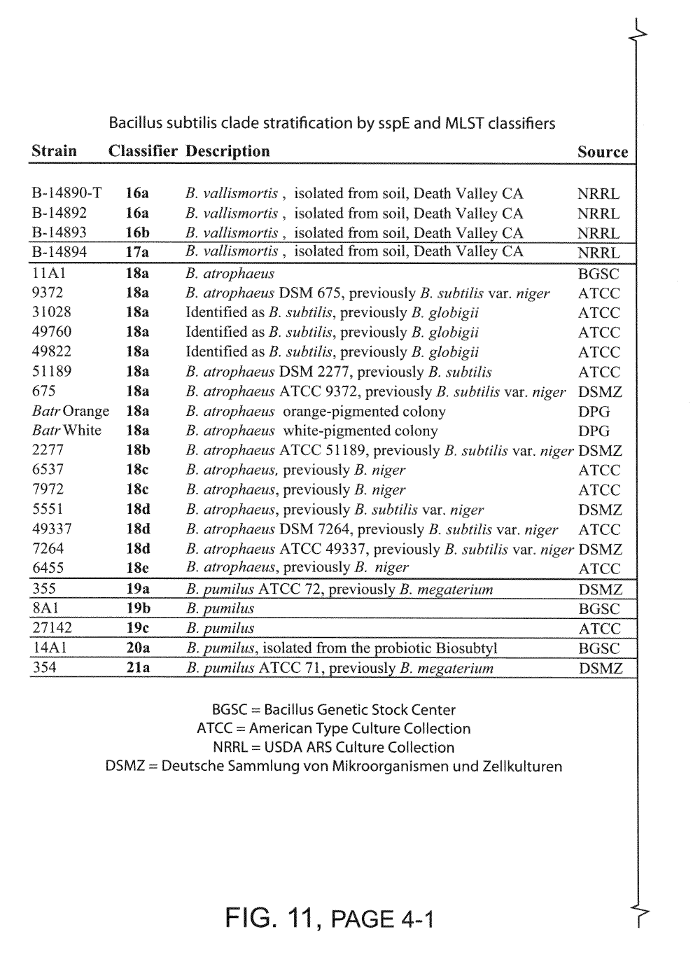

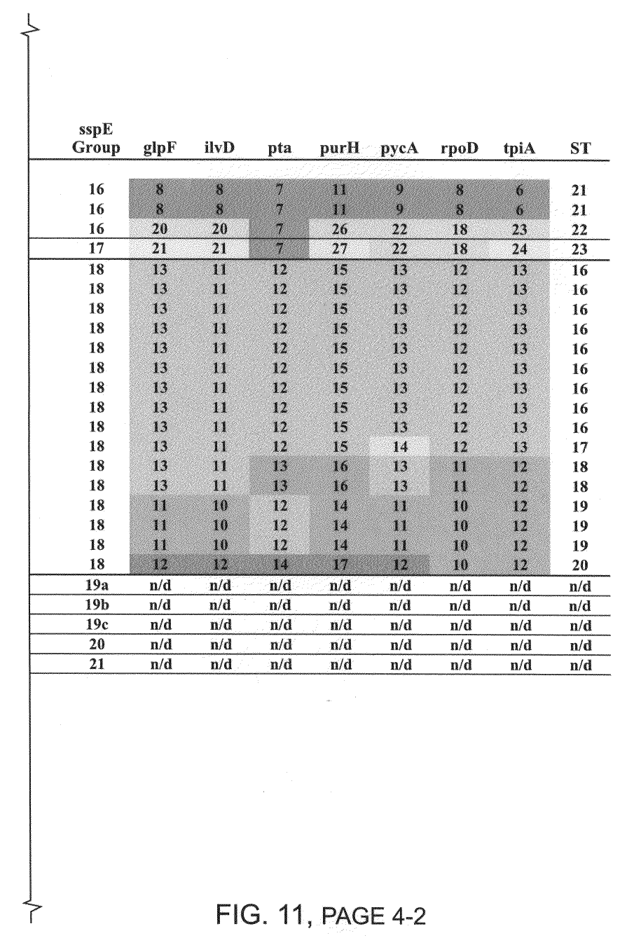

Exemplary results obtained from the subject methods are presented in FIGS. 10 and 11. Each strain of Bacillus bacterium listed in FIGS. 10 and 11 was first classified by its SspE proteotype, and then further classified by its sspE genotype which is possible only if a single SspE proteotype is encoded by several different nucleotide sequences. Each of the strains listed in FIGS. 10 and 11 was further classified by MLST analysis. Bacillus thuringiensis group bacteria (FIG. 10) were further classified using the MLST methods of Priest et al. (J Bacteriol, 2004, 186: 7959-7970), and the Bacillus subtilis group bacteria (FIG. 11) were further classified by the glpF, ilvD, pta, purH, pycA, rpoD and tpiA-based MLST methods described in greater detail below.

In certain embodiments, the methods may be employed to classify a Bacillus bacterium of unknown identity (e.g., an unclassified Bacillus bacterium) or a Bacillus bacterium whose identity is not certain. In other embodiments, the methods may be employed to confirm the identity of a Bacillus bacterium of known (e.g., presumed) identity. In particular embodiments and as will be described in greater detail below, the Bacillus bacterium may a Bacillus thuringiensis group bacterium or a Bacillus subtilis/licheniformis group bacterium.

In particular embodiments, a group into which a Bacillus bacterium is classified may be associated with a particular utility (e.g., production of a particular protein or group of proteins, anti-insecticidal or anti-fungal activity, etc.) or status (e.g., GRAS status). As such, in certain embodiments, the classification of a Bacillus bacterium may indicate a use for that bacterium, where the use is associated with its classification. In other embodiments, the classification of a Bacillus bacterium may indicate that the Bacillus bacterium has GRAS status.

In particular embodiments, a method comprising: a) classifying a Bacillus bacterium using a subject SspE-based classification method; and b) employing the Bacillus bacterium in a method indicated by the classification, is provided. Exemplary uses are described in greater detail below.

Also provided are a variety of computer-related embodiments. Specifically, the instant methods may be performed using a computer. Accordingly, also provided is a computer readable medium containing computer-readable instructions for performing the instant methods. In particular embodiments, the computer-readable medium may also contain a database of sspE nucleotide and/or amino acid sequences (e.g., including any one or more of the sspE sequences in the sequence listing) or a database of SspE classifying amino acid signatures, for example. The instructions may contain instructions for comparing sequences, e.g., may contain BLAST or ClUSTALW algorithms, or instructions for identifying patterns (e.g., amino acid signatures) in sequences. The computer readable medium may also contain instructions for analyzing MLST data. In one embodiment, the computer readable medium may also contain a database of MLST sequences (including any one of more of the MLST sequences in the sequences listing). In one embodiment, the instructions may be configured to receive sequence information, e.g., SspE and/or MLST information, as an input, and configured to provide a classification, e.g., a name or an identifier, as an output.

A set of oligonucleotide primers that can detect one or more classifying SspE amino acid signatures is also provided. Such SspE classifying primers may be designed so that when they are employed in a polymerase chain reaction using the genome of a Bacillus bacterium as a template to produce reaction products, the reaction products (e.g., the presence or absence of, or the sizes of the reaction products) classify the Bacillus bacterium. Given the sspE nucleotide and amino acid sequences in the sequence listing and the amino acid/nucleic acid signatures described above, such primers would be readily designable by one of skill in the art. In certain cases, a set of primers may contain 3, 4, 5, 6, 7, 8, 9, 10 or more primer pairs of a suitable length, e.g., 15-30 nucleotides, and the 3′ end of each primer of the set may hybridize with a diagnostic nucleotide in the sspE nucleotide sequence.

In certain embodiments, a subject oligonucleotide primer set may be employed in multiplex PCR reactions to identify SspE amino acid signature. Methods for performing multiplex PCR are known (see, e.g., Kim et al FEMS Immunol. Med. Microbiol. 2005 43:301-10; Elnifro, et al. Clinical Microbiology Reviews 2000 13: 559, Hidding and Schmitt, Forensic Sci. Int., 2000 113: 47; Bauer et al., Int. J. Legal Med. 2002 116: 39; Ouhibi, et al., Curr Womens Health Rep. 2001 1: 138; Rudi et al., Int J Food Microbiology 2002 78: 171 and Zarlenga and Higgins, Vet Parasitol. 2001 101: 215, among others), and may be readily adapted to the instant methods.

The subject SspE classifying primers found in kit, which, in certain cases may contain other components for polymerase chain reaction, including, but not limited to, nucleotides, buffer, and thermostable polymerase. In certain cases may also contain isolated Bacillus bacterium genomic DNA that may be employed as a control.

A composition comprising a re-classified isolate of Bacillus bacterium selected from the following table, used in accordance with its new classification, is also provided. Depending on the indicated use of the re-classified Bacillus bacterium, the composition may be formulated for application to, e.g., spraying onto, a plant, e.g., may contain a surfactant, to provide protection against a plant pathogen, e.g., a dipteran, lepidopteran, coleopteran, nematode or fungal pathogen or as a herbicide enhancer. In other embodiments, the re-classified Bacillus bacterium may be employed to produce a particular protein, such as, for example, so called “industrial enzymes” (such as in one embodiment, the secreted region may be an enzyme such as a carbohydrase, a protease, a lipase or esterase, an oxidoreductases, for example) a therapeutic protein, food additive or foodstuffs and the like. For example, the re-classified Bacillus bacterium may contain a recombinant nucleic acid for the production of that protein, or the Bacillus bacterium may be present in a fermentor, for example. In other exemplary embodiments, a re-classified Bacillus bacterium may be formulated as drain opener, cleaner or sanitizer. In another embodiment, a gene from a re-classified Bacillus bacterium may be cloned and employed as anti-insecticidal or anti-fungal agent, for example. The re-classified bacterium may be a previously unclassified Bacillus bacterium, or a mis-classified Bacillus bacterium, for example. Tables 11 and 12, entitled “Bacillus thuringiensis Group—reclassified” and “Bacillus subtilis Group—reclassified”, respectively, indicate several re-classified strains Bacillus bacteria, and the utility associated with their new classification.

TABLE 11
Bacillus thuringiensis Group-reclassified:

| Classifier | New Utility | |
|---|---|---|
| | | Source/Strain name |
| A1a | Insecticidal activity against order Diptera | BGSC 4G3, BGSC 4G5, BGSC 4I1, BGSC |
| | | 4I2, IB/A |
| A1d | Insecticidal activity against order Lepidoptera; | BGSC 4T1, ATCC 29730 |
| | Anti-helminthic, nematicide | |
| A1e | Anti-helminthic, nematicide | BGSC 6A1, BGSC 6A2 |
| A1f | Anti-helminthic, nematicide | ATCC 11778 |
| A1g | Anti-cancer activity; Anti-helminthic, | NRRL B-21619 |
| | nematicide | |
| A1g | Plant protection; Anti-helminthic, nematicide | BGSC 4R1 |
| A1g | Anti-cancer activity; Plant protection; Anti- | BGSC 4BQ1 |
| | helminthic, nematicide | |
| A1h | Anti-helminthic, nematicide | BGSC 4BF1 |
| A1i | Anti-helminthic, nematicide | BGSC 4AL1 |
| A1j | Anti-helminthic, nematicide | BGSC 4CA1 |
| A1k | Anti-helminthic, nematicide | BGSC 4S2, BGSC 4S3 |
| A1l | Anti-helminthic, nematicide | BGSC 4AR1 |
| A1m | Anti-helminthic, nematicide | BGSC 4AT1 |
| F1a | Insecticidal activity against order Diptera | BGSC 4AO1 |
| F2a | Insecticidal activity against order Coleoptera | BGSC 6A3, BGSC 6A4, BGSC 4BU1, ATCC |
| | | 27348, NRRL B-571 |
| H2a | Anti-cancer activity | BGSC 4AE1 |
| H2b | Insecticidal activity against orders Diptera & | BGSC 4AF1 |
| | Lepidoptera; Anti-cancer activity | |
| H2c | Insecticidal activity against order Lepidoptera; | BGSC 4U1 |
| | Anti-cancer activity | |
| H2d | Insecticidal activity against order Lepidoptera | BGSC 4BE1 |
| H2e | Insecticidal activity against order Lepidoptera | BGSC 4AN1 |
| H2f | Insecticidal activity against orders Diptera & | BGSC 4AQ1 |
| | Lepidoptera; Anti-cancer activity | |
| H2g | Insecticidal activity against orders Diptera & | Pey. 6 |
| | Lepidoptera; Anti-cancer activity | |
| H3a | Insecticidal activity against orders Diptera & | ATCC 53522, ATCC 55609 |
| | Lepidoptera | |
| H3b | Insecticidal activity against orders Diptera & | BGSC 4AG1 |
| | Lepidoptera; Crop protection | |
| H3c | Insecticidal activity against order Lepidoptera; | BGSC 4V1 |
| | Crop protection | |
| H3d | Insecticidal activity against order Diptera; Crop | BGSC 4Z1 |
| | protection | |
| H4a | Insecticidal activity against orders Coleoptera | 4D3 |
| | & Isoptera; Crop protection | |
| H4a | Insecticidal activity against order Isoptera; | 4A1, 4A2, 4A3, 4A4, 4A5, 4A6, 4A7, 4A8, |
| | Crop protection | DSM 2046^{T} |
| H4b | Insecticidal activity against orders Coleoptera, | ATCC 55000 |
| | Diptera, Lepidoptera & Isoptera | |
| H4c | Insecticidal activity against orders Coleoptera, | BGSC 4BB1 |
| | Diptera & Lepidoptera; Crop protection | |
| H4d | Insecticidal activity against orders Coleoptera, | BGSC 4BP1 |
| | Diptera, Lepidoptera & Isoptera; Crop | |
| | protection | |
| H4e | Insecticidal activity against order Isoptera; | BGSC 4A9 |
| | Crop protection | |
| H5a | Insecticidal activity against orders Diptera & | BGSC 4BS1 |
| | Lepidoptera; Anti-helminthic, nematicide | |
| H5b | Insecticidal activity against order Diptera; Anti- | BGSC 4AV1, BGSC 18A1 |
| | helminthic, nematicide | |
| | | Commercial/Insecticidal Utility |
| H5b | Anti-helminthic, nematicide | BGSC 4Q1, BGSC 4Q2, BGSC 4Q3, BGSC |
| | | 4Q4, BGSC 4Q5, BGSC 4Q6, BGSC 4Q7, |
| | | BGSC 4Q8, ATCC 35646 |
| H5c | Insecticidal activity against order Coleoptera | BGSC 4O1 |
| H5d | Anti-helminthic, nematicide | BGSC 4M1, BGSC 4M2, BGSC 4M3 |
| H5e | Anti-helminthic, nematicide | BGSC 4AK1 |
| H5g | Anti-helminthic, nematicide | BGSC 4BR1 |
| H5h | Anti-helminthic, nematicide | BGSC 4BZ1 |
| | | Source/Strain name |
| E1a | Crop protection e.g. herbicide enhancement; | BGSC 6A6, ATCC 15816, |
| | Medical & veterinary diagnostic | |
| E1b | Crop protection e.g. herbicide enhancement; | BGSC 4H1, ATCC 13061 |
| | Medical & veterinary diagnostic | |
| E1c | Medical & veterinary diagnostic | ATCC 55675 |
| E1d | Crop protection e.g. herbicide enhancement; | BGSC 6A9 |
| | Medical & veterinary diagnostic | |
| E2a | Medical & veterinary diagnostic | BGSC 4B1, BGSC 4B2 |
| E2b | Medical & veterinary diagnostic | ATCC 51912 |
| E3a | Medical & veterinary diagnostic | BGSC 4AH1 |
| E4a | Medical & veterinary diagnostic | DM55 |
| E4b | Medical & veterinary diagnostic | BGSC 6E1, BGSC 6E2 |
| E4c | Medical & veterinary diagnostic | 003, III, IB, IV, III-BL, III-BS, BuIB |
| E4d | Medical & veterinary diagnostic | S8553/2 |
| E5a | Medical & veterinary diagnostic | BGSC 4CD1 |
| E6a | Medical & veterinary diagnostic | BGSC 4BH1 |
| E7a | Medical & veterinary diagnostic | BGSC 4Y1 |
| E8a | Insecticidal activity against order Isoptera; | ATCC 4342 |
| | Medical & veterinary diagnostic | |
| E8b | Medical & veterinary diagnostic | BGSC 4BG1 |
| E9a | Medical & veterinary diagnostic | ATCC 10987 |
| E10a | Veterinary diagnostic | Strain G9241 |
| E11a | Medical diagnostic | Strain ZK (E33L) |
| K1a | Medical diagnostic | BGSC 4BC1 |
| K2a | Medical diagnostic | BGSC 4AY1 |
| K2b | Medical diagnostic | BGSC 4CC1 |
| K2c | Medical diagnostic | BGSC 4BA1 |
| K2e | Insecticidal activity against order Diptera; | BGSC 4AS1 |
| | Medical diagnostic | |
| K2e | Medical diagnostic | BGSC 4AU1 |
| K2f | Medical diagnostic | BGSC 4BY1 |
| K2g | Medical diagnostic | BGSC 4CB1 |
| K3a | Medical diagnostic | BGSC 4BJ1, BGSC 4BX1 |
| K3b | Medical diagnostic | BGSC 4BV1 |
| K3c | Medical diagnostic | BGSC 4BK1 |
| P1a | Medical & veterinary diagnostic | B. anthracis Western North America |
| | | USA6153 |

TABLE 12
Bacillus subtilis Group - reclassified:

| | | Source/Strain |
|---|---|---|
| Classifier | New Utility | Names |
| 1a | Biofungicide, drain opener, cleaner & sanitizer | DSM 5552 |
| 2a | Produces enzymes such as proteases, amylases, cellulases and | BGSC 1A1, BGSC |
| | lipases; purine nucleotides and nucleosides; D-ribose; lipopeptide | 1A3, BGSC 1A96, |
| | antibiotics; and the vitamin riboflavin | BGSC 1A747, BGSC |
| | | 3A1, BGSC 10A1, |
| | | RS2, RS1725, SB1058, |
| | | WB746, 3610, ATCC |
| | | 6051, DSM 10, |
| | | DSM4424 |
| 2b | Produces enzymes such as proteases, amylases, cellulases and | DSM 5660 |
| | lipases; purine nucleotides and nucleosides; D-ribose; lipopeptide | |
| | antibiotics; and the vitamin riboflavin | |
| 2c | Produces enzymes such as proteases, amylases, cellulases and | BGSC 27E1, ATCC |
| | lipases; purine nucleotides and nucleosides; D-ribose; lipopeptide | 7058, ATCC 15245, |
| | antibiotics; and the vitamin riboflavin | DSM 1088, DSM 4449, |
| | | DSM 4450, DSM 4451 |
| 2d | Produces enzymes such as proteases, amylases, cellulases and | DSM 1092 |
| | lipases; purine nucleotides and nucleosides; D-ribose; lipopeptide | |
| | antibiotics; and the vitamin riboflavin | |
| 2e | Produces enzymes such as proteases, amylases, cellulases and | ATCC 7059 |
| | lipases; purine nucleotides and nucleosides; D-ribose; lipopeptide | |
| | antibiotics; and the vitamin riboflavin | |
| 2f | Produces enzymes such as proteases, amylases, cellulases and | DSM 3257 |
| | lipases; purine nucleotides and nucleosides; D-ribose; lipopeptide | |
| | antibiotics; and the vitamin riboflavin | |
| 2g | Produces enzymes such as proteases, amylases, cellulases and | BGSC 3A18, BGSC |
| | lipases; purine nucleotides and nucleosides; D-ribose; lipopeptide | 3A19 |
| | antibiotics; and the vitamin riboflavin | |
| 2h | Produces enzymes such as proteases, amylases, cellulases and | BGSC 1A308, BGSC |
| | lipases; purine nucleotides and nucleosides; D-ribose; lipopeptide | 1A757, W168, NRRL |
| | antibiotics; and the vitamin riboflavin | B-642 |
| 2i | Produces enzymes such as proteases, amylases, cellulases and | BGSC 2A10 |
| | lipases; purine nucleotides and nucleosides; D-ribose; lipopeptide | |
| | antibiotics; and the vitamin riboflavin | |
| 2j | Produces enzymes such as proteases, amylases, cellulases and | BGSC 10A5T |
| | lipases; purine nucleotides and nucleosides; D-ribose; lipopeptide | |
| | antibiotics; and the vitamin riboflavin | |
| 6a | Produces enzymes such as alkaline proteases and amylases; | BGSC 5A1, BGSC |
| | produces α-acetolactate decarboxylase, amylase (thermostable), | 5A2, ATCC 11946, |
| | penicillinase, 2,3-butanediol and glycerol | MO1 |
| 6b | Produces enzymes such as alkaline proteases and amylases; | BGSC 5A13, BGSC |
| | produces α-acetolactate decarboxylase, amylase (thermostable), | 5A20, BGSC 5A21 |
| | penicillinase, 2,3-butanediol and glycerol | |
| 6c | Produces enzymes such as alkaline proteases and amylases; | BGSC 5A32, BGSC |
| | produces α-acetolactate decarboxylase, amylase (thermostable), | 5A36, ATCC 14580, |
| | penicillinase, 2,3-butanediol and glycerol | ATCC 6598 |
| 6e | Produces enzymes such as alkaline proteases and amylases; | NRRL B-23318 |
| | produces α-acetolactate decarboxylase, amylase (thermostable), | |
| | penicillinase, 2,3-butanediol and glycerol | |
| 6f | Produces enzymes such as alkaline proteases and amylases; | NRRL B-23325 |
| | produces α-acetolactate decarboxylase, amylase (thermostable), | |
| | penicillinase, 2,3-butanediol and glycerol | |
| 7b | Amino acid production; for example, produces the food additive 5- | NRRL B-23154-T, |
| | hydroxytryptophan | NRRL B-23160 |
| 7c | Amino acid production; for example, produces the food additive 5- | NRRL B-23157 |
| | hydroxytryptophan | |
| 7d | Amino acid production; for example, produces the food additive 5- | NRRL B-23155 |
| | hydroxytryptophan | |
| 7e | Amino acid production; for example, produces the food additive 5- | NRRL B-23158, NRRL |
| | hydroxytryptophan | B-23159, DSM 13780 |
| 7f | Amino acid production; for example, produces the food additive 5- | NRRL B-23161 |
| | hydroxytryptophan | |
| 8a | Biofungicide; antifungal activity; Produces antibiotics against & | DSM 1324 |
| | inhibits growth of certain plant pathogenic fungi & bacteria; drain | |
| | opener, cleaner & sanitizer; produces enzymes such as amylase & | |
| | inhibitors for glycoside hydrolases | |
| 8b | Produces antibiotics against & inhibits growth of certain plant | GB03 (Companion) |
| | pathogenic bacteria; produces enzymes such as amylase & | |
| | inhibitors for glycoside hydrolases | |
| 8c | Produces antibiotics against & inhibits growth of certain plant | DSM 8563, DSM 8564, |
| | pathogenic bacteria; produces enzymes such as amylase & | DSM 8565, BGSC |
| | inhibitors for glycoside hydrolases | 10A6 |
| 9a | Produces antibiotics against & inhibits growth of certain plant | NRRL B-21661 |
| | pathogenic bacteria; produces enzymes such as amylase & | |
| | inhibitors for glycoside hydrolases | |
| 10a | Produces enzymes such as amylase & inhibitors for glycoside | ATCC 55614 |
| | hydrolases; drain opener, cleaner & sanitizer | |
| 11a | Produces antibiotics against & inhibits growth of certain plant | DSM 7, BGSC 3A14 |
| | pathogenic fungi & bacteria | |
| 11b | Biofungicide; antifungal activity; Produces antibiotics against & | DSM 1060, ATCC |
| | inhibits growth of certain plant pathogenic fungi & bacteria | 55405, ATCC 55407 |
| 11c | Biofungicide; antifungal activity; Produces antibiotics against & | BGSC 3A23 |
| | inhibits growth of certain plant pathogenic fungi & bacteria; drain | |
| | opener, cleaner & sanitizer; produces enzymes such as amylase & | |
| | inhibitors for glycoside hydrolases | |
| 19a | Probiotic health supplement | DSM 355 |
| 19b | Probiotic health supplement | BGSC 8A1 |
| 19c | Probiotic health supplement | ATCC 27142 |
| 21a | Probiotic health supplement | DSM 354 |

As noted above, SspE-based methods for classifying a Bacillus bacterium are provided. After a general introduction to these SspE-based methods, SspE-based methods for classifying isolates from a) the Bacillus thuringiensis group and b) the Bacillus subtilis/licheniformis group, are discussed in more detail. Also as will be described in greater detail below, the methods may further include MLST analysis.

The following abbreviations will be used throughout this disclosure: Bc=Bacillus cereus, Bt=Bacillus thuringiensis, Ba=Bacillus anthracis, Bm=Bacillus mycoides, Bp=Bacillus pseudomycoides, Bw=Bacillus weihenstephanensis, Bs=Bacillus subtilis, Bat=Bacillus atrophaeus, Bmo=Bacillus mojavensis, Bv=Bacillus vallismortis, Bl=Bacillus licheniformis, Bson=Bacillus sonorensis, Bamy=Bacillus amyloliquefaciens, Bpum=Bacillus pumilus, Bsp=Bacillus species; n/d=not determined; ^{T}=Type strain, MLST=multilocus sequence typing, ST=MLST sequence type, sspE=the (nucleotide sequence of the) gene encoding gamma-type small acid soluble spore protein, SspE=the (translated amino acid sequence of the) gene encoding gamma-type small acid soluble spore protein. Greek letters used: α=alpha, β=beta, γ=gamma, δ=delta. The capital Greek letter delta (Δ) is used to represent a nucleotide or amino acid residue deletion in a sequence.

The sspE gene reliably reconstructs the natural genetic history of Bacillus strains at the species and subspecies or serovar level, and thus a single-gene method for the detection of assays for identification of commercially valuable Bacillus isolates. Certain embodiments provide an inexpensive, rapid and accurate method for the phylogenetic positioning of unknown isolates and can be modified for high-throughput screening. Isolates with an sspE gene sequence that places them within a clade containing strains of known commercial utility can be further parsed by MLST to determine their precise strain-level and population genetic relationships.

Certain embodiments of these methods are robust such that they can distinguish, phylogenetically stratify and cluster species, subspecies and serovars of the Bacillus thuringiensis clade, particularly insecticidal serovars of Bacillus thuringiensis (Bt) such as serovars kurstaki, israelensis, aizawai/pacificus and thuringiensis from one another as well as from Bacillus anthracis (Ba), Bacillus cereus (Bc), other non-insecticidal Bt, Bacillus mycoides (Bm), Bacillus pseudomycoides (Bp), Bacillus weihenstephanensis (Bw), and other strains of spore-forming bacteria including the Bacillus subtilis/licheniformis group.

A variety of methods have been utilized for classification of Bacillus species, subspecies, strains, serotypes and pathotypes including metabolic profiling (e.g. Biolog), fatty acid profiling (e.g. MIDI), immunotyping and DNA-based methods such as AFLP, VNTR, and ribosomal RNA analysis. However, to date, none of these assays are sufficiently robust to unambiguously discriminate amongst the aforementioned species. The Bacillus species show a high degree of genetic relatedness, and this genomic conservation has made specific discrimination within the Bacillus thuringiensis group challenging. PCR-based identification methods have utilized a number of chromosomal loci (e.g. nucleic acid metabolism genes), plasmid loci or virulence genes. Although ribosomal RNA typing is useful for coarse-grained classification, it is frequently unable to separate closely related species due to the slow rate of evolutionary divergence of these highly conserved molecules. Phenotypic or metabolic classification methods are unreliable as the traits used for discrimination are distantly related to the natural genetic history of the microorganisms of interest. AFLP is one method that had been employed for stratification of Bc group isolates and it is useful for discrimination among strains (fingerprinting) but is not capable of reconstructing the natural genetic history and population genetic relationships of strains of interest (genealogy). Many single gene chromosomal typing methods have failed to provide the desired fine-grained discrimination of closely related phylotypes due to the conservation of these genes across species and their disconnection from the ecogenetic processes that drive speciation.

The sspE gene, however, appears to have arisen and evolved within the Bacillus genus. In certain embodiments, phylogenetic analysis of sspE DNA and translated amino acid sequence have been used to reconstruct evolutionary and phylogenetic relationships of more than 380 isolates representing over a dozen species within this genus. SspE sequence information naturally stratifies and clusters isolates at bona fide species/subspecies resolution and is thus useful for species-level identification. The inventors are aware of no other assay, single-gene or otherwise, that provides such an unambiguous identification and phylogenetic positioning of a broad range of Bacillus species. Certain PCR methods described in this invention amplify the full-length gene, and in some cases flanking sequences of the gene, sspE, that is present on the chromosome of Bacillus species. This gene is useful for high-resolution genotyping as it appears to have arisen within the Bacillus genus, has a different sequence in ecologically distinct populations and has a rapid rate of sequence evolution that provides fine-grained phylogenetic discrimination.

Thus, certain embodiments of the present invention involve a tiered screening method to identify potential Bacillus microbial species of commercial importance by SspE (for example) proteotype analysis, followed by sspE (for example) genotype analysis and finally allelic typing by a method such as MLST. This approach to microbial identification has a high level of robustness and phylogenetic clustering power.

Certain embodiments of the methods include multilocus sequence typing (MLST), where multilocus sequence typing is a rapidly developing technology that infers phylogenies based on DNA sequence fragments from more than one gene, e.g., two, three, four, five, six, seven, eight or more than eight genes. Some MLST schemes have been described in the literature (Baker et al., 2004; Hanage et al., 2005; Maiden et al., 1998; McGregor et al., 2005; Priest et al., 2004; Spratt, 1999). Multiple genes (typically housekeeping) are sequenced and their sequences are concatenated for each isolate. Genes are identified as suitable for an MLST analysis scheme if they are present across the population of interest, evolve slowly (e.g., so called “slow-clock” genes such as housekeeping genes), are unlikely to be susceptible to recombination and have a continuous coding region (˜500 bp) containing a significant number of informative polymorphic sites, but no insertions or deletions. The incidence of polymorphic sites can't be too great because primers may be designed that can amplify the exact same region from a wide range of isolates. Furthermore, the genes should be dispersed in regions of the chromosome that would minimize the probability of co-inheritance or linkage with any of the other loci being studied with MLST. Typically, an internal fragment of the gene is used, rather than the whole gene or intergenic regions, and these fragments usually are 350-550 nucleotides in length. For each strain, the region of the allele analyzed must be identical and in coding frame. Each unique allele sequence (for each gene) is assigned an allele number 1−∞. These numbers are assigned at random by the researcher developing the scheme, and the DNA sequences corresponding to each allele number are stored in a database. Here, we describe two different 7-gene MLST schemes, one for the B. subtilis/licheniformis group, and another for the B. cereus group which is available at pubmlst.org/bcereus. Each of seven gene fragments was amplified and sequenced for all isolates, thus each isolate was assigned seven numbers (an allelic profile), corresponding to DNA sequences from fragments of seven housekeeping genes. The numbers in the allelic profile for each isolate must be in the same order to maintain consistency in the definition and interpretation of the allelic profile. The convention for allele concatenation order is usually alphabetical by locus name, which is what was used here. Each unique allelic profile is designated as a unique sequence type (ST), which is an eighth number randomly assigned 1−∞. Thus, the terms “allelic profile” and “ST” are related since they both describe the seven DNA sequence fragments of a particular isolate, though “allelic profile” refers to the seven allele numbers in alphabetical order and “ST” refers to one number that is assigned to each unique allelic profile.

Example 1: B. cereus strain T, which is designated as our holotype reference for the Bc SspE group, has been assigned to ST 29 in the Bc group MLST scheme. This corresponds to the allelic profile 20, 8, 8, 35, 8, 17, 17 and thus B. cereus strain T has allele (DNA) sequences that correspond to the glp-20, gmk-8, ilv-8, pta-35, pur-8, pyc-17 and tpi-17 allele sequences deposited in the pubmlst.org/bcereus database. Bc group ST 29 has a concatenated sequence length of 2829 bp: Example 2: B. subtilis strain W23, which is designated as our holotype reference for Bs SspE group, has been assigned to ST 7 in the Bs group MLST scheme. This corresponds to the allelic profile 9, 4, 6, 7, 5, 4, 5 and thus B. subtilis strain W23 has allele (DNA) sequences that correspond to the glp-9, ilv-4, pta-6, pur-7, pyc-5, rpo-4 and tpi-5 allele sequences. Bs group ST 7 has a concatenated sequence length of 371 1 * bp. Example 3: ST 1 in the Bc group MLST scheme corresponds to the allelic profile 1, 1, 1, 1, 1, 1, 1 and therefore has allele (DNA) sequences that correspond to the glp-1, gmk-1, ilv-1, pta-1, pur-1, pyc-1 and tpi-1 allele sequences in the pubmlst.org/bcereus database. Bc group ST 1 has a concatenated sequence length of 2829 bp and includes members such as B. anthracis Ames strain. Similarly, but with a completely different phylogenetic and taxonomic meaning, ST 1 in the Bs group MLST scheme corresponds to the allelic profile 1, 1, 1, 1, 1, 1, 1 and therefore has allele (DNA) sequences that correspond to the glp-1, ilv-1, pta-1, pur-1, pyc-1, rpo-1 and tpi-1 allele sequences. Bs group ST 1 has a concatenated sequence length of 3711 * bp and includes members such as B. subtilis laboratory strain 168. *note: we will be shortening all of the alleles in the Bs MLST scheme by 120 bp for the public database, thus the concatenated sequence will be 2871 bp.

MLST phylogenetic trees are created when all in-frame allele fragments of a particular isolate are merged [alphabetical] making one continuous concatenated DNA sequence which is multi-sequence aligned with similar ordered concatenations from other isolates and analyzed by a computer algorithm (ex., MEGA, PAUP or PHYML) that creates a phylogenetic tree. In some cases, a program called START is used and UPGMA trees are created from allelic profiles (with a separately uploaded file of numbered allele sequences for each locus) rather than concatenated sequences, but these trees are not as reliable as those that use the more information rich concatenated DNA sequence alignments.

### Part I

### Methods for the Classification of the Bacillus Thuringiensis Group Bacteria

Abbreviations: Bc=Bacillus cereus, Bt=Bacillus thuringiensis, Ba=Bacillus anthracis, Bm=Bacillus mycoides, Bp=Bacillus pseudomycoides, Bw=Bacillus weihenstephanensis, ^{T}=Type strain, MLST=multilocus sequence typing, ST=MLST sequence type, sspE=the (nucleotide sequence of the) gene encoding gamma-type small acid soluble spore protein, SspE=the (translated amino acid sequence of the) gene encoding gamma-type small acid soluble spore protein. Greek letters used: α=alpha, β=beta, γ=gamma, δ=delta. The capital Greek letter delta (Δ) is used to represent a nucleotide or amino acid residue deletion in a sequence.

The Bacillus thuringiensis group scheme: The Bt clade contains the Bc, Ba, Bt, Bm, Bp and Bw species. Bt isolates are further subdivided based upon their antigenic character into serotypes or serovars, while plasmid-encoded virulence factors, genes encoding enterotoxins or pathogenesis genes are methods used to distinguish Ba and Bc species. The Bt isolate nomenclature convention is that the serotype is a number and the serovar is a name e.g. Bt serovar. thuringiensis is serotype 1 and Bt serovar. kurstaki is serotype 3a, 3b, 3c. Generally, the serovar name, which is sometimes also referred to as “subspecies,” is directly interchangeable with the serotype number(s), though there are many cases where a Bt strain will react with multiple antisera. Usually in these cases multiple serotype numbers are used to describe the isolates, yet they cannot be assigned to any one serovar. The remaining species (Bm, Bp and Bw) are characterized by classic morphological, biochemical and microbiological assays. Thus, reliance on plasmid-encoded and horizontally transmitted traits is prevalent in Bc group taxonomy and could lead to misidentification of chromosomal lineages. The sspE gene, as well as all of the MLST loci employed here, are located on the Bacillus chromosome. sspE sequences from the Bacillus thuringiensis group isolates examined in this study have been deposited in the GenBank nucleotide sequence database with accession numbers AF359764-AF359821, AF359823-AF359843, AF359845, AF359847-AF359860, AF359862-AF359934, AF359936-AF359938 and DQ146892-146926. sspE nucleotide sequences for B. cereus strains ZK and G9241 were obtained from GenBank (www.ncbi.nlm.nih.gov/) and have accession numbers CP000001 and NZ_AAEK01000015, respectively. sspE nucleotide sequences for B. anthracis strains Ames and A2084 were obtained from GenBank and have accession numbers AE017025 and AE017334, respectively. sspE nucleotide sequences for B. anthracis strains A2012, A1055, Vollum, CNEVA-9066, Kruger B, Western North America USA6153 and Australia94 were obtained from TIGR (www.tigr.org/).

In addition to sspE phylogenetic analysis, more than 250 Bc group isolates were analyzed by a multilocus sequence typing (MLST) scheme detailed at pubmlst.org/bcereus/ and developed and described by F G Priest et al., J Bacteriol, 2004, 186(23), 7959-7970 [Database citation: “This publication made use of the Bacillus cereus Multi Locus Sequence Typing website (pubmlst.org/bcereus/) developed by Keith Jolley and sited at the University of Oxford (Jolley et al. 2004, BMC Bioinformatics, 5:86)]. MLST has particular utility for fine-grained subspecies and clonal type discrimination. MLST has been used to study the population biology of many pathogenic microbial groups. DNA sequences for MLST analysis were determined, with the exception of the two Bc strains and nine Ba strains mentioned above, which were obtained from their respective public databases (GenBank or TIGR). A significant problem with MLST, particularly for the Bc group, is that, when taken alone (and there are at least three MLST schemes published for Bc), the resolution is too fine, such that species- and in some cases subspecies-level discriminations are difficult, if not impossible to identify or define. In fact, Priest et al. concluded from their data that Bacillus cereus, thuringiensis and anthracis were not [chromosomally] distinct species.

Thus, the combination of sspE data, or data from any phylogenetically informative gene like sspE, with MLST data, whether the MLST data comes from the pubmlst.org scheme or any other, and whether the MLST data is from 3 or 4 or 7 (as we show here) or 11 or 20 genes. The orthogonal combination of these sspE and MLST methods provides a powerful means for identifying ecologically distinct bacterial populations of commercial importance. Certain embodiments of this method can be thought of as a digital identifier, similar to a zip code, for Bacillus, where sspE, or a gene with similar resolving capability, is the equivalent of a coarse species-level discriminator. Genotyping by MLST, or other comparable multi-gene schemes, provides fine-grained discriminatory power—but cannot be properly scaled beyond the infraspecies level without reference to sspE interspecies data. The classifiers listed in Tables 1 and 4 are essentially an abbreviated microbial digital identifier that specifies species, subspecies, and even strain or serotype. It is “abbreviated” because each unique allelic profile from seven genes is assigned one number designating it as a sequence type (ST), and the genes for each allelic profile are arranged in alphabetical order, rather than an order that corresponds to a digital address.

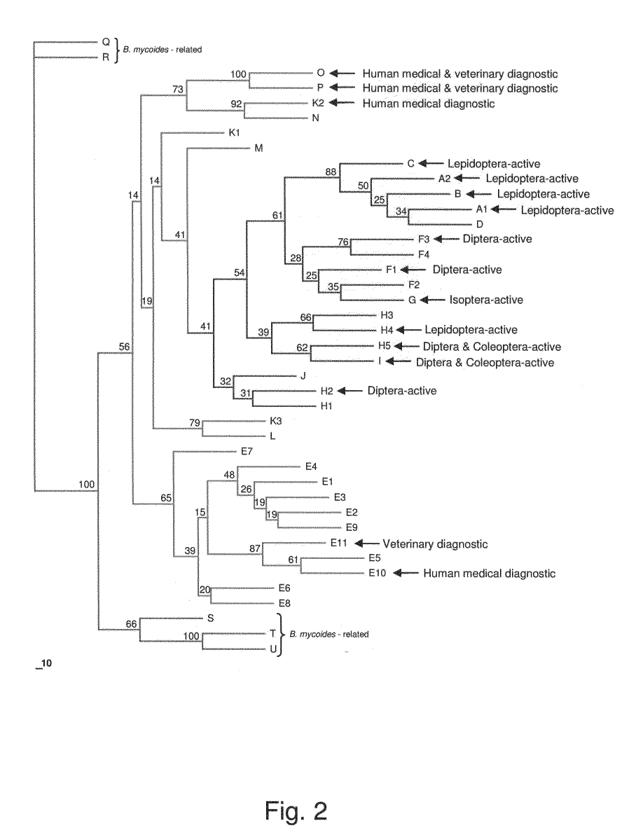

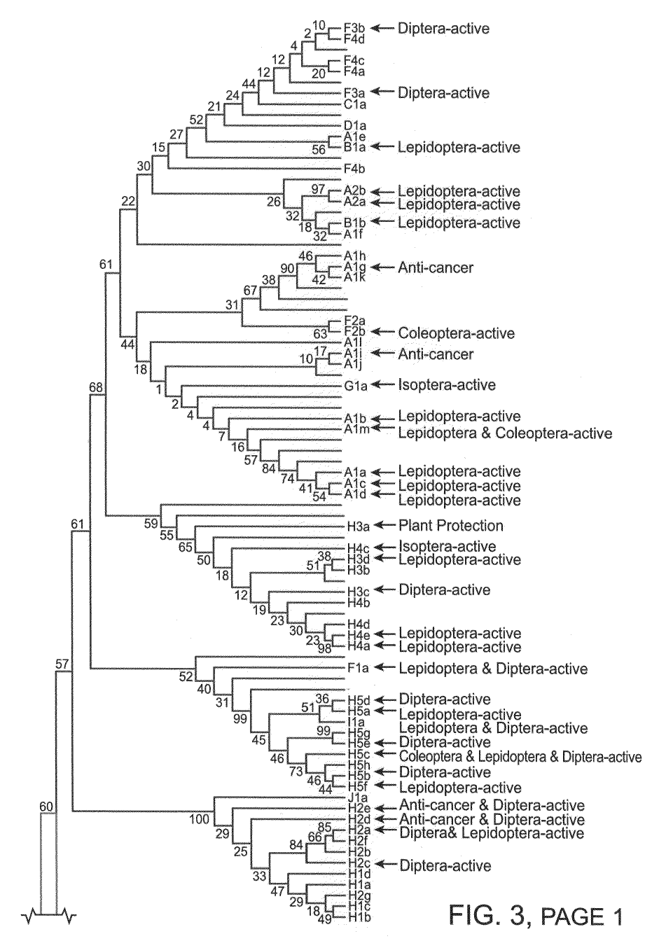

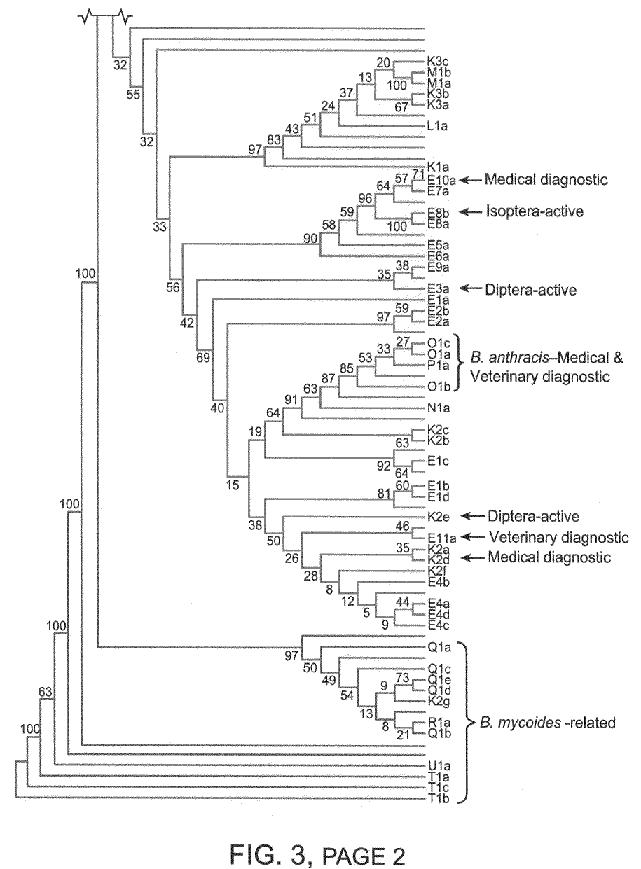

By color-coding the trees and tables, we illustrate the congruence of sspE and MLST phylogenetic clustering. We show in the following Tables 1-4 and FIGS. 2 and 3 that orthogonal MLST analysis maintains the species and subspecies phylogenetic separation provided by the sspE method and provides additional complementary resolution of subspecies and strain clusters. The complementarities and combined phylogenetic resolving power of these two methodologies are unexpected and highly useful for classification of known and unknown strains of this commercially important group of microorganisms. Classifiers (digital identifiers) in the tables and branches on the phylogenetic trees are color-coded to illustrate the equivalence of phylogenies from one scheme to another i.e. to validate sspE as a robust single-gene molecular chronometer for the Bacillus genus. Classifiers and branches remain consistent in that a strain in the sspE tree or strain table will not be in a different group for MLST STs, tree branches, or overall classifier, and vice versa. Specifically, in this study of more than 250 Bt group isolates, an ST that appeared in more than one sspE genotype or proteotype was never found, with the exception of B. anthracis Western NA which contained a SNP that altered one SspE amino acid residue with respect to all of the other Ba strains analyzed, though it maintained an allelic profile identical to that of the Ames strain of Ba.

### EXAMPLES OF PHYLOGENETIC ClUSTERING OF INSECTICIDAL BT SEROVARS

### Example 1

Insecticidally-active serovars of Bt that have established commercial value and importance cluster in the blue regions of the figures and strain tables. 19 isolates identified as serovar kurstaki were assayed and all of them clustered in sspE proteotype A and genotype A1 (see Tables 1-4 and FIG. 2). Of these, 18 kurstaki isolates had a unique MLST allelic profile corresponding to sequence type (ST) 8, and thus the unique classifier A1a was assigned to these isolates (see Table 1 and FIG. 3). Three other isolates, one strain of serovar galleriae and two of serovar entomocidus/subtoxicus, cluster in A1a, and though these serovars are not currently used as commercial insecticides, they have been observed to have (or produce Cry proteins that have) insecticidal activity against Lepidopteran larvae. Serovar kurstaki has a well documented toxicity to Lepidopteran larvae. Additionally, five kurstaki isolates from France, Iraq, Pakistan, Kenya and Australia cluster in ST8 (pubmlst.org/perl/mlstdbnet/mlstdbnet.pl?page=query&file=ba-isolates.xml), though sspE data is not available for these isolates.

There is one kurstaki outlier—it clusters in sspE genotype A1, though it is defined by ST 29, and thus the classifier A1e. This isolate is described by the culture collection from which it was obtained as “Cry-” and “no reaction with known Bt flagellar antisera.” This description could equally well describe a B. cereus strain, with which this isolate is solely clustered in A1e. Thus, it is plausible that this particular outlier was misclassified by the original investigator who isolated and deposited this strain.

### Example 2

Five isolates of Bt serovar aizawai/pacificus were assayed and also cluster in sspE proteotype A and genotype A1 (see Tables 1-4 and FIG. 2). This serovar is used in commercial insecticides that also target Lepidopteran larvae. Four of the five aizawai/pacificus isolates had a unique MLST-allelic profile corresponding to ST 15, and thus the unique classifier A1c was assigned to these isolates (see Table 1 and FIG. 3). One other isolate, a strain of serovar colmeri, clusters in A1c, and although this serovar is not currently used as a commercial insecticide, it has been observed to have (or produce Cry proteins that have) insecticidal activity against Lepidopteran and Dipteran larvae. Additionally, three aizawai isolates from France, Japan and Spain cluster in ST15 (pubmlst.org/perl/mlstdbnet/mlstdbnet.pl?page=query& file.ba-isolates.xml), though sspE data for these isolates is not available.

### Example 3

Studies have identified one aizawai outlier. As mentioned above, it clusters in sspE genotype A1, although it is defined by ST 13, and thus the classifier A1b. Two isolates of serovar kenyae, which has been shown to be insecticidal in the academic literature, also cluster in A1b. Additionally, five kenyae isolates from Iraq, Chile, Kenya and Bulgaria cluster in ST13 (pubmlst.org/perl/mlstdbnet/mlstdbnet.pl?page=query&file=ba-isolates.xml), although we do not have sspE data for these isolates. Thus, it is plausible that this particular outlier was misclassified by the original investigator who isolated and deposited this strain. Sharing an identical nucleic acid sequence for the sspE gene, Bt serovars aizawai/pacificus and kenyae are in very close phylogenetic proximity to one another, even at the strain/subspecies typing level, as is shown in FIG. 2. These results further validate the combined utility of sspE and MLST in Bacillus spp. typing.

### Example 4

Nine isolates of Bt serovar thuringiensis were assayed and cluster in sspE proteotype H and genotype H4 (see Tables 1-4 and FIG. 2). This serovar is used in commercial insecticides that also target Lepidopteran larvae. All nine thuringiensis isolates had a unique MLST allelic profile corresponding to ST 10, and thus the unique classifier H4a was assigned to these isolates (see Table 1 and FIG. 3). Additionally, five thuringiensis isolates from Canada, Bulgaria, USA, Chile and Switzerland cluster in ST10 (pubmlst.org/perl/mlstdbnet/mlstdbnet.pl?page=query&file=ba-isolates.xml), although we do not have sspE data for these isolates.

### Example 5

Nine isolates of Bt serovar israelensis were assayed and cluster within sspE proteotype H and genotype H5 (see Tables 1-3 and FIG. 2). This serovar is used in commercial insecticides that target Dipteran larvae. All nine israelensis isolates had a unique MLST allelic profile corresponding to ST 16, and thus the unique classifier H5b was assigned to these isolates (see Table 1 and FIG. 3). Additionally, one israelensis isolate from Brazil clusters in ST16 (pubmlst.org/perl/mlstdbnet/mlstdbnet.pl?page=query&file=ba-isolates.xml), although we do not have sspE data for this isolate.

Genotypic and phylogenetic placement by the combined methods of sspE and MLST provide utility in identifying Bt group strains at the species level (single gene sspE assay) that may be unrecognized insecticide candidates. Examples for genotypes A1, H4, and H5 are above, and details for all proteotypes, genotypes and classifiers are provided in the Bt group claims section that follows. MLST analysis may be also be utilized for subspecies or strain level discrimination.

An additional utility is the correct classification of microorganisms for EPA registration. For example, strain ATCC 55675 is identified and distributed by the ATCC as B. subtilis, an organism the EPA recognizes as GRAS (Generally Regarded as Safe). GRAS status for a microorganism allows easier registration, marketing and distribution, particularly in crop protection or other fields where humans or animals would come into contact with the product. Two U.S. Patents associated with ATCC 55675 (U.S. Pat. Nos. 5,650,372 & 6,232,270) describe its use for plant treatment and as a transport enhancer. We have identified this strain as a member of the B. cereus/thuringiensis group, clustering in sspE proteotype E and genotype E1 (see Tables 1-4 and FIG. 2). It has a unique MLST allelic profile and has been assigned ST 205 and unique classifier E1c (see Table 1 and FIG. 3).

Also identified are additional isolates that have been misclassified or misidentified, and they are highlighted in yellow in Table 1. For example, an isolate currently distributed by the USDA as B. licheniformis actually clusters within Bt SspE proteotype F. Also identified are isolates described as B. subtilis and B. megaterium that cluster within Bt group SspE proteotypes E and H, respectively, and a strain identified as B. mycoides (ATCC 19647) that clusters phylogenetically, both by sspE and MLST, with B. thuringiensis-related isolates, rather than with the B. mycoides and B. pseudomycoides strains analyzed. These are a few examples of cases of misidentification by culture collections or investigators and exemplify the power of the subject methods to accurately specify phylogenetic association and use.

Utility—Bacillus thuringiensis Group Scheme (See Also Table 1.)

The utility of this method embodies not only identification of Bacillus species which are of economic importance, but also genes which may be derived from these bacteria or their plasmids and which may be cloned into other bacteria, plants, etc. as well as derivatives or byproducts of substances produced by these bacteria.

1. EXEMPLARY UTILITY: Insecticidal activity against order Lepidoptera. Classifier A1d: Bacillus thuringiensis serovar galleriae (serotype 5a, 5b) has been identified as having anti-Lepidopteran^{2, 20, 29, 102} properties. 60% (3/5) of serovar galleriae (5a, 5b) isolates tested cluster within this classifier. The basis for claiming this group is the splitting of the galleriae (5a, 5b) serovar into classifier A1a; additionally, two other isolates fall into this group which have not yet been described as insecticidal: Bacillus thuringiensis serovar wuhanensis (no serotype), which lacks a flagellar antigen; and misidentified Bt strain ATCC 29730, which was deposited to the ATCC as Bt var. galleriae, but then later reclassified by the ATCC. Classifier A1d is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1d is assigned to proteotype A and genotype A1; amino acid and DNA sequences, respectively, are attached as appendices. A molecular signature for this group is an SspE translated protein sequence length of 93 amino acids, with the following residue characteristics: S at position 29, S at position 73, Q at position 87. The classifier A1d contains MLST ST 25¹⁰⁸. SspE proteotype B: Bacillus thuringiensis serovar entomocidus/subtoxicus (serotype 6) has been identified as having anti-Lepidopteran properties^{20, 31-32, 36, 41, 60, 87}. The basis for claiming this group is the splitting of the entomocidus/subtoxicus (6) serovar into classifier A1a. 60% (3/5) of serovar entomocidus/subtoxicus (6) isolates tested cluster in this classifier. Proteotype B amino acid and DNA sequences, respectively, are attached as appendices. A molecular signature for this group is SspE translated protein sequence length of 93 amino acids, with the following residue characteristics: K at position 87. Proteotype B has at least one genotype (B1) and at least two isolate STs 221 and 239¹⁰⁸.

2. EXEMPLARY UTILITY: Insecticidal activity against order Lepidoptera. EXEMPLARY UTILITY: Insecticidal activity against order Diptera. Classifier A1a: Bacillus thuringiensis serovar kurstaki (serotype 3a, 3b, 3c) has been identified as having anti-Lepidopteran^{1, 6-7, 14, 17, 20, 29, 31, 36, 41, 45, 47, 51, 55, 57-58, 80-84, 87, 92, 98} and anti-Dipteran^{36, 47, 52, 55, 58, 77, 98-99} properties; Bacillus thuringiensis serovar galleriae (5a, 5b) has been identified as having anti-Lepidopteran^{2, 20, 29, 102} properties; Bacillus thuringiensis serovar entomocidus/subtoxicus (6) has been identified as having anti-Lepidopteran^{20, 31-32, 36, 41, 60, 87} properties. The basis for claiming this group is the splitting of the galleriae (5a, 5b) and entomocidus/subtoxicus (6) serovars into classifier A1d and SspE proteotype B, respectively, as well as the occurrence of 2 kurstaki (3a, 3b, 3c) serovars that have been misidentified, falling into other classifiers [A1e (all other isolates in this classifier are “classic” laboratory Bacillus cereus strains) & H4a (all other isolates in this classifier are serotyped as Bacillus thuringiensis serovar thuringiensis (serotype 1))]. Additionally, the galleriae (5a, 5b) and entomocidus/subtoxicus (6) serovars have not been previously known to have anti-Lepidopteran activities. 91% (20/22) of serovar kurstaki (3a, 3b, 3c) isolates, 40% (2/5) of serovar galleriae (5a, 5b) isolates and 40% (2/5) of serovar entomocidus/subtoxicus (6) isolates tested cluster in this classifier. Classifier A1a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1a is assigned to proteotype A and genotype A1; amino acid and DNA sequences, respectively, are attached as appendices. A molecular signature for this group is SspE translated protein sequence length of 93 amino acids, with the following residue characteristics: S at position 29, S at position 73, Q at position 87. The classifier A1a contains ST 8¹⁰⁸. Classifier A1b: Bacillus thuringiensis serovar kenyae (serotype 4a, 4c) has been identified as having anti-Lepidopteran^{29, 31, 36, 41, 94} and anti-Dipteran⁷⁷ properties; 100% (4/4) of serovar kenyae (4a, 4c) isolates tested cluster in this classifier. The basis for claiming this group is the presence of a misidentified aizawai/pacificus (serotype 7) serovar in this classifier grouping. Classifier A1b is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1b is assigned to proteotype A and genotype A1; amino acid and DNA sequences, respectively, are attached as appendices. A molecular signature for this group is SspE translated protein sequence length of 93 amino acids, with the following residue characteristics: S at position 29, S at position 73, Q at position 87. The classifier A1b contains ST 13¹⁰⁸. Classifier A1c: Bacillus thuringiensis serovar. aizawai/pacificus (serotype 7) has been identified as having anti-Lepidopteran^{8, 20, 22, 24, 29, 31, 36, 46-47, 51, 55, 83} and anti-Dipteran^{22, 24, 47, 77, 89} activity; 80% (4/5) of serovar aizawai/pacificus (7) isolates tested cluster within this classifier. The basis for claiming this group is the presence of a misidentified aizawai/pacificus (7) serovar into classifier A1b. Included in this classifier claim is the sole serovar colmeri (serotype 21) isolate tested which has also been identified as having anti-Lepidopteran²³ and anti-Dipteran^{23, 100} properties. Classifier A1c is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1c is assigned to proteotype A and genotype A1; amino acid and DNA sequences, respectively, are attached as appendices. A molecular signature for this group is SspE translated protein sequence length of 93 amino acids, with the following residue characteristics: S at position 29, S at position 73, Q at position 87. The classifier A1c contains ST 15¹⁰⁸, Classifier H5a: Bacillus thuringiensis serovar sotto/dendrolimus (serotype 4a, 4b) has been identified as having anti-Lepidopteran^{20-21, 31, 86} and anti-Dipteran⁶⁵ activity. 50% (2/4) of serovar sotto/dendrolimus (4a, 4b) isolates tested cluster within this classifier. Bacillus thuringiensis serovar alesti (serotype 3a, 3c) has been identified as having anti-Lepidopteran^{20, 29, 92} and anti-Dipteran⁶⁵ activity. 100% (3/3) of serovar alesti (3a, 3c) isolates tested cluster in this classifier. The basis for claiming this group is the splitting of the sotto/dendrolimus (4a, 4b) serovar into classifier H5f, as well as the clustering of serovar palmanyolensis (serotype 55), which has not yet been described as insecticidal, into this classifier. Classifier H5a is SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H5a is assigned to proteotype H and genotype H5; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H5a contains ST 12¹⁰⁸. Classifier H5f: Bacillus thuringiensis serovar sotto/dendrolimus (serotype 4a, 4b) has been identified as having anti-Lepidopteran^{20-21, 31, 86} and anti-Dipteran⁶⁵ activity. 50% (2/4) of serovar sotto/dendrolimus (4a, 4b) isolates tested cluster in this classifier. The basis for claiming this group is the splitting of the sotto/dendrolimus (4a, 4b) serovar into classifier H5a. Classifier H5f is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H5f is assigned to proteotype H and genotype H5; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H5f contains ST 197¹⁰⁸.

3. EXEMPLARY UTILITY: Insecticidal activity against order Lepidoptera. EXEMPLARY UTILITY: Insecticidal activity against Diptera. EXEMPLARY UTILITY: Insecticidal activity against order Coleoptera. SspE proteotype I: Bacillus thuringiensis serovar morrisoni (serotype 8a, 8b) has been identified as having anti-Lepidopteran^{19, 21, 29, 36, 41, 67, 69}, anti-Dipteran^{18-19, 21, 51, 67, 70, 73} and anti-Coleopteran^{36, 74} activity. 25% (¼) of serovar morrisoni (8a, 8b) isolates tested cluster within this classifier. The basis for claiming this group is the splitting of a serovar morrisoni (8a, 8b) strain into SspE genotype H5 (classifier H5c). Proteotype I amino acid and DNA sequences, respectively, are attached as appendices. A molecular signature for this group is SspE translated protein sequence length of 93 amino acids, with the following residue characteristics: C at position 25, A at position 29, N at position 33, A at position 73, E at position 93. Proteotype I has at least one genotype (I1) and at least one isolate: MLST ST 257¹⁰⁸.

4. EXEMPLARY UTILITY: Insecticidal activity against order Lepidoptera. EXEMPLARY UTILITY: Insecticidal activity against Coleoptera. EXEMPLARY UTILITIES: Insecticidal activities against Diptera and Isoptera and crop protection. sspE genotype H4: Bacillus thuringiensis serovar thuringiensis (serotype 1) has been identified as having anti-Lepidopteran^{4, 20-21, 29, 31, 36, 4, 47, 83, 92}, anti-Coleopteran^{4, 36} and anti-Dipteran³⁸ activity; Bacillus thuringiensis serovar sooncheon (serotype 41) has been identified as having anti-Isopteran¹² activity; a patented strain [mis]identified as Bacillus megaterium (ATCC 55000) has been identified as having plant protection properties¹⁰⁵ such as biological control of crop fungal diseases. The basis for claiming this group is that serovar thuringiensis (1) is used widely commercially as an insecticide, yet one strain of serovar thuringiensis (1) tested differed from the major population [90% (9/10)] of thuringiensis (1) strains by a SNP in the pycA allele, thus placing it into classifier H4e; serovar thuringiensis (1) has not been previously known to have anti-Isopteran or plant protection properties; serovar sooncheon (41) has not been previously known to have anti-Lepidopteran, anti-Coleopteran, anti-Dipteran or plant protection properties; strain ATCC 55000 is misidentified as B. megaterium and has not been previously known to have insecticidal properties and serovar kim (serotype 52) has not been previously known to have insecticidal or plant protection properties. Genotype H4 is a unique SspE proteotype (primary isolate screening) and sspE genotype (secondary isolate screening) which, when used in combination, has a high level of phylogenetic clustering power. Genotype H4 is assigned to proteotype H; amino acid and DNA sequences, respectively, are attached as appendices. A molecular signature for this group is SspE translated protein sequence length of 93 amino acids, with the following residue characteristics: A at position 29, N at position 33, A at position 73, E at position 93. Genotype H4 is 282 nucleotides (nt) in length and is distinguished from other genotypes of proteotype H by the following nucleotide sequence characteristics: T at position 48, A at position 87, T at position 180, C at position 210, T at position 237, G at position 240. Genotype H4 contains at least five MLST STs: 10, 204, 229, 236, 256¹⁰⁸.

5. EXEMPLARY UTILITIES: Insecticidal activity against orders Diptera and Lepidoptera. EXEMPLARY UTILITY: plant protection (e.g. root rot) via secondary metabolites. sspE genotype H3: Bacillus thuringiensis serovar tohokuensis (serotype 17) has been identified as having anti-Dipteran⁷⁷ properties; Bacillus thuringiensis serovar ostriniae (serotype 8a, 8c) has been identified as having anti-Lepidopteran⁶⁹ properties; patented strains identified as Bacillus cereus (ATCC 53522 and ATCC 55609) have been identified as having plant protection properties¹⁰⁴ such as biological control of agricultural fungal diseases. The basis for claiming this group is that serovar tohokuensis (17) has not been previously known to have anti-Lepidopteran or plant protection properties; serovar ostriniae (8a, 8c) has not been previously known to have anti-Dipteran or plant protection properties; strains ATCC 53522 and ATCC 55609 have not been previously known to have insecticidal properties and serovar silo (serotype 26) has not been previously known to have insecticidal or plant protection properties. Genotype H3 is a unique SspE proteotype (primary isolate screening) and sspE genotype (secondary isolate screening) which, when used in combination with MLST, has a high level of phylogenetic clustering power. Genotype H3 is assigned to proteotype H; amino acid and DNA sequences, respectively, are attached as appendices. A molecular signature for this group is SspE translated protein sequence length of 93 amino acids, with the following residue characteristics: A at position 29, N at position 33, A at position 73, E at position 93. Genotype H3 is 282 nucleotides (nt) in length and is distinguished from other genotypes of proteotype H by the following nucleotide sequence characteristics: C at position 48, A at position 87, T at position 180, C at position 210, T at position 237, G at position 240. Genotype H3 contains at least four MLST STs: 206, 210, 242, 243¹⁰⁸.

6. EXEMPLARY UTILITY: Insecticidal activity against order Diptera. sspE genotype F3: Bacillus thuringiensis serovar canadensis (serotype 5a, 5c) has been identified as having anti-Dipteran^{21, 39, 73, 77} activity; Bacillus thuringiensis serovar mexicanensis (serotype 27) has also been identified as having anti-Dipteran⁷⁷ properties. The basis for claiming this group is the presence of a misidentified canadensis (5a, 5c) serovar into SspE proteotype E, which is a proteotype characteristic of bona fide Bacillus cereus strains and transitional/pathogenic Bc strains. Genotype F3 is a unique SspE proteotype (primary isolate screening) and sspE genotype (secondary isolate screening) which, when used in combination, has a high level of phylogenetic clustering power. Genotype F3 is assigned to proteotype F; amino acid and DNA sequences, respectively, are attached as appendices. A molecular signature for this group is SspE translated protein sequence length of 93 amino acids, with the following residue characteristics: A at position 29, N at position 33, A at position 73. Genotype F3 is 282 nucleotides (nt) in length and is distinguished from other genotypes of proteotype F by the following nucleotide sequence characteristics: A at position 12, A at position 81, A at position 87, T at position 180. Genotype F3 contains at least two MLST STs: 50, 224¹⁰⁸. Classifier H5b: Bacillus thuringiensis serovar israelensis (serotype 14) has been identified as having anti-Dipteran^{3, 9-11, 13, 18, 21, 34, 43-44, 49-50, 70, 78, 83, 90-93, 95, 98} activity. 100% (9/9) of serovar israelensis (14) isolates tested cluster in this classifier. The basis for claiming this group is that serovar malayensis (serotype 36) has not been previously known to have insecticidal properties as well as the presence of an unidentified strain BGSC 18A1, which has been distributed as Bacillus sp. Classifier H5b is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H5b is assigned to proteotype H and genotype H5; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H5b contains MLST ST 16¹⁰⁸. Classifier K2e: Bacillus thuringiensis serovar higo (serotype 44) has been identified as having anti-Dipteran^{35, 58, 64, 75-76, 78} activity. The basis for claiming this group is that serovar oswaldocruzi (serotype 38) clusters in this classifier and has not been previously known to have insecticidal properties. Classifier, K2e is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier K2e is assigned to proteotype K and genotype K2; amino acid and DNA sequences, respectively, are attached as appendices. A molecular signature for this group is SspE translated protein sequence, length of 93 amino acids, with the following residue characteristics: A at position 29, N at position 33, A at position 73, Q at position 80. The classifier K2e contains MLST ST 214¹⁰⁸.

7. EXEMPLARY UTILITY: Insecticidal activity against order Diptera. EXEMPLARY UTILITY: Insecticidal activity against order Lepidoptera. sspE genotype F1: Bacillus thuringiensis serovar fukuokaensis (serotype 3a, 3d, 3e) has been identified as having anti-Dipteran^{21, 39, 63, 73, 77-78, 101} and anti-Lepidopteran^{63, 96-97} activities; Bacillus thuringiensis serovar sumiyoshiensis (serotype 3a, 3d) has been identified as having anti-Lepidopteran^{36, 96-97} activity. The basis for claiming this group is that serovar sumiyoshiensis (3a, 3d) has not been previously known to have anti-Dipteran properties. Genotype F1 is a SspE proteotype (primary isolate screening) and sspE genotype (secondary isolate screening) which, when used in combination, has a high level of phylogenetic clustering power. Genotype F1 is assigned to proteotype F; amino acid and DNA sequences, respectively, are attached as appendices. A molecular signature for this group is SspE translated protein sequence length of 93 amino acids, with the following residue characteristics: A at position 29, N at position 33, A at position 73. Genotype F1 is 282 nucleotides (nt) in length and is distinguished from other genotypes of proteotype F by the following nucleotide sequence characteristics: G at position 12, G at position 81, G at position 87, C at position 180. Genotype F1 contains at least one MLST ST 213¹⁰⁸.

8. EXEMPLARY UTILITY: Insecticidal activity against order Diptera. EXEMPLARY UTILITY: Anti-cancer activity. EXEMPLARY UTILITY: Insecticidal activity against Lepidoptera. sspE genotype H2: Bacillus thuringiensis serovar amagiensis (serotype 29) has been identified as having anti-Dipteran⁷⁷ and anti-Lepidopteran³⁶ activities; Bacillus thuringiensis serovar kyushuensis (serotype 11a, 11c) has been identified as having anti-Dipteran^{21, 39, 48-50, 73, 77, 101} activity; Bacillus thuringiensis serovar neoleonensis (serotype 24a, 24b) has been identified as having anti-Dipteran^{72, 103} and anti-cancer⁶¹ activities; Bacillus thuringiensis serovar shandongiensis (serotype 22) has been identified as having anti-cancer^{53-54, 61, 66} and anti-Dipteran^{39, 77} activities. The basis for claiming this group is that serovar amagiensis (29) has not been previously known to have anti-cancer activity; serovar kyushuensis (11a, 11c) has not been previously known to have anti-Lepidopteran or anti-cancer properties; serovar neoleonensis (24a, 24b) has not been previously known to have anti-Lepidopteran properties; serovar shandongiensis (22) has not been previously known to have anti-Lepidopteran properties and serovars seoulensis (serotype 35) and cameroun (serotype 32) and natural isolate Pey6 have not been previously known to have insecticidal or anti-cancer properties. Genotype H2 is a unique SspE proteotype (primary isolate screening) and sspE genotype (secondary isolate screening) which, when used in combination, has a high level of phylogenetic clustering power. Genotype H2 is assigned to proteotype H; amino acid and DNA sequences, respectively, are attached as appendices. A molecular signature for this group is SspE translated protein sequence length of 93 amino acids, with the following residue characteristics: A at position 29, N at position 33, A at position 73, E at position 93. Genotype H2 is 282 nucleotides (nt) in length and is distinguished from other genotypes of proteotype H by the following nucleotide sequence characteristics: C at position 48, G at position 87, T at position 180, C at position 210, A at position 237, A at position 240. Genotype H2 contains at least seven MLST STs: 158, 208, 209, 227, 228, 233, 258¹⁰⁸.

9. EXEMPLARY UTILITY: Insecticidal activity against order Coleoptera. sspE genotype F2: Bacillus thuringiensis serovar kumamtoensis (serotype 18a, 18b) has been identified as having anti-Coleopteran⁷⁴ activity. The basis for claiming this group is that serovar pirenaica (serotype 57) has not been previously known to have anti-Coleopteran properties as well as the presence of misidentified strain NRRL B-571, which has been distributed by the USDA as Bacillus licheniformis. Genotype F2 is a unique SspE proteotype (primary isolate screening) and sspE genotype (secondary isolate screening) which, when used in combination, has a high level of phylogenetic clustering power. Genotype F2 is assigned to proteotype F; amino acid and DNA sequences, respectively, are attached as appendices. A molecular signature for this group is SspE translated protein sequence length of 93 amino acids, with the following residue characteristics: A at position 29, N at position 33, A at position 73. Genotype F2 is 282 nucleotides (nt) in length and is distinguished from other genotypes of proteotype F by the following nucleotide sequence characteristics: A at position 12, G at position 81, G at position 87, T at position 180. Genotype F2 contains at least two MLST STs: 33, 59¹⁰⁸.

10. EXEMPLARY UTILITY: Insecticidal activity against order Coleoptera. EXEMPLARY UTILITY: Insecticidal activity against Lepidoptera. EXEMPLARY UTILITY: Insecticidal activity against order Diptera. Classifier H5c: Bacillus thuringiensis serovar morrisoni, including biovars tenebrionis and san diego, (serotype 8a, 8b) has been identified as having anti-Coleopteran^{15, 21, 29, 36, 56-57, 74, 85}, anti-Lepidopteran^{19, 21, 29, 36, 41, 67, 69} and anti-Dipteran^{18-19, 21, 51, 67, 70, 73} activities. 75% (3/4) of serovar morrisoni (8a, 8b) isolates tested cluster in this classifier. The basis for claiming this group is that serovar thompsoni (serotype 12) has not been previously known to have anti-Coleopteran properties, though it has been described as Dipteran^{21, 59, 72-73} and Lepidopteran active, as well as the splitting of a serovar morrisoni (8a, 8b) strain into SspE proteotype I. Classifier H5c is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H5c is assigned to proteotype H and genotype H5; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H5c contains MLST ST 23¹⁰⁸.

11. EXEMPLARY UTILITY: Insecticidal activity against order Isoptera. sspE genotype E8: Bacillus thuringiensis serovar roskildiensis (serotype 45) has been identified as having anti-Isopteran¹² activity. The basis for claiming this group is the presence of a strain identified as Bacillus cereus that has' not been previously known to have insecticidal properties. Genotype E8 is a unique SspE proteotype (primary isolate screening) and sspE genotype (secondary isolate screening) which, when used in combination, has a high-level of phylogenetic clustering power. Genotype E8 is assigned to proteotype E; amino acid and DNA sequences, respectively, are attached as appendices. A molecular signature for this group is SspE translated protein sequence length of 93 amino acids, with the following residue characteristics: A at position 73, Q at position 80. Genotype E8 is 282 nucleotides (nt) in length and is distinguished from other genotypes of proteotype E by the following nucleotide sequence characteristics: G at position 30, T at position 42, G at position 102, A at position 114, C at position 123, A at position 126, G at position 138, A at position 147, Tat position 174, Tat position 180, A at position 189, Tat position 195, C at position 210, G at position 249. Genotype E8 contains at least two MLST STs: 38, 103¹⁰⁸.

12. EXEMPLARY UTILITY: Anti-cancer activity. EXEMPLARY UTILITY: Plant protection. Classifier A1g: Bacillus thuringiensis serovar dakota (serotype 15) has been identified as having anti-cancer^{40, 42, 61} activity; Bt strain NRRL B-21619 has been identified in two US patents as having broad antifungal and antibacterial properties useful in plant protection¹⁰⁷. Bacillus thuringiensis serovar asturiensis (serotype 53) clusters in this classifier and has not been identified as having either anti-cancer or plant protection properties. Classifier A1g is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1g is assigned to proteotype A and genotype A1; amino acid and DNA sequences, respectively, are attached as appendices. A molecular signature for this group is SspE translated protein sequence length of 93 amino acids, with the following residue characteristics: S at position 29, S at position 73, Q at position 87. The classifier A1g contains MLST ST 138¹⁰⁸.

13. EXEMPLARY UTILITY: Herbicide enhancement. sspE genotype E1: The basis for claiming this group is the presence of a misidentified Bacillus subtilis strain that has been patented by Micro Flo Company as a herbicide enhancer¹⁰⁶. Other isolates in this genotype group are bona fide B. cereus strains ATCC 15816, ATCC 13061 and BGSC 6A9 and a misidentified Bt serovar canadensis (serotype 5a, 5c) isolate. Genotype E1 is a unique SspE proteotype (primary isolate screening) and sspE genotype (secondary isolate screening) which, when used in combination, has a high level of phylogenetic clustering power. Genotype E1 is assigned to proteotype E; amino acid and DNA sequences, respectively, are attached as appendices. A molecular signature for this group is SspE translated protein sequence length of 93 amino acids, with the following residue characteristics: A at position 73, Q at position 80. Genotype E1 is 282 nucleotides (nt) in length and is distinguished from other genotypes of proteotype E by the following nucleotide sequence characteristics: G at position 30, T at position 42, G at position 102, A at position 114, C at position 123, A at position 126, G at position 138, A at position 147, C at position 174, T at position 180, T at position 189, T at position 195, C at position 210, A at position 249. Genotype E1 contains at least four MLST STs: 26, 164, 205, 266¹⁰⁸.

14. EXEMPLARY UTILITY: Medical and veterinary diagnostic. SspE proteotype E: The strains that cluster into SspE proteotype E are very closely related to Bacillus anthracis and could be considered transitional pathogens. Specifically, two very important pathogenic strains that have been identified as Bacillus cereus, carrying plasmid-associated [and] pathogenic activity against both human³⁰ and veterinary (zebra²⁵) hosts cluster in this non-Bacillus anthracis group. Proteotype E amino acid and DNA sequences, respectively, are attached as appendices. A molecular signature for this group is SspE translated protein sequence length of 93 amino acids, with the following residue characteristics: A at position 73, Q at position 80. Proteotype E contains at least eleven genotypes (E1-11) and at least eighteen MLST STs: 26, 32, 38, 75, 78, 103, 104, 108, 109, 163, 164, 171, 205, 211, 219, 234, 246, 266¹⁰⁸. SspE proteotype O: The claim is based on the splitting of one Bacillus anthracis strain into the proteotype P group. Proteotype O amino acid and DNA sequences, respectively, are attached as appendices. A molecular signature for this group is SspE translated protein sequence length of 95 amino acids, with the following residue characteristics: A at position 29, N at position 33, S at position 54, I at position 55, T at position 59, A at position 75, Q at position 82. Proteotype O has at least one genotype (O1) and at least three MLST STs: 1, 2, 3¹⁰⁸. SspE proteotype P: The claim is based on the splitting of one Bacillus anthracis strain into the proteotype P group. Proteotype P amino acid and DNA sequences, respectively, are attached as appendices. A molecular signature for this group is SspE translated protein sequence length of 95 amino acids, with the following residue characteristics: A at position 29, N at position 33, S at position 54, V at position 55, T at position 59, A at position 75, Q at position 82. Proteotype P contains at least one genotype (P1) and at least one MLST ST: 1¹⁰⁸.

15. EXEMPLARY UTILITY: Medical diagnostic. SspE proteotype K: The strains that cluster into SspE proteotype K are very closely related to Bacillus anthracis and could be considered transitional pathogens. Specifically, one important pathogenic strain identified as Bacillus thuringiensis serovar konkukian^{25, 26} (serotype 34) clusters in this non-Bacillus anthracis group. This strain was isolated from the leg of a wounded soldier which required amputation due to the severity of the infection. Proteotype K amino acid and DNA sequences, respectively, are attached as appendices. A molecular signature for this group is SspE translated protein sequence length of 93 amino acids, with the following residue characteristics: A at position 29, N at position 33, A at position 73, Q at position 80. Proteotype K contains at least four genotypes (K1-4) and at least twelve MLST STs: 76, 106, 110, 112, 113, 214, 216, 237, 247, 250, 254, 262¹⁰⁸.

16. EXEMPLARY UTILITY: Anti-helminthic, nematicide. sspE genotypes A1 and H5 and SspE proteotypes B and C: Bacillus thuringiensis strains possessing the Cry5 toxin have been identified as having anti-helminthic and nematicidal activity¹⁰⁹⁻¹¹¹. The Cry5 toxin has been shown to be toxic to the nematode Caenorhabditis elegans¹¹⁰, the hookworm parasite Ancylostoma ceylanicum¹⁰⁹, the liver fluke Fasicola hepatica¹⁰² and the plant parasitic species Pratylenchus¹⁰². sspE genotype A1 contains Bt serovars kurstaki (serotype 3a, 3b, 3c), kenyae (serotype 4a, 4c), galleriae (serotype 5a, 5b), aizawai (serotype 7), entomocidus (serotype 6) and colmeri (serotype 21) which have been identified as having anti-helminthic¹⁰⁹⁻¹¹¹. activity. Other isolates cluster within this group which have not yet been described as nematicidal: Bt serovars asturiensis (serotype 53), dakota (serotype 15), londrina (serotype 10a, 10c), coreanensis (serotype 25), yosoo (serotype 18a, 18c), indiana (serotype 16), jinghongiensis (serotype 42), japonensis (serotype 23) and wuhanensis (no serotype), as well as ATCC strains 11778 and 29730 and NRRL strain B-21619. Genotype A1 is a unique SspE proteotype (primary isolate screening) and sspE genotype (secondary isolate screening) which when used in combination, have a high level of phylogenetic clustering power. Genotype A1 is assigned to proteotype A; amino acid and DNA sequences, respectively, are attached as appendices. A molecular signature for this group is an SspE translated protein sequence length of 93 amino acids, with the following residue characteristics: S at position 29, S at position 73, Q at position 87. Genotype A1 is 282 nucleotides (nt) in length and is distinguished from other genotypes of proteotype A by the following nucleotide sequence characteristics: A at position 147. Genotype A1 contains at least thirteen MLST STs: 8, 13, 15, 25, 29, 34, 138, 225, 232, 238, 241, 251, 263¹⁰⁸. sspE genotype H5 contains Bt serovars alesti (serotype 3a, 3c), dendrolimus (serotype 4a, 4b), morrisoni (serotype 8a, 8b) and thompsoni (serotype 12) which have been identified as having anti-helminthic¹⁰⁹⁻¹¹¹ activity. Other isolates cluster within this group which have not yet been described as nematicidal: Bt serovars palmanyolensis (serotype 55), malayensis (serotype 36), israelensis (serotype 14), darmstadiensis (serotype 10a, 10b), leesis (serotype 33), poloniensis (serotype 54) and zhaodongensis (serotype 62), as well as BGSC strain 18A1. Genotype H5 is a unique SspE proteotype (primary isolate screening) and sspE genotype (secondary isolate screening) which when used in combination, have a high level of phylogenetic clustering power. Genotype H5 is assigned to proteotype H; amino acid and DNA sequences, respectively, are attached as appendices. A molecular signature for this group is an SspE translated protein sequence length of 93 amino acids, with the following residue characteristics: A at position 29, N at position 33, A at position 73, E at position 93. Genotype H5 is 282 nucleotides (nt) in length and is distinguished from other genotypes of proteotype H by the following nucleotide sequence characteristics: C at position 48, G at position 87, C at position 180, C at position 210, T at position 237, G at position 240. Genotype H5 contains at least eight MLST STs: 12, 16, 23, 56, 197, 230, 264, 265¹⁰⁸. SspE proteotype B contains Bt serovar entomocidus (serotype 6) and has been identified as having anti-helminthic¹⁰⁹⁻¹¹¹ activity. Proteotype B is a unique SspE proteotype (primary isolate screening) and sspE genotype (secondary isolate screening) which has substantial phylogenetic clustering power. The basis for claiming this group is the splitting of the entomocidus (6) serovar into classifier A1a. 60% (3/5) of serovar entomocidus (6) isolates tested cluster in this classifier. Proteotype B amino acid and DNA sequences, respectively, are attached as appendices. A molecular signature for this group is an SspE translated protein sequence length of 93 amino acids, with the following residue characteristics: K at position 87. Proteotype B has at least one genotype (B1) and at least two isolate STs 221 and 239¹⁰⁸. SspE proteotype C contains Bt serovar tolworthi (serotype 9) which has been identified as having anti-helminthic¹⁰⁹⁻¹¹¹ activity. Proteotype C is a unique SspE proteotype (primary isolate screening) and sspE genotype (secondary isolate screening) which when used in combination, have substantial phylogenetic clustering power. Proteotype C amino acid and DNA sequences, respectively, are attached as appendices. A molecular signature for this group is SspE translated protein sequence length of 93 amino acids, with the following residue characteristics: S at position 29, A at position 73, Q at position 87. Proteotype C contains at least one genotype (C1) and at least one MLST ST 22¹⁰⁸.

Screening/Molecular Diagnostic Targets—Bacillus thuringiensis Group Scheme (See Also Table 1.)

1. SCREENING/MOLECULAR DIAGNOSTIC TARGETS i: Classifier A1i: Target for Bacillus thuringiensis serovar coreanensis (serotype 25) (1/1 isolates); Anti-cancer activity⁶¹. Classifier A1i is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1i is assigned to proteotype A and genotype A1; amino acid and DNA sequences, respectively, are attached as appendices. A molecular signature for this group is SspE translated protein sequence length of 93 amino acids, with the following residue characteristics: S at position 29, S at position 73, Q at position 87. The classifier A1i contains MLST ST 232¹⁰⁸. Classifier A1m: Target for Bacillus thuringiensis serovar japonensis (serotype 23) (1/1 isolates); Insecticidal activity against Lepidoptera^{29, 96-97} and Coleoptera^{33, 62, 79} orders. Classifier A1m is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1m is assigned to proteotype A and genotype A1; amino acid and DNA sequences, respectively, are attached as, appendices. A molecular signature for this group is SspE translated protein sequence length of 93 amino acids, with the following residue characteristics: S at position 29, S at position 73, Q at position 87. The classifier A1m contains MLST ST 263¹⁰⁸. sspE genotype A2: Target for Bacillus thuringiensis serovar nigeriae aka nigeriensis (serotype 8b, 8d) (3/3 isolates); Insecticidal activity against Lepidoptera^{36, 69}. Genotype A2 is a unique SspE proteotype (primary isolate screening) and sspE genotype (secondary isolate screening) which, when used in combination, has a high level of phylogenetic clustering power. Genotype A2 is assigned to proteotype A; amino acid and DNA sequences, respectively, are attached as appendices. A molecular signature for this group is SspE translated protein sequence length of 93 amino acids, with the following residue characteristics: S at position 29, S at position 73, Q at position 87. Genotype A2 is 282 nucleotides (nt) in length and is distinguished from other genotypes of proteotype A by the following nucleotide sequence characteristics: G at position 147. Genotype A2 contains at least two MLST STs: 226, 244¹⁰⁸. SspE proteotype C: Target for Bacillus thuringiensis serovar tolworthi (serotype 9) (3/3 isolates); Insecticidal activity against Lepidoptera^{20, 29, 69, 92}, Coleoptera^{15, 74, 88} and Diptera⁷⁷. Proteotype C amino acid and DNA sequences, respectively, are attached as appendices. A molecular signature for this group is SspE translated protein sequence length of 93 amino acids, with the following residue characteristics: S at position 29, A at position 73, Q at position 87. Proteotype C contains at least one genotype (C1) and at least one MLST ST 22¹⁰⁸. Classifier F3a: Target for Bacillus thuringiensis serovar canadensis (serotype 5a, 5c) (1/2 isolates); Insecticidal activity against Diptera^{21, 39, 73, 77} (see claim for genotype F3 above) Misidentified canadensis is in SspE proteotype E. Classifier F3a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier F3a is assigned to proteotype F and genotype F3; amino acid and DNA sequences, respectively, are attached as appendices. A molecular signature for this group is SspE translated protein sequence length of 93 amino acids, with the following residue characteristics: A at position 29, N at position 33, A at position 73. The classifier F3a contains MLST ST 50¹⁰⁸. Classifier F3b: Target for Bacillus thuringiensis serovar mexicanensis (serotype 27) (1/1 isolates); Insecticidal activity against Diptera⁷⁷. (see claim for genotype F3 above). Classifier F3b is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier F3b is assigned to proteotype F and genotype F3; amino acid and DNA sequences, respectively, are attached as appendices. A molecular signature for this group is SspE translated protein sequence length of 93 amino acids, with the following residue characteristics: A at position 29, N at position 33, A at position 73. The classifier F3b contains MLST ST 224¹⁰⁸. SspE proteotype G (classifier G1a): Target for Bacillus thuringiensis serovar. yunnanensis (serotype 20a, 20b) (1/1 isolates); Insecticidal activity against Isoptera¹². Proteotype G amino acid and DNA sequences, respectively, are attached as appendices. A molecular signature for this group is SspE translated protein sequence length of 93 amino acids, with the following residue characteristics: A at position 29, N at position 33, K at position 55, A at position 73. Proteotype G has at least one genotype (G1) and at least one MLST ST 212¹⁰⁸. Classifier H2a: Target for Bacillus thuringiensis serovar amagiensis (serotype 29) (1/1 isolates); Insecticidal activity against Diptera⁷⁷ and Lepidoptera³⁶. (see claim for genotype H2 above). Classifier H2a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H2a is assigned to proteotype H and genotype H2; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H2a contains MLST ST 208¹⁰⁸. Classifier H2c: Target for Bacillus thuringiensis serovar kyushuensis (serotype 11a, 11c) (1/1 isolates); Insecticidal activity against Diptera^{21, 39, 48-50, 73, 77, 101}. (see claim for genotype H2 above). Classifier H2c is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H2c is assigned to proteotype H and genotype H2; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H2c contains MLST ST 227¹⁰⁸. Classifier H2d: Target for Bacillus thuringiensis serovar neoleonensis (serotype 24a, 24b) (1/1 isolates); Insecticidal activity against Diptera^{72, 103} and anti-cancer⁶¹ activity. (see claim for genotype H2 above). Classifier H2d is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H2d is assigned to proteotype H and genotype H2; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H2d contains MLST ST 228¹⁰⁸. Classifier H2e: Target for Bacillus thuringiensis serovar shandongiensis (serotype 22) (1/1 isolates); Insecticidal activity against Diptera^{39, 77} and anti-cancer^{53-5, 61, 66}. (see claim for genotype H2 above). Classifier H2e is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H2e is assigned to proteotype H and genotype H2; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H2e contains MLST ST 233¹⁰⁸. Classifier H3a: Target for strain useful in biological control of plant fungal diseases. (see claim for genotype H3 above). Classifier H3a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H3a is assigned to proteotype H and genotype H3; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H3a contains MLST ST 206¹⁰⁸. Classifier H3c: Target for Bacillus thuringiensis serovar tohokuensis (serotype 17) (1/1 isolates); Insecticidal activity against Diptera⁷⁷. (see claim for genotype H3 above). Classifier H3c is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H3c is assigned to proteotype H and genotype H3; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H3c contains MLST ST 242¹⁰⁸. Classifier H3d: Target for Bacillus thuringiensis serovar ostriniae (serotype 8a, 8c) (1/1 isolates); Insecticidal activity against Lepidoptera⁶⁹. (see claim for genotype H3 above). Classifier H3d is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H3d is assigned to proteotype H and genotype H3; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H3d contains MLST ST 243108. Classifier H4b: Target for strain useful in biological control of plant fungal diseases. (see claim for genotype H4 above). Classifier H4b is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H4b is assigned to proteotype H and genotype H4; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H4b contains MLST ST 204¹⁰⁸. Classifier H4c: Target for Bacillus thuringiensis serovar sooncheon (serotype 41) (1/1 isolates); Insecticidal activity against Isoptera¹². (see claim for genotype H4 above). Classifier H4c is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H4c is assigned to proteotype H and genotype H4; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H4c contains MLST ST 229¹⁰⁸. Classifier H5d: Target for Bacillus thuringiensis serovar darmstadiensis (serotype 10a, 10b) (3/3 isolates); Insecticidal activity against Diptera^{16, 21, 39, 49, 68, 72-73, 77, 101, 103} & Lepidoptera^{38, 96-97}. Classifier H5d is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H5d is assigned to proteotype H and genotype H5; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H5d contains MLST ST 56¹⁰⁸. Classifier H5e: Target for Bacillus thuringiensis serovar leesis (serotype 33) (1/1 isolates); Insecticidal activity against Diptera^{21, 28} Classifier H5e is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H5e is assigned to proteotype H and genotype H5; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H5e contains MLST ST 230¹⁰⁸. sspE genotype E3 (classifier E3a): Target for Bacillus thuringiensis serovar. konkukian (serotype 34) (1/2 isolates); Insecticidal activity against Diptera¹⁰⁰. (see claim for proteotype E above). Genotype E3 is a unique SspE proteotype (primary isolate screening) and sspE genotype (secondary isolate screening) which, when used in combination, has a high level of phylogenetic clustering power. Genotype E3 is assigned to proteotype E; amino acid and DNA sequences, respectively, are attached as appendices. A molecular signature for this group is SspE translated protein sequence length of 93 amino acids, with the following residue characteristics: A at position 73, Q at position 80. Genotype E3 is 282 nucleotides (nt) in length and is distinguished from other genotypes of proteotype E by the following nucleotide sequence characteristics: A at position 30, T at position 42, G at position 102, A at position 114, C at position 123, A at position 126, A at position 138, A at position 147, C at position 174, T at position 180, T at position 189, T at position 195, C at position 210, A at position 249. Genotype E3 contains at least one MLST ST 211¹⁰⁸. sspE genotype E10 (classifier E10a): Screening/molecular medical diagnostic target for Bacillus cereus³⁰ (1/1 isolates); Human medical diagnostic target. (see claim for proteotype E above). Genotype E10 is a unique SspE proteotype (primary isolate screening) and sspE genotype (secondary isolate screening) which, when used in combination, has a high level of phylogenetic clustering power. Genotype E10 is assigned to proteotype E; amino acid and DNA sequences, respectively, are attached as appendices. A molecular signature for this group is SspE translated protein sequence length of 93 amino acids, with the following residue characteristics: A at position 73, Q at position 80. Genotype E10 is 282 nucleotides (nt) in length and is distinguished from other genotypes of proteotype E by the following nucleotide sequence characteristics: A at position 30, T at position 42, G at position 102, A at position. 114, C at position 123, A at position 126, A at position 138, G at position 147, T at position 174, C at position 180, A at position 189, T at position 195, T at position 210, A at position 249. Genotype E10 contains at least one MLST ST 78¹⁰⁸. sspE genotype E11 (classifier Ella): Screening/molecular medical diagnostic target for Bacillus cereus²⁵ (1/1 isolates); Veterinary diagnostic target (zebra). (see claim for proteotype E above). Genotype E11 is a unique SspE proteotype (primary isolate screening) and sspE genotype (secondary isolate screening) which, when used in combination, has a high level of phylogenetic clustering power. Genotype E11 is assigned to proteotype E; amino acid and DNA sequences, respectively, are attached as appendices. A molecular signature for this group is SspE translated protein sequence length of 93 amino acids, with the following residue characteristics: A at position 73, Q at position 80. Genotype E11 is 282 nucleotides (nt) in length and is distinguished from other genotypes of proteotype E by the following nucleotide sequence characteristics: A at position 30, T at position 42, G at position 102, A at position 114, C at position 123, A at position 126, A at position 138, G at position 147, T at position 174, C at position 180, A at position 189, T at position 195, C at position 210, A at position 249. Genotype E11 has at least one MLST ST: “268”. Classifier K2d: Target for Bacillus thuringiensis strain 97-27-like isolates [identified as serovar. konkukian^{25, 26} (serotype 34)] (1/1 isolates). Classifier K2d is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier K2d is assigned to proteotype K and genotype K2; amino acid and DNA sequences, respectively, are attached as appendices. A molecular signature for this group is SspE translated protein sequence length of 93 amino acids, with the following residue characteristics: A at position 29, N at position 33, A at position 73, Q at position 80. The classifier K2d contains MLST ST 113108 SspE proteotype O: Target for Bacillus anthracis (1/1 isolates). Proteotype O amino acid and DNA sequences, respectively, are attached as appendices. A molecular signature for this group is. SspE translated protein sequence length of 95 amino acids, with the following residue characteristics: A at position 29, N at position 33, S at position 54, I at position 55, T at position 59, A at position 75, Q at position 82. Proteotype O has at least one genotype (O1) and at least three MLST STs: 1, 2, 3¹⁰⁸. (see claim for proteotype O above). SspE proteotype P: Target for Bacillus anthracis (1/1 isolates). Proteotype P amino acid and DNA sequences, respectively, are attached as appendices. A molecular signature for this group is SspE translated protein sequence length of 95 amino acids, with the following residue characteristics: A at position 29, N at position 33, S at position 54, V at position 55, T at position 59, A at position 75, Q at position 82. Proteotype P has at least one genotype (P1) and at least one MLST ST 1. (see claim for proteotype P above)

2. SCREENING/MOLECULAR DIAGNOSTIC TARGETS 2: Classifier A1h: Target for Bacillus thuringiensis serovar londrina (serotype 10a, 10c) (1/1 isolates). Classifier A1h is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1h is assigned to proteotype A and genotype A1; amino acid and DNA sequences, respectively, are attached as appendices. A molecular signature for this group is SspE translated protein sequence length of 93 amino acids, with the following residue characteristics: S at position 29, S at position 73, Q at position 87. The classifier A1h contains MLST ST 225. Classifier A1j: Target for Bacillus thuringiensis serovar yosoo (serotype 18a, 18c) (1/1 isolates). Classifier A1j is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1j is assigned to proteotype A and genotype A1; amino acid and DNA sequences, respectively, are attached as appendices. A molecular signature for this group is SspE translated protein sequence length of 93 amino acids, with the following residue characteristics: S at position 29, S at position 73, Q at position 87. The classifier A1j contains MLST ST 238¹⁰⁸. Classifier A1k: Target for Bacillus thuringiensis serovar indiana (serotype 16) (2/2 isolates). Classifier A1k is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1k is assigned to proteotype A and genotype A1; amino acid and DNA sequences, respectively, are attached as appendices. A molecular signature for this group is SspE translated protein sequence length of 93 amino acids, with the following residue characteristics: S at position 29, S at position 73, Q at position 87. The classifier A1k contains MLST ST 241¹⁰⁸. Classifier A1l: Target for Bacillus thuringiensis serovar jinghongiensis (serotype 42) (1/1 isolates). Classifier A1l is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier A1l is assigned to proteotype A and genotype A1; amino acid and DNA sequences, respectively, are attached as appendices. A molecular signature for this group is SspE translated protein sequence length of 93 amino acids, with the following residue characteristics: S at position 29, S at position 73, Q at position 87. The classifier A1l contains MLST ST 251¹⁰⁸. Classifier F4b: Target for Bacillus thuringiensis serovar pakistani (serotype 13) (1/1 isolates). Classifier F4b is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier F4b is assigned to proteotype F and genotype F4; amino acid and DNA sequences, respectively, are attached as appendices. A molecular signature for this group is SspE translated protein sequence length of 93 amino acids, with the following residue characteristics: A at position 29, N at position 33, A at position 73. The classifier F4b contains MLST ST 17¹⁰⁸. Classifier F4c: Target for Bacillus thuringiensis serovar iberica (serotype 59) (1/1 isolates). Classifier F4c is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier F4c is assigned to proteotype F and genotype F4; amino acid and DNA sequences, respectively, are attached as appendices. A molecular signature for this group is SspE translated protein sequence length of 93 amino acids, with the following residue characteristics: A at position 29, N at position 33, A at position 73. The classifier F4c contains MLST ST 142¹⁰⁸. Classifier F4d: Target for Bacillus thuringiensis serovars vazensis (serotype 67) and rongseni (serotype 56) (1/1 isolates each). Classifier F4d is a unique SspE proteotype (primary isolate screening), sspE genotype (secondary isolate screening) and MLST sequence type (ST)¹⁰⁸ (tertiary isolate screening or “fingerprinting”) which, when used in combination, has a high level of phylogenetic clustering power. Classifier F4d is assigned to proteotype F and genotype F4; amino acid and DNA sequences, respectively, are attached as appendices. A molecular signature for this group is SspE translated protein sequence length of 93 amino acids, with the following residue characteristics: A at position 29, N at position 33, A at position 73. The classifier F4d contains MLST ST 220¹⁰⁸. sspE genotype H1 (classifier H1b): Target for Bacillus thuringiensis serovar xiaguangiensis (serotype 51) (1/1 isolates). Genotype H1 is a unique SspE proteotype (primary isolate screening) and sspE genotype (secondary isolate screening) which, when used in combination, has a high level of phylogenetic clustering power. Genotype H1 is assigned to proteotype H; amino acid and DNA sequences, respectively, are attached as appendices. A molecular signature for this group is SspE translated protein sequence length of 93 amino acids, with the following residue characteristics: A at position 29, N at position 33, A at position 73, E at position 93. Genotype H1 is 282 nucleotides (nt) in length and is distinguished from other genotypes of proteotype H by the following nucleotide sequence characteristics: C at position 48, G at position 87, T at position 180, T at position 210, A at position 237, A at position 240. Genotype H1 has at least four isolate fingerprints: STs 111, 218, 223, 249¹⁰⁸. Classifier H2b: Target for Bacillus thuringiensis serovar cameroun (serotype 32) (1/1 isolates). (see claim for genotype H2 above). Classifier H2b is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H2b is assigned to proteotype H and genotype H2; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H2b contains MLST ST 209¹⁰⁸. Classifier H2f: Target for Bacillus thuringiensis serovar seoulensis (serotype 35) (1/1 isolates). (see claim for genotype H2 above). Classifier H2f is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H2f is assigned to proteotype H and genotype H2; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H2f contains MLST ST 158¹⁰⁸. Classifier H3b: Target for Bacillus thuringiensis serovar. silo (serotype 26) (1/1 isolates). (see claim for genotype H3 above). Classifier H3b is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H3b is assigned to proteotype H and genotype H3; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H3b contains MLST ST 210¹⁰⁸ Classifier H4a: Target for Bacillus thuringiensis serovar thuringiensis (serotype 1) (9/10 isolates). (see claim for genotype H4 above). Classifier H4a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H4a is assigned to proteotype H and genotype H4; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H4a contains MLST ST 10¹⁰⁸. Classifier H4d: Target for Bacillus thuringiensis serovar kim (serotype 52) (1/1 isolates). (see claim for genotype H4 above). Classifier H4d is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H4d is assigned to proteotype H and genotype H4; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H4d contains MLST ST 236¹⁰⁸. Classifier H4e: Target for Bacillus thuringiensis serovar thuringiensis (serotype 1) (1/10 isolates). (see claim for genotype H4 above). Classifier H4e is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H4e is assigned to proteotype H and genotype H4; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H4e contains MLST ST 256¹⁰⁸. Classifier H5g: Target for Bacillus thuringiensis serovar. poloniensis (serotype 54) (1/1 isolates). Classifier H5g is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H5g is assigned to proteotype H and genotype H5; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H5g contains MLST ST 264¹⁰⁸. Classifier H5h: Target for Bacillus thuringiensis serovar zhaodongensis (serotype 62) (1/1 isolates). Classifier H5h is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier H5h is assigned to proteotype H and genotype H5; amino acid and DNA sequences, respectively, are attached as appendices. A molecular signature for this group is SspE translated protein sequence length of 93 amino acids, with the following residue characteristics: A at position 29, N at position 33, A at position 73, E at position 93. The classifier H5h contains MLST ST 265¹⁰⁸. Classifier E2a: Target for Bacillus thuringiensis serovar finitimus (serotype 2) (2/2 isolates). (see claim for proteotype E above). Classifier E2a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier E2a is assigned to proteotype E and genotype E2; amino acid and DNA sequences, respectively, are attached as appendices. A molecular signature for this group is SspE translated protein sequence length of 93 amino acids, with the following residue characteristics: A at position 73, Q at position 80. The classifier E2a contains MLST ST 171¹⁰⁸ Classifier E5a: Target for Bacillus thuringiensis serovar graciosensis (serotype 66) (1/1 isolates). (see claim for proteotype E above). Classifier E5a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier E5a is assigned to proteotype E and genotype E5; amino acid and DNA sequences, respectively, are attached as appendices. A molecular signature for this group is SspE translated protein sequence length of 93 amino acids, with the following residue characteristics: A at position 73, Q at position 80. The classifier E5a contains MLST ST 219¹⁰⁸. Classifier E6a: Target for Bacillus thuringiensis serovar chanpaisis (serotype 46) (1/1 isolates). (see claim for proteotype E above). Classifier E6a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier E6a is assigned to proteotype E and genotype E6; amino acid and DNA sequences, respectively, are attached as appendices. A molecular signature for this group is SspE translated protein sequence length of 93 amino acids, with the following residue characteristics: A at position 73, Q at position 80. The classifier E6a contains MLST ST 234¹⁰⁸. Classifier E7a: Target for Bacillus thuringiensis serovar tochigiensis (serotype 19) (1/1 isolates). (see claim for proteotype E above). Classifier E7a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier E7a is assigned to proteotype E and genotype E7; amino acid and DNA sequences, respectively, are attached as appendices. A molecular signature for this group is SspE translated protein sequence length of 93 amino acids, with the following residue characteristics: A at position 73, Q at position 80. The classifier E7a contains MLST ST 104¹⁰⁸. sspE genotype K1 (classifier K1a): target for Bacillus thuringiensis serovar guiyangiensis (serotype 43) (1/1 isolates). Genotype K1 is a unique SspE proteotype (primary isolate screening) and sspE genotype (secondary isolate screening) which, when used in combination, has a high level of phylogenetic clustering power. Genotype K1 is assigned to proteotype K; amino acid and DNA sequences, respectively, are attached as appendices. A molecular signature for this group is SspE translated protein sequence length of 93 amino acids, with the following residue characteristics: A at position 29, N at position 33, A at position 73, Q at position 80. Genotype K1 is 282 nucleotides (nt) in length and is distinguished from other genotypes of proteotype K by the following nucleotide sequence characteristics: T at position 48, T at position 57, C at position 123, A at position 138, A at position 147, C at position 174, T at position 189, T at position 195, C at position 210, A at position 237, C at position 238, A at position 240, T at position 270. Genotype K1 has at least one isolate fingerprint: ST 247¹⁰⁸. Classifier K2a: Target for Bacillus thuringiensis serovar brasilensis (serotype 39) (1/1 isolates). Classifier K2a is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier K2a is assigned to proteotype K and genotype K2; amino acid and DNA sequences, respectively, are attached as appendices. A molecular signature for this group is SspE translated protein sequence length of 93 amino acids, with the following residue characteristics: A at position 29, N at position 33, A at position 73, Q at position 80. The classifier K2a contains MLST ST 106¹⁰⁸. Classifier K2b: Target for Bacillus thuringiensis serovar. pulsiensis (serotype 65) (1/1 isolates). Classifier K2b is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier K2b is assigned to proteotype K and genotype K2; amino acid and DNA sequences, respectively, are attached as appendices. A molecular signature for this group is SspE translated protein sequence length of 93 amino acids, with the following residue characteristics: A at position 29, N at position 33, A at position 73, Q at position 80. The classifier K2b contains MLST ST 110¹⁰⁸. Classifier K2c: Target for Bacillus thuringiensis serovar. pondicheriensis (serotype 20a, 20c) (1/1 isolates). Classifier K2c is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier K2c is assigned to proteotype K and genotype K2; amino acid and DNA sequences, respectively, are attached as appendices. A molecular signature for this group is SspE translated protein sequence length of 93 amino acids, with the following residue characteristics: A at position 29, N at position 33, A at position 73, Q at position 80. The classifier K2c contains MLST ST 112¹⁰⁸. Classifier K2f: Target for Bacillus thuringiensis serovar sylvestriensis (serotype 61) (1/1 isolates). Classifier K2f is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier K2f is assigned to proteotype K and genotype K2; amino acid and DNA sequences, respectively, are attached as appendices. A molecular signature for this group is SspE translated protein sequence length of 93 amino acids, with the following residue characteristics: A at position 29, N at position 33, A at position 73, Q at position 80. The classifier K2f contains MLST ST 237¹⁰⁸ Classifier K2g: Target for Bacillus thuringiensis serovar azorensis (serotype 64) (1/1 isolates). Classifier K2g is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier K2g is assigned to proteotype K and genotype K2; amino acid and DNA sequences, respectively, are attached as appendices. A molecular signature for this group is SspE translated protein sequence length of 93 amino acids, with the following residue characteristics: A at position 29, N at position 33, A at position 73, Q at position 80. The classifier K2g contains MLST ST 254¹⁰⁸. Classifier K3b: Target for Bacillus thuringiensis serovar argentinensis (serotype 58) (1/1 isolates). Classifier K3b is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier K3b is assigned to proteotype K and genotype K3; amino acid and DNA sequences, respectively, are attached as appendices. A molecular signature for this group is SspE translated protein sequence length of 93 amino acids, with the following residue characteristics: A at position 29, N at position 33, A at position 73, Q at position 80. The classifier K3b contains MLST ST 250¹⁰⁸. Classifier K3c: Target for Bacillus thuringiensis serovar balearica (serotype 48) (1/1 isolates). Classifier K3c is a unique SspE proteotype (primary isolate screening), sspE genotype (secondary isolate screening) and MLST sequence type (ST)¹⁰⁸ (tertiary isolate population genetic screening) which, when used in combination, has a high level of phylogenetic clustering power. Classifier K3c is assigned to proteotype K and genotype K3; amino acid and DNA sequences, respectively, are attached as appendices. A molecular signature for this group is SspE translated protein sequence length of 93 amino acids, with the following residue characteristics: A at position 29, N at position 33, A at position 73, Q at position 80. The classifier K3c contains MLST ST 262¹⁰⁸. SspE proteotype L (classifier L1a): Target for Bacillus thuringiensis serovar toguchini (serotype 31) (1/1 isolates). Proteotype L amino acid and DNA sequences, respectively, are attached as appendices. A molecular signature for this group is SspE translated protein sequence length of 93 amino acids, with the following residue characteristics: A at position 29, N at position 33, A at position 34, A at position 73, Q at position 80. Proteotype L has at least one genotype (L1) and at least one MLST ST 207¹⁰⁸. SspE proteotype M (classifier M1a): Target for Bacillus thuringiensis serovar muju (serotype 49) (1/1 isolates). Proteotype M amino acid and DNA sequences, respectively, are attached as appendices. A molecular signature for this group is SspE translated protein sequence length of 93 amino acids, with the following residue characteristics: A at position 29, N at position 33, A at position 73, Q at position 80, E at position 93. Proteotype M has at least one genotype (M1) and at least two MLST STs: 217, 245¹⁰⁸. SspE proteotype N (classifier N1a): Target for Bacillus thuringiensis serovar monterrey (serotype 28a, 28b) (1/1 isolates). Proteotype N amino acid and DNA sequences, respectively, are attached as appendices. A molecular signature for this group is SspE translated protein sequence length of 92 amino acids, with the following residue characteristics: A at position 29, N at position 33, A at position 73, Q at position 80. Proteotype N has at least one genotype (N1) and at least one MLST ST 107¹⁰⁸.

TABLE 1
Bt group strains organized by classifier

| | | | SspE | | |
|---|---|---|---|---|---|
| | SspE aa | sspE nt | size | | |
| Classifier^{a} | group^{b} | group^{c} | (AA)^{d} | MLST ST^{e} | Commercial/Insecticidal Utility^{f} |
| A1a | A | A1 | 93 | 8 | [symbol] (Lepidoptera^{1,6-7,14,17,20,29,31,36,41,45,47,51,55,57-58,80-84,87,92,98}) & |
| | | | | | (Diptera^{36,47,55,58,77,98-99}); [symbol] (Lepidoptera^{2,20,29,102}); |
| | | | | | [symbol] (Lepidoptera^{20,31-32,36,41,60,87}) |
| A1b | A | A1 | 93 | 13 | [symbol] (Lepidoptera^{29,31,36,41,94}) & (Diptera⁷⁷); [symbol] |
| | | | | | (misidentified) |
| A1c | A | A1 | 93 | 15 | [symbol] (Lepidoptera^{8,20,22,24,29,31,36,46-47,51,55,83}) & |
| | | | | | (Diptera^{22,24,47,77,89}); [symbol] (Diptera^{23,100}) & (Lepidoptera²³) |
| A1d | A | A1 | 93 | 25 | [symbol] (Lepidoptera^{2,20,29,102}); wuhanensis; ATCC 29730 |
| A1e | A | A1 | 93 | 29 | kurstaki (misidentified^{g}) |
| A1f | A | A1 | 93 | 34 | ATCC 11778 |
| A1g | A | A1 | 93 | 138 | [symbol] (Anti-cancer^{40,42,61}); B-21619 (Plant Protection¹⁰⁷); asturiensis |
| A1h | A | A1 | 93 | 225 | londrina²¹ |
| A1i | A | A1 | 93 | 232 | [symbol] ²¹ (Anti-cancer⁶¹) |
| A1j | A | A1 | 93 | 238 | yosoo |
| A1k | A | A1 | 93 | 241 | indiana |
| A1l | A | A1 | 93 | 251 | jinghongiensis²¹ |
| A1m | A | A1 | 93 | 263 | [symbol] (Lepidoptera^{29,96-97}) & (Coleoptera^{33,62,79}) |
| A2a | A | A2 | 93 | 226 | [symbol] & [symbol] (Lepidoptera^{36,69}) |
| A2b | A | A2 | 93 | 244 | [symbol] (Lepidoptera^{36,69}) |
| B1a | B | B1 | 93 | 221 | [symbol] (Lepidoptera^{20,31-32,36,41,60,87}) |
| B1b | B | B1 | 93 | 239 | [symbol] (Lepidoptera^{20,31-32,36,41,60,87}) |
| C1a | C | C1 | 93 | 22 | [symbol] (Lepidoptera^{20,29,69,92}) & (Coleoptera^{15,74,88}) & (Diptera⁷⁷) |
| D1a | D | D1 | 93 | 255 | ATCC 13472 |
| F1a | F | F1 | 93 | 213 | [symbol] (Diptera^{21,39,63,73,77-78,101}) & (Lepidoptera^{63,96-97}); |
| | | | | | [symbol] (Lepidoptera^{36,96-97}) |
| F2a | F | F2 | 93 | 33 | pirenaica⁷¹; B. licheniformis NRRL B-571 (misidentified) |
| F2b | F | F2 | 93 | 59 | [symbol] (Coleoptera⁷⁴) |
| F3a | F | F3 | 93 | 50 | [symbol] (Diptera^{21,39,73,77}) |
| F3b | F | F3 | 93 | 224 | [symbol] (Diptera⁷⁷) |
| F4a | F | F4 | 93 | 4 | ATCC 14579^{T} |
| F4b | F | F4 | 93 | 17 | pakistani²¹ |
| F4c | F | F4 | 93 | 142 | iberica⁷¹ |
| F4d | F | F4 | 93 | 220 | vazensis; rongseni |
| G1a | G | G1 | 93 | 212 | [symbol] (Isoptera¹²) |
| H1a | H | H1 | 93 | 111 | Pey9 & 3466-8.1 - no serotype, natural isolates |
| H1b | H | H1 | 93 | 218 | xiaguangiensis |
| H1c | H | H1 | 93 | 223 | 2A6 &2C1 - no serotype, natural isolates |
| H1d | H | H1 | 93 | 249 | Pey8 - no serotype, natural isolate |
| H2a | H | H2 | 93 | 208 | [symbol] (Diptera⁷⁷) & (Lepidoptera³⁶) |
| H2b | H | H2 | 93 | 209 | cameroun²¹ |
| H2c | H | H2 | 93 | 227 | [symbol] (Diptera^{21,39,48-50,73,77,101}) |
| H2d | H | H2 | 93 | 228 | [symbol] (Diptera^{72,103}) & (Anti-cancer⁶¹) |
| H2e | H | H2 | 93 | 233 | [symbol] (Anti-cancer^{53-54,61,66}) & (Diptera^{39,77}) |
| H2f | H | H2 | 93 | 158 | seoulensis |
| H2g | H | H2 | 93 | 258 | Pey6 - no serotype, natural isolate |
| H3a | H | H3 | 93 | 206 | ATCC 53522; ATCC 55609 (Plant Protection¹⁰⁴) |
| H3b | H | H3 | 93 | 210 | silo²¹ |
| H3c | H | H3 | 93 | 242 | [symbol] (Diptera⁷⁷) |
| H3d | H | H3 | 93 | 243 | [symbol] (Lepidoptera⁶⁹) |
| H4a | H | H4 | 93 | 10 | [symbol] (Lepidoptera^{4,20-21,29,31,36,41,47,83,92}) & (Coleoptera^{4,36}) & |
| | | | | | (Diptera³⁸); kurstaki (misidentified) |
| H4b | H | H4 | 93 | 204 | B. megaterium ATCC 55000 (Plant Protection¹⁰⁵) (misidentified) |
| H4c | H | H4 | 93 | 229 | [symbol] (Isoptera¹²) |
| H4d | H | H4 | 93 | 236 | kim |
| H4e | H | H4 | 93 | 256 | [symbol] (misidentified) (Lepidoptera^{4,20-21,29,31,36,41,47,83,92}) & |
| | | | | | (Coleoptera^{4,36}) & (Diptera³⁸) |
| H5a | H | H5 | 93 | 12 | [symbol] (Lepidoptera^{20-21,31,86}) & (Diptera⁶⁵); [symbol] |
| | | | | | (Lepidoptera^{20,29,92}) & (Diptera⁶⁵); palmanyolensis |
| H5b | H | H5 | 93 | 16 | [symbol] (Diptera^{3,9-11,13,18,21,34,43-44,49-50,70,78,83,90-93,95,98}); malayensis; |
| | | | | | Bacillus sp. BGSC 18A1 (reclassified) |
| H5c | H | H5 | 93 | 23 | [symbol] (Coleoptera^{15,21,29,36,56-57,74,85}) & (Lepidoptera^{19,21,29,36,41,67,69}) |
| | | | | | & (Diptera^{18-19,21,51,67,73}); [symbol] (Diptera^{21,59,72-73}) & (Lepidoptera⁵) |
| H5d | H | H5 | 93 | 56 | [symbol] (Diptera^{16,21,39,49,68,72-73,77,101,103})& (Lepidoptera^{38,96-97}) |
| H5e | H | H5 | 93 | 230 | [symbol] (Diptera^{21,28}) |
| H5f | H | H5 | 93 | 197 | [symbol] (Lepidoptera^{20-21,31,86})& (Diptera⁶⁵) |
| H5g | H | H5 | 93 | 264 | poloniensis |
| H5h | H | H5 | 93 | 265 | zhaodongensis |
| I1a | I | I1 | 93 | 257 | [symbol] (misidentified) (Lepidoptera^{19,21,29,36,41,67,69})& |
| | | | | | (Diptera^{18-19,21,51,67,73}) & (Coleoptera^{36,74}) |
| J1a | J | J1 | 93 | 231 | B. mycoides ATCC 19647 (misidentified) |
| E1a | E | E1 | 93 | 26 | ATCC 15816 |
| E1b | E | E1 | 93 | 164 | Bc ATCC 13061; canadensis (misidentified) |
| E1c | E | E1 | 93 | 205 | B. subtilis ATCC 55675 (Plant Protection¹⁰⁶) (misidentified) |
| E1d | E | E1 | 93 | 266 | BGSC 6A9 |
| E2a | E | E2 | 93 | 171 | finitimus²¹ |
| E2b | E | E2 | 93 | 246 | Bacillus sp. ATCC 51912 (reclassified) |
| E3a | E | E3 | 93 | 211 | [symbol] (Diptera¹⁰⁰) |
| E4a | E | E4 | 93 | 75 | DM55 - no serotype, natural isolate |
| E4b | E | E4 | 93 | 108 | BGSC 6E1; BGSC 6E2 |
| E4c | E | E4 | 93 | 109 | 003,IB, BuIB, III, III-BL, III-BS, IV - no serotypes, natural isolates |
| E4d | E | E4 | 93 | 163 | S8553/2 - no serotype, natural isolate |
| E5a | E | E5 | 93 | 219 | graciosensis |
| E6a | E | E6 | 93 | 234 | chanpaisis |
| E7a | E | E7 | 93 | 104 | tochigiensis |
| E8a | E | E8 | 93 | 38 | ATCC 4342 |
| E8b | E | E8 | 93 | 103 | [symbol] (Isoptera¹²) |
| E9a | E | E9 | 93 | 32 | ATCC 10987 |
| E10a | E | E10 | 93 | 78 | strain G9241 (medical diagnostic - human³⁰) |
| E11a | E | E11 | 93 | “268” | strain ZK (E33L) (veterinary diagnostic - zebra²⁵) |
| K1a | K | K1 | 93 | 247 | guiyangiensis²¹ |
| K2a | K | K2 | 93 | 106 | brasilensis |
| K2b | K | K2 | 93 | 110 | pulsiensis |
| K2c | K | K2 | 93 | 112 | pondicheriensis |
| K2d | K | K2 | 93 | 113 | [symbol] strain 97-27 (medical diagnostic - human^{25,26}) |
| K2e | K | K2 | 93 | 214 | [symbol] (Diptera^{35,58,64,75-76,78}); oswaldocruzi²¹ |
| K2f | K | K2 | 93 | 237 | sylvestriensis |
| K2g | K | K2 | 93 | 254 | azorensis |
| K3a | K | K3 | 93 | 216 | wratislaviensis; pingluonsis |
| K3b | K | K3 | 93 | 250 | argentinensis |
| K3c | K | K3 | 93 | 262 | balearica³⁷ |
| L1a | L | L1 | 93 | 207 | toguchini^{21,52} |
| M1a | M | M1 | 93 | 217 | muju |
| M1b | M | M1 | 93 | 245 | I2 - no serotype, natural isolate |
| N1a | N | N1 | 92 | 107 | monterrey²¹ |
| O1a | O | O1 | 95 | 1 | B. anthracis |
| O1b | O | O1 | 95 | 2 | B. anthracis |
| O1c | O | O1 | 95 | 3 | B. anthracis |
| P1a | P | P1 | 95 | 1 | B. anthracis (strain Western NA) |
| Q1a | Q | Q1 | 93 | 115 | B. weihenstephanensis DSM 11821^{T} |
| Q1b | Q | Q1 | 93 | 116 | B. mycoides ATCC 6462^{T} |
| Q1c | Q | Q1 | 93 | 215 | novosibirsk (misidentified) |
| Q1d | Q | Q1 | 93 | 235 | navarrensis³⁷ (misidentified) |
| Q1e | Q | Q1 | 93 | 248 | B. mycoides ATCC 11986 |
| R1a | R | R1 | 93 | 222 | B. mycoides ATCC 23258 |
| S1a | S | S1 | 92 | “267” | B. mycoides ATCC 21929 |
| T1a | T | T1 | 95 | 259 | B. mycoides ATCC 10206 |
| T1b | T | T1 | 95 | 260 | B. mycoides ATCC 31101 |
| T1c | T | T1 | 95 | 261 | B. mycoides ATCC 31102 |
| U1a | U | U1 | 95 | 114 | B. pseudomycoides DSM 12442^{T} |

Table 1 Footnotes.
^{a}Classifiers are color-coded, bold typed, and describe species, subspecies and serovars of the B. thuringiensis clade by combined sspE (capital letter and number) and MLST (lower case letter corresponds to a sequence type [ST]) within a particular sspE type. A color-coded phylogenetic tree generated from MLST data and labeled with these classifiers is shown in FIG. 3. The data used to generate the tree topology was obtained from all available species and serovars in pubmlst.org/bcereus; only data for which we also have definitive sspE identification and thus a complete classifier are labeled on the tree.
^{b}Translated nucleic acid sequence of the sspE gene gives us SspE proteotype groups A-U.
^{c}Nucleic acid sequences of the sspE gene are assigned (color-coded) genotypes A1-x through U1-x, where the letter corresponds to the SspE proteotype and the number corresponds to a unique nucleic acid sequence of that proteotype. For example, we currently have only one genotype identified for proteotype U, and we currently have 5 genotypes identified for SspE proteotype H (thus, the five H genotypes all have silent mutations with respect to each other). A color-coded phylogenetic tree generated from sspE nucleic acid sequences for the B. thuringiensis group is shown in FIG. 2. sspE sequence data from this study has been deposited in the GenBank nucleotide sequence database with accession numbers AF359764-AF359821, AF359823-AF359843,AF359845,AF359847-AF359860,AF359862-AF359934, AF359936-AF359938 and DQ146892-146926.
^{d}Length of the SspE protein (92-95 amino acids, Bc group).
^{e}The MLST sequence type (ST) is a number assigned to a unique allelic profile from nucleotide sequences of seven housekeeping gene fragments. The genes used in this scheme are glpF, gmk, ilvD, pta, purH, pycA and tpiA, and information including primer sequences, allelic profiles and STs, allele sequences and isolate information is available at pubmlst.org/bcereus. Allelic profiles for STs “267” and “268” have not yet been uploaded to the pubmlst/bcereus website.
^{f}Serovars currently used commercially as insecticides or that are registered for use with the USEPA or that are described in scientific literature as insecticidal are indicated in bold italic font. Species or serovars that are misidentified or misclassified are indicated.
^{g}This “kurstaki” isolate was likely misidentified by the researchers who isolated it. The culture collection agrees that, based on the methods used to isolate this strain, and that it has no reaction to any known Bt antisera, it is very likely B. cereus.

TABLE 2
Amino acid alterations of Bt group strains organized by proteotype and subdivided into genotypes

| | | | | 2 | | 7 | 25 | 29 | 33 | 34 | 38 | 39 | 40 | 47 | 51 | 53 | 53 | | | |
|---|---|---|---|---|---|---|---|---|---|---|---|---|---|---|---|---|---|---|---|---|
| | Pro- | Gen- | | S | | G | G | S | D | V | K | Q | A | K | A | G | G | | | |
| Species | teo- | o- | | ↓ | 7 | ↓ | ↓ | ↓ | ↓ | ↓ | ↓ | ↓ | ↓ | ↓ | ↓ | ↓ | ↓ | Insertion^{†} | | |
| Group | type | type | Strain | N | G | A | C | A | N | A | Q | K | Q | Q | T | A | S | SI | SV | GV |
| B. cereus | A | 1 | 4AL1, 4AR1, 4AT1, 4BF1, 4BQ1, | | | | | | | | | | | | | | | | | |
| T- | | | 4CA1, 4D1, 4D2, 4D4, 4D5, 4D6, | | | | | | | | | | | | | | | | | |
| Related | | | 4D7, 4D8, 4D9, 4D10, 4D11, 4D12, | | | | | | | | | | | | | | | | | |
| Strains | | | 4D14, 4D15, 4D16, 4D17, 4D18, | | | | | | | | | | | | | | | | | |
| | | | 4D19, 4D20, 4D21, | | | | | | | | | | | | | | | | | |
| | | | 4D22, 4F1, 4F2, | | | | | | | | | | | | | | | | | |
| | | | 4F3, 4F4, 4G1, 4G2, 4G3, 4G4, 4G5, | | | | | | | | | | | | | | | | | |
| | | | 4G6, 4I1, 4I2, 4J1, 4J2, 4J3, 4J4, | | | | | | | | | | | | | | | | | |
| | | | 4J5, 4R1, 4S2, 4S3, | | | | | | | | | | | | | | | | | |
| | | | 4T1, 4X1, 6A1, | | | | | | | | | | | | | | | | | |
| | | | 6A2, IB/A, A11778, A29730, B- | | | | | | | | | | | | | | | | | |
| | | | 21619 | | | | | | | | | | | | | | | | | |
| | | 2 | 4AZ1, D6021, D6076 | | | | | | | | | | | | | | | | | |
| | B | | 4I3, 4I4, 4I5 | | | | | | | | | | | | | | | | | |
| | C | | 4L1, 4L2, 4L3 | | | | | | | | | | | | | | | | | |
| | D | | A13472 | | | | | • | | | | | | | | | | | | |
| B. | F | 1 | 4AO1, 4AP1 | | | | | • | • | | | | | | | | | | | |
| thu- | | 2 | 6A3, 6A4, 4BU1, 4W1, A27348, B- | | | | | | | | | | | | | | | | | |
| ringien- | | | 571 | | | | | | | | | | | | | | | | | |
| sis- | | 3 | 4AC1, 4H2 | | | | | | | | | | | | | | | | | |
| Related | | 4 | 6A5, 4BT1, 4BW1, 4CE1, 4P1, | | | | | | | | | | | | | | | | | |
| Strains | | | A14579^{T} | | | | | | | | | | | | | | | | | |
| | G | | 4AM1 | | | | | • | • | | | | | | | | | | | |
| | H | 1 | 3466-8.1, 2A6, 2C1, Pey. 8, Pey. 9, | | | | | • | • | | | | | | | | | | | |
| | | | 4BN1, 6A7, 6A8 | | | | | | | | | | | | | | | | | |
| | | 2 | Pey. 6, 4AE1, 4AF1, 4AN1, 4AQ1, | | | | | | | | | | | | | | | | | |
| | | | 4BE1, 4U1 | | | | | | | | | | | | | | | | | |
| | | 3 | 4AG1, 4V1, 4Z1, A53522, A55609 | | | | | | | | | | | | | | | | | |
| | | 4 | 4A1, 4A2, 4A3, 4A4, 4A5, 4A6, | | | | | | | | | | | | | | | | | |
| | | | 4A7, 4A8, 4A9, 4BB1, 4BP1, 4D3, | | | | | | | | | | | | | | | | | |
| | | | D2046T, A55000 | | | | | | | | | | | | | | | | | |
| | | 5 | 4AA1, 4AB1, 4AK1, 4AV1, 4BR1, | | | | | | | | | | | | | | | | | |
| | | | 4BS1, 4BZ1, 4C1, 4C2, 4C3, 4E1, | | | | | | | | | | | | | | | | | |
| | | | 4E2, 4E3, 4E4, 4E5, 4K1, 4M1, | | | | | | | | | | | | | | | | | |
| | | | 4M2, 4M3, 4O1, 4Q1, 4Q2, 4Q3, | | | | | | | | | | | | | | | | | |
| | | | 4Q4, 4Q5, 4Q6, 4Q7, 4Q8, | | | | | | | | | | | | | | | | | |
| | | | A35646T, 18A1 | | | | | | | | | | | | | | | | | |
| | I | | 4K3 | | | | • | • | • | | | | | | | | | | | |
| | J | | A19647 | | | | | • | • | | | | | | • | | | | | |

| | | | | 55 | 57 | 68 | 72 | 73 | 76 | 80 | 84 | 84 | 85 | 87 | 91 | 93 | |
|---|---|---|---|---|---|---|---|---|---|---|---|---|---|---|---|---|---|
| | Pro- | Gen- | | Q | A | E | H | S | K | K | K | K | Q | Q | S | Q | |
| Species | teo- | o- | | ↓ | ↓ | ↓ | ↓ | ↓ | ↓ | ↓ | ↓ | ↓ | ↓ | ↓ | ↓ | ↓ | 93 |
| Group | type | type | Strain | K | T | D | Q | A | Q | Q | T | N | K | K | T | E | Q |
| B. cereus | A | 1 | 4AL1, 4AR1, 4AT1, 4BF1, 4BQ1, | | | | | | | | | | | | | | |
| T- | | | 4CA1, 4D1, 4D2, 4D4, 4D5, 4D6, | | | | | | | | | | | | | | |
| Related | | | 4D7, 4D8, 4D9, 4D10, 4D11, 4D12, | | | | | | | | | | | | | | |
| Strains | | | 4D14, 4D15, 4D16, 4D17, 4D18, | | | | | | | | | | | | | | |
| | | | 4D19, 4D20, 4D21, | | | | | | | | | | | | | | |
| | | | 4D22, 4F1, 4F2, | | | | | | | | | | | | | | |
| | | | 4F3, 4F4, 4G1, 4G2, 4G3, 4G4, 4G5, | | | | | | | | | | | | | | |
| | | | 4G6, 4I1, 4I2, 4J1, 4J2, 4J3, 4J4, | | | | | | | | | | | | | | |
| | | | 4J5, 4R1, 4S2, 4S3, | | | | | | | | | | | | | | |
| | | | 4T1, 4X1, 6A1, | | | | | | | | | | | | | | |
| | | | 6A2, IB/A, A11778, A29730, B- | | | | | | | | | | | | | | |
| | | | 21619 | | | | | | | | | | | | | | |
| | | 2 | 4AZ1, D6021, D6076 | | | | | | | | | | | | | | |
| | B | | 4I3, 4I4, 4I5 | | | | | | | | | | | • | | | |
| | C | | 4L1, 4L2, 4L3 | | | | | • | | | | | | | | | |
| | D | | A13472 | | | | | | | | | | | | | | |
| B. | F | 1 | 4AO1, 4AP1 | | | | | • | | | | | | | | | |
| thu- | | 2 | 6A3, 6A4, 4BU1, 4W1, A27348, B- | | | | | | | | | | | | | | |
| ringien- | | | 571 | | | | | | | | | | | | | | |
| sis- | | 3 | 4AC1, 4H2 | | | | | | | | | | | | | | |
| Related | | 4 | 6A5, 4BT1, 4BW1, 4CE1, 4P1, | | | | | | | | | | | | | | |
| Strains | | | A14579^{T} | | | | | | | | | | | | | | |
| | G | | 4AM1 | • | | | | • | | | | | | | | | |
| | H | 1 | 3466-8.1, 2A6, 2C1, Pey. 8, Pey. 9, | | | | | • | | | | | | | | • | |
| | | | 4BN1, 6A7, 6A8 | | | | | | | | | | | | | | |
| | | 2 | Pey. 6, 4AE1, 4AF1, 4AN1, 4AQ1, | | | | | | | | | | | | | | |
| | | | 4BE1, 4U1 | | | | | | | | | | | | | | |
| | | 3 | 4AG1, 4V1, 4Z1, A53522, A55609 | | | | | | | | | | | | | | |
| | | 4 | 4A1, 4A2, 4A3, 4A4, 4A5, 4A6, | | | | | | | | | | | | | | |
| | | | 4A7, 4A8, 4A9, 4BB1, 4BP1, 4D3, | | | | | | | | | | | | | | |
| | | | D2046T, A55000 | | | | | | | | | | | | | | |
| | | 5 | 4AA1, 4AB1, 4AK1, 4AV1, 4BR1, | | | | | | | | | | | | | | |
| | | | 4BS1, 4BZ1, 4C1, 4C2, 4C3, 4E1, | | | | | | | | | | | | | | |
| | | | 4E2, 4E3, 4E4, 4E5, 4K1, 4M1, | | | | | | | | | | | | | | |
| | | | 4M2, 4M3, 4O1, 4Q1, 4Q2, 4Q3, | | | | | | | | | | | | | | |
| | | | 4Q4, 4Q5, 4Q6, 4Q7, 4Q8, | | | | | | | | | | | | | | |
| | | | A35646T, 18A1 | | | | | | | | | | | | | | |
| | I | | 4K3 | | | | | • | | | | | | | | • | |
| | J | | A19647 | | | | | • | | | | | | | | • | |

TABLE 3
Amino acid alterations of Bt group strains organized by proteotype and subdivided into genotypes

| | | | | 2 | | 7 | 25 | 29 | 33 | 34 | 38 | 39 | 40 | 47 | 51 | 53 | 53 | | | |
|---|---|---|---|---|---|---|---|---|---|---|---|---|---|---|---|---|---|---|---|---|
| | Pro- | Gen- | | S | | G | G | S | D | V | K | Q | A | K | A | G | G | | | |
| Species | teo- | o- | | ↓ | 7 | ↓ | ↓ | ↓ | ↓ | ↓ | ↓ | ↓ | ↓ | ↓ | ↓ | ↓ | ↓ | Insertion^{†} | | |
| Group | type | type | Strain | N | G | A | C | A | N | A | Q | K | Q | Q | T | A | S | SI | SV | GV |
| B. cereus/ | E | 1 | 4H1, 6A6, 6A9, A13061, A15816, | | | | | | | | | | | | | | | | | |
| thu- | | | A55675 | | | | | | | | | | | | | | | | | |
| ringien- | | 2 | A51912, 4B1, 4B2 | | | | | | | | | | | | | | | | | |
| sis- | | 3 | 4AH1 | | | | | | | | | | | | | | | | | |
| Related | | 4 | 003, 6E1, 6E2, DM55, III, IB, IV, | | | | | | | | | | | | | | | | | |
| Strains | | | III-BL, III-BS, S8553/2, BuIB | | | | | | | | | | | | | | | | | |
| | | 5 | 4CD1 | | | | | | | | | | | | | | | | | |
| | | 6 | 4BH1 | | | | | | | | | | | | | | | | | |
| | | 7 | 4Y1 | | | | | | | | | | | | | | | | | |
| | | 8 | 4BG1, A4342 | | | | | | | | | | | | | | | | | |
| | | 9 | A10987 | | | | | | | | | | | | | | | | | |
| | | 10 | G9241 | | | | | | | | | | | | | | | | | |
| | | 11 | [symbol] ZK | | | | | | | | | | | | | | | | | |
| B. | K | 1 | 4BC1 | | | | | • | • | | | | | | | | | | | |
| anthracis- | | 2 | 97-27, 4AS1, 4AU1, 4AY1, 4BA1, | | | | | | | | | | | | | | | | | |
| Related | | | 4BY1, 4CB1, 4CC1 | | | | | | | | | | | | | | | | | |
| Strains | | 3 | 4BJ1, 4BK1, 4BV1, 4BX1 | | | | | | | | | | | | | | | | | |
| | L | | 4AD1 | | | | | • | • | • | | | | | | | | | | |
| | M | | 4BL1, I2 | | | | | • | • | | | | | | | | | | | |
| | N | | 4AJ1 | | | | | • | • | | | | | | | | | | | |
| | O | | A14578T, A14185, A14186, Sterne, | | | | | • | • | | | | | | | | | • | | |
| | | | CAU-1, CAU-2, CAU-3, CN1, | | | | | | | | | | | | | | | | | |
| | | | CN2, BC, Pasteur #2, Ames, | | | | | | | | | | | | | | | | | |
| | | | A2012, A2084, A1055, Vollum, | | | | | | | | | | | | | | | | | |
| | | | CNEVA-9066, Kruger B, | | | | | | | | | | | | | | | | | |
| | | | Australia94 | | | | | | | | | | | | | | | | | |
| | P | | Western NA USA6153 | | | | | • | • | | | | | | | | | | • | |
| B. | Q | | A6462^{T}, A11986, 4AX1, 4BM1, | | | | | • | • | | | | | • | | • | | | | |
| mycoides- | | | D11821^{T} | | | | | | | | | | | | | | | | | |
| Related | R | | A23258 | | | | | • | • | | | | | • | | • | | | | |
| Strains | S | | A21929 | • | Δ | | | • | • | | • | • | • | | | | | | | |
| | T | | A10206, A31101, A31102 | | | | | • | • | | | | • | | | | • | | | • |
| | U | | D12442^{T} | | | • | | • | • | | | | • | | | | • | | | • |

| | | | | 55 | 57 | 68 | 72 | 73 | 76 | 80 | 84 | 84 | 85 | 87 | 91 | 93 | |
|---|---|---|---|---|---|---|---|---|---|---|---|---|---|---|---|---|---|
| | Pro- | Gen- | | Q | A | E | H | S | K | K | K | K | Q | Q | S | Q | |
| Species | teo- | o- | | ↓ | ↓ | ↓ | ↓ | ↓ | ↓ | ↓ | ↓ | ↓ | ↓ | ↓ | ↓ | ↓ | 93 |
| Group | type | type | Strain | K | T | D | Q | A | Q | Q | T | N | K | K | T | E | Q |
| B. cereus/ | E | 1 | 4H1, 6A6, 6A9, A13061, A15816, | | | | | • | | • | | | | | | | |
| thu- | | | A55675 | | | | | | | | | | | | | | |
| ringien- | | 2 | A51912, 4B1, 4B2 | | | | | | | | | | | | | | |
| sis- | | 3 | 4AH1 | | | | | | | | | | | | | | |
| Related | | 4 | 003, 6E1, 6E2, DM55, III, IB, IV, | | | | | | | | | | | | | | |
| Strains | | | III-BL, III-BS, S8553/2, BuIB | | | | | | | | | | | | | | |
| | | 5 | 4CD1 | | | | | | | | | | | | | | |
| | | 6 | 4BH1 | | | | | | | | | | | | | | |
| | | 7 | 4Y1 | | | | | | | | | | | | | | |
| | | 8 | 4BG1, A4342 | | | | | | | | | | | | | | |
| | | 9 | A10987 | | | | | | | | | | | | | | |
| | | 10 | G9241 | | | | | | | | | | | | | | |
| | | 11 | [symbol] ZK | | | | | | | | | | | | | | |
| B. | K | 1 | 4BC1 | | | | | • | | • | | | | | | | |
| anthracis- | | 2 | 97-27, 4AS1, 4AU1, 4AY1, 4BA1, | | | | | | | | | | | | | | |
| Related | | | 4BY1, 4CB1, 4CC1 | | | | | | | | | | | | | | |
| Strains | | 3 | 4BJ1, 4BK1, 4BV1, 4BX1 | | | | | | | | | | | | | | |
| | L | | 4AD1 | | | | | • | | • | | | | | | | |
| | M | | 4BL1, I2 | | | | | • | | • | | | | | | • | |
| | N | | 4AJ1 | | | | | • | | • | | | | | | | Δ |
| | O | | A14578T, A14185, A14186, Sterne, | | • | | | • | | • | | | | | | | |
| | | | CAU-1, CAU-2, CAU-3, CN1, | | | | | | | | | | | | | | |
| | | | CN2, BC, Pasteur #2, Ames, | | | | | | | | | | | | | | |
| | | | A2012, A2084, A1055, Vollum, | | | | | | | | | | | | | | |
| | | | CNEVA-9066, Kruger B, | | | | | | | | | | | | | | |
| | | | Australia94 | | | | | | | | | | | | | | |
| | P | | Western NA USA6153 | | • | | | • | | • | | | | | | | |
| B. | Q | | A6462^{T}, A11986, 4AX1, 4BM1, | | | | | • | | • | | | | | | • | |
| mycoides- | | | D11821^{T} | | | | | | | | | | | | | | |
| Related | R | | A23258 | | | | | • | | • | • | | | | | • | |
| Strains | S | | A21929 | | | | • | • | | • | | • | • | | | | |
| | T | | A10206, A31101, A31102 | | | • | • | • | • | • | | | • | | • | | |
| | U | | D12442^{T} | | | • | • | • | • | • | | | • | | • | | |

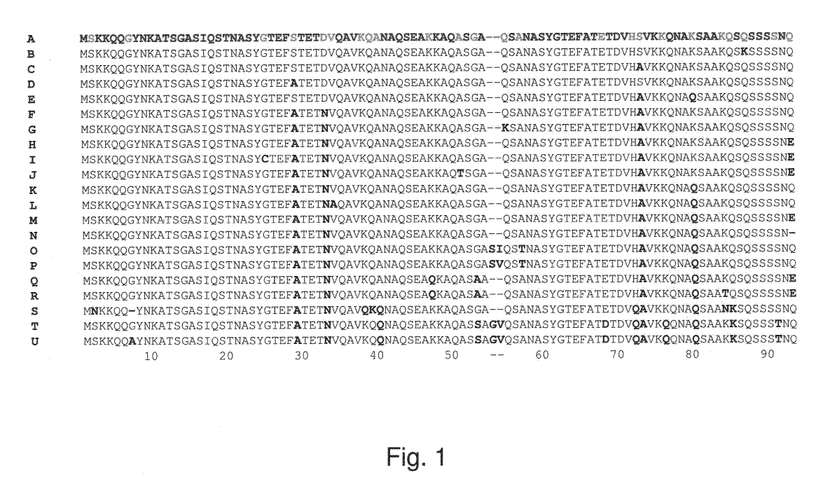

Tables 2 and 3. sspE genotypic and proteotypic clustering of Bc group isolates. This table was developed from the ClUSTALW multisequence alignment of Bc group amino acid sequences (see FIG. 1 below). The SspE sequence of B. cereus strain T (represented by BGSC 6 μl) was chosen as the holotype reference sequence to which all other Bc clade SspE sequences are compared. The numbers at the top of the table indicate amino acid position in the reference SspE sequence. Just below these numbers, the letters indicate the specific residue change from the 6A1 holotype reference sequence (top letter, above arrow). Residue changes are indicated by dots in the grid of the table. Colors are used to highlight similarities among proteotypes. The capital Greek letter delta (Δ) symbolizes a residue deletion at the indicated position with respect to the 6A1 holotype reference sequence.

To illustrate an example of grouping and segregation of commercially valuable Bc group strains by SspE sequence similarity clustering, groups are color coded as in the previous classifier table (Table 1). Selected isolates are indicated.

Insecticidal Bt serovar kurstaki (BGSC 4D#) isolates are clustered in group A1 as are insecticidal Bt serovar aizawai/pacificus (BGSC 4J#) isolates. These strains are indicated in bold blue type in Table 2. Insecticidal Bt serovar thuringiensis (BGSC 4A# & DSM 2046) isolates are clustered in group H4. These strains are indicated in Table 2. Insecticidal Bt serovar israelensis (BGSC 4Q# & ATCC 35646) isolates are clustered in group H5. These strains are indicated in Tables 2 and 3.

Isolates of B. anthracis, the causative agent of anthrax in animals and humans, cluster in sspE groups 0 and P and are indicated in bold type in Table 2. Pathogenic strains identified as B. cereus that were isolated from human and animal victims cluster in SspE proteotype E with genotypes 10 and 11, respectively, and are indicated in bold type in Table 2. Strain 97-27 is phylogenetically proximate to B. anthracis (see FIGS. 2 and 3) and is indicated in bold type in sspE genotype K2 in Table 2. Strain 97-27 was isolated from a war wound requiring limb amputation. Strain 97-27 has subsequently been shown to be highly lethal murine models. These strains have not been shown to be insecticidal, rather they are mammalian pathogens. ^{†} Proteotypes O, P (B. anthracis), T (B. mycoides) and U (B. pseudomycoides) have insert sequences of two amino acid residues between positions 54 and 55 of the proteotype A reference sequence.

TABLE 4
Bacillus thuringiensis group Strain Table
Table 4. List of strains used in the Bacillus thuringiensis group scheme.
Most strains were acquired from culture collections.

| Classifier^{a} | Strain |
|---|---|
| A1a | BGSC 4D1, BGSC 4D2, BGSC 4D4, BGSC 4D5, BGSC 4D6, BGSC 4D7, BGSC 4D8, BGSC 4D9, |
| | BGSC 4D10, BGSC 4D12, BGSC 4D14, BGSC 4D15, BGSC 4D16, BGSC 4D17, BGSC 4D18, |
| | BGSC 4D19, BGSC 4D20, BGSC 4D21, BGSC 4D22, BGSC 4G3, BGSC 4G5, BGSC 4I1, BGSC |
| | 4I2, IB/A |
| A1b | BGSC 4F1, BGSC 4F2, BGSC 4F3, BGSC 4F4, BGSC 4J5 |
| A1c | BGSC 4J1, BGSC 4J2, BGSC 4J3, BGSC 4J4, BGSC 4X1 |
| A1d | BGSC 4G1, BGSC 4G2, BGSC 4G4, BGSC 4G6, BGSC 4T1, ATCC 29730 |
| A1e | BGSC 4D11, BGSC 6A1, BGSC 6A2 |
| A1f | ATCC 11778 |
| A1g | BGSC 4BQ1, BGSC 4R1, NRRL B-21619 |
| A1h | BGSC 4BF1 |
| A1i | BGSC 4AL1 |
| A1j | BGSC 4CA1 |
| A1k | BGSC 4S2, BGSC 4S3 |
| A1l | BGSC 4AR1 |
| A1m | BGSC 4AT1 |
| A2a | BGSC 4AZ1, DSM 6021 |
| A2b | DSM 6076 |
| B1a | BGSC 4I3 |
| B1b | BGSC 4I4, BGSC 4I5 |
| C1a | BGSC 4L1, BGSC 4L2, BGSC 4L3 |
| D1a | ATCC 13472 |
| E1a | BGSC 6A6, ATCC 15816, |
| E1b | BGSC 4H1, ATCC 13061 |
| E1c | ATCC 55675 |
| E1d | BGSC 6A9 |
| E2a | BGSC 4B1, BGSC 4B2 |
| E2b | ATCC 51912 |
| E3a | BGSC 4AH1 |
| E4a | DM55 |
| E4b | BGSC 6E1, BGSC 6E2 |
| E4c | 003, III, IB, IV, III-BL, III-BS, BuIB |
| E4d | S8553/2 |
| E5a | BGSC 4CD1 |
| E6a | BGSC 4BH1 |
| E7a | BGSC 4Y1 |
| E8a | ATCC 4342 |
| E8b | BGSC 4BG1 |
| E9a | ATCC 10987 |
| E10a | Strain G9241 |
| E11a | Strain ZK (E33L) |
| F1a | BGSC 4AO1, BGSC 4AP1 |
| F2a | BGSC 6A3, BGSC 6A4, BGSC 4BU1, ATCC 27348, NRRL B-571 |
| F2b | BGSC 4W1 |
| F3a | BGSC 4H2 |
| F3b | BGSC 4AC1 |
| F4a | BGSC 6A5, ATCC 14579 |
| F4b | BGSC 4P1 |
| F4c | BGSC 4BW1 |
| F4d | BGSC 4BT1, BGSC 4CE1 |
| G1a | BGSC 4AM1 |
| H1a | BGSC 6A7, BGSC 6A8, 3466-8.1, Pey. 9 |
| H1b | BGSC 4BN1 |
| H1c | 2A6, 2C1 |
| H1d | Pey. 8 |
| H2a | BGSC 4AE1 |
| H2b | BGSC 4AF1 |
| H2c | BGSC 4U1 |
| H2d | BGSC 4BE1 |
| H2e | BGSC 4AN1 |
| H2f | BGSC 4AQ1 |
| H2g | Pey. 6 |
| H3a | ATCC 53522, ATCC 55609 |
| H3b | BGSC 4AG1 |
| H3c | BGSC 4V1 |
| H3d | BGSC 4Z1 |
| H4a | 4A1, 4A2, 4A3, 4A4, 4A5, 4A6, 4A7, 4A8, 4D3, DSM 2046^{T} |
| H4b | ATCC 55000 |
| H4c | BGSC 4BB1 |
| H4d | BGSC 4BP1 |
| H4e | BGSC 4A9 |
| H5a | BGSC 4BS1, BGSC 4C1, BGSC 4C2, BGSC 4C3, BGSC 4E3, BGSC 4E4, BGSC 4E5 |
| H5b | BGSC 4AV1, BGSC 4Q1, BGSC 4Q2, BGSC 4Q3, BGSC 4Q4, BGSC 4Q5, BGSC 4Q6, BGSC 4Q7, |
| | BGSC 4Q8, BGSC 18A1, ATCC 35646^{T} |
| H5c | BGSC 4AA1, BGSC 4AB1, BGSC 4K1, BGSC 4O1 |
| H5d | BGSC 4M1, BGSC 4M2, BGSC 4M3 |
| H5e | BGSC 4AK1 |
| H5f | BGSC 4E1, BGSC 4E2 |
| H5g | BGSC 4BR1 |
| H5h | BGSC 4BZ1 |
| I1a | BGSC 4K3 |
| J1a | ATCC 19647 |
| K1a | BGSC 4BC1 |
| K2a | BGSC 4AY1 |
| K2b | BGSC 4CC1 |
| K2c | BGSC 4BA1 |
| K2d | 97-27 |
| K2e | BGSC 4AS1, BGSC 4AU1 |
| K2f | BGSC 4BY1 |
| K2g | BGSC 4CB1 |
| K3a | BGSC 4BJ1, BGSC 4BX1 |
| K3b | BGSC 4BV1 |
| K3c | BGSC 4BK1 |
| L1a | BGSC 4AD1 |
| M1a | BGSC 4BL1 |
| M1b | I2 |
| N1a | BGSC 4AJ1 |
| O1a | ATCC 14578^{T}, Sterne, CAU-1, CAU-2, CAU-3, BC, Pasteur #2, Ames, A2084, A0039, Vollum |
| O1b | ATCC 14185, ATCC 14186 |
| O1c | CN1, CN2, CNEVA-9066, Kruger B |
| P1a | B. anthracis Western North America USA6153 |
| Q1a | DSM 11821 |
| Q1b | ATCC 6462 |
| Q1c | BGSC 4AX1 |
| Q1d | BGSC 4BM1 |
| Q1e | ATCC 11986 |
| R1a | ATCC 23258 |
| S1a | ATCC 21929 |
| T1a | ATCC 10206 |
| T1b | ATCC 31101 |
| T1c | ATCC 31102 |
| U1a | DSM 12442 |

BGSC = Bacillus Genetic Stock Center (Department of Biochemistry, The Ohio State University, 484 West Twelfth Avenue, Columbus, OH 43210, USA);
ATCC = American Type Culture Collection (P.O. Box 1549, Manassas, VA 20108, USA);
NRRL = the USDA ARS (NRRL) Culture Collection (National Center for Agricultural Utilization Research, Peoria, Illinois, USA);
DSM = DSMZ = Deutsche Sammlung von Mikroorganismen und Zellkulturen GmbH (Mascheroder Weg 1b, 38124 Braunschweig, Germany);
^{T}= Type Strain.

Bacillus anthracis strains CAU-1, CAU-2 and CAU-3 were isolated from human patients in South Korea, strain CN1 was isolated from a cow in South Korea, strain CN2 was isolated from soil in South Korea, strain BC was isolated in Boncheon, China and strain Pasteur#2 was acquired from the National Veterinary Research and Quarantine Service (Anyang-si, Kyeonggi-do, South Korea). DNA sequences of these isolates were provided by Dr. Kijeong Kim at Chung-Ang University, Seoul, South Korea.

Bacillus anthracis Sterne strain was obtained from Colorado Serum Company (P.O. Box 16428, Denver, Colo. 80216, USA). Strain DM55 was isolated in Egypt and was obtained from Dr. Ehab El-Helow at University of Alexandria, Alexandria 21526, Egypt. Strains 97-27, 3466-8.1, S8553/2, 2A6, 2C1, Pey. 6, Pey. 8, Pey. 9, 12, BuIB, IB, III, III-BL, III-BS, IV and 003 were obtained from the Pasteur Institute, Paris, France.

DNA sequences for Bacillus anthracis strains Ames, A2084, A0039, Vollum, CNEVA-9066, Kruger B, Western North America USA6153 and Bacillus cereus strains ZK (E33L) and G9241 were obtained from GenBank or TIGR databases as previously noted.

### PART I REFERENCES

1. Adang, M. J., Staver, M. J., Rocheleau, T. A., Leighton, J., Barker, R. F., & Thompson, D. V. (1985). Characterized full-length and truncated plasmid clones of the crystal protein of Bacillus thuringiensis subsp. kurstaki HD-73 and their toxicity to Manduca sexta. Gene 36, 289-300. PubMed ID 3000881
2. Alberghini, S., Filippini, R., Marchetti, E., Dindo, M. L., Shevelev, A. B., Battisti, A. & Squartini, A. (2005). Construction of a Pseudomonas sp. derivative carrying the cry9Aa gene from Bacillus thuringiensis and a proposal for new standard criteria to assess entomocidal properties of bacteria. Res Microbiol 156, 690-699. PubMed ID 15950125
3. Bourgouin, C., Delécluse, A., Ribier, J., Klier, A. & Rapoport, G. (1988). A Bacillus thuringiensis subsp. israelensis gene encoding a 125-kilodalton larvicidal polypeptide is associated with inverted repeat sequences. J Bacteriol 170, 3575-3583. PubMed ID 2841292
4. Bradley, D., Harkey, M. A., Kim M.-K., Biever, K. D. & Bauer, L. S. (1995). The insecticidal CryIB crystal protein of Bacillus thuringiensis ssp. thuringiensis has dual specificity to coleopteran and lepidopteran larvae. J Invertebr Pathol 65, 162-173. PubMed ID 7722342
5. Brown, K. L. & Whiteley, H. R. (1992). Molecular characterization of two novel crystal protein genes from Bacillus thuringiensis subsp. thompsoni. J Bacteriol 174, 549-557. PubMed ID 1729243
6. Bulla, L. A. Jr., Kramer K. J., Cox, D. J., Jones B. L., Davidson, L. I. & Lookhart, G. L. (1981). Purification and characterization of the entomocidal protoxin of Bacillus thuringiensis. J Biol Chem 256, 3000-3004. PubMed ID 7204384
7. Bulla, L. A. Jr., Kramer K. J. & Davidson, L. I. (1977). Characterization of the entomocidal parasporal crystal of Bacillus thuringiensis. J Bacteriol 130, 375-383. PubMed ID
8. Chambers, J. A., Jelen, A., Gilbert, M. P., Jany, C. S., Johnson, T. B. & Gawron-Burke, C. (1991). Isolation and characterization of a novel insecticidal crystal protein gene from Bacillus thuringiensis subsp. aizawai. J Bacteriol 173, 3966-3976. PubMed ID 2061280
9. Chilcott, C. N. & Ellar, D. J. (1988). Comparative toxicity of Bacillus thuringiensis var. israelensis crystal proteins in vivo and in vitro. J Gen Microbiol 134, 2551-2558. PubMed ID
10. Chilcott, C. N., Wigley P. J., Broadwell, A. H., Park, D. J. & Ellar, D. J. (1998). Activities of Bacillus thuringiensis insecticidal crystal proteins Cyt1Aa and Cyt2Aa against three species of sheep blowfly. Appl Environ Microbiol 64, 4060-4061. PubMed ID 9758842.
11. Chungjatupornchai, W., Höfte H., Seurinck, J., Angsuthanasombat, C. & Vaeck, M. (1988). Common features of Bacillus thuringiensis toxins specific for diptera and lepidoptera. Eur J Biochem 173, 9-16. PubMed ID 2833395
12. de Castilhos-Fortes, R., Matsumura, A. T. S., Diehl, E. & Fiuza, L. M. (2002). Susceptibility of Nasutitermes ehrhardti (Isoptera: Termitidae) to Bacillus thuringiensis subspecies. Braz J Microbiol 33, 219-222. PubMed ID none
13. Donovan, W. P., Dankocsik, C. & Gilbert, M. P. (1988). Molecular characterization of a gene encoding a 72-kilodalton mosquito-toxic crystal protein from Bacillus thuringiensis subsp. israelensis. J Bacteriol 170, 4732-4738. PubMed ID 2902069
14. Donovan, W. P., Dankocsik, C. C., Gilbert, M. P., Gawron-Burke, M. C., Groat, R. G. & Carlton, B. C. (1988). Amino acid sequence and entomocidal activity of the P2 crystal protein. An insect toxin from Bacillus thuringiensis var. kurstaki. J Biol Chem 263, 561-567. PubMed ID 3121615
15. Donovan, W. P., Rupar, M. J., Slaney, A. C., Malvar, T., Gawron-Burke, M. C. & Johnson, T. B. (1992). Characterization of two genes encoding Bacillus thuringiensis insecticidal crystal proteins toxic to Coleoptera species. Appl Environ Microbiol 58, 3921-3927. PubMed ID 1476436
16. Drobniewski, F. A. & Ellar, D. J. (1989). Purification and properties of a 28-kilodalton hemolytic and mosquitocidal protein toxin of Bacillus thuringiensis subsp. darmstadiensis 73-E10-2. J Bacteriol 171, 3060-3067. PubMed ID 2566594
17. Geiser, M., Schweitzer, S. & Grimm, C. (1986). The hypervariable region in the genes coding for entomopathogenic crystal proteins of Bacillus thuringiensis: nucleotide sequence of the kurhd1 gene of subsp. kurstaki HD1. Gene 48, 109-118. PubMed ID 3557124
18. Gill, S. S., Hornung, J. M., Ibarra, J. E., Singh, G. J. & Federici, B. A. (1987). Cytolytic activity and immunological similarity of the Bacillus thuringiensis subsp. israelensis and Bacillus thuringiensis subsp. morrisoni isolate PG-14 toxins. Appl Environ Microbiol 53, 1251-1256. PubMed ID 3300548
19. Granum, P. E., Pinnavaia, S. M. & Ellar, D. J. (1988). Comparison of the in vivo and in vitro activity of the δ-endotoxin of Bacillus thuringiensis var. morrisoni (HD-12) and two of its constituent proteins after cloning and expression in Escherichia coli. Eur J Biochem 172, 731-738. PubMed ID 2832170
20. Gringorten, J. L., Sohi, S. S. & Masson, L. (1999). Activity spectra of Bacillus thuringiensis δ-endotoxins against eight insect cell lines. In Vitro Cell Dev Biol Anim 35, 299-303. PubMed ID 10475277
21. Guerchicoff, A., Delécluse, A. & Rubinstein, C. P. (2001). The Bacillus thuringiensis cyt genes for hemolytic endotoxins constitute a gene family. Appl Environ Microbiol 67, 1090-1096. PubMed ID 11229896
22. Haider, M. Z. & Ellar, D. J. (1987). Characterization of the toxicity and cytopathic specificity of a cloned Bacillus thuringiensis crystal protein using insect cell culture. Mol Microbiol 1, 59-66. PubMed ID 3330756
23. Haider, M. Z., Knowles, B. H. & Ellar, D. J. (1986). Specificity of Bacillus thuringiensis var. colmeri insecticidal δ-endotoxin is determined by differential proteolytic processing of the protoxin by larval gut proteases. Eur J Biochem 156, 531-540. PubMed ID 3009187
24. Haider, M. Z., Ward, E. S. & Ellar, D. J. (1987). Cloning and heterologous expression of an insecticidal delta-endotoxin gene from Bacillus thuringiensis var. aizawai IC1 toxic to both lepidoptera and diptera. Gene 52, 285-290. PubMed ID 3038691
25. Han, C. S., Xie, G., Challacombe, J. F., Altherr, M. R., Bhotika, S. S., Bruce, D., Campbell, C. S., Campbell, M. L., Chen, J., Chertkov, O., Cleland, C., Dimitrijevic, M., Doggett, N. A., Fawcett, J. J., Glavina, T., Goodwin, L. A., Hill, K. K., Hitchcock, P., Jackson, P. J., Keim, P., Kewalramani, A. R., Longmire, J., Lucas, S., Malfatti, S., McMurry, K., Meincke, L. J., Misra, M., Moseman, B. L., Mundt, M., Munk, A. C., Okinaka, R. T., Parson-Quintana, B., Reilly, L. P., Richardson, P., Robinson, D. L., Rubin, E., Saunders, E., Tapia, R., Tesmer, J. G., Thayer, N., Thompson, L. S., Tice, H., Ticknor, L. O., Wills, P. L., Brettin, T. S. & Gilna, P. (2006). Pathogenomic sequence analysis of Bacillus cereus and Bacillus thuringiensis isolates closely related to Bacillus anthracis. J Bacteriol 188, 3382-3390. PubMed ID 16621833
26. Hernandez, E., Ramisse, F., Ducoureau, J.-P., Cruel, T. & Cavallo, J.-D. (1998). Bacillus thuringiensis subsp. konkukian (Serotype H34) superinfection: case report and experimental evidence of pathogenicity in immunosuppressed mice. J Clin Microbiol 36, 2138-2139. PubMed ID 9650985
27. Herrnstadt, C., Gilroy, T. E., Sobieski, D. A., Bennett, B. D. & Gaertner, F. H. (1987). Nucleotide sequence and deduced amino acid sequence of a coleopteran-active delta-endotoxin gene from Bacillus thuringiensis subsp. san diego. Gene 57, 37-46. PubMed ID 2828180
28. Higuchi, K., Saitoh, H., Mizuki, E., Hwang, S. H. & Ohba, M. (1998). A novel isolate of Bacillus thuringiensis serovar leesis that specifically exhibits larvicidal activity against the moth-fly, Telmatoscopus albipunctatus. Syst Appl Microbiol 21, 144-150. PubMed ID 9741119
29. Higuchi, K., Saitoh, H., Mizuki, E., Ichimatsu, T. & Ohba, M. (2000). Larval susceptibility of the diamondback moth, Plutella xylostella (Diptera: Plutellidae), to Bacillus thuringiensis H serovars isolated in Japan. Microbiol Res 155, 23-29. PubMed ID 10830896
30. Hoffmaster, A. R., Ravel, J., Rasko, D. A., Chapman, G. D., Chute, M. D., Marston, C. K., De, B. K., Sacchi, C. T., Fitzgerald, C., Mayer, L. W., Maiden, M. C., Priest, F. G., Barker, M., Jiang, L., Cer, R. Z., Rilstone, J., Peterson, S, N., Weyant, R. S., Galloway, D. R., Read, T. D., Popovic, T. & Fraser, C. M. (2004). Identification of anthrax toxin genes in a Bacillus cereus associated with an illness resembling inhalation anthrax. Proc Natl Acad Sci USA 101, 8449-8454. PubMed ID 15155910
31. Höfte, H., Van Rie, J., Jansens, S., Van Houtven, A., Vanderbruggen, H. & Vaeck, M. (1988). Monoclonal antibody analysis and insecticidal spectrum of three types of lepidopteran-specific insecticidal crystal proteins of Bacillus thuringiensis. Appl Environ Microbiol 54, 2010-2017. PubMed ID 16347711
32. Honée, G., van der Salm, T. & Visser, B. (1988). Nucleotide sequence of crystal protein gene isolated from B. thuringiensis subspecies entomocidus 60.5 coding for a toxin highly active against Spodoptera species. Nucleic Acids Res 16, 6240. PubMed ID 3399402
33. Hori, H., Suzuki, N., Ogiwara, K., Himejima, M., Indrasith, L. S., Minami, M., Asano, S., Sato, R., Ohba, M. & Iwahana, H. (1994). Characterization of a larvicidal toxin protein from Bacillus thuringiensis serovar japonensis strain Buibui specific for scarabaeid beetles. J Appl Bacteriol 76, 307-313. PubMed ID 8200856
34. Hurley, J. M., Bulla, L. A. Jr. & Andrews, R. E. Jr. (1987). Purification of the mosquitocidal and cytolytic proteins of Bacillus thuringiensis subsp. israelensis. Appl Environ Microbiol 53, 1316-1321. PubMed ID 3606108
35. Hwang, S. H., Saitoh, H., Mizuki, E., Higuchi, K. & Ohba, M. (1998). A novel class of mosquitocidal delta-endotoxin, Cry19B, encoded by a Bacillus thuringiensis serovar higo gene. Syst Appl Microbiol 21, 179-184. PubMed ID 9704107
36. Iriarte, J., Bel, Y., Ferrandis, M. D., Andrew, R., Murillo, J., Ferré, J. & Caballero, P. (1998). Environmental distribution and diversity of Bacillus thuringiensis in Spain. System Appl Microbiol 21, 97-106. PubMed ID 9741114
37. Iriarte, J., Dumanoir, V. C., Bel, Y., Porcar, M., Ferrandis, M. D., Lecadet, M., Ferré, J. & Caballero, P. (2000). Characterization of Bacillus thuringiensis ser. balearica (serotype H48) and ser. navarrensis (serotype H50): two novel serovars isolated in Spain. Curr Microbiol 40, 17-22. PubMed ID 10568798
38. Iriarte, J., Porcar, M., Lecadet, M.-M. & Caballero, P. (2000). Isolation and characterization of Bacillus thuringiensis strains from aquatic environments in Spain. Curr Microbiol 40, 402-408. PubMed ID 10827284
39. Ishii, T. & Ohba, M. (1993). Diversity of Bacillus thuringiensis environmental isolates showing larvicidal activity specific for mosquitoes. J Gen Microbiol 139, 2849-2854. PubMed ID 7903978
40. Ito, A., Sasaguri, Y., Kitada, S., Kusaka, Y., Kuwano, K., Masutomi, K., Mizuki, E., Akao, T. & Ohba, M. (2004). A Bacillus thuringiensis crystal protein with selective cytocidal action to human cells. J Biol Chem 279, 21282-21286. PubMed ID 15026424
41. Jaquet, F., Hütter, R. & Lüthy, P. (1987). Specificity of Bacillus thuringiensis delta-endotoxin. Appl Environ Microbiol 53, 500-504. PubMed ID 16347299
42. Kim H.-S., Yamashita, S., Akao, T., Saitoh, H., Higuchi, K., Park, Y. S., Mizuki, E. & Ohba, M. (2000). In vitro cytotoxicity of non-Cyt inclusion proteins of a Bacillus thuringiensis isolate against human cells, including cancer cells. J Appl Microbiol 89, 16-23. PubMed ID
43. Klowden, M. J. & Bulla, L. A. Jr. (1984). Oral toxicity of Bacillus thuringiensis subsp. israelensis to adult mosquitoes. Appl Environ Microbiol 48, 665-667. PubMed ID 6149725
44. Klowden, M. J., Held, G. A. & Bulla, L. A. Jr. (1983). Toxicity of Bacillus thuringiensis subsp. israelensis to adult Aedes aegypti mosquitoes. Appl Environ Microbiol 46, 312-315. PubMed ID 6625566
45. Knowles, B. H. & Ellar, D. J. (1986). Characterization and partial purification of a plasma membrane receptor for Bacillus thuringiensis var. kurstaki Lepidopteran-specific δ-endotoxin. J Cell Sci 83, 89-101. PubMed ID 3027110
46. Knowles, B. H. & Ellar, D. J. (1988). Differential specificity of two insecticidal toxins from Bacillus thuringiensis var. aizawai. Mol Microbiol 2, 153-157. PubMed ID 2835588
47. Knowles, B. H., Francis, P. H. & Ellar, D. J. (1986). Structurally related Bacillus thuringiensis δ-endotoxins display major differences in insecticidal activity in vivo and in vitro. J Cell Sci 84, 221-236. PubMed ID 3027111
48. Knowles, B. H., White, P. J., Nicholls, C. N. & Ellar, D. J. (1992). A broad-spectrum cytolytic toxin from Bacillus thuringiensis var. kyushuensis. Proc R Soc Lond 248, 1-7. PubMed ID 1355907
49. Kondo, S., Fujiwara, M., Ohba, M. & Ishii, T. (1995). Comparative larvicidal activities of the four Bacillus thuringiensis serovars against a chromoid midge, Paratanytarsus grimmii (Diptera: Chironomidae). Microbiol Res 150, 425-428. PubMed ID 8564369
50. Koni, P. A. & Ellar, D. J. (1994). Biochemical characterization of Bacillus thuringiensis cytolytic delta-endotoxins. Microbiology 140, 1869-1880. PubMed ID 7921240
51. Lacey, L. A., Lacey, C. M. & Padua, L. E. (1988). Host range and selected factors influencing the mosquito larvicidal activity of the PG-14 isolate of Bacillus thuringiensis var. morrisoni. J Am Mosq Control Assoc 4, 39-43. PubMed ID 3193097
52. Lee, D.-H., Shisa, N., Wasano, N., Ohgushi, A. & Ohba, M. (2003). Characterization of flagellar antigens and insecticidal activities of Bacillus thuringiensis populations in animal feces. Curr Microbiol 46, 287-290. PubMed ID 12732979
53. Lee, D.-W., Katayama, H., Akao, T., Maeda, M., Tanaka, R., Yamashita, S., Saitoh, H., Mizuki, E. & Ohba, M. (2001). A 28 kDa protein of Bacillus thuringiensis serovar. shandongiensis isolate 89-T-34-22 induces a human leukemic cell-specific cytotoxicity. Biochim Biophys Acta 1547, 57-63. PubMed ID 11343791
54. Lee, D.-W., Akao, T., Maeda, M., Yamashita, S., Katayama, H., Maeda, M., Saitoh, H., Mizuki, E. & Ohba, M. (2000). Noninsecticidal parasporal proteins of a Bacillus thuringiensis serovar. shandongiensis isolate exhibit a preferential cytotoxicity against human leukemic T cells. Biochem Biophys Res Commun 272, 218-223. PubMed ID 10872830
55. Lee, I. H., Je, Y. H., Chang, J. H., Roh, J. Y., Oh, H. W., Lee, S. G., Shin, S. C. & Boo, K. S. (2001). Isolation and characterization of a Bacillus thuringiensis ssp. Kurstaki strain toxic to Spodoptera exigua and Culex pipens. Curr Microbiol 43, 284-287. PubMed ID 11683364
56. MacIntosh, S. C., McPherson, S. L., Perlak, F. J., Marrone, P. G. & Fuchs, R. L. (1990). Purification and characterization of Bacillus thuringiensis var. tenebrionis insecticidal proteins produced in E. coli. Biochem Biophys Res Commun 170, 665-672. PubMed ID 2116794
57. MacIntosh, S. C., Stone, T. B., Sims, S. R., Hunst, P. L., Greenplate, J. T., Marrone, P. G., Perlak, F. J., Fischoff, D. A. & Fuchs, R. L. (1990). Specificity and efficacy of purified Bacillus thuringiensis proteins against agronomically important insects. J Invertebr Pathol 56, 258-266. PubMed ID 2273290
58. Maeda, M., Mizuki, E., Nakamura, Y., Hatano, T. & Ohba, M. (2000). Recovery of Bacillus thuringiensis from marine sediments of Japan. Curr Microbiol 40, 418-422. PubMed ID
59. Manonmani, A. & Balaraman K. (2001). A highly mosquitocidal Bacillus thuringiensis var. thompsoni. Curr Sci 80, 779-781. PubMed ID none
60. Masson, L., Marcotte, P., Préfontaine, G. & Brousseau, R. (1989). Nucleotide sequence of a gene cloned from Bacillus thuringiensis subspecies entomocidus coding for an insecticidal protein toxic for Bombyx mori. Nucleic Acids Res 17, 446. PubMed ID 2911478
61. Mizuki, E., Ohba, M., Akao, T., Yamashita, S., Saitoh, H. & Park, Y. S. (1999). Unique activity associated with non-insecticidal Bacillus thuringiensis parasporal inclusions: in vitro cell-killing action on human cancer cells. J Appl Bacteriol 86, 477-486. PubMed ID 10196753
62. Ogiwara, K., Hori, H., Minami, M., Takeuchi, K., Sato, R., Ohba, M. & Iwahana, H. (1995). Nucleotide sequence of the gene encoding novel delta-endotoxin from Bacillus thuringiensis serovar japonensis strain Buibui specific to scarabaeid beetles. Curr Microbiol 30, 227-235. PubMed ID 7765896
63. Ohba, M. & Aizawa, K. (1990). Occurrence of two pathotypes in Bacillus thuringiensis subsp. fukuokaensis (flagellar serotype 3a:3d:3e). J Invertebr Pathol 55, 293-294. PubMed ID
64. Ohba, M., Saitoh, H., Miyamoto, K., Higuchi, K. & Mizuki, E. (1995). Bacillus thuringiensis serovar higo (flagellar serotype 44), a new serogroup with a larvicidal activity preferential for the anopheline mosquito. Lett Appl Microbiol 21, 316-318. PubMed ID 7576527
65. Ohgushi, A., Wasano, N., Shisa, N., Saitoh, H., Mizuki, E., Maeda, M. & Ohba, M. (2003). Characterization of a mosquitocidal Bacillus thuringiensis serovar sotto strain from Okinawa, Japan. J Appl Microbiol 95, 982-989. PubMed ID none
66. Okumura, S., Akao, T., Higuchi, K., Saitoh, H., Mizuki, E., Ohba, M. & Inouye, K. (2004). Bacillus thuringiensis serovar shandongiensis strain 89-T-34-22 produces multiple cytotoxic proteins with similar molecular masses against human cancer cells. Lett Appl Microbiol 39, 89-92. PubMed ID 15189293
67. Padua, L. E. & Federici, B. A. (1990). Development of mutants of the mosquitocidal bacterium Bacillus thuringiensis subspecies morrisoni (PG-14) toxic to lepidopterous or dipterous insects. FEMS Microbiol Lett 54, 257-262. PubMed ID 1969828
68. Padua, L. E., Ohba, M. & Aizawa, K. (1980). The isolates of Bacillus thuringiensis serotype 10 with a highly preferential toxicity to mosquito larvae. J Invertebr Pathol 36, 180-186. PubMed ID 6108343
69. Polanczyk, R. A., da Silva, R. F. P. & Fiuza, L. M. (2003). Screening of Bacillus thuringiensis isolates pathogenic to Spodoptera frugiperda (J. E. Smith) (Lepidoptera: Noctuidae). Arq Inst Biol 70, 69-72.
70. Polanczyk, R. A., Garcia, M. deO. & Alves, S. B. (2003). Potential of Bacillus thuringiensis Berliner for controlling Aedes aegypti. Rev Saude Publica 37, 813-816. PubMed ID
71. Porcar, M., Iriarte, J., Cosmao Dumanoir, V., Ferrandis, M. D., Lecadet, M., Ferre, J. & Caballero, P. (1999). Identification and characterization of the new Bacillus thuringiensis serovars pirenaica (serotype H57) and iberica (serotype H59). J Appl Microbiol 87, 640-648. PubMed ID 10594703
72. Promdonkoy, B., Chewawiwat, N., Tanapongpipat, S., Luxananil, P. & Panyim, S. (2003). Cloning and characterization of a cytolytic and mosquito larvicidal δ-endotoxin from Bacillus thuringiensis subsp. darmstadiensis. Curr Microbiol 46, 94-98. PubMed ID 12520362
73. Ragni, A., Thiéry, I. & Delécluse, A. (1996). Characterization of six highly mosquitocidal Bacillus thuringiensis strains that do not belong to H-14 serotype. Curr Microbiol 32, 48-54. PubMed ID 8555944
74. Rupar, M. J., Donovan, W. P., Groat, R. G., Slaney, A. C., Mattison, J. W., Johnson, T. B., Charles, J.-F., Dumanoir, V. C. & de Barjac, H. (1991). Two novel strains of Bacillus thuringiensis toxic to coleopterans. Appl Environ Microbiol 57, 3337-3344. PubMed ID 1781691
75. Saitoh, H., Hwang, S. H., Park, Y. S., Higuchi, K., Mizuki, E. & Ohba, M. (2000). Cloning and characterization of a Bacillus thuringiensis serovar higo gene encoding a novel class of the delta-endotoxin protein, Cry27A, specifically active on the Anopheles mosquito. Syst Appl Microbiol 23, 25-30. PubMed ID 10879975
76. Saitoh, H., Higuchi, K., Mizuki, E., Hwang, S. H. & Ohba, M. (1998). Characterization of mosquito larvicidal parasporal inclusions of a Bacillus thuringiensis serovar higo strain. J Appl Microbiol 84, 883-888. PubMed ID 9674143
77. Saitoh, H., Higuchi, K., Mizuki, E. & Ohba, M. (1996). Larvicidal activity of Bacillus thuringiensis natural isolates, indigenous to Japan, against two nematoceran insect pests occurring in urban sewage environments. Microbiol Res 151, 263-271. PubMed ID 8817917
78. Saitoh, H., Higuchi, K., Mizuki, E. & Ohba, M. (1998). Larvicidal toxicity of Japanese Bacillus thuringiensis against the mosquito Anopheles stephensi. Med Vet Entomol 12, 98-102. PubMed ID 9513945
79. Sato, R., Takeuchi, K., Ogiwara, K., Minami, M., Kaji, Y., Suzuki, N., Hori, H., Asano, S., Ohba, M. & Iwahana, H. (1994). Cloning, heterologous expression, and localization of a novel crystal protein gene from Bacillus thuringiensis serovar japonensis strain buibui toxic to scarabaeid beetles. Curr Microbiol 28, 15-19. PubMed ID 7764305
80. Schesser, J. H. & Bulla, L. A. Jr. (1978). Toxicity of Bacillus thuringiensis to the tobacco hornworm, Manduca sexta. Appl Environ Microbiol 35, 121-123. PubMed ID 623457
81. Schesser, J. H. & Bulla, L. A. Jr. (1979). Toxicity of parasporal crystals of Bacillus thuringiensis to the Indian meal moth, Plodia interpunctella. Appl Environ Microbiol 37, 1012-1015. PubMed ID 485134
82. Schesser, J. H., Kramer K. J. & Bulla, L. A. Jr. (1977). Bioassay for homogenous parasporal crystal of Bacillus thuringiensis using the tobacco hornworm, Manduca sexta. Appl Environ Microbiol 33, 878-880. PubMed ID 869534
83. Schnepf, E., Crickmore, N., Van Rie, J., Lereclus, D., Baum, J., Feitelson, J., Zeigler, D. & Dean, D. H. (1998). Bacillus thuringiensis and its pesticidal crystal proteins. Microbiol Mol Biol Rev 62, 775-806. PubMed ID 9729609
84. Schnepf, H. E., Tomczak, K., Ortega, J. P. & Whiteley, H. R. (1990). Specificity-determining regions of a lepidopteran-specific insecticidal protein produced by Bacillus thuringiensis. J Biol Chem 265, 20923-20930. PubMed ID 2250000
85. Sekar, V., Thompson, D. V., Maroney, M. J., Brookland, R. G. & Adang, M. J. (1987). Molecular cloning and characterization of the insecticidal crystal protein gene of Bacillus thuringiensis var. tenebrionis. Proc Natl Acad Sci USA 84, 7036-7040. PubMed ID 16593878
86. Shibano, Y., Yamagata, A., Nakamura, N., Iizuka, T., Sugisaki, H. & Takanami, M. (1985). Nucleotide sequence coding for the insecticidal fragment of the Bacillus thuringiensis crystal protein. Gene 34, 243-251. PubMed ID 2989108
87. Shin, B.-S., Park S.-H., Choi, S.-K., Koo, B.-T., Lee, S.-T. & Kim, J.-I. (1995). Distribution of cryV-type insecticidal protein genes in Bacillus thuringiensis and cloning of cryV-type genes from Bacillus thuringiensis subsp. kurstaki and Bacillus thuringiensis subsp. entomocidus. Appl Environ Microbiol 61, 2402-2407. PubMed ID 7793960
88. Sick, A., Gaertner, F. & Wong, A. (1989). Nucleotide sequence of a coleopteran-active toxin gene from a new isolate of Bacillus thuringiensis subsp. tolworthi. Nucleic Acids Res 18, 1305. PubMed ID 2320431
89. Smith, G. P., Merrick, J. D., Bone, E. J. & Ellar, D. J. (1996). Mosquitocidal activity of the CryIC δ-endotoxin from Bacillus thuringiensis subsp. aizawai. Appl Environ Microbiol 62, 680-684. PubMed ID 8593070
90. Temeyer K. B. (1984). Larvicidal activity of Bacillus thuringiensis subsp. israelensis in the dipteran Haematobia irritans. Appl Environ Microbiol 47, 952-955. PubMed ID 6742837
91. Thomas W. E. & Ellar, D. J. (1983). Mechanism of action of Bacillus thuringiensis var. israelensis insecticidal δ-endotoxin. FEBS Lett 154, 362-368. PubMed ID 6832375
92. Tyrell, D. J., Bulla, L. A. Jr., Andrews, R. E. Jr., Kramer, K. J., Davidson, L. I. & Nordin, P. (1981). Comparative biochemistry of entomocidal parasporal crystals of selected Bacillus thuringiensis strains. J Bacteriol 145, 1052-1062. PubMed ID 7462158
93. Tyrell, D. J., Davidson, L. I., Bulla, L. A. Jr. & Ramoska, W. A. (1979). Toxicity of parasporal crystals of Bacillus thuringiensis subsp. israelensis to mosquitoes. Appl Environ Microbiol 38, 656-658. PubMed ID 44177
94. Von Tersch, M. A., Robbins, H. L., Jany, C. S. & Johnson, T. B. (1991). Insecticidal toxins from Bacillus thuringiensis subsp. kenyae: gene cloning and characterization and comparison with B. thuringiensis subsp. kurstaki CryIA(c) toxins. Appl Environ Microbiol 57, 349-358. PubMed ID 2014985
95. Ward, E. S. & Ellar, D. J. (1988). Cloning and expression of two homologous genes of Bacillus thuringiensis subsp. israelensis which encode 130-kilodalton mosquitocidal proteins. J Bacteriol 170, 727-735. PubMed ID 2828321
96. Wasano, N. & Ohba, M. (1998). Assignment of δ-endotoxin genes of the four Lepidoptera-specific Bacillus thuringiensis strains that produce spherical parasporal inclusions. Curr Microbiol 37, 408-411. PubMed ID 9806979
97. Wasano, N., Kim, K.-H & Ohba, M. (1998). Delta-endotoxin proteins associated with spherical parasporal inclusions of the four Lepidoptera-specific Bacillus thuringiensis strains. J Appl Microbiol 84, 501-508. PubMed ID 9633648
98. Widner, W. R. & Whiteley, H. R. (1989). Two highly related insecticidal crystal proteins of Bacillus thuringiensis subsp. kurstaki possess different host range specificities. J Bacteriol 171, 965-974. PubMed ID 2914879
99. Yamamoto, T. & McLaughlin, R. E. (1981). Isolation of a protein from the parasporal crystal of Bacillus thuringiensis var. kurstaki toxic to the mosquito larva, Aedes taeniorhynchus. Biochem Biophys Res Commun 103, 414-421. PubMed ID 7332548
100. Yasutake, K., Binh, N. D., Kagoshima, K., Uemori, A., Ohgushi, A., Maeda, M., Mizuki, E., Yu, Y. M. & Ohba, M. (2006). Occurrence of parasporin-producing Bacillus thuringiensis in Vietnam. Can J Microbiol 52, 365-372. PubMed ID 16699587
101. Yu, Y.-M., Ohba, M. & Gill, S. S. (1991). Characterization of mosquitocidal activity of Bacillus thuringiensis subsp. fukuokaensis crystal proteins. Appl Environ Microbiol 57, 1075-1081. PubMed ID 2059032
102. Zeigler, D. R. (1999). Bacillus Genetic Stock Center Catalog of strains; Bacillus thuringiensis & Bacillus cereus, 7^{th} Edition, vol 2.
103. http://www.nal.usda.gov/bic/BTTOX/BT-patents/05298245.html
104. U.S. Pat. Nos. 5,049,379 and 5,543,301 and 5,869,042
105. U.S. Pat. No. 5,403,583
106. U.S. Pat. No. 6,232,270
107. U.S. Pat. Nos. 5,919,447 and 6,077,506
108. This publication made use of the Bacillus cereus Multi Locus Sequence Typing website (http://pubmlst.org/bcereus/) developed by Keith Jolley and sited at the University of Oxford (Jolley et al. 2004, BMC Bioinformatics, 5:86). The development of this site has been funded by the Wellcome Trust.
109. Cappello, M., Bungiro, R. D., Harrison, L. M., Bischof, L. J., Griffitts, J. S., Barrows, B. D. & Aroian, R. V. (2006). A purified Bacillus thuringiensis crystal protein with therapeutic activity against the hookworm parasite Ancylostoma ceylanicum. Proc Natl Acad Sci USA 103, 15154-15159. PubMed ID 17005719
110. Marroquin, L. D., Elyassnia, D., Griffitts, J. S., Feitelson, J. S. & Aroian, R. V. (2000). Bacillus thuringiensis (Bt) toxin susceptibility and isolation of resistance mutants in the nematode Caenorhabditis elegans. Genetics 155, 1693-1699. PubMed ID 10924467
111. Shin, B.-S., Park, S.-H., Choi, S.-K., Koo, B.-T., Lee, S.-T. & Kim, J.-I. (1995). Distribution of cryV-type insecticidal protein genes in Bacillus thuringiensis and cloning of cry V-type genes from Bacillus thuringiensis subsp. kurstaki and Bacillus thuringiensis subsp. entomocidus. Appl Environ Microbiol 61, 2402-2407. PubMed ID 7793960

### PART II ClASSIFYING BACILLUS BACTERIA IN THE BACILLUS SUBTILIS/LICHENIFORMIS GROUP

Abbreviations: Bs=Bacillus subtilis, Bat=Bacillus atrophaeus, Bmo=Bacillus mojavensis, Bv=Bacillus vallismortis, Bl=Bacillus licheniformis, Bson=Bacillus sonorensis, Bamy=Bacillus amyloliquefaciens, Bpum=Bacillus pumilus, Bsp=Bacillus species; n/d=not determined; ^{T}=Type strain, MLST=multilocus sequence typing, ST=MLST sequence type, sspE=the (nucleotide sequence of the) gene encoding gamma-type small acid soluble spore protein, SspE=the (translated amino acid sequence of the) gene encoding gamma-type small acid soluble spore protein. Greek letters used: α=alpha, β=beta, γ=gamma, δ=delta. The capital Greek letter delta (Δ) is used to represent a nucleotide or amino acid residue deletion in a sequence.

The Bacillus subtilis/licheniformis group scheme: The Bs clade contains the Bs, Bl, Bat, Bmoj, Bv, Bson, Bamy and Bpum species. Though easily distinguished from the Bc clade, the species within the Bs group are not readily differentiated from one another, even with extensive biochemical and microbiological analyses. Often, DNA-DNA hybridization assays are the only means of species-level assignment within the Bs group. sspE sequences from the Bacillus subtilis/licheniformis group isolates examined in this study will be deposited in the GenBank nucleotide sequence database.

In addition to sspE phylogenetic analysis, we analyzed approximately 135 Bs group isolates by a multilocus sequence typing (MLST) scheme. Although several MLST schemes have been developed for the Bc group, which is of particular interest because B. anthracis is a member of this clade, and other groups of pathogenic organisms, less attention has been paid to the avirulent B. subtilis clade. There are several reasons for this situation: (1) these organisms are relatively harmless to humans, livestock, insects etc. (2) identification within the Bs group has been difficult because they lack flagellar antigens (which are essential for serotyping Bt isolates), (3) Bs group strains frequently lack plasmids, enterotoxins or plasmid-associated virulence factors (like Bc and Ba), and (4) the morphological and biochemical similarity of Bs group strains has prevented species-level discrimination in many cases. Thus, species that are beneficial to agriculture, industry and human health have not been well-characterized genetically and there remains profound confusion in much of the Bacillus community regarding distinction of species, subspecies and strains within this group. The only means currently available for identification of beneficial B. subtilis group bacteria are tedious and costly biochemical and microbiological assays. Molecular assays, such as 16S rRNA, have limited, utility due to the coarse resolution provided by this slowly evolving gene. The utility of phylogenetic placement and identification by sspE and MLST is unprecedented for this group of organisms and is an invaluable means of discovery in the growing biofungicide and agricultural protection industries, as well as in the massive industrial enzyme, health, and probiotic industries.

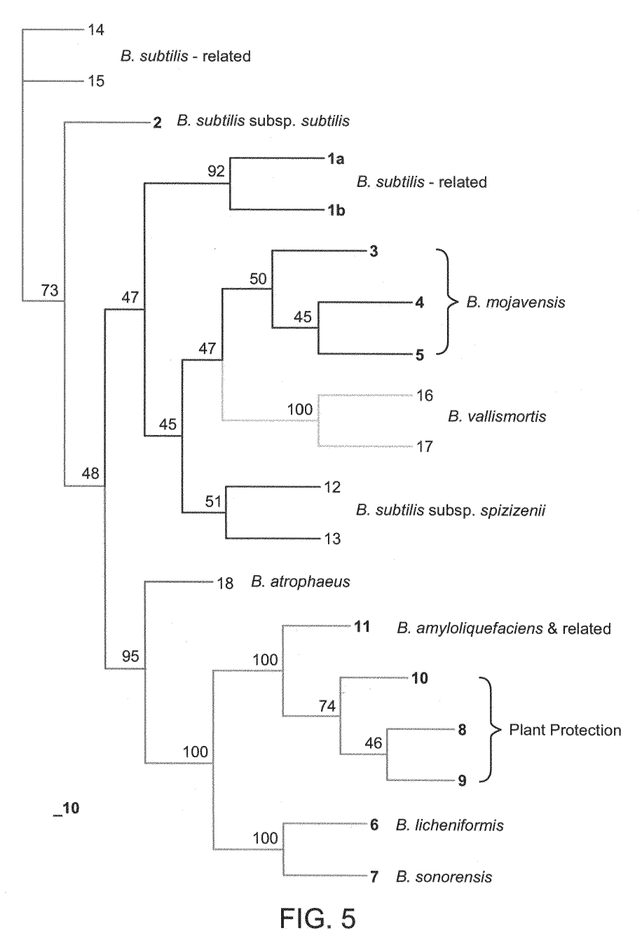

By color-coding the trees and tables, we illustrate the congruence of sspE and MLST phylogenetic clustering. We show in the following color-coded (violet, coral, gold, dark teal, gray, leaf green and aqua) Tables 5 and 6 and FIGS. 5-6 and 8-9 that orthogonal MLST analysis maintains the bona fide species and subspecies phylogenetic affiliation provided by the sspE method and additionally provides complementary resolution of subspecies and strain clusters. The complementarities and phylogenetic resolving power of these two orthogonal methodologies are unexpected and highly useful for classification of known and unknown strains of this commercially important group of microorganisms. Classifiers in the tables and groups/branches on the trees are color-coded to illustrate the equivalence of the phylogenies from one scheme to another i.e. to validate sspE as a robust single-gene molecular chronometer for the Bacillus genus. Color-coded (violet, coral, gold, dark teal, gray, leaf green and aqua) groups, classifiers and branches remain consistent in that a species or subspecies sspE cluster that is color-coded coral, for example, in the sspE tree or table will not be in the violet or leaf green groups for MLST STs, tree branches, or overall classifier, and vice versa. Specifically, in our study of 135 Bs group isolates, comprising seven bona fide species and including two bona fide subspecies of B. subtilis, STs uniquely cluster within sspE genotype or proteotype, and as in the case of the Bc group, sspE tree topology and clustering are congruent with the MLST tree topology.

There are, however, several isolates in the Bs group that have been misclassified or misidentified. For example, an isolate currently identified as B. licheniformis clusters with B. sonorensis and three isolates identified as B. subtilis cluster in the B. atrophaeus group⁵. These examples of misidentification demonstrate the power of the described invention assay to correctly assign isolate to bona fide species. Depicted in Table 5 and highlighted in yellow are specific instances of misidentified or misclassified B. subtilis group isolates in the following classifier groups: 1b, 2c, 2d, 2h, 2l, 2j, 7a, 8a, 8b, 8c, 9a, 10a, 11a, 11b, 11c and 18a.

Utility—Bacillus subtilis Group Scheme (See Also Table 5.)

The utility of this method covers not only identification of Bacillus species which are of economic importance, but also the use of genes which may be removed from these bacteria or their plasmids which may be cloned into other bacteria, plants, etc. as well as derivatives or byproducts of substances produced by these bacteria.

1. EXEMPLARY UTILITY—biofungicide, drain opener, cleaner and sanitizer. SspE proteotype 1 contains a strain misidentified as Bacillus licheniformis that is patented for use as a biofungicide, drain opener, cleaner and sanitizer^{8, 11, 20}. This strain, ATCC 55406, is also available commercially as Ecoguard®. Also in proteotype 1 is Bacillus subtilis strain DSM 5552 which is not currently known to have commercial utility. A molecular signature for this group is SspE translated protein sequence length of 85 amino acids, with the following residue characteristics: S at position 7, K at position 43, A at position 67.

2. EXEMPLARY UTILITY—produces enzymes of commercial interest such as proteases, amylases, cellulases and lipases; purine nucleotides and nucleosides; D-Ribose; lipopeptide antibiotics; and the vitamin riboflavin. SspE proteotype 2 is a Bacillus subtilis cluster that contains the laboratory strain 168 and Bacillus subtilis natto strains, both of which are well-known to produce enzymes of commercial interest. Two isolates in this cluster, DSM 1970 and DSM 1971, are patented for enzyme production²⁵⁻²⁶, including alkaline proteases and subtilisins. Recently, NZyme Pharmaceuticals, Inc. announced a pending patent application for Subtilisin NAT (derived from Natto, the Japanese food product, which is made by fermenting soybeans with Bacillus subtilis “natto”) which “decreases whole blood viscosity in the central therapeutic role of preventing and treating vascular disease such as heart attacks and ischemic strokes, essential hypertension and deep vein thrombosis.” Three misidentified isolates cluster in this group: NRRL B-642 (previously identified as B. licheniformis), BGSC 10A5T (previously identified as B. amyloliquefaciens) and BGSC 2A10 (previously identified as B. subtilis subsp. spizizenii). A molecular signature for this group is SspE translated protein sequence length of 84 amino acids, with the following residue characteristics: G at position 54, A at position 66.

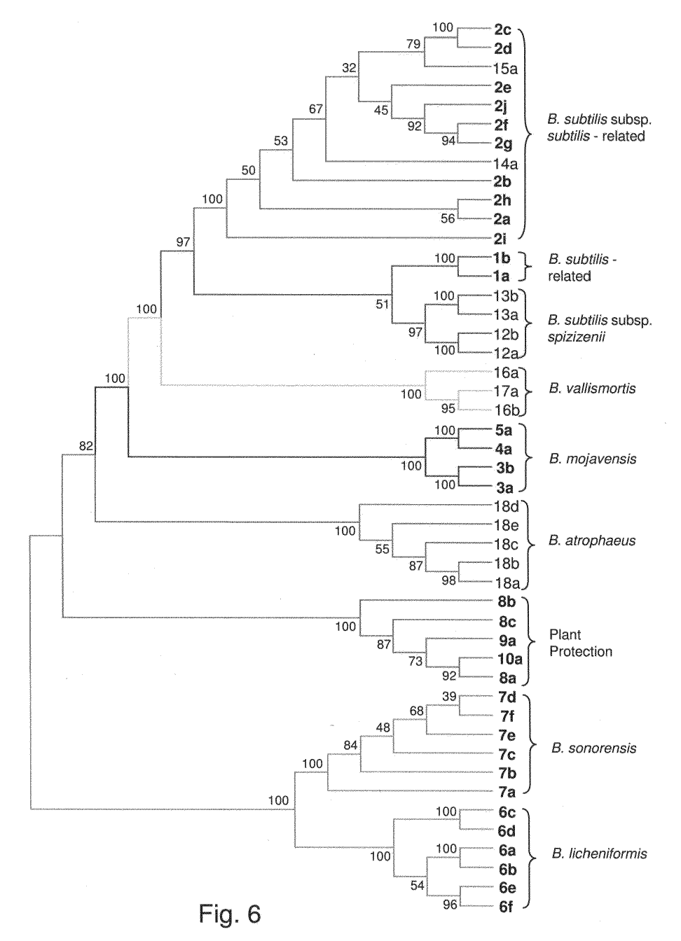

3. EXEMPLARY UTILITY—produces enzymes of commercial interest such as alkaline proteases and amylases. Bacillus licheniformis and Bacillus sonorensis are two very closely related species, yet SspE and MLST phylogenetic analyses readily distinguish the species (see proteotypes 6 (Bl) and 7 (Bson), aqua branches in FIGS. 5 and 6). Bacillus licheniformis SspE proteotype 6 contains a strain, DSM 1969, patented for enzyme production³⁶, including alkaline proteases. Also in proteotype 6 are fourteen other B. licheniformis isolates which are not currently known to have commercial utility. Three misidentified B. licheniformis strains cluster elsewhere (proteotypes 1, 2 and 7). A molecular signature for this group is SspE translated protein sequence length of 54 amino acids, with the following residue characteristics: Q at position 41, K at position 49.

4. EXEMPLARY UTILITY—produces amino acids of commercial interest. Bacillus sonorensis and Bacillus licheniformis are two very closely related species, yet SspE and MLST phylogenetic analyses readily distinguish the species (see proteotypes 6 (Bl) and 7 (Bson), aqua branches in FIGS. 5 and 6). Bacillus sonorensis SspE proteotype 7 contains a strain, DSM 1913, patented for amino acid production⁷, including the food additive 5-hydroxytryptophan. This strain is misidentified as B. licheniformis and clusters both by SspE and MLST phylogenetic analysis with all eight B. sonorensis strains assayed. Bacillus sonorensis is not currently known to have any commercial utility. A molecular signature for this group is SspE translated protein sequence length of 54 amino acids, with the following residue characteristics: K at position 41, N at position 49.

5. EXEMPLARY UTILITY—plant protection, enzyme production, drain opener, cleaner and sanitizer; SspE proteotypes 8-11. This cluster of strains that we designate as the plant protection group is potentially the most commercially important and valuable plant protection and enzyme production cluster in the Bacillus group (see proteotypes 8-11 leaf green branches in FIG. 5 and proteotypes 8-10 leaf green branches in FIG. 6). This group of strains is characterized by the following molecular signatures in the SspE translated protein sequence: translated protein sequence length of 56 amino acids, with the following residue characteristics: A or E at position 2, D at position 10, V at position 11, K at position 15, K or R at position 16, S at position 23, D at position 37, A or V at position 38. SspE proteotype 8 strain GB03, misidentified as B. subtilis, is available commercially in two plant protection (biofungicide^{4, 15}) products: Kodiak® (Gustafson, Plano, Tex.) and Companion® (Growth Products, White Plains, N.Y.). Three other strains misidentified as B. subtilis that cluster in proteotype 8, DSM 8563, DSM 8564 and DSM 8565, are reported to have antifungal activity²⁴, though we have not located patents or commercial products for these strains. BGSC strain 10A6, identified as B. amyloliquefaciens, clusters in proteotype 8 and has also reportedly has antifungal properties¹⁶. Strain DSM 1324, identified only as Bacillus sp., also clusters in this proteotype, is not currently known to have any commercial utility. Strain NRRL B-21619, also known as AQ713 and QST 713 and misidentified as Bacillus subtilis, belongs to proteotype 9 and has the above molecular signature. It is available commercially as Serenade® and Rhapsody® biofungicide products from AgraQuest (Davis, Calif.)^{12-14, 18}. This strain recently (Jul. 14, 2006) received approval for inclusion into Annex 1 of Directive 91/414/EEC at the European Union Standing Committee on the Food Chain and Animal Health meeting according to an AgraQuest Sep. 13, 2006 press release. Currently, Serenade® is “registered on a provisional basis in France and Italy where it is used commercially on grapes to prevent botrytis bunch rot control [and] in Italy to protect apple crops from scab and fire blight,” according to the company. Strain ATCC 55614, also misidentified as Bacillus subtilis, belongs to proteotype 10 and has the above molecular signature. It is a patented strain (Agritope, Inc., Portland, Oreg.) that produces antibiotics and inhibits growth of plant pathogenic fungi and bacteria, and thus can be used for treating and protecting plants from disease²²⁻²³. Two strains identified as Bacillus amyloliquefaciens belong to proteotype 11 and have the above molecular signature. DSM 7 and DSM 1060 are patented strains³⁷ that produce enzymes of commercial importance such as amylase and α-amylase. Two other strains in proteotype 11, ATCC 55405 and ATCC 55407 are misidentified as Bacillus subtilis and Paenibacillus polymyxa, respectively. They are both patented by Sybron Chemical Holdings, Inc. (Wilmington, Del.) for use as a drain opener, cleaner and sanitizer^{11, 20}. Proteotype 11 strains with no known commercial utility include BGSC strains 3A14 and 3A23.

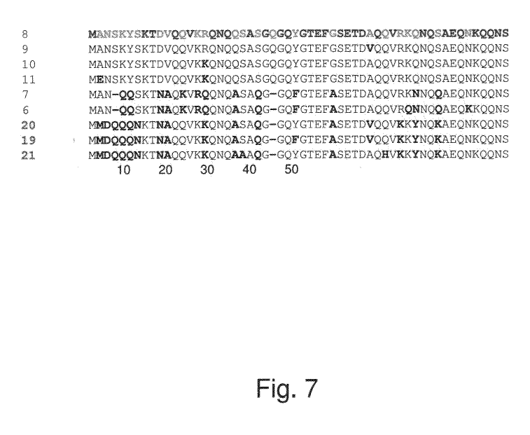

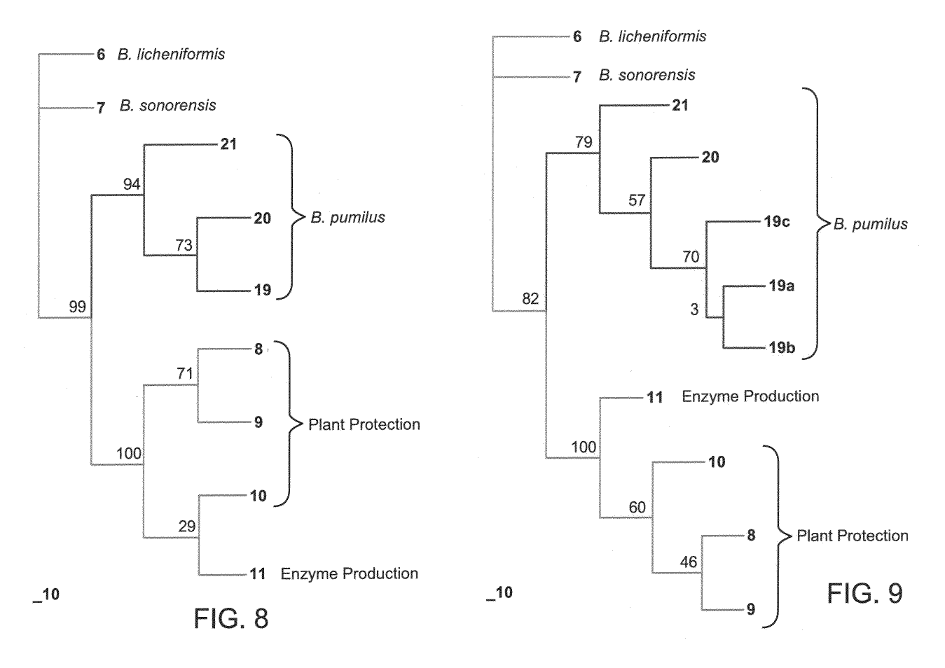

6. EXEMPLARY UTILITY—probiotic health supplement. Five Bacillus pumilus strains cluster phylogenetically intermediate to the B. licheniformis/sonorensis and the Bacillus species clusters by SspE proteotype analysis (see proteotypes 19-21 brown branches in FIG. 8 and Table 8 and genotypes 19a-c, 20 and 21 brown branches in FIG. 9). This group of strains is characterized by the following molecular signatures in the SspE translated protein sequence: translated protein sequence length of 55 amino acids, with the following residue characteristics: M at positions 1 and 2, D at position 3, Q at position 4, N at position 7, S or A at position 21, Y or F at position 27, A or V at position 37, Q or H at position 39, K at position 41, Y at position 43, K at position 46. SspE proteotype 20 strain BGSC 14A1 was isolated from the commercial probiotic Biosubtyl (Biophar Co. Ltd., Vietnam)^{10, 30}. Four other strains identified as Bacillus pumilus, DSM 354, DSM 355, ATCC 27142 and BGSC 8A1, also cluster in this group (see Table 8 and FIGS. 7-9) but are not currently known to have any commercial utility. Phylogenetic analysis of the B. pumilus group was done separately due to the unusual sspE coding sequence containing two potential methionine residues at the N-terminus. Furthermore, B. pumilus is distantly related to other organisms in the Bs/Bl group that were typeable by MLST and hence forms a separate cluster with an indeterminate SspE N-terminus and incomplete MLST data due to unsuccessful priming at several MLST loci.

Molecular Diagnostic Screening Targets—Bacillus subtilis Group Scheme (See Also Table 5.)

6. Bacillus mojavensis isolates cluster in SspE proteotypes 3, 4 and 5 (see FIGS. 5 and 6, dark teal branches). This species has been described in the literature to have antifungal activity^{2, 3, 28}, and thus has potential utility for crop protection. As far as we are aware, none of the isolates that we have genotyped have been tested for antifungal activity. All Bacillus mojavensis isolates studied cluster into a coherent and distinct phylogenetic clade by SspE protein, DNA and concatenated MLST DNA fragment analysis with no misidentified or mischaracterized strains. Thus, sspE sequence typing is a rapid and inexpensive means for unambiguous discrimination of B. mojavensis strains. A molecular signature for this group is SspE translated protein sequence length of 85 amino acids, which are distinctively identified by having a Q residue at position 4. Bacillus mojavensis isolates also have the following residue characteristics: A (proteotypes 4 and 5) or V (proteotype 3) at position 39, D (proteotype 4) or N (proteotypes 3 and 5) at position 66.

7. Bacillus vallismortis isolates cluster in SspE proteotypes 16 and 17 (see FIGS. 5 and 6, gold branches). We are unaware of any currently known commercial utility for this species, and thus SspE can be used as a screening/molecular diagnostic target for this species. All Bacillus vallismortis isolates we studied cluster into a coherent and distinct phylogenetic clade by SspE protein, DNA and concatenated MLST DNA fragment analysis with no misidentified or mischaracterized strains. Thus, sspE sequence typing is a rapid and inexpensive means for unambiguous discrimination of B. vallismortis strains. A molecular signature for this group is SspE translated protein sequence length of 84 amino acids, which are distinctively identified by having a Q residue at position 4, a V residue at position 38 and an N residue at position 65. Bacillus vallismortis isolates also have the following residue characteristics: K (proteotype 16) or N (proteotype 17) at position 16.

8. Bacillus atrophaeus isolates cluster in SspE proteotype 18 (see FIGS. 5 and 6, gray branches). We are unaware of any currently known commercial utility for this species, except for its use as a bioindicators for sterilization processes, and thus SspE can be used as a screening/molecular diagnostic target for this species. All Bacillus atrophaeus isolates we studied cluster into a coherent and distinct phylogenetic clade by SspE protein, DNA and concatenated MLST DNA fragment analysis with no misidentified or mischaracterized strains. Three strains currently identified as Bacillus subtilis cluster with B. atrophaeus, and it has been suggested that these strains be reclassified to the latter species on the basis of AFLP typing⁵. Thus, sspE sequence typing is a rapid and inexpensive means for unambiguous discrimination of B. atrophaeus strains. A molecular signature for this group is SspE translated protein sequence length of 82 amino acids, which are distinctively identified by having an S residue at position 22, a V residue at position 37 and an A residue at position 64.

Uses for Bacillus subtilis Group Species
Bacillus subtilis
Fermentation of chocolate, aquatic farming, production of enzymes for detergents, an antidote in Europe for dysentery, contained in the antibiotic Bacitracin. Source: Companion (Growth Products) advertising supplement.
Produces subtilisin, which can be used as a grease and waste digester for biological drain control. Source: Clean Control Corporation.
Produces the useful enzymes amylase, lipase, gelatin and casein (ATCC strains 202137, 202138 and 202139). Source: Lawler, et al. U.S. Pat. No. 6,177,012.
Produces β-glucanase. Industry: beverage. Source: Schallmey, et al. 2004.
Produces cellulase. Source: Schallmey, et al. 2004.
Produces purine nucleotides. Application: flavor enhancers, medicine. Source: Schallmey, et al. 2004.
Produces riboflavin. Application: vitamin ingredient for health food. Source: Schallmey, et al. 2004.
Produces D-ribose. Application: flavor enhancer in food, health food, pharmaceuticals, cosmetics. Source: Schallmey, et al. 2004.
Produces thaumatin. Application: sweet-tasting protein for applications in food and pharmaceuticals. Source: Schallmey, et al. 2004.
Produces streptavidin. Application: biotin-binding protein, applications in high density biochips. Source: Schallmey, et al. 2004.
Produces alkaline protease. Application: [laundry and dishwashing] detergent additives (enable the release of proteinaceous material from stains/dishes); products: Alkazym (Novodan A/S, Copenhagen, Denmark), Terg-a-zyme (Alconox, Inc., New York, USA), Ultrasil 53 (Henkel KGaA, Dusseldorf, Germany), and P3-paradigm (Henkel-Ecolab GmbH, Dusseldorf, Germany). Application: tannery industry (biotreatment of leather, especially the dehairing and bating of skins and hides). Application: silver recovery (bioprocessing of used X-ray films for silver recovery—enzymatic hydrolysis of the gelatin layers on the X-ray film enables recycling of the polyester film base in addition to the silver). Application: medical uses (treatment of burns, purulent wounds, carbuncles, furuncles, deep abscesses and as a thrombolytic agent). Application: food industry (production of hydrolysates of well-defined peptide profile, meat tenderization). Application: waste treatment for various food processing industries and household activities (for example, processing of waste feathers from poultry slaughterhouses into a high protein source used as a food additive, degrading waste keratinous material in household refuse or as a depilatory agent to remove hair in bathtub drains, to name a few). Application: chemical industry (biocatalysts in synthetic chemistry). Source: Kumar & Takagi 1999.
Produces poly(glutamic acid). Application: medical industry. Facilitates drug delivery by attaching drug to the hydrophobic segment (drugs may include anti-cancer drugs, drugs for central nervous system, drugs for circulatory organs, and so forth). Source: Sakurai, et al. 1997.
Produces poly(glutamic acid). Application: medical industry. Facilitates cytotoxic drug delivery by attaching cytotoxic drug to the PGA for delivery to tumor cells where the PGA carrier is biodegraded and the cytotoxic agent is released, resulting in selective destruction of tumor cells. Source: Myers, et al. 1992.
Produces poly-(γ-glutamic acid). Application: water and wastewater treatment [removal of heavy metals and radionucleides (metal chelates or absorbents) & substitutes for polyacrylamide (bioflocculants)]. Application: food industry [viscosity enhancement for fruit juice beverages & sports drinks (thickener), cryoprotectant (for frozen food), relief of bitter taste by amino acids, peptides, quinine, caffeine, minerals, etc. (bitterness relieving agents), use in bakery products and noodles for the prevention of aging, improvement of textures (aging inhibitor or texture enhancer), used to promote absorption of minerals*, increase the strength of egg shells, decrease body fat*, etc. (animal feed additives*)]. Application: medical [use as a drug carrier or for sustained release of materials (gene therapy, cancer drugs), use for curable biological adhesive and hemostatic, medical bonding or suture thread (substitutes for fibrin)]. Application: cosmetics industry (humectant—absorbs water from the air). Source: Shih & Van 2001, Shih & Yu 2005.
Produces poly(L-glutamic acid). Application: medical industry. Facilitates delivery of paclitaxel, an anti-cancer drug, to tumors. Source: Li, et al. 2000.
Produces poly(glutamic acid). Application: medical industry. Facilitates delivery of drugs, used as a biological glue. Source: Richard & Margaritis 2002.
Produces levan. Applications: cosmetics, foods and pharmaceuticals, used as an industrial gum, a blood plasma extender, and a sweetener. Levan has potential applications as an emulsifier, a formulation aid, a stabilizer, a thickener, a surface-finishing agent, an encapsulating agent, and a carrier for flavor and fragrances. Source: Shih, et al. 2005, Shih & Yu 2005.
   Bacillus pumilus (See Supplementary FIGS. 7-9 and Tables 7-8)
Can be used to degrade grease for biological drain control. Source: Alken-Murray Corporation.
Produces the useful enzyme lipase (ATCC strain 202136). Source: Lawler, et al. U.S. Pat. No. 6,177,012
Produces D-ribose. Application: flavor enhancer in food, health food, pharmaceuticals, cosmetics. Source: Schallmey, et al. 2004.
Produces alkaline protease. Application: [laundry and dishwashing] detergent additives (enable the release of proteinaceous material from stains/dishes); products: Alkazym (Novodan A/S, Copenhagen, Denmark), Terg-a-zyme (Alconox, Inc., New York, USA), Ultrasil 53 (Henkel KGaA, Dusseldorf, Germany), and P3-paradigm (Henkel-Ecolab GmbH, Dusseldorf, Germany). Application: tannery industry (biotreatment of leather, especially the dehairing and bating of skins and hides). Application: silver recovery (bioprocessing of used X-ray films for silver recovery—enzymatic hydrolysis of the gelatin layers on the X-ray film enables recycling of the polyester film base in addition to the silver). Application: medical uses (treatment of burns, purulent wounds, carbuncles, furuncles, deep abscesses and as a thrombolytic agent). Application: food industry (production of hydrolysates of well-defined peptide profile, meat tenderization). Application: waste treatment for various food processing industries and household activities (for example, processing of waste feathers from poultry slaughterhouses into a high protein source used as a food additive, degrading waste keratinous material in household refuse or as a depilatory agent to remove hair in bathtub drains, to name a few). Application: chemical industry (biocatalysts in synthetic chemistry). Source: Kumar & Takagi 1999.
   Bacillus amyloliquefaciens
Produces the useful enzymes amylase, lipase, gelatin and casein (ATCC strains 202133 and 202134). Source: Lawler, et al. U.S. Pat. No. 6,177,012.
Produces alkaline proteases. Industry: detergent. Bacillus proteases dominate the market. This gene may also be cloned into B. subtilis for production. Source: Schallmey, et al. 2004.
Produces amylase. Application: beverage industry. Source: Schallmey, et al. 2004.
Produces alkaline protease. Application: [laundry and dishwashing] detergent additives (enable the release of proteinaceous material from stains/dishes); products: Alkazym (Novodan A/S, Copenhagen, Denmark), Terg-a-zyme (Alconox, Inc., New York, USA), Ultrasil 53 (Henkel KGaA, Dusseldorf, Germany), and P3-paradigm (Henkel-Ecolab GmbH, Dusseldorf, Germany). Application: tannery industry (biotreatment of leather, especially the dehairing and bating of skins and hides). Application: silver recovery (bioprocessing of used X-ray films for silver recovery—enzymatic hydrolysis of the gelatin layers on the X-ray film enables recycling of the polyester film base in addition to the silver). Application: medical uses (treatment of burns, purulent wounds, carbuncles, furuncles, deep abscesses and as a thrombolytic agent). Application: food industry (production of hydrolysates of well-defined peptide profile, meat tenderization). Application: waste treatment for various food processing industries and household activities (for example, processing of waste feathers from poultry slaughterhouses into a high protein source used as a food additive, degrading waste keratinous material in household refuse or as a depilatory agent to remove hair in bathtub drains, to name a few). Application: chemical industry (biocatalysts in synthetic chemistry). Source: Kumar & Takagi 1999.
   Bacillus licheniformis
Produces alkaline proteases. Removal of starch stains. Source: Schallmey, et al. 2004.
Produces α-amylase. Industry: starch. This gene may also be cloned into B. subtilis for production. Source: Schallmey, et al. 2004.
Produces amylase. Application: beverage industry. Source: Schallmey, et al. 2004.
Produces keratinase. This gene may also be cloned into B. subtilis for production. Source: Schallmey, et al. 2004.
Produces the antibiotic Bacitracin which inhibits cell wall synthesis. Source: Schallmey, et al. 2004.
Produces alkaline protease. Application: [laundry and dishwashing] detergent additives (enable the release of proteinaceous material from stains/dishes); products: Alkazym (Novodan A/S, Copenhagen, Denmark), Terg-a-zyme (Alconox, Inc., New York, USA), Ultrasil 53 (Henkel KGaA, Dusseldorf, Germany), and P3-paradigm (Henkel-Ecolab GmbH, Dusseldorf, Germany). Application: tannery industry (biotreatment of leather, especially the dehairing and bating of skins and hides). Application: silver recovery (bioprocessing of used X-ray films for silver recovery—enzymatic hydrolysis of the gelatin layers on the X-ray film enables recycling of the polyester film base in addition to the silver). Application: medical uses (treatment of burns, purulent wounds, carbuncles, furuncles, deep abscesses and as a thrombolytic agent). Application: food industry (production of hydrolysates of well-defined peptide profile, meat tenderization). Application: waste treatment for various food processing industries and household activities (for example, processing of waste feathers from poultry slaughterhouses into a high protein source used as a food additive, degrading waste keratinous material in household refuse or as a depilatory agent to remove hair in bathtub drains, to name a few). Application: chemical industry (biocatalysts in synthetic chemistry). Source: Kumar & Takagi 1999.
Produces poly(glutamic acid). Application: medical industry. Facilitates drug delivery by attaching drug to the hydrophobic segment (drugs may include anti-cancer drugs, drugs for central nervous system, drugs for circulatory organs, and so forth). Source: Sakurai, et al. 1997.
Produces poly(glutamic acid). Application: medical industry. Facilitates cytotoxic drug delivery by attaching cytotoxic drug to the PGA for delivery to tumor cells where the PGA carrier is biodegraded and the cytotoxic agent is released, resulting in selective destruction of tumor cells. Source: Myers, et al. 1992.
Produces poly-(γ-glutamic acid). Application: water and wastewater treatment [removal of heavy metals and radionucleides (metal chelates or absorbents) & substitutes for polyacrylamide (bioflocculants)]. Application: food industry [viscosity enhancement for fruit juice beverages & sports drinks (thickener), cryoprotectant (for frozen food), relief of bitter taste by amino acids, peptides, quinine, caffeine, minerals, etc. (bitterness relieving agents), use in bakery products and noodles for the prevention of aging, improvement of textures (aging inhibitor or texture enhancer), used to promote absorption of minerals*, increase the strength of egg shells, decrease body fat*, etc. (animal feed additives*)]. Application: medical [use as a drug carrier or for sustained release of materials (gene therapy, cancer drugs), use for curable biological adhesive and hemostatic, medical bonding or suture thread (substitutes for fibrin)]. Application: cosmetics industry (humectant—absorbs water from the air). Source: Shih & Van 2001, Shih & Yu 2005.
Produces poly(L-glutamic acid). Application: medical industry. Facilitates delivery of paclitaxel, an anti-cancer drug, to tumors. Source: Li, et al. 2000.
Produces poly(glutamic acid). Application: medical industry. Facilitates delivery of drugs, used as a biological glue. Source: Richard & Margaritis 2002.

### Bacillus sp.

Produces pectate lyases, alkaline amylase, mannanase. Source: Schallmey, et al. 2004.
Produces poly(glutamic acid). Application: medical industry. Facilitates drug delivery by attaching drug to the hydrophobic segment (drugs may include anti-cancer drugs, drugs for central nervous system, drugs for circulatory organs, and so forth). Source: Sakurai, et al. 1997.
Produces poly(glutamic acid). Application: medical industry. Facilitates cytotoxic drug delivery by attaching cytotoxic drug to the PGA for delivery to tumor cells where the PGA carrier is biodegraded and the cytotoxic agent is released, resulting in selective destruction of tumor cells. Source: Myers, et al. 1992.

TABLE 5

| | | | SspE | | |
|---|---|---|---|---|---|
| | SspE aa | sspE nt | size | | |
| Classifier^{a} | group^{b} | group^{c} | (AA)^{d} | MLST ST^{e} | Commercial Utility^{f} |
| 1a | 1 | 1a | 85 | 12 | [symbol] B. subtilis |
| 1b | 1 | 1b | 85 | 13 | Biofungicide, drain opener, cleaner & sanitizer^{8,11,20} B. licheniformis |
| | | | | | (misidentified) |
| 2a | 2 | 2 | 84 | 1 | [symbol] B. subtilis subsp. subtilis (strain 168,Marburg) |
| 2b | 2 | 2 | 84 | 2 | [symbol] B. subtilis subsp. subtilis |
| 2c | 2 | 2 | 84 | 4 | Produces enzymes²⁵⁻²⁶ B. subtilis (strain natto) (reclassified) |
| 2d | 2 | 2 | 84 | 5 | [symbol] B. subtilis (strain natto) (reclassified) |
| 2e | 2 | 2 | 84 | 6 | [symbol] B. subtilis |
| 2f | 2 | 2 | 84 | 9 | [symbol] B. subtilis subsp. subtilis |
| 2g | 2 | 2 | 84 | 10 | [symbol] B. subtilis |
| 2h | 2 | 2 | 84 | 35 | [symbol] B. subtilis subsp. subtilis (strain W168); B. licheniformis (misidentified) |
| 2i | 2 | 2 | 84 | 43 | [symbol] B. subtilis subsp. spizizenii (misidentified) |
| 2j | 2 | 2 | 84 | 44 | [symbol] B. amyloliquefaciens (reclassified) |
| 3a | 3 | 3 | 85 | 24 | ✓ B. mojavensis^{2,3,28} |
| 3b | 3 | 3 | 85 | 25 | ✓ B. mojavensis^{2,3,28} |
| 4a | 4 | 4 | 85 | 26 | ✓ B. mojavensis^{2,3,28} |
| 5a | 5 | 5 | 85 | 36 | ✓B. mojavensis^{2,3,28} |
| 6a | 6 | 6 | 54 | 27 | [symbol] B. licheniformis |
| 6b | 6 | 6 | 54 | 28 | [symbol] B. licheniformis |
| 6c | 6 | 6 | 54 | 29 | Produces enzyme B. licheniformis |
| 6d | 6 | 6 | 54 | 37 | Produces enzyme³⁵ B. licheniformis |
| 6e | 6 | 6 | 54 | 45 | [symbol] B. licheniformis |
| 6f | 6 | 6 | 54 | 46 | [symbol] B. licheniformis |
| 7a | 7 | 7 | 54 | 30 | Produces 5-hydroxy-L-tryptophan⁷ B. licheniformis (misidentified) |
| 7b | 7 | 7 | 54 | 31 | [symbol] B. sonorensis |
| 7c | 7 | 7 | 54 | 33 | [symbol] B. sonorensis |
| 7d | 7 | 7 | 54 | 34 | [symbol] B. sonorensis |
| 7e | 7 | 7 | 54 | 38 | [symbol] B. sonorensis |
| 7f | 7 | 7 | 54 | 47 | [symbol] B. sonorensis |
| 8a | 8 | 8 | 56 | 32 | [symbol] Bacillus sp. (unidentified) |
| 8b | 8 | 8 | 56 | 40 | Biofungicide^{4,15} B. subtilis (misidentified) |
| 8c | 8 | 8 | 56 | 41 | Antifungal activity B. subtilis (misidentified) |
| 9a | 9 | 9 | 56 | 42 | Biofungicide^{12-14,18} B. subtilis (misidentified) |
| 10a | 10 | 10 | 56 | 39 | Produces antibiotics against & inhibits growth of certain plant pathogenic |
| | | | | | fungi & bacteria²²⁻²³ B. subtilis (misidentified) |
| 11a^{g} | 11 | 11 | 56 | A^{g} | Produces enzymes³⁷ B. amyloliquefaciens; B. subtilis (misidentified) |
| 11b^{g} | 11 | 11 | 56 | B^{g} | Drain opener, cleaner & sanitizer^{11,20}; Produces amylase, inhibitors for |
| | | | | | glycoside hydrolases³⁷ B. amyloliquefaciens; B. subtilis (misidentified); |
| | | | | | P. polymyxa (misidentified) |
| 11c^{g} | 11 | 11 | 56 | C^{g} | [symbol] B. subtilis (misidentified) |
| 12a | 12 | 12 | 85 | 7 | B. subtilis subsp. spizizenii (strain W23) |
| 12b | 12 | 12 | 85 | 14 | B. subtilis |
| 13a | 13 | 13 | 85 | 8 | B. subtilis subsp. spizizenii |
| 13b | 13 | 13 | 85 | 15 | B. subtilis strain N10 degrades Tween-80⁹ |
| 14a | 14 | 14 | 84 | 3 | [symbol] B. subtilis |
| 15a | 15 | 15 | 84 | 11 | [symbol] B. subtilis (var. lactipan) |
| 16a | 16 | 16 | 84 | 21 | B. vallismortis |
| 16b | 16 | 16 | 84 | 22 | B. vallismortis |
| 17a | 17 | 17 | 84 | 23 | B. vallismortis |
| 18a | 18 | 18 | 82 | 16 | B. atrophaeus; B. subtilis (3/19)^{f} (misidentified)⁵ |
| 18b | 18 | 18 | 82 | 17 | B. atrophaeus |
| 18c | 18 | 18 | 82 | 18 | B. atrophaeus |
| 18d | 18 | 18 | 82 | 19 | B. atrophaeus |
| 18e | 18 | 18 | 82 | 20 | B. atrophaeus |

Table 5 Footnotes.
^{a}Classifiers (digital identifiers) are bold typed; these depict species, subspecies and strains of the B. subtilis/licheniformis clade by combined SspE (number) and MLST sequence type, represented by a lower case letter that corresponds to a ST within that particular SspE type. A color-coded phylogenetic tree generated from MLST data and labeled with these classifiers is shown in FIG. 6. This MLST scheme was developed and all data was generated in our lab; all data (allelic profiles, STs, primer sequences, allele sequence data, DNA sequence chromatograms, etc.) will be publicly available at pubmlst.org/bsubtilis.
^{b}Translated nucleic acid sequence of the sspE gene gives us proteotype SspE groups 1-18.
^{c}Nucleic acid sequences of the sspE gene are assigned (color-coded) genotypes 1a-x through 18a-x, where the number corresponds to the SspE proteotype and the lowercase letter corresponds to a unique nucleic acid sequence of that proteotype. For the Bs/Bl clade of organisms, only one sspE genotype corresponds to each proteotype, with the exception of B. subtilis-related proteotype 1 for which we have found two associated genotypes. A color-coded phylogenetic tree generated from sspE nucleic acid sequences for the B. subtilis/licheniformis group is shown in FIG. 5. sspE sequence data from this study will be deposited in the GenBank nucleotide sequence database.
^{d}Length of the SspE protein (54-85 amino acids, Bs/Bl group).
^{e}The MLST sequence type (ST) is a number assigned to a unique allelic profile from nucleotide sequences of seven housekeeping gene fragments. The genes used in this scheme are glpF, ilvD, pta, purH, pycA, rpoD and tpiA, and information including primer sequences, allelic profiles and STs, allele sequences and isolate information will be available at pubmlst.org/bsubtilis. All STs are novel sequence types found in our collection and have not been published or publicly disclosed.
^{f}Isolates identified by their classifier that are currently used commercially as biofungicides or enzyme producers are indicated by claimed or marketed utility and relevant patent numbers are highlighted in bold font. Isolates that have not yet been associated with a commercially valuable & patented strain are indicated with [symbol] if they are phylogenetically proximate to at least one commercial classifier (see FIGS. 5 and 6). Strains of B. molavensis, which have been described in literature^{2-3,28} as having antifungal activity on plants, are indicated by ✓. Fractions in parentheses represent the number of isolates of a particular bona fide species or subspecies within the classifier over the total number of that species or subspecies examined in this work.
^{g}Isolates clustered in this SspE proteotype have partial allelic profiles. Thus, they are not included in the FIG. 6 MLST tree and have been assigned letters A-C to describe their unique partial allelic profiles. We were able to assign classifiers 11a-c to these isolates because they all share a single unique SspE sequence and their partial allelic profiles from genes glpF, pta, purH, rpoD and tpiA contain allele sequences that are unique to this cluster and are not found in any other SspE types or STs to date.

TABLE 6

| | | | | 2 | 4 | | 6 | 6 | 7 | 7 | | | 11 | 12 | 14 |
|---|---|---|---|---|---|---|---|---|---|---|---|---|---|---|---|
| | | | | A | S | | N | N | F | F | | | N | A | Q |
| | Proteo- | Geno- | | ↓ | ↓ | 5 | ↓ | ↓ | ↓ | ↓ | 7 | 8 | ↓ | ↓ | ↓ |
| Species | type | type | Strain | E | Q | N | K | Q | S | Y | F | S | D | V | K |
| Bs | 12 | | 2A1, 2A2, 2A3, 2A6, 2A9, 3A13, A6633, | | | | | | | | | | | | |
| | | | D347, D618, D1087, D6395, D6399, D6405, | | | | | | | | | | | | |
| | | | D8439, W23 | | | | | | | | | | | | |
| | 13 | | D15029^{T}, 2A8^{T}, 3A17, B23049^{T} | | | | | | | | | | | | |
| | 1 | 1a | D5552 | | | | | | • | | | | | | |
| | | 1b | A55406 | | | | | | | | | | | | |
| | 14 | | D5611 | | | | | | | | | | | | |
| | 2 | | RS2, RS1725, W168, SB1058, WB746, 3610, | | | | | | | | | | | | |
| | | | 1A1, 1A3, 1A96, 1A308, 1A747, 1A757, | | | | | | | | | | | | |
| | | | 2A10, 3A1, 3A18, 3A19, 10A5T, 27E1, | | | | | | | | | | | | |
| | | | A6051^{T}, A7058, A7059, A15245, B642, D10^{T}, | | | | | | | | | | | | |
| | | | D1088, D1092, D1970, D1971, D3257, | | | | | | | | | | | | |
| | | | D4424, D4449, D4450, D4451, D5660, FB20, | | | | | | | | | | | | |
| | | | FB60, FB61, FB68, FB72, FB86, FB87, | | | | | | | | | | | | |
| | | | FB113, PS533, PS578, PS832, PS2307, | | | | | | | | | | | | |
| | | | PS2318, PS2319, PS3394 | | | | | | | | | | | | |
| | 15 | | 3A16 | | | | | | | | | | | | |
| Bat | 18 | | 11A1, A6455, A6537, A7972, A9372, | | | | | | | | Δ | Δ | | | |
| | | | A31028, A49337^{T}, A49760, A49822, A51189, | | | | | | | | | | | | |
| | | | D675, D2277, D5551, D7264^{T}, DPG Batr, | | | | | | | | | | | | |
| | | | BatrO, BatrW | | | | | | | | | | | | |
| Bmo | 3 | | [symbol] , [symbol] | | | | | | | | | | | | |
| | 4 | | [symbol] | | | | | | | | | | | | |
| | 5 | | [symbol] | | | | | | | | | | | | |
| Bv | 16 | | B14890^{T}, B14892, B14893 | | • | Δ | | | | | | | | | |
| | 17 | | B14894 | | • | Δ | | | | | | | | | |
| Bl | 6 | | A6598, A11946, A14580^{T}, 5A1, 5A2, 5A13, | | • | Δ | | • | • | | | Δ | | | • |
| | | | 5A20, 5A21, 5A32, 5A36^{T}, D1969, D8785, | | | | | | | | | | | | |
| | | | B23318, B23325, MO1 | | | | | | | | | | | | |
| Bson | 7 | | D1913, D13780, B23154^{T}, B23155, | | • | Δ | | • | • | | | Δ | | | • |
| | | | B23157, B23158, B23159, B23160, B23161 | | | | | | | | | | | | |
| Bsp | 8 | | D1324, [symbol] , [symbol] , [symbol] , GB03, 10A6 | | | Δ | • | | | • | | | • | • | |
| | 9 | | QST 713, B21661 | | | Δ | • | | | • | | | • | • | |
| | 10 | | A55614 | | | Δ | • | | | • | | | • | • | |
| | 11 | | 3A14, 3A23, A55405, A55407, D7T, D1060 | • | | Δ | • | | | • | | | • | • | |
| | | | | 16 | 17 | 17 | 17 | 21 | 24 | 24 | | 29 | 34 | 38 | 39 |
| | | | | R | K | K | K | Q | A | A | | F | A | N | A |
| | Proteo- | Geno- | | ↓ | ↓ | ↓ | ↓ | ↓ | ↓ | ↓ | 26 | ↓ | ↓ | ↓ | ↓ |
| Species | type | type | Strain | K | N | R | Q | A | Q | S | Q | Y | G | D | V |
| Bs | 12 | | 2A1, 2A2, 2A3, 2A6, 2A9, 3A13, A6633, | | | | | | | | | | | | |
| | | | D347, D618, D1087, D6395, D6399, D6405, | | | | | | | | | | | | |
| | | | D8439, W23 | | | | | | | | | | | | |
| | 13 | | D15029^{T}, 2A8^{T}, 3A17, B23049^{T} | | | | | | | | | | | | |
| | 1 | 1a | D5552 | | | | | | | | | | | | |
| | | 1b | A55406 | | | | | | | | | | | | |
| | 14 | | D5611 | | | | | | | | | | | | |
| | 2 | | RS2, RS1725, W168, SB1058, WB746, 3610, | | | | | | | | | | | | |
| | | | 1A1, 1A3, 1A96, 1A308, 1A747, 1A757, | | | | | | | | | | | | |
| | | | 2A10, 3A1, 3A18, 3A19, 10A5T, 27E1, | | | | | | | | | | | | |
| | | | A6051^{T}, A7058, A7059, A15245, B642, D10^{T}, | | | | | | | | | | | | |
| | | | D1088, D1092, D1970, D1971, D3257, | | | | | | | | | | | | |
| | | | D4424, D4449, D4450, D4451, D5660, FB20, | | | | | | | | | | | | |
| | | | FB60, FB61, FB68, FB72, FB86, FB87, | | | | | | | | | | | | |
| | | | FB113, PS533, PS578, PS832, PS2307, | | | | | | | | | | | | |
| | | | PS2318, PS2319, PS3394 | | | | | | | | | | | | |
| | 15 | | 3A16 | | | | | | | | | | | | |
| Bat | 18 | | 11A1, A6455, A6537, A7972, A9372, | | | | | | | | | | | | • |
| | | | A31028, A49337^{T}, A49760, A49822, A51189, | | | | | | | | | | | | |
| | | | D675, D2277, D5551, D7264^{T}, DPG Batr, | | | | | | | | | | | | |
| | | | BatrO, BatrW | | | | | | | | | | | | |
| Bmo | 3 | | [symbol] , [symbol] | | | | | | | | | | | | • |
| | 4 | | [symbol] | | | | | | | | | | | | |
| | 5 | | [symbol] | | | | | | | | | | | | |
| Bv | 16 | | B14890^{T}, B14892, B14893 | | | | | | | | | | | | • |
| | 17 | | B14894 | | • | | | | | | | | | | • |
| Bl | 6 | | A6598, A11946, A14580^{T}, 5A1, 5A2, 5A13, | | | | • | • | • | | Δ | | | • | |
| | | | 5A20, 5A21, 5A32, 5A36^{T}, D1969, D8785, | | | | | | | | | | | | |
| | | | B23318, B23325, MO1 | | | | | | | | | | | | |
| Bson | 7 | | D1913, D13780, B23154^{T}, B23155, | | | | • | • | • | | Δ | | | • | |
| | | | B23157, B23158, B23159, B23160, B23161 | | | | | | | | | | | | |
| Bsp | 8 | | D1324, [symbol] , [symbol] , [symbol] , GB03, 10A6 | • | | • | | | | • | | • | • | • | |
| | 9 | | QST 713, B21661 | • | | • | | | | • | | • | • | • | • |
| | 10 | | A55614 | • | | | | | | • | | • | • | • | |
| | 11 | | 3A14, 3A23, A55405, A55407, D7T, D1060 | • | | | | | | • | | • | • | • | |
| | | | | 43 | 44 | 45 | | | | 55 | 66 | 67 | 67 | 76 | 80 |
| | | | | R | K | Q | | | | G | D | V | V | S | N |
| | Proteo- | Geno- | | ↓ | ↓ | ↓ | | 51 | 53 | ↓ | ↓ | ↓ | ↓ | ↓ | ↓ |
| Species | type | type | Strain | K | Q | N | 48-75 | A | Q | S | N | A | T | Q | K |
| Bs | 12 | | 2A1, 2A2, 2A3, 2A6, 2A9, 3A13, A6633, | | | | | | | | | | | | |
| | | | D347, D618, D1087, D6395, D6399, D6405, | | | | | | | | | | | | |
| | | | D8439, W23 | | | | | | | | | | | | |
| | 13 | | D15029^{T}, 2A8^{T}, 3A17, B23049^{T} | | | | | | | | | • | | | |
| | 1 | 1a | D5552 | • | | | | | | | | • | | | |
| | | 1b | A55406 | | | | | | | | | | | | |
| | 14 | | D5611 | | | | | Δ | | | | | | | |
| | 2 | | RS2, RS1725, W168, SB1058, WB746, 3610, | | | | | Δ | | | | • | | | |
| | | | 1A1, 1A3, 1A96, 1A308, 1A747, 1A757, | | | | | | | | | | | | |
| | | | 2A10, 3A1, 3A18, 3A19, 10A5T, 27E1, | | | | | | | | | | | | |
| | | | A6051^{T}, A7058, A7059, A15245, B642, D10^{T}, | | | | | | | | | | | | |
| | | | D1088, D1092, D1970, D1971, D3257, | | | | | | | | | | | | |
| | | | D4424, D4449, D4450, D4451, D5660, FB20, | | | | | | | | | | | | |
| | | | FB60, FB61, FB68, FB72, FB86, FB87, | | | | | | | | | | | | |
| | | | FB113, PS533, PS578, PS832, PS2307, | | | | | | | | | | | | |
| | | | PS2318, PS2319, PS3394 | | | | | | | | | | | | |
| | 15 | | 3A16 | | | | | Δ | | • | | | | | |
| Bat | 18 | | 11A1, A6455, A6537, A7972, A9372, | | | | | | Δ | | | • | | | |
| | | | A31028, A49337^{T}, A49760, A49822, A51189, | | | | | | | | | | | | |
| | | | D675, D2277, D5551, D7264^{T}, DPG Batr, | | | | | | | | | | | | |
| | | | BatrO, BatrW | | | | | | | | | | | | |
| Bmo | 3 | | [symbol] , [symbol] | | | | | | | | • | | ∘ | | |
| | 4 | | [symbol] | | | | | | | | | | ∘ | | |
| | 5 | | [symbol] | | | | | | | | • | | ∘ | | |
| Bv | 16 | | B14890^{T}, B14892, B14893 | | | | | | | | • | | | | |
| | 17 | | B14894 | | | | | | | | • | | | | |
| Bl | 6 | | A6598, A11946, A14580^{T}, 5A1, 5A2, 5A13, | | • | • | Δ | | | | | | | • | • |
| | | | 5A20, 5A21, 5A32, 5A36^{T}, D1969, D8785, | | | | | | | | | | | | |
| | | | B23318, B23325, MO1 | | | | | | | | | | | | |
| Bson | 7 | | D1913, D13780, B23154^{T}, B23155, | | | • | Δ | | | | | | | • | |
| | | | B23157, B23158, B23159, B23160, B23161 | | | | | | | | | | | | |
| Bsp | 8 | | D1324, [symbol] , [symbol] , [symbol] , GB03, 10A6 | | | | Δ | | | | | | | | |
| | 9 | | QST 713, B21661 | | | | Δ | | | | | | | | |
| | 10 | | A55614 | | | | Δ | | | | | | | | |
| | 11 | | 3A14, 3A23, A55405, A55407, D7T, D1060 | | | | Δ | | | | | | | | |

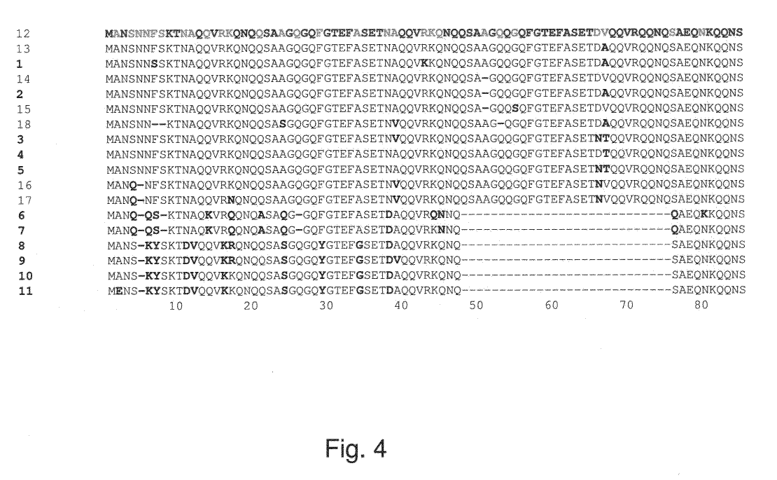

Table 6. Clustering of Bs/Bl group isolates by sspE genotype (proteotype 1 only) and translated proteotype. This table was developed from the ClUSTALW multisequence alignment of Bs group translated amino acid sequences (see FIG. 4 below). The SspE sequence of B. subtilis strain W23 was selected as the reference (holotype) sequence to which all other Bs group sequences are compared. The numbers at the top of the table indicate amino acid position in the SspE reference sequence. Just below these numbers, the letters indicate the specific residue change from the W23 holotype reference sequence (top letter). Residue changes are indicated by dots in the grid of the table. Colors are used to highlight similarities among proteotypes, but have no particular designated meaning. The Greek letter delta (A) symbolizes a residue deletion at the indicated position with respect to the holotype W23 reference sequence. SspE proteotype numbering (1-18) is consistent with Bs/Bl group SspE proteotype/genotype and classifier numbering in Table 5 and FIGS. 4-6. Proteotypes 1-11, indicated in bold type, contain at least one commercially available or patented strain. Patented strain names are indicated in bold type. Gray highlighted strains in SspE proteotype 8 may have European patents, which we could not locate. Gray highlighted B. mojavensis strains in SspE proteotypes 3-5 have been documented numerous times in academic and USDA literature as having antifungal activity, though we have not located patents for these strains. In SspE proteotype 9, strain B21661 is an independent isolate (obtained from the USDA's NRRL culture collection) of strain QST 713, which we isolated from AgraQuest's (http://www.agraquest.com/) plant protection product Serenade®. The same strain is an active ingredient in the product Rhapsody®, also by AgraQuest (Davis, Calif.). In SspE proteotype 8, strain GB63 was isolated from the Growth Products (White Plains, N.Y.) (http://www.growthproducts.com/) plant protection product Companion®. The same strain is an active ingredient in the Gustafson LLC (Plano, Tex.) product Kodiak®.

TABLE 7
Strain Table
Table 7. List of strains used in the Bacillus subtilis group scheme. Most strains were
acquired from culture collections.

| Classifier¹ | Strain |
|---|---|
| 1a | DSM 5552 |
| 1b | ATCC 55406 |
| 2a | BGSC 1A1, BGSC 1A3, BGSC 1A96, BGSC 1A747, BGSC 3A1, BGSC 10A1, RS2, RS1725, SB1058, WB746, |
| | 3610, ATCC 6051, DSM 10, DSM4424 |
| 2b | DSM 5660 |
| 2c | BGSC 27E1, ATCC 7058, ATCC 15245, DSM 1088, DSM 1970, DSM 1971, DSM 4449, DSM 4450, DSM 4451 |
| 2d | DSM 1092 |
| 2e | ATCC 7059 |
| 2f | DSM 3257 |
| 2g | BGSC 3A18, BGSC 3A19 |
| 2h | BGSC 1A308, BGSC 1A757, W168, NRRL B-642, PS533, PS578, PS2307, PS2318, PS2319, PS3394, |
| | FB20, FB60, FB61, FB68, FB72, FB87, FB113 |
| 2i | BGSC 2A10 |
| 2j | BGSC 10A5T |
| 3a | NRRL B-14698-T |
| 3b | NRRL B-14701 |
| 4a | NRRL B-14699 |
| 5a | DSM 9206 |
| 6a | BGSC 5A1, BGSC 5A2, ATCC 11946, MO1 |
| 6b | BGSC 5A13, BGSC 5A20, BGSC 5A21 |
| 6c | BGSC 5A32, BGSC 5A36, ATCC 14580, ATCC 6598, DSM 8785 |
| 6d | DSM 1969 |
| 6e | NRRL B-23318 |
| 6f | NRRL B-23325 |
| 7a | DSM 1913 |
| 7b | NRRL B-23154-T, NRRL B-23160 |
| 7c | NRRL B-23157 |
| 7d | NRRL B-23155 |
| 7e | NRRL B-23158, NRRL B-23159, DSM 13780 |
| 7f | NRRL B-23161 |
| 8a | DSM 1324 |
| 8b | Companion (GB03) |
| 8c | DSM 8563, DSM 8564, DSM 8565, BGSC 10A6 |
| 9a | Serenade, NRRL B-21661 |
| 10a | ATCC 55614 |
| 11a | DSM 7, BGSC 3A14 |
| 11b | DSM 1060, ATCC 55405, ATCC 55407 |
| 11c | BGSC 3A23 |
| 12a | BGSC 2A1, BGSC 2A2, BGSC 2A3, BGSC 2A6, BGSC 2A9, DSM 347, DSM 618, DSM 1087, DSM 6395, |
| | DSM 6399, DSM 6405, DSM 8439, W23, ATCC 6633 |
| 12b | BGSC 3A13 |
| 13a | BGSC 2A8, DSM 15029, NRRL B-23049 |
| 13b | BGSC 3A17 |
| 14a | DSM 5611 |
| 15a | BGSC 3A16 |
| 16a | NRRL B-14890-T, NRRL B-14892 |
| 16b | NRRL B-14893 |
| 17a | NRRL B-14894 |
| 18a | BGSC 11A1, ATCC 9372, ATCC 31028, ATCC 49760, ATCC 49822, ATCC 51189, DSM 675 |
| 18b | DSM 2277 |
| 18c | ATCC 6537, ATCC 7972 |
| 18d | ATCC 49337, DSM 5551, DSM 7264 |
| 18e | ATCC 6455 |
| 19a | DSM 355 |
| 19b | BGSC 8A1 |
| 19c | ATCC 27142 |
| 20a | BGSC 14A1 |
| 21a | DSM 354 |

BGSC = Bacillus Genetic Stock Center (Department of Biochemistry, The Ohio State University, 484 West Twelfth Avenue, Columbus, OH 43210, USA);
ATCC = American Type Culture Collection (P.O. Box 1549, Manassas, VA 20108, USA);
NRRL = the USDA ARS (NRRL) Culture Collection (National Center for Agricultural Utilization Research, Peoria, Illinois, USA);
DSM = DSMZ = Deutsche Sammlung von Mikroorganismen und Zellkulturen GmbH (Mascheroder Weg 1b, 38124 Braunschweig, Germany);
^{T}= Type Strain.

TABLE 8

| | | | | 2 | 2 | 3 | | 4 | 5 | 6 | 7 | 10 | 11 | 13 | 15 | 16 | 16 | 20 |
|---|---|---|---|---|---|---|---|---|---|---|---|---|---|---|---|---|---|---|
| | | | | A | A | N | | S | K | Y | S | D | V | Q | K | R | R | Q |
| | | | | ↓ | ↓ | ↓ | 4 | ↓ | ↓ | ↓ | ↓ | ↓ | ↓ | ↓ | ↓ | ↓ | ↓ | ↓ |
| Species | Proteotype | Genotype | Strain | E | M | D | S | Q | Q | Q | N | N | A | K | R | K | Q | A |
| Bsp | 8 | | D1324, D8563, | | | | | | | | | | | | | | | |
| | | | D8564, D8565, GB03, | | | | | | | | | | | | | | | |
| | | | 10A6 | | | | | | | | | | | | | | | |
| | 9 | | QST 713, B21661 | | | | | | | | | | | | | | | |
| | 10 | | A55614 | | | | | | | | | | | | | • | | |
| | 11 | | 3A14, 3A23, A55405, | • | | | | | | | | | | | | • | | |
| | | | A55407, D7^{T}, D1060 | | | | | | | | | | | | | | | |
| Bl | 6 | | A6598, A11946, | | | | Δ | | • | • | | • | • | • | • | | • | • |
| | | | A14580^{T}, 5A1, 5A2, | | | | | | | | | | | | | | | |
| | | | 5A13, 5A20, 5A21, | | | | | | | | | | | | | | | |
| | | | 5A32, 5A36^{T}, D1969, | | | | | | | | | | | | | | | |
| | | | D8785, B23318, | | | | | | | | | | | | | | | |
| | | | B23325, MO1 | | | | | | | | | | | | | | | |
| Bson | 7 | | D1913, D13780, | | | | Δ | | • | • | | • | • | • | • | | • | • |
| | | | B23154^{T}, B23155, | | | | | | | | | | | | | | | |
| | | | B23157, B23158, | | | | | | | | | | | | | | | |
| | | | B23159, B23160, | | | | | | | | | | | | | | | |
| | | | B23161 | | | | | | | | | | | | | | | |
| Bpum | 19 | a | D355 | | • | • | | • | • | • | • | • | • | | | • | | • |
| | | b | 8A1 | | | | | | | | | | | | | | | |
| | | c | A27142 | | | | | | | | | | | | | | | |
| | 20 | | 14A1 | | • | • | | • | • | • | • | • | • | | | • | | • |
| | 21 | | D354 | | • | • | | • | • | • | • | • | • | | | • | | • |

| | | | | 21 | 23 | | 28 | 33 | 38 | 40 | 42 | 43 | 44 | 44 | 47 | 47 | 51 |
|---|---|---|---|---|---|---|---|---|---|---|---|---|---|---|---|---|---|
| | | | | S | S | | Y | G | A | Q | R | K | Q | Q | S | S | N |
| | | | | ↓ | ↓ | 25 | ↓ | ↓ | ↓ | ↓ | ↓ | ↓ | ↓ | ↓ | ↓ | ↓ | ↓ |
| Species | Proteotype | Genotype | Strain | A | Q | Q | F | A | V | H | K | Q | N | Y | K | Q | K |
| Bsp | 8 | | D1324, D8563, | | | | | | | | | | | | | | |
| | | | D8564, D8565, GB03, | | | | | | | | | | | | | | |
| | | | 10A6 | | | | | | | | | | | | | | |
| | 9 | | QST 713, B21661 | | | | | | • | | | | | | | | |
| | 10 | | A55614 | | | | | | | | | | | | | | |
| | 11 | | 3A14, 3A23, A55405, | | | | | | | | | | | | | | |
| | | | A55407, D7^{T}, D1060 | | | | | | | | | | | | | | |
| Bl | 6 | | A6598, A11946, | | • | Δ | • | • | | | | • | • | | | • | • |
| | | | A14580^{T}, 5A1, 5A2, | | | | | | | | | | | | | | |
| | | | 5A13, 5A20, 5A21, | | | | | | | | | | | | | | |
| | | | 5A32, 5A36^{T}, D1969, | | | | | | | | | | | | | | |
| | | | D8785, B23318, | | | | | | | | | | | | | | |
| | | | B23325, MO1 | | | | | | | | | | | | | | |
| Bson | 7 | | D1913, D13780, | | • | Δ | • | • | | | | | • | | | • | |
| | | | B23154^{T}, B23155, | | | | | | | | | | | | | | |
| | | | B23157, B23158, | | | | | | | | | | | | | | |
| | | | B23159, B23160, | | | | | | | | | | | | | | |
| | | | B23161 | | | | | | | | | | | | | | |
| Bpum | 19 | a | D355 | | • | Δ | • | • | • | | • | | | • | • | | |
| | | b | 8A1 | | | | | | | | | | | | | | |
| | | c | A27142 | | | | | | | | | | | | | | |
| | 20 | | 14A1 | | • | Δ | | • | • | | • | | | • | • | | |
| | 21 | | D354 | • | • | Δ | | • | | • | • | | | • | • | | |

Table 8. Clustering of Bsp, Bl, Bson and Bpum isolates by sspE genotype (proteotype 19 only) and translated proteotype. This table was developed from the ClUSTALW multisequence alignment of Bs group translated amino acid sequences (see FIG. 7 above). The SspE sequence of Bacillus spp. biofungicidal strain GB03 was selected as the reference (holotype) sequence to which the other sequences are compared. The numbers at the top of the table indicate amino acid position in the SspE reference sequence. Just below these numbers; the letters indicate the specific residue change from the GB03 holotype reference sequence (top letter). Residue changes are indicated by dots in the grid of the table. Colors are used to highlight similarities among proteotypes, but have no particular designated meaning. The Greek capital letter delta (Δ) symbolizes a residue deletion at the indicated position with respect to the holotype GB03 reference sequence. SspE proteotype numbering (6-11 and 19-21) is consistent with SspE proteotype/genotype and classifier numbering in Tables 5-7 and FIGS. 4-9. Proteotype 19-21 B. pumilus strains are indicated in bold brown type.

### PART II REFERENCES

1. Alken-Murray Corp. Promotional/fact sheet. Comparison of lipase and esterase enzyme production in two Alken-Murray formulas.
2. Bacon, C. W. & Hinton D. M. (2004). Techniques for manipulating the bacterial endophyte Bacillus mojavensis. In: Methods in Biotechnology: Environmental Microbiology: Methods and Protocols, Spencer, J. F. F. & Ragout de Spencer, A. L., Eds. Humana Press, Totowa, N.J., p. 359-377.
3. Bacon, C. W. & Hinton D. M. (2005). Tentative identification of Bacillus mojavensis antifungal inhibitor [abstract]. Phytopathology 95, S5.
4. Bergstrom, G. C. & Corio da Luz, W. (2005). Biocontrol for plants with Bacillus subtilis, Pseudomonas putida, and Sporobolomyces roseus. Cornell Research Foundation, Inc.; Embrapa Trigo. U.S. Pat. No. 6,896,883.
5. Burke, S. A., Wright, J. D., Robinson, M. K., Bronk, B. V. & Warren, R. L. (2004). Detection of molecular diversity in Bacillus atrophaeus by amplified fragment length polymorphism analysis. Appl Environ Microbiol 70, 2786-2790. PubMed ID 15128533
6. Chen, X.-H., Vater, J., Piel, J., Franke, P., Scholz, R., Schneider, K., Koumoutsi, A., Hitzeroth, G., Grammel, N., Strittmatter, A. W., Gottschalk, G., Süssmuth, R. D. & Borriss, R. (2006). Structural and functional characterization of three polyketide synthase gene clusters in Bacillus amyloliquefaciens strain FZB 42. J Bacteriol 188, 4024-4036. PubMed ID 16707694
7. Daum, J. (1974). Process for the preparation of 5-hydroxy-L-tryptophan. Schering Aktiengesellschaft. U.S. Pat. No. 3,830,696.
8. Drahos, D. J. & West, L. (2004). Bacillus licheniformis biofungicide. Novozymes Biologicals, Inc. U.S. Pat. No. 6,824,772.
9. E1-Helow, E. R. (2001). Identification and molecular characterization of a novel Bacillus strain capable of degrading Tween-80. FEMS Microbiol Lett 15, 119-122. PubMed ID 11267766
10. Green, D. H., Wakeley, P. R., Page, A., Barnes, A., Baccigalupi, L., Ricca, E. & Cutting, S. M. (1999). Characterization of two Bacillus probiotics. Appl Environ Microbiol 65, 4288-4291. PubMed ID 10473456
11. Griffin, W. M., Ritter, R. T. & Dent, D. A. (1995). Drain opener formulation. Sybron Chemical Holdings, Inc. U.S. Pat. No. 5,449,619.
12. Heins, S. D., Manker, D. C., Jimenez, D. R., McCoy, R. J., Marrone, P. G. & Orjala, J. E. (2000). Compositions and methods for controlling plant pests. AgraQuest, Inc. U.S. Pat. No. 6,103,228.
13. Heins, S. D., Manker, D. C., Jimenez, D. R., McCoy, R. J., Marrone, P. G. & Orjala, J. E. (2001). Strain of Bacillus for controlling plant diseases and corn rootworm. AgraQuest, Inc. U.S. Pat. No. 6,291,426.
14. Heins, S. D., Manker, D. C., Jimenez, D. R., McCoy, R. J., Marrone, P. G. & Orjala, J. E. (2003). Compositions and methods for controlling plant pests. AgraQuest, Inc. U.S. Pat. No. 6,638,910.
15. Kloepper, J. W., Rodriguez-Kabana, R. & Kenney, D. S. (2003). Biological compositions and methods for enhancing plant growth and health and producing disease-suppressive plants. Auburn University; Gustafson, LLC. U.S. Pat. No. 6,524,998.
16. Koumoutsi, A., Chen, X.-H., Henne, A., Liesegang, H., Hitzeroth, G., Franke, P., Vater, J. & Borriss, R. (2004). Structural and functional characterization of gene clusters directing nonribosomal synthesis of bioactive cyclic lipopeptides in Bacillus amyloliquefaciens strain FZB42. J Bacteriol 186, 1084-1096. PubMed ID 14762003
17. Kumar, C. G. & Takagi, H. (1999). Microbial alkaline proteases: from a bioindustrial viewpoint. Biotechnol Adv 17, 561-594. PubMed ID 14538129
18. Lehman, L. J., McCoy, R. J., Messenger, B. J., Manker, D. C., Orjala, J. E., Lindhard, D., Marrone, P. G. & Jimenez, D. R. (2003). Strain of Bacillus for controlling plant diseases. AgraQuest, Inc. U.S. Pat. No. 6,635,245.
19. Li, C., Ke, S., Wu, Q.-P., Tansey, W., Hunter, N., Buchmiller, L. M., Milas, L., Charnsangavej, C. & Wallace, S. (2000). Tumor irradiation enhances the tumor-specific distribution of poly(L-glutamic acid)-conjugated paclitaxel and its antitumor efficacy. Clin Cancer Res 6, 2829-2834. PubMed ID 10914731.
20. Lin, J.-E. & Dent, D. A. (1999). Cleaner and sanitizer formulation. Sybron Chemical Holdings, Inc. U.S. Pat. No. 5,863,882.
21. Lawler, D. & Smith, S. (2001). Enzyme-producing strain of Bacillus bacteria. Roebic Laboratories, Inc. U.S. Pat. No. 6,177,012.
22. Marrone, P. G., Heins, S. D., Manker, D. C., Jimenez, D. R., Bestwick, R. K. & Vandemark, G. J. (1998). Antibiotic-producing strain of Bacillus and methods for controlling plant disease. Agritope, Inc. U.S. Pat. No. 5,753,222.
23. Marrone, P. G., Heins, S. D., Manker, D. C., Jimenez, D. R., Bestwick, R. K. & Vandemark, G. J. (1999). Biological control of plant fungal infections. Agritope, Inc. U.S. Pat. No. 6,004,774.
24. Marten, P., Smalla, K. & Berg, G. (2000). Genotypic and phenotypic differentiation of and antifungal biocontrol strain belonging to Bacillus subtilis. J Appl Microbiol 89, 463-471. PubMed ID 11021578
25. Murau, S. (1971). Alkaline protease. E. R. Squibb & Sons, Inc. U.S. Pat. No. 3,576,719.
26. Murau, S. (1971) (2). Production of a new protease. Sanraku-Ocean Co., Ltd. U.S. Pat. No. 3,622,458.
27. Myers, A. E. & Bichon, D. (1992). Cytotoxic drug conjugates and their delivery to tumor cells. Battelle Memorial Institute. U.S. Pat. No. 5,087,616.
28. Nair, J. R., Singh, G. & Sekar V. (2002). Isolation and characterization of a novel Bacillus strain from coffee phyllosphere showing antifungal activity. J Appl Microbiol 93 772-780. PubMed ID 12392522
29. Richard, A. & Margaritis, A. (2003). Rheology, oxygen transfer, and molecular weight characteristics of poly(glutamic acid) fermentation by Bacillus subtilis. Biotechnol Bioeng 82, 299-305. PubMed ID 12599256
30. Sanders M. E., Morelli, L. & Tompkins, T. A. (2003). Sporeformers as human probiotics: Bacillus, Sporolactobacillus, and Brevibacillus. Comprehensive Rev Food Sci Food Safety 2, 101-10. PubMed ID none
31. Sakurai, Y., Okano, T., Kataoka, K., Yamada, N., Inoue, S. & Yokoyama, M. (1997). Water soluble high molecular weight polymerized drug preparation. Research Development Corporation of Japan. U.S. Pat. No. 5,693,751.
32. Schallmey, M., Singh, A. & Ward, O. P. (2004). Developments in the use of Bacillus species for industrial production. Can J Microbiol 50, 1-17. PubMed ID 15052317
33. Shih, I.-L. & Van, Y.-T. (2001). The production of poly(γ-glutamic acid) from microorganisms and its various applications. Bioresour Technol 79, 207-225. PubMed ID 11499575
34; Shih, I.-L. & Yu, Y.-T. (2005). Simultaneous and selective production of levan and poly(γ-glutamic acid) by Bacillus subtilis. Biotechnol Lett 27, 103-106. PubMed ID 15703872
35. Shih, I.-L., Yu, Y.-T., Shieh, C.-J. & Hsieh C.-Y. (2005). Selective production and characterization of levan by Bacillus subtilis (Natto) Takahashi. J Agric Food Chem 53, 8211-8215. PubMed ID 16218666
36. Viccaro, J. P. (1973). Alkaline protease, method for its production, and detergent composition. Lever Brothers Company. U.S. Pat. No. 3,748,233.
37. British Patent 155,409

The following table is a look up table that matches sequence identifiers with sspE identifiers and/or MLS allele information.

| SEQ ID NO | note |
|---|---|
| SEQ ID NO: 49 | SspE_A_93aa |
| SEQ ID NO: 50 | SspE_B_93aa |
| SEQ ID NO: 51 | SspE_C_93aa |
| SEQ ID NO: 52 | SspE_D_93aa |
| SEQ ID NO: 53 | SspE_E_93aa |
| SEQ ID NO: 54 | SspE_F_93aa |
| SEQ ID NO: 55 | SspE_G_93aa |
| SEQ ID NO: 56 | SspE_H_93aa |
| SEQ ID NO: 57 | SspE_I_93aa |
| SEQ ID NO: 58 | SspE_J_93aa |
| SEQ ID NO: 59 | SspE_K_93aa |
| SEQ ID NO: 60 | SspE_L_93aa |
| SEQ ID NO: 61 | SspE_M_93aa |
| SEQ ID NO: 62 | SspE_N_92aa |
| SEQ ID NO: 63 | SspE_O_95aa |
| SEQ ID NO: 64 | SspE_P_95aa |
| SEQ ID NO: 65 | SspE_Q_93aa |
| SEQ ID NO: 66 | SspE_R_93aa |
| SEQ ID NO: 67 | SspE_S_92aa |
| SEQ ID NO: 68 | SspE_T_95aa |
| SEQ ID NO: 69 | SspE_U_95aa |
| SEQ ID NO: 70 | sspE_A1_282nt |
| SEQ ID NO: 71 | sspE_A2_282nt |
| SEQ ID NO: 72 | sspE_B_282nt |
| SEQ ID NO: 73 | sspE_C_282nt |
| SEQ ID NO: 74 | sspE_D_282nt |
| SEQ ID NO: 75 | sspE_E1_282nt |
| SEQ ID NO: 76 | sspE_E2_282nt |
| SEQ ID NO: 77 | sspE_E3_282nt |
| SEQ ID NO: 78 | sspE_E4_282nt |
| SEQ ID NO: 79 | sspE_E5_282nt |
| SEQ ID NO: 80 | sspE_E6_282nt |
| SEQ ID NO: 81 | sspE_E7_282nt |
| SEQ ID NO: 82 | sspE_E8_282nt |
| SEQ ID NO: 83 | sspE_E9_282nt |
| SEQ ID NO: 84 | sspE_E10_282nt |
| SEQ ID NO: 85 | sspE_E11_282nt |
| SEQ ID NO: 86 | sspE_F1_282nt |
| SEQ ID NO: 87 | sspE_F2_282nt |
| SEQ ID NO: 88 | sspE_F3_282nt |
| SEQ ID NO: 89 | sspE_F4_282nt |
| SEQ ID NO: 90 | sspE_G_282nt |
| SEQ ID NO: 91 | sspE_H1_282nt |
| SEQ ID NO: 92 | sspE_H2_282nt |
| SEQ ID NO: 93 | sspE_H3_282nt |
| SEQ ID NO: 94 | sspE_H4_282nt |
| SEQ ID NO: 95 | sspE_H5_282nt |
| SEQ ID NO: 96 | sspE_I_282nt |
| SEQ ID NO: 97 | sspE_J_282nt |
| SEQ ID NO: 98 | sspE_K1_282nt |
| SEQ ID NO: 99 | sspE_K2_282nt |
| SEQ ID NO: 100 | sspE_K3_282nt |
| SEQ ID NO: 101 | sspE_L_282nt |
| SEQ ID NO: 102 | sspE_M_282nt |
| SEQ ID NO: 103 | sspE_N_279nt |
| SEQ ID NO: 104 | sspE_O_288nt |
| SEQ ID NO: 105 | sspE_P_288nt |
| SEQ ID NO: 106 | sspE_Q_282nt |
| SEQ ID NO: 107 | sspE_R_282nt |
| SEQ ID NO: 108 | sspE_S_279nt |
| SEQ ID NO: 109 | sspE_T_288nt |
| SEQ ID NO: 110 | sspE_U_288nt |
| SEQ ID NO: 111 | SspE_1_85aa |
| SEQ ID NO: 112 | SspE_2_84aa |
| SEQ ID NO: 113 | SspE_3_85aa |
| SEQ ID NO: 114 | SspE_4_85aa |
| SEQ ID NO: 115 | SspE_5_85aa |
| SEQ ID NO: 116 | SspE_6_54aa |
| SEQ ID NO: 117 | SspE_7_54aa |
| SEQ ID NO: 118 | SspE_8_56aa |
| SEQ ID NO: 119 | SspE_9_56aa |
| SEQ ID NO: 120 | SspE_10_56aa |
| SEQ ID NO: 121 | SspE_11_56aa |
| SEQ ID NO: 122 | SspE_12_85aa |
| SEQ ID NO: 123 | SspE_13_85aa |
| SEQ ID NO: 124 | SspE_14_84aa |
| SEQ ID NO: 125 | SspE_15_84aa |
| SEQ ID NO: 126 | SspE_16_84aa |
| SEQ ID NO: 127 | SspE_17_84aa |
| SEQ ID NO: 128 | SspE_18_82aa |
| SEQ ID NO: 129 | SspE_19_55aa |
| SEQ ID NO: 130 | SspE_20_55aa |
| SEQ ID NO: 131 | SspE_21_55aa |
| SEQ ID NO: 132 | sspE_1a_258nt |
| SEQ ID NO: 133 | sspE_1b_258nt |
| SEQ ID NO: 134 | sspE_2_255nt |
| SEQ ID NO: 135 | sspE_3_258nt |
| SEQ ID NO: 136 | sspE_4_258nt |
| SEQ ID NO: 137 | sspE_5_258nt |
| SEQ ID NO: 138 | sspE_6_165nt |
| SEQ ID NO: 139 | sspE_7_165nt |
| SEQ ID NO: 140 | sspE_8_171nt |
| SEQ ID NO: 141 | sspE_9_171nt |
| SEQ ID NO: 142 | sspE_10_171nt |
| SEQ ID NO: 143 | sspE_11_171nt |
| SEQ ID NO: 144 | sspE_12_258nt |
| SEQ ID NO: 145 | sspE_13_258nt |
| SEQ ID NO: 146 | sspE_14_255nt |
| SEQ ID NO: 147 | sspE_15_255nt |
| SEQ ID NO: 148 | sspE_16_255nt |
| SEQ ID NO: 149 | sspE_17_255nt |
| SEQ ID NO: 150 | sspE_18_249nt |
| SEQ ID NO: 151 | sspE_19a_168nt |
| SEQ ID NO: 152 | sspE_19b_168nt |
| SEQ ID NO: 153 | sspE_19c_168nt |
| SEQ ID NO: 154 | sspE_20_168nt |
| SEQ ID NO: 155 | sspE_21_168nt |
| SEQ ID NO: 156 | glp-1 |
| SEQ ID NO: 157 | glp-2 |
| SEQ ID NO: 158 | glp-3 |
| SEQ ID NO: 159 | glp-4 |
| SEQ ID NO: 160 | glp-5 |
| SEQ ID NO: 161 | glp-6 |
| SEQ ID NO: 162 | glp-7 |
| SEQ ID NO: 163 | glp-8 |
| SEQ ID NO: 164 | glp-9 |
| SEQ ID NO: 165 | glp-10 |
| SEQ ID NO: 166 | glp-11 |
| SEQ ID NO: 167 | glp-12 |
| SEQ ID NO: 168 | glp-13 |
| SEQ ID NO: 169 | glp-14 |
| SEQ ID NO: 170 | glp-15 |
| SEQ ID NO: 171 | glp-16 |
| SEQ ID NO: 172 | glp-17 |
| SEQ ID NO: 173 | glp-18 |
| SEQ ID NO: 174 | glp-19 |
| SEQ ID NO: 175 | glp-20 |
| SEQ ID NO: 176 | glp-21 |
| SEQ ID NO: 177 | glp-22 |
| SEQ ID NO: 178 | glp-23 |
| SEQ ID NO: 179 | glp-24 |
| SEQ ID NO: 180 | glp-25 |
| SEQ ID NO: 181 | glp-26 |
| SEQ ID NO: 182 | glp-27 |
| SEQ ID NO: 183 | glp-28 |
| SEQ ID NO: 184 | glp-29 |
| SEQ ID NO: 185 | glp-30 |
| SEQ ID NO: 186 | glp-31 |
| SEQ ID NO: 187 | ilv-1 |
| SEQ ID NO: 188 | ilv-2 |
| SEQ ID NO: 189 | ilv-3 |
| SEQ ID NO: 190 | ilv-4 |
| SEQ ID NO: 191 | ilv-5 |
| SEQ ID NO: 192 | ilv-6 |
| SEQ ID NO: 193 | ilv-7 |
| SEQ ID NO: 194 | ilv-8 |
| SEQ ID NO: 195 | ilv-9 |
| SEQ ID NO: 196 | ilv-10 |
| SEQ ID NO: 197 | ilv-11 |
| SEQ ID NO: 198 | ilv-12 |
| SEQ ID NO: 199 | ilv-13 |
| SEQ ID NO: 200 | ilv-14 |
| SEQ ID NO: 201 | ilv-15 |
| SEQ ID NO: 202 | ilv-16 |
| SEQ ID NO: 203 | ilv-17 |
| SEQ ID NO: 204 | ilv-18 |
| SEQ ID NO: 205 | ilv-19 |
| SEQ ID NO: 206 | ilv-20 |
| SEQ ID NO: 207 | ilv-21 |
| SEQ ID NO: 208 | ilv-22 |
| SEQ ID NO: 209 | ilv-23 |
| SEQ ID NO: 210 | ilv-24 |
| SEQ ID NO: 211 | ilv-25 |
| SEQ ID NO: 212 | ilv-26 |
| SEQ ID NO: 213 | ilv-27 |
| SEQ ID NO: 214 | ilv-28 |
| SEQ ID NO: 215 | ilv-29 |
| SEQ ID NO: 216 | ilv-30 |
| SEQ ID NO: 217 | ilv-31 |
| SEQ ID NO: 218 | ilv-32 |
| SEQ ID NO: 219 | pta-1 |
| SEQ ID NO: 220 | pta-2 |
| SEQ ID NO: 221 | pta-3 |
| SEQ ID NO: 222 | pta-4 |
| SEQ ID NO: 223 | pta-5 |
| SEQ ID NO: 224 | pta-6 |
| SEQ ID NO: 225 | pta-7 |
| SEQ ID NO: 226 | pta-8 |
| SEQ ID NO: 227 | pta-9 |
| SEQ ID NO: 228 | pta-10 |
| SEQ ID NO: 229 | pta-11 |
| SEQ ID NO: 230 | pta-12 |
| SEQ ID NO: 231 | pta-13 |
| SEQ ID NO: 232 | pta-14 |
| SEQ ID NO: 233 | pta-15 |
| SEQ ID NO: 234 | pta-16 |
| SEQ ID NO: 235 | pta-17 |
| SEQ ID NO: 236 | pta-18 |
| SEQ ID NO: 237 | pta-19 |
| SEQ ID NO: 238 | pta-20 |
| SEQ ID NO: 239 | pta-21 |
| SEQ ID NO: 240 | pta-22 |
| SEQ ID NO: 241 | pta-23 |
| SEQ ID NO: 242 | pta-24 |
| SEQ ID NO: 243 | pta-25 |
| SEQ ID NO: 244 | pta-26 |
| SEQ ID NO: 245 | pta-27 |
| SEQ ID NO: 246 | pta-28 |
| SEQ ID NO: 247 | pta-29 |
| SEQ ID NO: 248 | pta-30 |
| SEQ ID NO: 249 | pta-31 |
| SEQ ID NO: 250 | pta-32 |
| SEQ ID NO: 251 | pta-33 |
| SEQ ID NO: 252 | pta-34 |
| SEQ ID NO: 253 | pta-35 |
| SEQ ID NO: 254 | pta-36 |
| SEQ ID NO: 255 | pur-1 |
| SEQ ID NO: 256 | pur-2 |
| SEQ ID NO: 257 | pur-3 |
| SEQ ID NO: 258 | pur-4 |
| SEQ ID NO: 259 | pur-5 |
| SEQ ID NO: 260 | pur-6 |
| SEQ ID NO: 261 | pur-7 |
| SEQ ID NO: 262 | pur-8 |
| SEQ ID NO: 263 | pur-9 |
| SEQ ID NO: 264 | pur-10 |
| SEQ ID NO: 265 | pur-11 |
| SEQ ID NO: 266 | pur-12 |
| SEQ ID NO: 267 | pur-13 |
| SEQ ID NO: 268 | pur-14 |
| SEQ ID NO: 269 | pur-15 |
| SEQ ID NO: 270 | pur-16 |
| SEQ ID NO: 271 | pur-17 |
| SEQ ID NO: 272 | pur-18 |
| SEQ ID NO: 273 | pur-19 |
| SEQ ID NO: 274 | pur-20 |
| SEQ ID NO: 275 | pur-21 |
| SEQ ID NO: 276 | pur-22 |
| SEQ ID NO: 277 | pur-23 |
| SEQ ID NO: 278 | pur-24 |
| SEQ ID NO: 279 | pur-25 |
| SEQ ID NO: 280 | pur-26 |
| SEQ ID NO: 281 | pur-27 |
| SEQ ID NO: 282 | pur-28 |
| SEQ ID NO: 283 | pur-29 |
| SEQ ID NO: 284 | pur-30 |
| SEQ ID NO: 285 | pur-31 |
| SEQ ID NO: 286 | pur-32 |
| SEQ ID NO: 287 | pur-33 |
| SEQ ID NO: 288 | pur-34 |
| SEQ ID NO: 289 | pur-35 |
| SEQ ID NO: 290 | pur-36 |
| SEQ ID NO: 291 | pur-37 |
| SEQ ID NO: 292 | pur-38 |
| SEQ ID NO: 293 | pur-39 |
| SEQ ID NO: 294 | pur-40 |
| SEQ ID NO: 295 | pyc-1 |
| SEQ ID NO: 296 | pyc-2 |
| SEQ ID NO: 297 | pyc-3 |
| SEQ ID NO: 298 | pyc-4 |
| SEQ ID NO: 299 | pyc-5 |
| SEQ ID NO: 300 | pyc-6 |
| SEQ ID NO: 301 | pyc-7 |
| SEQ ID NO: 302 | pyc-8 |
| SEQ ID NO: 303 | pyc-9 |
| SEQ ID NO: 304 | pyc-10 |
| SEQ ID NO: 305 | pyc-11 |
| SEQ ID NO: 306 | pyc-12 |
| SEQ ID NO: 307 | pyc-13 |
| SEQ ID NO: 308 | pyc-14 |
| SEQ ID NO: 309 | pyc-15 |
| SEQ ID NO: 310 | pyc-16 |
| SEQ ID NO: 311 | pyc-17 |
| SEQ ID NO: 312 | pyc-18 |
| SEQ ID NO: 313 | pyc-19 |
| SEQ ID NO: 314 | pyc-20 |
| SEQ ID NO: 315 | pyc-21 |
| SEQ ID NO: 316 | pyc-22 |
| SEQ ID NO: 317 | pyc-23 |
| SEQ ID NO: 318 | pyc-24 |
| SEQ ID NO: 319 | pyc-25 |
| SEQ ID NO: 320 | pyc-26 |
| SEQ ID NO: 321 | pyc-27 |
| SEQ ID NO: 322 | pyc-28 |
| SEQ ID NO: 323 | pyc-29 |
| SEQ ID NO: 324 | pyc-30 |
| SEQ ID NO: 325 | pyc-31 |
| SEQ ID NO: 326 | pyc-32 |
| SEQ ID NO: 327 | pyc-33 |
| SEQ ID NO: 328 | rpo-1 |
| SEQ ID NO: 329 | rpo-2 |
| SEQ ID NO: 330 | rpo-3 |
| SEQ ID NO: 331 | rpo-4 |
| SEQ ID NO: 332 | rpo-5 |
| SEQ ID NO: 333 | rpo-6 |
| SEQ ID NO: 334 | rpo-7 |
| SEQ ID NO: 335 | rpo-8 |
| SEQ ID NO: 336 | rpo-9 |
| SEQ ID NO: 337 | rpo-10 |
| SEQ ID NO: 338 | rpo-11 |
| SEQ ID NO: 339 | rpo-12 |
| SEQ ID NO: 340 | rpo-13 |
| SEQ ID NO: 341 | rpo-14 |
| SEQ ID NO: 342 | rpo-15 |
| SEQ ID NO: 343 | rpo-16 |
| SEQ ID NO: 344 | rpo-17 |
| SEQ ID NO: 345 | rpo-18 |
| SEQ ID NO: 346 | rpo-19 |
| SEQ ID NO: 347 | rpo-20 |
| SEQ ID NO: 348 | rpo-21 |
| SEQ ID NO: 349 | rpo-22 |
| SEQ ID NO: 350 | rpo-23 |
| SEQ ID NO: 351 | rpo-24 |
| SEQ ID NO: 352 | rpo-25 |
| SEQ ID NO: 353 | rpo-26 |
| SEQ ID NO: 354 | rpo-27 |
| SEQ ID NO: 355 | rpo-28 |
| SEQ ID NO: 356 | tpi-1 |
| SEQ ID NO: 357 | tpi-2 |
| SEQ ID NO: 358 | tpi-3 |
| SEQ ID NO: 359 | tpi-4 |
| SEQ ID NO: 360 | tpi-5 |
| SEQ ID NO: 361 | tpi-6 |
| SEQ ID NO: 362 | tpi-7 |
| SEQ ID NO: 363 | tpi-8 |
| SEQ ID NO: 364 | tpi-9 |
| SEQ ID NO: 365 | tpi-10 |
| SEQ ID NO: 366 | tpi-11 |
| SEQ ID NO: 367 | tpi-12 |
| SEQ ID NO: 368 | tpi-13 |
| SEQ ID NO: 369 | tpi-14 |
| SEQ ID NO: 370 | tpi-15 |
| SEQ ID NO: 371 | tpi-16 |
| SEQ ID NO: 372 | tpi-17 |
| SEQ ID NO: 373 | tpi-18 |
| SEQ ID NO: 374 | tpi-19 |
| SEQ ID NO: 375 | tpi-20 |
| SEQ ID NO: 376 | tpi-21 |
| SEQ ID NO: 377 | tpi-22 |
| SEQ ID NO: 378 | tpi-23 |
| SEQ ID NO: 379 | tpi-24 |
| SEQ ID NO: 380 | tpi-25 |
| SEQ ID NO: 381 | tpi-26 |
| SEQ ID NO: 382 | tpi-27 |
| SEQ ID NO: 383 | tpi-28 |
| SEQ ID NO: 384 | tpi-29 |
| SEQ ID NO: 385 | tpi-30 |
| SEQ ID NO: 386 | tpi-31 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of classifying a Bacillus bacterium, comprising:
analyzing a nucleic acid encoding an SspE protein of said Bacillus bacterium to determine an SspE proteotype; and
classifying said Bacillus bacterium on the basis of said SspE proteotype.

2. The method of claim 1, wherein said method further includes:
analyzing said nucleic acid to determine an sspE genotype; and
further classifying Bacillus bacterium on the basis of said sspE genotype.

3. The method of claim 2, wherein said method further includes:
subjecting said Bacillus bacterium to multi-locus sequence typing (MLST) analysis to provide an MLST type; and
further classifying said Bacillus bacterium on the basis of said MLST type.

4. The method of claim 1, wherein said method includes sequencing said nucleic acid to provide a nucleic acid sequence.

5. The method of claim 4, wherein said method includes analyzing said nucleic acid sequence.

6. The method of claim 4, further including translating said nucleic acid to provide an SspE amino acid sequence.

7. The method of claim 1, wherein said analyzing includes identifying an SspE classifying amino acid signature in said SspE protein.

8. The method of claim 6, wherein said analyzing includes comparing said SspE amino acid sequence to a plurality of known Bacillus SspE amino acid sequences.

9. The method of claim 1, wherein said analyzing includes detection using Bacillus classifying primers, which primers specifically detect a classifying SspE amino acid signature.

10. The method of claim 1, wherein said Bacillus bacterium is of unknown identity.

11. The method of claim 1, wherein method is employed to confirm the identity of a Bacillus bacterium of presumed identity.

12. The method of claim 1, wherein classifying identifies a use for said Bacillus bacterium.

13. The method of claim 1, wherein said Bacillus bacterium is a Bacillus subtilis group or Bacillus thuringiensis group bacterium.

14. A method comprising:
a) classifying a Bacillus bacterium using the method of claim 1; and
b) employing said Bacillus bacterium in a method consistent with said classification.

15. A computer readable medium comprising:
instructions for performing the method of claim 1.

16. The computer readable medium of claim 15, wherein said computer readable medium further comprises a database of sspE nucleotide or amino acid sequences.

17. A set of oligonucleotide primers that detects a specific classifying SspE amino acid signature.

18. The set of oligonucleotide primers of claim 17, wherein said primers are designed so that when they are employed in a polymerase chain reaction using the genome of a Bacillus bacterium as a template to provide reaction products, the reaction products classify said Bacillus bacterium.

19. A composition comprising a re-classified isolate of Bacillus bacterium selected from Tables 11 and 12, used in accordance with its new classification.

20. The composition of claim 19, wherein said Bacillus bacterium is a previously unclassified Bacillus bacterium.

21. The composition of claim 19, wherein said Bacillus bacterium is a mis-classified Bacillus bacterium.


SEQUENCE LISTING (386 sequences)

SEQ ID NO: 1 - protein, 93 aa, BACILLUS SP.
MSKKQQGYNKATSGASIQSTNASYGTEFSTETDVQAVKQANAQSEAKKAQASGAQSANASYGTEFATETDVHSVKKQNAKSAAKQSQSSSSNQ

SEQ ID NO: 2 - protein, 93 aa, BACILLUS SP.
MSKKQQGYNKATSGASIQSTNASYGTEFSTETDVQAVKQANAQSEAKKAQASGAQSANASYGTEFATETDVHSVKKQNAKSAAKQSKSSSSNQ

SEQ ID NO: 3 - protein, 93 aa, BACILLUS SP.
MSKKQQGYNKATSGASIQSTNASYGTEFSTETDVQAVKQANAQSEAKKAQASGAQSANASYGTEFATETDVHAVKKQNAKSAAKQSQSSSSNQ

SEQ ID NO: 4 - protein, 93 aa, BACILLUS SP.
MSKKQQGYNKATSGASIQSTNASYGTEFATETDVQAVKQANAQSEAKKAQASGAQSANASYGTEFATETDVHSVKKQNAKSAAKQSQSSSSNQ

SEQ ID NO: 5 - protein, 93 aa, BACILLUS SP.
MSKKQQGYNKATSGASIQSTNASYGTEFSTETDVQAVKQANAQSEAKKAQASGAQSANASYGTEFATETDVHAVKKQNAQSAAKQSQSSSSNQ

SEQ ID NO: 6 - protein, 93 aa, BACILLUS SP.
MSKKQQGYNKATSGASIQSTNASYGTEFATETNVQAVKQANAQSEAKKAQASGAQSANASYGTEFATETDVHAVKKQNAKSAAKQSQSSSSNQ

SEQ ID NO: 7 - protein, 93 aa, BACILLUS SP.
MSKKQQGYNKATSGASIQSTNASYGTEFATETNVQAVKQANAQSEAKKAQASGAKSANASYGTEFATETDVHAVKKQNAKSAAKQSQSSSSNQ

SEQ ID NO: 8 - protein, 93 aa, BACILLUS SP.
MSKKQQGYNKATSGASIQSTNASYGTEFATETNVQAVKQANAQSEAKKAQASGAQSANASYGTEFATETDVHAVKKQNAKSAAKQSQSSSSNE

SEQ ID NO: 9 - protein, 93 aa, BACILLUS SP.
MSKKQQGYNKATSGASIQSTNASYCTEFATETNVQAVKQANAQSEAKKAQASGAQSANASYGTEFATETDVHAVKKQNAKSAAKQSQSSSSNE

SEQ ID NO: 10 - protein, 93 aa, BACILLUS SP.
MSKKQQGYNKATSGASIQSTNASYGTEFATETNVQAVKQANAQSEAKKAQTSGAQSANASYGTEFATETDVHAVKKQNAKSAAKQSQSSSSNE

SEQ ID NO: 11 - protein, 93 aa, BACILLUS SP.
MSKKQQGYNKATSGASIQSTNASYGTEFATETNVQAVKQANAQSEAKKAQASGAQSANASYGTEFATETDVHAVKKQNAQSAAKQSQSSSSNQ

SEQ ID NO: 12 - protein, 93 aa, BACILLUS SP.
MSKKQQGYNKATSGASIQSTNASYGTEFATETNAQAVKQANAQSEAKKAQASGAQSANASYGTEFATETDVHAVKKQNAQSAAKQSQSSSSNQ

SEQ ID NO: 13 - protein, 93 aa, BACILLUS SP.
MSKKQQGYNKATSGASIQSTNASYGTEFATETNVQAVKQANAQSEAKKAQASGAQSANASYGTEFATETDVHAVKKQNAQSAAKQSQSSSSNE

SEQ ID NO: 14 - protein, 92 aa, BACILLUS SP.
MSKKQQGYNKATSGASIQSTNASYGTEFATETNVQAVKQANAQSEAKKAQASGAQSANASYGTEFATETDVHAVKKQNAQSAAKQSQSSSSN

SEQ ID NO: 15 - protein, 95 aa, BACILLUS SP.
MSKKQQGYNKATSGASIQSTNASYGTEFATETNVQAVKQANAQSEAKKAQASGASIQSTNASYGTEFATETDVHAVKKQNAQSAAKQSQSSSSNQ

SEQ ID NO: 16 - protein, 95 aa, BACILLUS SP.
MSKKQQGYNKATSGASIQSTNASYGTEFATETNVQAVKQANAQSEAKKAQASGASVQSTNASYGTEFATETDVHAVKKQNAQSAAKQSQSSSSNQ

SEQ ID NO: 17 - protein, 93 aa, BACILLUS SP.
MSKKQQGYNKATSGASIQSTNASYGTEFATETNVQAVKQANAQSEAQKAQASAAQSANASYGTEFATETDVHAVKKQNAQSAAKQSQSSSSNE

SEQ ID NO: 18 - protein, 93 aa, BACILLUS SP.
MSKKQQGYNKATSGASIQSTNASYGTEFATETNVQAVKQANAQSEAQKAQASAAQSANASYGTEFATETDVHAVKKQNAQSAATQSQSSSSNE

SEQ ID NO: 19 - protein, 92 aa, BACILLUS SP.
MNKKQQYNKATSGASIQSTNASYGTEFATETNVQAVQKQNAQSEAKKAQASGAQSANASYGTEFATETDVQAVKKQNAQSAANKSQSSSSNQ

SEQ ID NO: 20 - protein, 95 aa, BACILLUS SP.
MSKKQQGYNKATSGASIQSTNASYGTEFATETNVQAVKQQNAQSEAKKAQASSAGVQSANASYGTEFATDTDVQAVKQQNAQSAAKKSQSSSTNQ

SEQ ID NO: 21 - protein, 95 aa, BACILLUS SP.
MSKKQQAYNKATSGASIQSTNASYGTEFATETNVQAVKQQNAQSEAKKAQASSAGVQSANASYGTEFATDTDVQAVKQQNAQSAAKKSQSSSTNQ

SEQ ID NO: 22 - protein, 85 aa, BACILLUS SP.
MANSNNFSKTNAQQVRKQNQQSAAGQGQFGTEFASETNAQQVRKQNQQSAAGQQGQFGTEFASETDVQQVRQQNQSAEQNKQQNS

SEQ ID NO: 23 - protein, 85 aa, BACILLUS SP.
MANSNNFSKTNAQQVRKQNQQSAAGQGQFGTEFASETNAQQVRKQNQQSAAGQQGQFGTEFASETDAQQVRQQNQSAEQNKQQNS

SEQ ID NO: 24 - protein, 85 aa, BACILLUS SP.
MANSNNSSKTNAQQVRKQNQQSAAGQGQFGTEFASETNAQQVKKQNQQSAAGQQGQFGTEFASETDAQQVRQQNQSAEQNKQQNS

SEQ ID NO: 25 - protein, 84 aa, BACILLUS SP.
MANSNNFSKTNAQQVRKQNQQSAAGQGQFGTEFASETNAQQVRKQNQQSAGQQGQFGTEFASETDVQQVRQQNQSAEQNKQQNS

SEQ ID NO: 26 - protein, 84 aa, BACILLUS SP.
MANSNNFSKTNAQQVRKQNQQSAAGQGQFGTEFASETNAQQVRKQNQQSAGQQGQFGTEFASETDAQQVRQQNQSAEQNKQQNS

SEQ ID NO: 27 - protein, 84 aa, BACILLUS SP.
MANSNNFSKTNAQQVRKQNQQSAAGQGQFGTEFASETNAQQVRKQNQQSAGQQSQFGTEFASETDVQQVRQQNQSAEQNKQQNS

SEQ ID NO: 28 - protein, 82 aa, BACILLUS SP.
MANSNNKTNAQQVRKQNQQSASGQGQFGTEFASETNVQQVRKQNQQSAAGQGQFGTEFASETDAQQVRQQNQSAEQNKQQNS

SEQ ID NO: 29 - protein, 85 aa, BACILLUS SP.
MANSNNFSKTNAQQVRKQNQQSAAGQGQFGTEFASETNVQQVRKQNQQSAAGQQGQFGTEFASETNTQQVRQQNQSAEQNKQQNS

SEQ ID NO: 30 - protein, 85 aa, BACILLUS SP.
MANSNNFSKTNAQQVRKQNQQSAAGQGQFGTEFASETNAQQVRKQNQQSAAGQQGQFGTEFASETDTQQVRQQNQSAEQNKQQNS

SEQ ID NO: 31 - protein, 85 aa, BACILLUS SP.
MANSNNFSKTNAQQVRKQNQQSAAGQGQFGTEFASETNAQQVRKQNQQSAAGQQGQFGTEFASETNTQQVRQQNQSAEQNKQQNS

SEQ ID NO: 32 - protein, 84 aa, BACILLUS SP.
MANQNFSKTNAQQVRKQNQQSAAGQGQFGTEFASETNVQQVRKQNQQSAAGQQGQFGTEFASETNVQQVRQQNQSAEQNKQQNS

SEQ ID NO: 33 - protein, 84 aa, BACILLUS SP.
MANQNFSKTNAQQVRNQNQQSAAGQGQFGTEFASETNVQQVRKQNQQSAAGQQGQFGTEFASETNVQQVRQQNQSAEQNKQQNS

SEQ ID NO: 34 - protein, 54 aa, BACILLUS SP.
MANQQSKTNAQKVRQQNQASAQGGQFGTEFASETDAQQVRQNNQQAEQKKQQNS

SEQ ID NO: 35 - protein, 54 aa, BACILLUS SP.
MANQQSKTNAQKVRQQNQASAQGGQFGTEFASETDAQQVRKNNQQAEQNKQQNS

SEQ ID NO: 36 - protein, 56 aa, BACILLUS SP.
MANSKYSKTDVQQVKRQNQQSASGQGQYGTEFGSETDAQQVRKQNQSAEQNKQQNS

SEQ ID NO: 37 - protein, 56 aa, BACILLUS SP.
MANSKYSKTDVQQVKRQNQQSASGQGQYGTEFGSETDVQQVRKQNQSAEQNKQQNS

SEQ ID NO: 38 - protein, 56 aa, BACILLUS SP.
MANSKYSKTDVQQVKKQNQQSASGQGQYGTEFGSETDAQQVRKQNQSAEQNKQQNS

SEQ ID NO: 39 - protein, 56 aa, BACILLUS SP.
MENSKYSKTDVQQVKKQNQQSASGQGQYGTEFGSETDAQQVRKQNQSAEQNKQQNS

SEQ ID NO: 40 - protein, 56 aa, BACILLUS SP.; identical to SEQ ID NO: 36

SEQ ID NO: 41 - protein, 56 aa, BACILLUS SP.; identical to SEQ ID NO: 37

SEQ ID NO: 42 - protein, 56 aa, BACILLUS SP.; identical to SEQ ID NO: 38

SEQ ID NO: 43 - protein, 56 aa, BACILLUS SP.; identical to SEQ ID NO: 39

SEQ ID NO: 44 - protein, 54 aa, BACILLUS SP.; identical to SEQ ID NO: 35

SEQ ID NO: 45 - protein, 54 aa, BACILLUS SP.; identical to SEQ ID NO: 34

SEQ ID NO: 46 - protein, 55 aa, BACILLUS SP.
MMDQQQNKTNAQQVKKQNQASAQGGQYGTEFASETDVQQVKKYNQKAEQNKQQNS

SEQ ID NO: 47 - protein, 55 aa, BACILLUS SP.
MMDQQQNKTNAQQVKKQNQASAQGGQFGTEFASETDVQQVKKYNQKAEQNKQQNS

SEQ ID NO: 48 - protein, 55 aa, BACILLUS SP.
MMDQQQNKTNAQQVKKQNQAAAQGGQYGTEFASETDAQHVKKYNQKAEQNKQQNS

SEQ ID NO: 49 - protein, 93 aa, BACILLUS SP.; identical to SEQ ID NO: 1

SEQ ID NO: 50 - protein, 93 aa, BACILLUS SP.; identical to SEQ ID NO: 2

SEQ ID NO: 51 - protein, 93 aa, BACILLUS SP.; identical to SEQ ID NO: 3

SEQ ID NO: 52 - protein, 93 aa, BACILLUS SP.; identical to SEQ ID NO: 5

SEQ ID NO: 53 - protein, 93 aa, BACILLUS SP.; identical to SEQ ID NO: 4

SEQ ID NO: 54 - protein, 93 aa, BACILLUS SP.; identical to SEQ ID NO: 6

SEQ ID NO: 55 - protein, 93 aa, BACILLUS SP.; identical to SEQ ID NO: 7

SEQ ID NO: 56 - protein, 93 aa, BACILLUS SP.; identical to SEQ ID NO: 8

SEQ ID NO: 57 - protein, 93 aa, BACILLUS SP.; identical to SEQ ID NO: 9

SEQ ID NO: 58 - protein, 93 aa, BACILLUS SP.; identical to SEQ ID NO: 10

SEQ ID NO: 59 - protein, 93 aa, BACILLUS SP.; identical to SEQ ID NO: 11

SEQ ID NO: 60 - protein, 93 aa, BACILLUS SP.; identical to SEQ ID NO: 12

SEQ ID NO: 61 - protein, 93 aa, BACILLUS SP.; identical to SEQ ID NO: 13

SEQ ID NO: 62 - protein, 92 aa, BACILLUS SP.; identical to SEQ ID NO: 14

SEQ ID NO: 63 - protein, 95 aa, BACILLUS SP.; identical to SEQ ID NO: 15

SEQ ID NO: 64 - protein, 95 aa, BACILLUS SP.; identical to SEQ ID NO: 16

SEQ ID NO: 65 - protein, 93 aa, BACILLUS SP.; identical to SEQ ID NO: 17

SEQ ID NO: 66 - protein, 93 aa, BACILLUS SP.; identical to SEQ ID NO: 18

SEQ ID NO: 67 - protein, 92 aa, BACILLUS SP.; identical to SEQ ID NO: 19

SEQ ID NO: 68 - protein, 95 aa, BACILLUS SP.; identical to SEQ ID NO: 20

SEQ ID NO: 69 - protein, 95 aa, BACILLUS SP.; identical to SEQ ID NO: 21

SEQ ID NO: 70 - DNA, 282 bp, BACILLUS SP.
atgagtaaaaaacaacaaggttataacaaagcaacttctggtgctagcattcaaagtacaaatgctagttatggtacagagttttcaactgaaacagatgtacaagctgtaaaacaagcaaacgcacaatcagaagcaaagaaagcacaagcttctggtgcacaaagtgcaaacgctagttatggtacagaatttgcaactgaaacagacgtgcattctgtgaaaaaacaaaatgctaagtcagctgcaaaacaatcacaatcttctagctcaaatcagtaa

SEQ ID NO: 71 - DNA, 282 bp, BACILLUS SP.
atgagtaaaaaacaacaaggttataacaaagcaacttctggtgctagcattcaaagtacaaatgctagttatggtacagagttttcaactgaaacagatgtacaagctgtaaaacaagcaaacgcacaatcagaagcaaagaaagcgcaagcttctggtgcacaaagtgcaaacgctagttatggtacagaatttgcaactgaaacagacgtgcattctgtgaaaaaacaaaatgctaagtcagctgcaaaacaatcacaatcttctagctcaaatcagtaa

SEQ ID NO: 72 - DNA, 282 bp, BACILLUS SP.
atgagtaaaaaacaacaaggttataacaaagcaacttctggtgctagcattcaaagtacaaatgctagttatggtacagagttttcaactgaaacagatgtacaagctgtaaaacaagcaaacgcacaatcagaagcaaagaaagcacaagcttctggtgcacaaagtgcaaacgctagttatggtacagaatttgcaactgaaacagacgtgcattctgtgaaaaaacaaaatgctaagtcagctgcaaaacaatcaaaatcttctagctcaaatcagtaa

SEQ ID NO: 73 - DNA, 282 bp, BACILLUS SP.
atgagtaaaaaacaacaaggttataacaaagcaacttctggtgctagcattcaaagtacaaatgctagttatggtacagagttttcaactgaaacagatgtacaagctgtaaaacaagcaaacgcacaatcagaagcaaagaaagcacaagcttctggtgcacaaagtgcaaacgctagttatggtacagaatttgcaactgaaacagacgtgcatgctgtgaaaaaacaaaatgctaagtcagctgcaaaacaatcacaatcttctagctcaaatcagtaa

SEQ ID NO: 74 - DNA, 282 bp, BACILLUS SP.
atgagtaaaaaacaacaaggttataacaaagcaacttctggtgctagcattcaaagtacaaatgctagttatggtacagagtttgcaactgaaacagatgtacaagctgtaaaacaagcaaacgcacaatcagaagcaaagaaagcacaagcttctggtgcacaaagtgcaaacgctagttatggtacagaatttgcaactgaaacagacgtgcattctgtgaaaaaacaaaatgctaagtcagctgcaaaacaatcacaatcttctagctcaaatcagtaa

SEQ ID NO: 75 - DNA, 282 bp, BACILLUS SP.
atgagtaaaaaacaacaaggttataacaaggcaacttctggtgctagcattcaaagtacaaatgctagttatggtacagagttttcaactgaaacagatgtgcaagcagtaaaacaagcaaacgcacaatcagaagcgaagaaagcacaagcttctggtgcacaaagtgcaaacgctagttatggtactgaatttgcaactgaaacagacgtgcatgctgtgaaaaaacaaaatgcacaatcagctgcaaaacaatcacaatcttctagttcaaatcagtaa

SEQ ID NO: 76 - DNA, 282 bp, BACILLUS SP.
atgagtaaaaaacaacaaggttataacaaggcaacttctggtgctagcattcaaagtacaaatgctagttatggtacagagttttcaactgaaacagatgtgcaagcagtaaaacaagcaaacgcgcaatcagaagcaaagaaagcacaagcttctggtgcacaaagtgcaaacgctagttatggtactgaatttgcaactgaaacagacgtgcatgctgtgaaaaaacaaaatgcacaatcagctgcaaaacaatcacaatcttctagttcaaatcagtaa

SEQ ID NO: 77 - DNA, 282 bp, BACILLUS SP.
atgagtaaaaaacaacaaggttataacaaagcaacttctggtgctagcattcaaagtacaaatgctagttatggtacagagttttcaactgaaacagatgtgcaagcagtaaaacaagcaaacgcacaatcagaagcaaagaaagcacaagcttctggtgcacaaagtgcaaacgctagttatggtactgaatttgcaactgaaacagacgtgcatgctgtgaaaaaacaaaatgcacaatcagctgcaaaacaatcacaatcttctagttcaaatcagtaa

SEQ ID NO: 78 - DNA, 282 bp, BACILLUS SP.
atgagtaaaaaacaacaaggttataacaaggcaacttctggcgctagcattcaaagtacaaatgctagttatggtacagagttttcaactgaaacagatgtgcaagcagtaaaacaagcaaatgcacaatcagaagcaaagaaagcacaagcttctggtgcacaaagtgcaaacgctagttatggtactgaatttgcaactgaaacagatgtgcatgctgtgaaaaaacaaaatgcacaatcagctgcaaaacaatcacaatcttctagttcaaatcagtaa

SEQ ID NO: 79 - DNA, 282 bp, BACILLUS SP.
atgagtaaaaaacaacaaggttataacaaagcaacttctggtgctagcattcaaagtacaaatgctagttatggtacagagttttcaactgaaacagatgtgcaagcagtaaaacaagcaaacgcacaatcagaagcaaagaaagcgcaagcttctggtgcacaaagtgcaaatgctagctatggtacagaattcgcaactgaaacagatgtgcatgctgtgaaaaaacaaaatgcacaatcagctgcaaaacaatcacaatcttctagttcaaatcagtaa

SEQ ID NO: 80 - DNA, 282 bp, BACILLUS SP.
atgagtaaaaaacaacaaggttataacaaggcaacttctggtgctagcattcaaagtacaaatgctagttatggtacagagttttcaactgaaacagatgtgcaagcagtaaagcaagcaaacgcacaatcagaagcgaagaaagcacaagcttctggtgcacaaagtgcaaatgctagttatggtacagaatttgcaactgaaacagacgtgcatgctgtgaaaaaacaaaatgcacaatcagctgcaaaacaatcacaatcttctagttcaaatcagtaa

SEQ ID NO: 81 - DNA, 282 bp, BACILLUS SP.
atgagtaaaaaacaacaaggttataacaaggcaacttctggtgctagcattcaaagtacaaatgctagttatggtacagagttttcaactgaaacagatgtacaagcagtaaaacaagcaaacgcacaatcagaagcgaagaaagcacaagcttctggtgcacaaagtgcaaatgctagttatggtacagaatttgcaactgaaacagacgtgcatgctgtgaaaaaacaaaatgcacaatcagctgcaaaacaatcacaatcttctagttcaaatcagtaa

SEQ ID NO: 82 - DNA, 282 bp, BACILLUS SP.
atgagtaaaaaacaacaaggttataacaaggcaacttctggtgctagcattcaaagtacaaatgctagttatggtacagagttttcaactgaaacagatgtgcaagcagtaaaacaagcaaacgcacaatcagaagcgaagaaagcacaagcttctggtgcacaaagtgcaaatgctagttatggtacagaatttgcaactgaaacagacgtgcatgctgtgaaaaaacaaaatgcacaatcagctgcgaaacaatcacaatcttctagttcaaatcagtaa

SEQ ID NO: 83 - DNA, 282 bp, BACILLUS SP.
atgagtaaaaaacaacaaggttataacaaggcaacttctggtgctagcattcaaagtacaaatgctagttatggtacagagttttcaactgaaacagatgtgcaagcagtaaaacaagcaaacgcacaatcagaagcaaagaaagcacaagcttctggtgcacaaagtgcaaacgctagttatggtactgaatttgcaactgaaacagacgtgcatgctgtgaaaaaacaaaatgcacaatcagctgcaaaacaatcacaatcttctagttcaaatcagtaa

SEQ ID NO: 84 - DNA, 282 bp, BACILLUS SP.
atgagtaaaaaacaacaaggttataacaaagcaacttctggtgctagcattcaaagtacaaatgctagttatggtacagagttttcaactgaaacagatgtgcaagcagtaaaacaagcaaacgcacaatcagaagcaaagaaagcgcaagcttctggtgcacaaagtgcaaatgctagctatggtacagaatttgcaactgaaacagatgtgcatgctgtgaaaaaacaaaatgcacaatcagctgcaaaacaatcacaatcttctagttcaaatcagtaa

SEQ ID NO: 85 - DNA, 282 bp, BACILLUS SP.
atgagtaaaaaacaacaaggttataacaaagcaacttctggtgctagcattcaaagtacaaatgctagttatggtacagagttttcaactgaaacagatgtgcaagcagtaaaacaagcaaacgcacaatcagaagcaaagaaagcgcaagcttctggtgcacaaagtgcaaatgctagctatggtacagaatttgcaactgaaacagacgtgcatgctgtgaaaaaacaaaatgcacaatcagctgcaaaacaatcacaatcttctagttcaaatcagtaa

SEQ ID NO: 86 - DNA, 282 bp, BACILLUS SP.
atgagtaaaaagcaacaaggttataacaaggcaacttctggtgctagcattcaaagtacaaatgctagttatggtacagagtttgcgactgaaacaaatgtacaagcagtaaaacaagcaaacgcacaatcagaagcaaagaaagcacaagcttctggtgcacaaagtgcaaacgctagctatggtacagaatttgcaactgaaacagacgtgcatgctgtgaaaaaacaaaatgctaagtcagctgcaaaacaatcacaatcttctagctcaaatcagtaa

SEQ ID NO: 87 - DNA, 282 bp, BACILLUS SP.
atgagtaaaaaacaacaaggttataacaaggcaacttctggtgctagcattcaaagtacaaatgctagttatggtacagagtttgcgactgaaacaaatgtacaagcagtaaaacaagcaaacgcacaatcagaagcaaagaaagcacaagcttctggtgcacaaagtgcaaacgctagttatggtacagaatttgcaactgaaacagacgtgcatgctgtgaaaaaacaaaatgctaagtcagctgcaaaacaatcacaatcttctagctcaaatcagtaa

SEQ ID NO: 88 - DNA, 282 bp, BACILLUS SP.
atgagtaaaaaacaacaaggttataacaaggcaacttctggtgctagcattcaaagtacaaatgctagttatggtacagaatttgcaactgaaacaaatgtacaagcagtaaaacaagcaaacgcacaatcagaagcaaagaaagcacaagcttctggtgcacaaagtgcaaacgctagttatggtacagaatttgcaactgaaacagacgtgcatgctgtgaaaaaacaaaatgctaagtcagctgcaaaacaatcacaatcttctagctcaaatcagtaa

SEQ ID NO: 89 - DNA, 282 bp, BACILLUS SP.
atgagtaaaaaacaacaaggttataacaaggcaacttctggtgctagcattcaaagtacaaatgctagttatggtacagaatttgctactgaaacaaatgtacaagcagtaaaacaagcaaacgcacaatcagaagcaaagaaagcacaagcttctggtgcacaaagtgcaaacgctagttatggtacagaatttgcaactgaaacagacgtgcatgctgtgaaaaaacaaaatgctaagtcagctgcaaaacaatcacaatcttctagctcaaatcagtaa

SEQ ID NO: 90 - DNA, 282 bp, BACILLUS SP.
atgagtaaaaaacaacaaggttataacaaggcaacttctggtgctagcattcaaagtacaaatgctagttatggtacagagtttgcgactgaaacaaatgtacaagcagtaaaacaagcaaacgcacaatcagaagcaaagaaagcacaagcttctggtgcaaaaagtgcaaacgctagttatggtacagaatttgcaactgaaacagacgtgcatgctgtgaaaaaacaaaatgctaagtcagctgcaaaacaatcacaatcttctagctcaaatcagtaa

SEQ ID NO: 91 - DNA, 282 bp, BACILLUS SP.
atgagtaaaaaacaacaaggttataacaaggcaacttctggtgctagcattcaaagtacaaatgctagttatggtacagagtttgcgactgaaacaaatgtacaagcagtaaaacaagcaaacgcacaatcagaagcaaagaaagcacaagcttctggtgcacaaagtgcaaacgctagttatggtacagaatttgcaactgaaacagatgtgcatgctgtgaaaaaacaaaatgcaaaatcagctgcaaaacaatcacaatcttctagttcaaacgagtaa

SEQ ID NO: 92 - DNA, 282 bp, BACILLUS SP.
atgagtaaaaaacaacaaggttataacaaggcaacttctggtgctagcattcaaagtacaaatgctagttatggtacagagtttgcgactgaaacaaatgtacaagcagtaaaacaagcaaacgcacaatcagaagcaaagaaagcacaagcttctggtgcacaaagtgcaaacgctagttatggtacagaatttgcaactgaaacagacgtgcatgctgtgaaaaaacaaaatgcaaaatcagctgcaaaacaatcacaatcttctagttcaaacgagtaa

SEQ ID NO: 93 - DNA, 282 bp, BACILLUS SP.
atgagtaaaaaacaacaaggttataacaaggcaacttctggtgctagcattcaaagtacaaatgctagttatggtacagagtttgcaactgaaacaaatgtacaagcagtaaaacaagcaaacgcacaatcagaagcaaagaaagcacaagcttctggtgcacaaagtgcaaacgctagttatggtacagaatttgcaactgaaacagacgtgcatgctgtgaaaaaacaaaatgctaagtcagctgcaaaacaatcacaatcttctagttcaaacgagtaa

SEQ ID NO: 94 - DNA, 282 bp, BACILLUS SP.
atgagtaaaaaacaacaaggttataacaaggcaacttctggtgctagtattcaaagtacaaatgctagttatggtacagagtttgcaactgaaacaaatgtacaagcagtaaaacaagcaaacgcacaatcagaagcaaagaaagcacaagcttctggtgcacaaagtgcaaacgctagttatggtacagaatttgcaactgaaacagacgtgcatgctgtgaaaaaacaaaatgctaagtcagctgcaaaacaatcacaatcttctagttcaaacgagtaa

SEQ ID NO: 95 - DNA, 282 bp, BACILLUS SP.
atgagtaaaaaacaacaaggttataacaaggcaacttctggtgctagcattcaaagtacaaatgctagttatggtacagagtttgcgactgaaacaaatgtacaagcagtaaaacaagcaaacgcacaatcagaagcaaagaaagcacaagcttctggtgcacaaagtgcaaacgctagctatggtacagaatttgcaactgaaacagacgtgcatgctgtgaaaaaacaaaatgctaagtcagctgcaaaacaatcacaatcttctagttcaaacgagtaa

SEQ ID NO: 96 - DNA, 282 bp, BACILLUS SP.
atgagtaaaaaacaacaaggttataacaaggcaacttctggtgctagcattcaaagtacaaatgctagttattgtacagagtttgcgactgaaacaaatgtacaagcagtaaaacaagcaaacgcacaatcagaagcaaagaaagcacaagcttctggtgcacaaagtgcaaacgctagctatggtacagaatttgcaactgaaacagacgtgcatgctgtgaaaaaacaaaatgctaagtcagctgcaaaacaatcacaatcttctagttcaaacgagtaa

SEQ ID NO: 97 - DNA, 282 bp, BACILLUS SP.; identical to SEQ ID NO: 74

SEQ ID NO: 98 - DNA, 282 bp, BACILLUS SP.
atgagtaaaaaacaacaaggttataacaaggcaacttctggtgctagtattcaaagtacaaatgctagctatggtacagagtttgcgactgaaacaaatgtacaagcggtaaaacaagcaaacgcacaatcagaagcaaagaaagcacaagcttctggtgcacaaagtgcaaacgctagttatggtactgaatttgcaactgaaacagacgtgcatgctgtgaaaaaacaaaatgcacaatcagctgcaaaacaatcacaatcttctagttcaaatcagtaa

SEQ ID NO: 99 - DNA, 282 bp, BACILLUS SP.
atgagtaaaaaacaacaaggttataacaaggcaacttctggtgctagcattcaaagcacaaatgctagttatggtacagagtttgcgactgaaacaaatgtacaagcagtaaaacaagcaaacgcacaatcagaagctaagaaagcgcaagcttctggtgcacaaagtgcaaatgctagttatggtacagaatttgcaactgaaacagacgtgcatgctgtgaaaaaacaaaatgcacaatcagctgcaaaacaatcacaatcttctagttcaaatcagtaa

SEQ ID NO: 100 - DNA, 282 bp, BACILLUS SP.
atgagtaaaaaacaacaaggttataacaaggcaacttctggtgctagcattcaaagtacaaatgctagttatggtacagagttcgcgactgaaacaaatgtacaagcagtaaaacaagcaaatgcacaatcagaagcaaagaaagcacaagcttctggtgcacaaagtgcaaacgctagttatggtacagaattcgcaactgaaacagatgtgcatgctgtgaaaaaacaaaatgcacaatcagctgcaaaacaatcacaatcttctagttcaaatcagtaa

SEQ ID NO: 101 - DNA, 282 bp, BACILLUS SP.
atgagtaaaaaacaacaaggttataacaaggcaacttctggtgctagcattcaaagtacaaatgctagttatggtacagagttcgcaactgaaacaaatgcacaagcagtaaaacaagcaaacgcacaatcagaagcaaagaaagcacaagcttccggtgcacaaagtgcaaacgctagttatggtacagaattcgcaactgaaacagatgtgcacgctgtgaaaaaacaaaatgcacaatcagctgcaaaacaatcacaatcttctagttcaaatcagtaa

SEQ ID NO: 102 - DNA, 282 bp, BACILLUS SP.
atgagtaaaaaacaacaaggttataacaaggcaacttctggtgctagcattcaaagtacaaatgctagttatggtacagagtttgcgactgaaacaaatgtacaagcagtaaaacaagcgaatgcacaatcagaagcaaagaaagcacaagcttctggtgcacaaagtgcaaacgctagttatggtacagaatttgcaactgaaacagacgtgcatgctgtgaaaaaacaaaatgcacaatcagctgcaaaacaatcacaatcttctagttcaaacgagtaa

SEQ ID NO: 103 - DNA, 282 bp, BACILLUS SP.
atgagtaaaaaacaacaaggttataacaaggcaacttctggtgctagcattcaaagcacaaatgctagttatggtacagagtttgcgactgaaacaaatgtacaagcagtaaaacaagcaaacgcacaatcagaagctaagaaagcgcaagcttctggtgcacaaagtgcaaatgctagttatggtacagaatttgcaactgaaacagacgtgcatgctgtgaaaaaacaaaatgcacaatcagctgcaaaacaatcacaatcttctagttcaaattagtaa

SEQ ID NO: 104 - DNA, 288 bp, BACILLUS SP.
atgagtaaaaaacaacaaggttataacaaggcaacttctggtgctagcattcaaagcacaaatgctagttatggtacagagtttgcgactgaaacaaatgtacaagcagtaaaacaagcaaacgcacaatcagaagctaagaaagcgcaagcttctggtgctagcattcaaagcacaaatgctagttatggtacagaatttgcaactgaaacagacgtgcatgctgtgaaaaaacaaaatgcacaatcagctgcaaaacaatcacaatcttctagttcaaatcagtaa

SEQ ID NO: 105 - DNA, 288 bp, BACILLUS SP.
atgagtaaaaaacaacaaggttataacaaggcaacttctggtgctagcattcaaagcacaaatgctagttatggtacagagtttgcgactgaaacaaatgtacaagcagtaaaacaagcaaacgcacaatcagaagctaagaaagcgcaagcttctggtgctagcgttcaaagcacaaatgctagttatggtacagaatttgcaactgaaacagacgtgcatgctgtgaaaaaacaaaatgcacaatcagctgcaaaacaatcacaatcttctagttcaaatcagtaa

SEQ ID NO: 106 - DNA, 282 bp, BACILLUS SP.
atgagtaaaaaacaacaaggttataataaggcaacttctggtgctagcattcaaagtacaaatgctagctatggtacagagtttgcaactgaaacgaatgtacaagcagtaaaacaagcgaacgcacaatcagaggcacagaaagcacaagcttctgctgctcaaagtgcaaatgctagttatggtacagagtttgcaactgaaacagatgtgcatgcagtgaaaaaacaaaatgcacaatcagctgcaaaacaatcacaatcttctagttcaaacgagtaa

SEQ ID NO: 107 - DNA, 282 bp, BACILLUS SP.
atgagtaaaaaacaacaaggttataataaggcaacttctggtgctagcattcaaagtacaaatgctagctatggtacagagtttgcaactgaaacgaatgtacaagcagtaaaacaagcgaacgcacaatcagaggcacagaaagcacaagcttctgctgctcaaagtgcaaatgctagttatggtacagagtttgcaactgaaacagatgtgcatgcagtgaaaaaacaaaatgcacaatcagctgcaacacaatcacaatcttctagttcaaacgagtaa

SEQ ID NO: 108 - DNA, 279 bp, BACILLUS SP.
atgaacaaaaaacaacaatataataaggcaacttctggtgctagcattcaaagtacaaatgctagctacggtacagaatttgcaactgaaacgaatgtacaagcagtgcaaaagcaaaacgcacaatcagaggcaaagaaagcacaagcttctggtgctcaaagtgcaaatgctagttacggtacagaatttgcaactgaaacagatgtgcaagctgtgaaaaaacaaaatgcacagtcagctgcaaacaaatcacaatcttctagctcaaatcagtaa

SEQ ID NO: 109 - DNA, 288 bp, BACILLUS SP.
atgagtaaaaaacaacaaggttataacaaagcaacttctggtgctagcattcaaagtacaaatgctagctatggtacagagtttgcaactgagacaaatgtacaagcagtaaaacaacaaaatgcacaatcagaagcgaaaaaagcacaggcttctagcgctggtgttcaaagtgcaaatgctagctacggcacagagtttgcaactgacacagatgtgcaagcagtgaaacaacaaaatgcacaatcagcagcaaaaaaatcacaatcttctagcacgaatcaataa

SEQ ID NO: 110 - DNA, 288 bp, BACILLUS SP.
atgagtaaaaaacaacaagcttataacaaagcaacttctggtgctagcattcaaagtacaaatgctagctatggtacagagtttgcaactgagacaaatgtacaagcagtaaaacaacaaaatgcacaatcagaagcgaaaaaagcacaggcttctagcgctggtgttcaaagtgcaaatgctagctacggcacagagtttgcaactgacacagatgtgcaagcagtgaaacaacaaaatgcacaatcagcagcaaaaaaatcacaatcttctagcacgaatcaataa

SEQ ID NO: 111 - protein, 85 aa, BACILLUS SP.; identical to SEQ ID NO: 24

SEQ ID NO: 112 - protein, 84 aa, BACILLUS SP.; identical to SEQ ID NO: 26

SEQ ID NO: 113 - protein, 85 aa, BACILLUS SP.; identical to SEQ ID NO: 29

SEQ ID NO: 114 - protein, 85 aa, BACILLUS SP.; identical to SEQ ID NO: 30

SEQ ID NO: 115 - protein, 85 aa, BACILLUS SP.; identical to SEQ ID NO: 31

SEQ ID NO: 116 - protein, 54 aa, BACILLUS SP.; identical to SEQ ID NO: 34

SEQ ID NO: 117 - protein, 54 aa, BACILLUS SP.; identical to SEQ ID NO: 35

SEQ ID NO: 118 - protein, 56 aa, BACILLUS SP.; identical to SEQ ID NO: 36

SEQ ID NO: 119 - protein, 56 aa, BACILLUS SP.; identical to SEQ ID NO: 37

SEQ ID NO: 120 - protein, 56 aa, BACILLUS SP.; identical to SEQ ID NO: 38

SEQ ID NO: 121 - protein, 56 aa, BACILLUS SP.; identical to SEQ ID NO: 39

SEQ ID NO: 122 - protein, 85 aa, BACILLUS SP.; identical to SEQ ID NO: 22

SEQ ID NO: 123 - protein, 85 aa, BACILLUS SP.; identical to SEQ ID NO: 23

SEQ ID NO: 124 - protein, 84 aa, BACILLUS SP.; identical to SEQ ID NO: 25

SEQ ID NO: 125 - protein, 84 aa, BACILLUS SP.; identical to SEQ ID NO: 27

SEQ ID NO: 126 - protein, 84 aa, BACILLUS SP.; identical to SEQ ID NO: 32

SEQ ID NO: 127 - protein, 84 aa, BACILLUS SP.; identical to SEQ ID NO: 33

SEQ ID NO: 128 - protein, 82 aa, BACILLUS SP.; identical to SEQ ID NO: 28

SEQ ID NO: 129 - protein, 55 aa, BACILLUS SP.; identical to SEQ ID NO: 47

SEQ ID NO: 130 - protein, 55 aa, BACILLUS SP.; identical to SEQ ID NO: 46

SEQ ID NO: 131 - protein, 55 aa, BACILLUS SP.; identical to SEQ ID NO: 48

SEQ ID NO: 132 - DNA, 258 bp, BACILLUS SP.
atggctaactcaaataactcaagcaaaactaacgctcaacaagttagaaaacaaaaccaacaatcagctgctggtcaaggtcaatttggcactgaatttgctagcgaaacaaacgctcagcaagtcaaaaaacaaaaccagcaatcagctgctggccaacaaggtcaattcggcactgaattcgctagtgaaactgacgcacagcaggtaagacagcaaaaccaatctgctgaacaaaacaaacaacaaaacagctaa

SEQ ID NO: 133 - DNA, 258 bp, BACILLUS SP.
atggctaactcaaataattcaagcaaaactaacgctcaacaagttagaaaacaaaaccaacaatcagctgctggtcaaggtcaatttggcactgaatttgctagcgaaacaaacgctcagcaagtcaaaaaacaaaaccagcaatcagctgctggccaacaaggtcaattcggcactgaattcgctagtgaaactgacgcacagcaggtaagacagcaaaaccaatctgctgagcaaaacaaacaacaaaacagctaa

SEQ ID NO: 134 - DNA, 255 bp, BACILLUS SP.
atggctaactcaaataacttcagcaaaacaaacgctcaacaagttagaaaacaaaaccaacaatcagctgctggtcaaggtcaatttggcactgaatttgctagcgaaacaaacgctcagcaagtcagaaaacaaaaccagcaatcagctggacaacaaggtcaattcggcactgaattcgctagtgaaactgacgcacagcaggtaagacagcaaaaccaatctgctgaacaaaacaaacaacaaaacagctaa

SEQ ID NO: 135 - DNA, 279 bp, BACILLUS SP.
atgaccgtggaggtgataacaatggctaactcaaataatttcagcaaaactaacgctcaacaagttagaaaacaaaaccaacaatcagctgctggtcaaggtcaattcggtactgaatttgctagcgaaacaaacgttcagcaagtcagaaaacaaaaccaacaatcagctgctggccaacaaggtcaattcggcactgaattcgcaagcgaaacaaacactcagcaggtaagacagcaaaaccaatctgctgaacaaaacaaacaacaaaacagctaa

SEQ ID NO: 136 - DNA, 279 bp, BACILLUS SP.
atgaccgtggaggtgataacaatggctaactcaaataacttcagcaaaacaaacgctcaacaagtcagaaaacaaaaccaacaatcagctgctggtcaaggtcaattcggcactgaatttgctagcgaaacaaacgctcagcaagtcagaaaacaaaaccaacaatcagctgctggccaacaaggtcaattcggcactgaattcgcaagcgaaacagacacacagcaggtgagacagcaaaaccaatctgctgaacaaaacaaacaacaaaacagctaa

SEQ ID NO: 137 - DNA, 279 bp, BACILLUS SP.
atgaccgtggaggtgataacaatggctaactcaaataacttcagcaaaacaaacgctcaacaagtcagaaaacaaaaccaacaatcagctgctggtcaaggtcaattcggcactgaatttgctagcgaaacaaacgctcagcaagtcagaaaacaaaaccaacaatcagctgctggccaacaaggtcaattcggcactgaattcgcaagcgaaactaacacacagcaggtaagacagcaaaaccaatctgctgaacaaaacaaacaacaaaacagctaa

SEQ ID NO: 138 - DNA, 165 bp, BACILLUS SP.
atggctaaccaacaatccaaaacaaacgcacaaaaagtaagacaacaaaaccaagcttctgcacaaggtggtcagttcggcactgaatttgccagcgaaactgacgcgcagcaagtaagacaaaataaccaacaagctgagcaaaaaaaacagcaaaacagctaa

SEQ ID NO: 139 - DNA, 165 bp, BACILLUS SP.
atggctaaccaacaatccaaaacaaacgcacaaaaagtaagacaacaaaaccaagcttctgctcaaggtggtcagttcggcactgaatttgccagcgaaactgacgcgcagcaagtgagaaaaaataaccagcaagctgagcaaaacaaacagcaaaacagctaa

SEQ ID NO: 140 - DNA, 171 bp, BACILLUS SP.
atggcaaactcaaaatacagcaaaacagacgttcagcaggtgaaaagacaaaaccagcaatcagcttccggtcagggtcaatacggcactgaattcggcagtgaaactgatgcgcagcaggttagaaagcaaaaccaatctgctgaacaaaacaaacagcaaaacagctaa

SEQ ID NO: 141 - DNA, 171 bp, BACILLUS SP.
atggcaaactcaaaatacagcaaaacagacgttcagcaggtgaaaagacaaaaccagcaatcagcttccggtcagggtcaatacggcactgaattcggcagtgaaactgatgtgcagcaggtaagaaagcaaaaccaatctgctgaacaaaacaaacagcaaaacagctaa

SEQ ID NO: 142 - DNA, 171 bp, BACILLUS SP.
atggcaaactcaaaatacagcaaaacagacgttcagcaggtgaaaaaacaaaaccagcaatcagcttccggtcagggtcaatacggcactgaattcggcagtgaaactgatgcgcagcaggttagaaagcaaaaccaatctgctgaacaaaacaaacagcaaaacagctaa

SEQ ID NO: 143 - DNA, 171 bp, BACILLUS SP.
atggaaaactcaaaatacagcaaaactgacgttcagcaggtgaaaaaacaaaaccagcaatcagcttccggccaaggtcaatacggtactgaattcggcagtgaaactgatgcgcagcaggtgagaaagcaaaaccaatctgctgagcaaaacaaacaacaaaacagctaa

SEQ ID NO: 144 - DNA, 258 bp, BACILLUS SP.
atggctaactcaaacaatttcagcaaaacaaacgcacaacaagttagaaaacaaaaccaacaatcagctgctggtcaaggtcaattcggcactgaatttgctagcgaaacaaacgctcagcaagtcagaaaacaaaaccagcaatcagctgctggccaacaaggtcaattcggcactgaattcgctagtgaaactgacgtacagcaggtaagacagcaaaaccaatctgctgaacaaaacaaacaacaaaacagctaa

SEQ ID NO: 145 - DNA, 258 bp, BACILLUS SP.
atggctaactcaaacaatttcagcaaaacaaacgcacaacaagttagaaaacaaaaccaacaatcagctgctggtcaaggtcaattcggcactgaatttgctagcgaaacaaacgctcagcaagtcagaaaacaaaaccagcaatcagctgctggccaacaaggtcaattcggcactgaattcgctagtgaaactgacgcacagcaggtaagacagcaaaaccaatctgctgaacaaaacaaacaacaaaacagctaa

SEQ ID NO: 146 - DNA, 255 bp, BACILLUS SP.
atggctaactcaaataacttcagcaaaacaaacgctcaacaagttagaaaacaaaaccaacaatcagctgctggtcaaggtcaatttggcactgaatttgctagcgaaacaaacgctcagcaagtcagaaaacaaaaccagcaatcagctggccaacaaggtcaattcggcactgaattcgctagtgaaactgacgtacagcaggtaagacagcaaaaccaatctgctgaacaaaacaaacaacaaaacagctaa

SEQ ID NO: 147 - DNA, 255 bp, BACILLUS SP.
atggctaactcaaataacttcagcaaaacaaacgctcaacaagttagaaaacaaaaccaacaatcagctgctggtcaaggtcaatttggcactgaatttgctagcgaaacaaacgctcagcaagtcagaaaacaaaaccagcaatcagctggccaacaaagtcaattcggcactgaattcgctagtgaaactgacgtacagcaggtaagacagcaaaaccaatctgctgaacaaaacaaacaacaaaacagctaa

SEQ ID NO: 148 - DNA, 255 bp, BACILLUS SP.
atggcaaaccaaaatttcagcaaaacaaacgcacaacaagtcagaaaacaaaaccaacaatcagctgctggtcaaggccaattcggcactgaatttgctagcgaaacaaacgttcagcaagtcagaaaacaaaaccagcaatcagctgctggccaacaaggtcaattcggcactgaattcgctagcgaaacaaacgtacagcaggtaagacagcaaaaccaatctgctgaacaaaacaaacaacaaaacagctaa

SEQ ID NO: 149 - DNA, 255 bp, BACILLUS SP.
atggcaaaccaaaatttcagcaaaacaaacgcacaacaagtcagaaatcaaaaccaacaatcagctgctggtcaaggccaattcggcactgaatttgctagcgaaacaaacgtacagcaagtcagaaaacaaaaccagcaatcagctgctggccaacaaggtcaattcggcactgaattcgctagcgaaacaaacgtacagcaggtaagacagcaaaaccaatctgctgaacaaaacaaacaacaaaacagctaa

SEQ ID NO: 150 - DNA, 249 bp, BACILLUS SP.
atggctaactcaaacaacaaaacaaacgctcaacaagtaagaaaacaaaaccaacaatcagcatctggccaaggtcagtttggtacagaatttgctagcgaaacaaacgtacaacaagtaagaaaacaaaaccaacaatcagctgctggacaaggacaattcggcactgaattcgctagtgaaactgatgctcagcaagtaagacagcaaaaccaatctgctgaacaaaacaaacaacaaaacagctaa

SEQ ID NO: 151 - DNA, 168 bp, BACILLUS SP.
atgatggatcaacaacaaaacaaaacaaacgcacaacaagtgaagaaacaaaaccaagcttctgcacaaggcggtcaatttggtactgaatttgctagtgagactgacgtacagcaagtaaaaaaatacaaccaaaaagctgagcaaaacaaacaacaaaacagctaa

SEQ ID NO: 152 - DNA, 168 bp, BACILLUS SP.
atgatggatcaacaacaaaacaaaacaaacgcgcaacaagtgaagaaacaaaaccaagcttctgcacaaggcggtcaatttggtactgaatttgctagtgagactgacgtacagcaagtaaaaaaatacaaccaaaaagctgagcaaaacaaacaacaaaacagctaa

SEQ ID NO: 153 - DNA, 168 bp, BACILLUS SP.
atgatggatcaacaacaaaacaaaacaaacgcacaacaagtgaagaaacaaaaccaagcttctgcacaaggcggtcaatttggtactgaatttgctagcgagactgacgtacagcaagtaaaaaaatacaaccaaaaagctgagcaaaacaaacaacaaaacagctaa

SEQ ID NO: 154 - DNA, 168 bp, BACILLUS SP.
atgatggatcaacaacaaaacaaaacaaacgcacaacaagtgaagaaacaaaaccaagcttctgcacaaggcggtcaatacggcactgaatttgctagtgagactgacgtacagcaagtaaaaaaatacaaccaaaaagctgagcaaaacaaacaacaaaacagctaa

SEQ ID NO: 155 - DNA, 168 bp, BACILLUS SP.
atgatggatcaacaacaaaacaaaacaaacgcacaacaagtgaagaaacaaaaccaagctgctgcacaaggtggtcaatacggcactgaatttgctagcgagactgacgcacagcacgtaaaaaaatacaaccaaaaagctgagcaaaacaaacaacaaaacagctaa

SEQ ID NO: 156 - DNA, 504 bp, BACILLUS SP.
atcatttttggtgcaggtgtttgtgcaggtgtcaatttaaagaaatcgctttcattccagtctggctggattgttgttgtatttggatggggattgggtgttgccatggcggcatacgcggttggcggcatcagcggtgcccatttgaatccggcgctaacgatagcgcttgcatttgtaggagattttccttggaaagaggttccggtttatattgcggcgcaaatgatcggagcgatcatcggtgcggtgattatttatctgcattacctcccgcactggaagtcaacggatgatcccgctgccaagctgggtgttttctcaacagggcccagcattccgcatacatttgcaaacgttttaagcgaagtgattgggacatttgtccttgtacttggaatcttggccataggtgcaaatcaatttacagaaggacttaatcctttaatcgtcggtttcctcattgtagcaatcggtatttctctgggaggcaccaccggctat

SEQ ID NO: 157 - DNA, 504 bp, BACILLUS SP.
atcatttttggtgcaggtgtttgtgcaggtgtcaatttaaagaaatcgctttcattccagtctggctggattgttgttgtatttggatggggattgggtgttgcgatggcggcatacgcggttggcggcatcagcggtgcccatttgaatccggcgctaacgatagcgcttgcatttgtaggagattttccttggaaagaggttccggtttatattgcggcgcaaatgatcggagcgatcatcggtgcggtgattatctatctgcactacctcccgcactggaagtcaacggatgatcccgctgccaagctgggtgttttctcaacagggcccagcattccgcatacatttgcaaacgttttaagcgaagtgattgggacatttgtccttgtacttggaatcttggccataggtgcaaatcaatttacagaaggacttaatcctttaatcgtcggtttcctcattgtagcaatcggtatttctctgggaggcaccaccggctat

SEQ ID NO: 158 - DNA, 504 bp, BACILLUS SP.
atcatttttggtgcaggtgtttgtgcaggtgtcaatttaaagaaatcgctttcattccagtctggctggattgttgttgtatttggatggggattgggtgttgccatggcggcatacgcggttggcggcatcagcggtgcccatttgaatccggcgctaacgatagcgcttgcatttgtaggagattttccttggaaagaggttccggtttatattgcggcgcaaatgatcggagcgatcatcggtgcggtgattatttatctgcattacctcccgcactggaagtcaacggatgatcccgctgccaagctgggtgttttctcaacagggcccagcattccgcatacatttgcaaacgttttaagcgaagtgattgggacatttgtccttgtacttggaatcttggccattggggcaaatcaatttacagaaggacttaatcctttaatcgtcggtttcctcattgtagcaatcggtatttctctgggaggcaccaccggctat

SEQ ID NO: 159 - DNA, 504 bp, BACILLUS SP.
atcatttttggtgcaggtgtttgtgcaggtgtcaatttaaagaaatcgctttcattccagtctggctggattgttgttgtatttggatggggattgggtgttgcaatggcggcatacgcggttggcggcatcagcggtgcccatttgaatccggcgctaacgatagcgcttgcatttgtaggagattttccttggaaagaggttccggtttatattgcggcgcaaatgatcggagcgatcatcggtgcggtgattatttatctgcattacctcccgcactggaagtcaacggatgatcccgctgccaagctgggtgttttctcaacagggcccagcattccgcatacatttgcaaacgttttaagcgaagtgattgggacatttgtccttgtgcttggaatcttggccattggggcaaatcaatttacagaaggacttaatcctttaatcgtcggtttcctcattgtagcaatcggtatttctctgggaggcaccaccggctat

SEQ ID NO: 160 - DNA, 504 bp, BACILLUS SP.
atcatttttggggcaggtgtttgtgcaggtgtcaatttaaagaaatcgctttcattccagtctggctggattgttgttgtatttgggtggggattgggtgttgccatgggcgcttatgcagttggcggcatcagcggagcccatttgaatccggcgctaacgatagcgcttgcatttgtgggagattttccatggaaagaggttccggtttatattgcggcgcaaatgatcggagcgatcatcggtgcggtgattatttatctgcattacctgccgcattggaagtcaacggatgatcctgctgccaagctgggtgttttctcaacaggtcccagcattccgcatacatttgcaaacgttttaagtgaagtcattgggacatttgtccttgtgcttggaatcttggccattggggcaaatcaatttacagaagggcttaatcctttaatcgtcggtttcctcattgtagcgatcggtatttctctaggaggcaccaccggctat

SEQ ID NO: 161 - DNA, 504 bp, BACILLUS SP.
atcatttttggagcaggggtttgtgcaggtgtcaatttaaagaaatcgctttcattccagtctggctggattgttgttgtatttgggtggggattgggtgttgccatggcggcttatgcagttggcggcataagcggagcacacttgaatccggcgctaacgatagcgcttgcatttgttggagattttccgtgggaagaggttcccgtttatattgcggcgcaaatgatcggagcgatcatcggtgcggtgattatctatttgcattaccttccgcattggaagtcaacggatgatcccgctgccaagctgggggttttctcaacaggccccagcattccgcatacatttgcaaacgttttaagtgaagtgatcgggacttttgtccttgtgcttggaatcctggctattggggcaaatcaatttacagaagggcttaatcctttaatcgtcggtttcctcattgtggcgatcggtatctctcttggaggcaccaccggctat

SEQ ID NO: 162 - DNA, 504 bp, BACILLUS SP.
gttatttttggggcaggtgtttgtgcaggtgtcaatttaaagaaatcgctttcattccagtctggctggattgttgttgtatttgggtggggattgggtgttgccatggcggcttatgcagttggcggcataagcggagcacacttgaatccggcgctaacgatagcgcttgcatttgttggagattttccgtgggaagaggttcccgtttatattgcggcgcaaatgatcggagcgatcatcggtgcggtgattatctatttgcattaccttccgcattggaagtcaacggatgatcctgctgccaagctgggggttttctcaacaggccccagcattccgcatacatttgcaaacgttttaagtgaagtaattgggacttttgtccttgtgcttggaatcctggccattggggcaaatcaatttacagaagggcttaatcctttaatcgtcggtttcctcattgtggcgatcggtatctctcttggaggcaccaccggctat

SEQ ID NO: 163 - DNA, 504 bp, BACILLUS SP.
atcatttttggggcaggtgtttgtgcgggtgtcaatttaaagaaatcgctttcattccagtctggctggattgttattgtattcggatggggattgggtgttgccatggcggcttatgcggttggcggcatcagcggagcccacttgaatccggcgctaacgatagcgcttgcatttgttggggattttccgtggaaagaggttccggtttatattgcggcgcaaatgatcggtgcgatcatcggtgcggttgtgatctatctgcattaccttccgcattggaaaacaacggatgatcccgctgccaagctgggtgttttctcaaccggacccagcattccgcacacattttcaaacgtgttaagtgaagtgatcgggacatttgtccttgtgcttggcatcttgtccattggggcaaatcaatttacagaagggcttaaccctttaatcgttggttttctcattgtggcgatcggtatttctctggggggcaccaccggctat

SEQ ID NO: 164 - DNA, 504 bp, BACILLUS SP.
gtcatttttggtgcaggtgtttgtgcaggagtcaatttaaagaaatcgctttcatatcaatctggctggattgttgttgtatttggatggggattgggtgtggcaatggccgcttatgcggtcggcggcatcagcggcgcccatcttaatcccgcgttaacgatagcatttgcttttgttggtgattttccgtggaaagaggttccggtttacattgcggcgcaaatgattggagcaataatcggtgcggtgattatctatctgcattacctgccgcattggaagtcaacggatgatcccgctgccaaactgggtgttttctcaaccgggcccagcattccgcatacgtttgcaaacgttttaagtgaagtgatcgggacatttgtccttgtgcttggaatcttggccattggggcaaatgagtttacagagggacttaaccctttaatcgtcggttttctcattgttgctatcggtatttctcttggaggaaccacgggatat

SEQ ID NO: 165 - DNA, 504 bp, BACILLUS SP.
gttatttttggcgcaggtgtttgtgcaggagttaatttaaagaaatcgctttcatatcaatctggctggattgttattgtatttggatggggattgggtgtggcaatggccgcttatgcggtcggcggcatcagcggcgcccatctcaatcccgcgttaacaatagcatttgcttttattggtgattttccgtggaaagaggttccggtatacattgcggcgcagatgatcggagcaatgatcggtgcggtggttgtttaccttcattacctgccgcattggaagtctaccgatgatcccgctgccaagctgggtgttttctcaactggacccagcattccgcatacgtttgcaaacgttttaagtgaggttattgggacatttgtccttgtgcttggaattttggcgattggggcaaatgagtttacagagggacttaaccctttaatcgtcggttttctcattgttgctatcggtatttctcttggaggaactacgggatat

SEQ ID NO: 166 - DNA, 504 bp, BACILLUS SP.
gtcatttttggtgcaggtgtttgtgcaggagtcaatttgaaaaaatcgctttcttatcaatctggctggattgtcattgtgtttggctggggcttgggtgttgcaatggcggcttatgctgttggaggcatcagcggcgcccacctcaatccggcgttaactataggtcttgcgtttgtaggagattttccttgggaacaggttccggcgtatattgccgcacaaatgatcggagcgattatcggcgcggttattgtatttctgcattaccttccgcactggaaggcaactgatgatccggctgcaaagcttggcgttttttccacaggcccgagtataccgcatctattcgctaacgtgttaagtgaagttgttgggacatttgtccttgtgctcggcatattagcgatcggtgcaaatgaatttactcaaggactgaatccgctgattgtcggctttctcattgtggccatcggaatttctcttggaggaacaacaggctac

SEQ ID NO: 167 - DNA, 504 bp, BACILLUS SP.
gtcatttttggtgcaggtgtttgtgcaggagtcaatttgaaaaaatcgctttcttatcaatctggctggattgtcattgtgtttggctggggcttgggtgttgcaatggcggcttatgctgttggaggcatcagcggcgcccacctcaatccggcgttaactataggtcttgcgtttgtaggagattttccttgggaacaggttccggcgtatattgccgcacaaatgatcggagccattatcggcgcggttattgtatttctgcattaccttccgcactggaaggcaactgatgatccggctgcaaagcttggcgttttttccacaggcccgagtataccgcatctattcgctaacgtgttaagtgaagttgttgggacatttgtccttgtgctcggcatattagcgatcggtgcaaatgaatttactcaaggactgaatccgctgattgtcggctttctcattgtggccatcggaatttctcttggaggaacaacaggctac

SEQ ID NO: 168 - DNA, 504 bp, BACILLUS SP.
gtcatttttggtgcaggtgtttgtgcaggagtcaatttgaaaaaatcgctttcttatcaatctggctggattgtcattgtgtttggctggggcttgggtgttgcaatggcggcttatgctgttggaggcatcagcggcgcccatctcaatccggcgttaactataggtcttgcgtttgtaggagattttccttgggaacaggttccggcgtatattgccgcacaaatgatcggagccattatcggcgcggttattgtatttctgcattaccttccgcactggaaggcaactgatgatccggctgcaaagcttggcgttttttccacaggcccgagtatcccgcatctattcgctaacgtgttaagtgaagttgttgggacatttgtccttgtgctcggaatattagcgatcggtgcaaatgaatttactcaaggactgaatccgctgattgtcggctttctcattgtggccatcggaatttctcttggaggaacaacaggctac

SEQ ID NO: 169 - DNA, 504 bp, BACILLUS SP.
gttattttcggcggcggcgtttgtgcgggagtcaatttaaagaaatcgctttcataccaatccggatggattgttattgtattcggctggggattgggagtagccatggcggcatatgccgtcggcagcatcagcggcgcccacttgaatccggctttaacgataggccttgcgcttgaaggcagctttccttggaaagaggtacctgtttatattgcgggacagatgatcggagcaatgatcggtgcggttgttgtgtaccttcattatctgccgcattggaaggcgacggatgatccggccgctaaactgggcgttttctcgaccggcccgagtattcctcatacatttgggaatatgataagtgaagtcattggtacgtttgtgcttgtgctcggcattttggcaatcggagcgaatgaatttacaaaagggctgaacccgctgattgtcggattcctcatcgttgccatcgggatttctcttggagggacgaccggatat

SEQ ID NO: 170 - DNA, 504 bp, BACILLUS SP.
atcgtctttggagctggagtttgtgcaggagttaatttgaaaaaatcgctgtcccatcaatccggatggattgtgatcgtcttcggctgggggcttggcgtggccatggcggtatatgccgtcggcggcatcagcggagcgcatttaaatcccgccgttacattggggctggcatttgtcggagattttccttggaaagaagtgccttcctatattttgggacagatgatcggcgcatttttaggagcggtgctcgtttttcttcactacttgccgcactggaaagaaaccgaagatcaaggcgcgaagctgggagtattttcgacagggccggcgattccaaatacatttgcaaacctgttcagtgaaacattggggacttttgttctcgttctcggacttttagcgatcggtgcaaacaagtttactgacggactgaatccgctcgttgtcggatttctgatcgtggcgatcgggctctcgctcggcggaacaacaggctat

SEQ ID NO: 171 - DNA, 504 bp, BACILLUS SP.
atcgtctttggagctggagtttgtgcaggagttaatttgaaaaaatcgctgtcccatcaatccggatggattgtgatcgtcttcggctgggggcttggcgtggccatggcggtatatgccgtcggcggcatcagcggagcgcatttaaatcctgccgtgacattggggctggcatttgtcggagattttccttggaaagaagtgccttcctatattttaggacagatgatcggcgcatttttaggagcgttgctcgtttttcttcactacttgccgcactggaaagaaaccgaagatcaaggcgcgaagctgggagtattttcgacagggccggcgattccaaatacatttgcaaacctgttcagtgaaacattggggacttttgttctcgttctcggacttttagcgatcggtgcaaacaagtttactgacggactgaatccgctcgttgtcggatttctgatcgtggcgatcgggctttcgctcggcggaacaacaggctat

SEQ ID NO: 172 - DNA, 504 bp, BACILLUS SP.
atcgtctttggagctggagtttgtgcaggagttaatttgaaaaaatcgctgtcccatcaatccggatggattgtgatcgtcttcggctgggggcttggcgtggccatggcggtatatgccgtcggcggcatcagcggagcgcatttaaatccggccgttacattggggctggcatttgtcggagattttccttgggaagaagtgccttcatatattttgggacagatgatcggcgcatttttaggagcggtgctcgtttttcttcactacttgccgcactggaaagaaaccgaggatcaaggcgcgaagcttggagtattttcgacaggtccggcgattccaaatacatttgcaaacctgttcagtgaaacattggggacttttattctcgttctcggacttttaacgatcggtgcaaacaagtttactgacggactgaatcctcttgttgtcggatttctgatcgtggcgatcggtatctcgctcggcggaacaacaggctat

SEQ ID NO: 173 - DNA, 504 bp, BACILLUS SP.
atcgtatttggcgcaggagtttgcgcaggggtaaatttgaaaaaatcgctttcccatcaatcagggtggatcgttatcgcatttggctgggggcttggcgtagccatggccgtatatgccgtcggcaatatcagcggagctcatttaaatcccgctgtcacattgggactggcgtttgtcggcgatttcccttgggagcaagtgccttcttatattttcggacaaatgctcggcgcatttttgggagccgctctcatttttcttcattatttgccgcactggaaggaaacagaagaccaaggagcaaagcttggtgtgttttcgaccgggccggccattccaaatacttttgccaacctgttcagtgaaactttgggaacgttcattcttgtgctcggccttttggcgatcggcgcaaacaaattcacagatggcttgaatccgcttgtcgtcggttttctgatcgtggcaatcggtctgtcgctaggaggaacgaccggttat

SEQ ID NO: 174 - DNA, 504 bp, BACILLUS SP.
atcatttttggggcaggtgtttgtgcaggtgtcaatttaaagaaatcgctttcattccagtctggctggattgttgttgtatttgggtggggattgggtgttgccatgggcgcttatgcagttggcggcatcagcggagcccatttgaatccggcgctaacgatagcgcttgcatttgtgggagattttccatggaaagaggttccagtttatattgcggcgcaaatgatcggagcgatcatcggtgcggtgattatttatctgcattacctgccgcattggaagtcaacggatgatcccgctgccaagctgggtgttttctcaacaggtcccagcattccgcatacatttgcaaacgttttaagtgaagtcattgggacatttgtccttgtgcttggaatcttggccattggggcaaatcaatttacagaagggcttaatcctttaatcgtcggtttcctcattgtagcgatcggtatttctctaggaggcaccaccggctat

SEQ ID NO: 175 - DNA, 504 bp, BACILLUS SP.
atcatttttggggcaggtgtttgtgcgggtgtcaatttaaagaaatcgctttcataccagtctggctggattgttattgtattcggatggggattgggtgttgccatgggggcttatgcggttggcggcatcagcggagctcacctgaatccagcgctaacgatagcgcttgcatttgtcggggattttccgtggaaagaggttccggtttatattatggcgcaaatgatcggagcgatcatcggtgcagtgattatctatctgcattaccttccgcattggaaggcaacggatgatcccgctgccaagctgggtgttttctcaaccggacccagcattccgcacacattttcaaacgttttaagtgaagtgatcgggacatttgtccttgtgcttggcatcttgtccattgggacaaatcaatttacagaagggcttaaccctttaatcgtcggttttctcattgtggcgatcggtatttctctggggggcaccaccggctat

SEQ ID NO: 176 - DNA, 504 bp, BACILLUS SP.
atcatttttggggcaggtgtttgtgcgggtgtcaatttaaagaaatcgctttcataccagtctggctggattgttattgtattcggatggggattgggtgttgccatgggggcttatgcggttggcggcatcagcggagctcacctgaatccagcgctaacgatagcgcttgcatttgtcggggattttccgtggaaagaggttccggtttatattatggcgcaaatgatcggagcgatcatcggtgcagtgattatctatctgcattaccttccgcattggaaggcaacggatgatcccgctgccaagctgggtgttttctcaaccggacccagcattccgcacacattttcaaacgttttaagtgaagtgatcgggacatttgtccttgtgcttggcatcttgtccattggggcaaatcaatttacagaagggcttaaccctttaatcgtcggttttctcattgtggcgatcggtatttctctggggggcaccaccggctat

SEQ ID NO: 177 - DNA, 504 bp, BACILLUS SP.
gttatttttggcgcaggtgtttgtgcaggagtcaatttgaagaaatcgctttcatatcaatctggctggattgttgttgtatttggatggggattaggtgtggcaatggccgcttatgcggtcggcggcatcagcggcgcccatctcaatcccgcgttaacgatagcatttgcttttgttggtgattttccgtggaaagaggttccggtttacattgcggcgcaaatgatcggtgcaatgatcggtgcggtgattgtttaccttcattacctgccgcattggaagtctaccgatgatcccgctgccaagctgggtgttttttcaactggacccagcattccgcatacgtttgcaaacgttttaagtgaggttattgggacatttgtccttgtgcttggaattttggcgattggggcaaatgagtttacagagggacttaaccctttaatcgtcggttttctcatcgttgctatcggtatttctcttggaggaactacgggatat

SEQ ID NO: 178 - DNA, 504 bp, BACILLUS SP.
gttatttttggcgcaggtgtttgtgcaggagttaatttaaagaaatcgctttcatatcaatctggctggattgttattgtatttggatggggattgggtgtggcaatggccgcttatgcggtcggcggcatcagcggcgcccatctcaatcccgcgttaacaatagcatttgcttttattggtgattttccgtggaaagaggttccggtatacattgcggcgcagatgatcggagcaatgatcggtgcggtgattgtttaccttcattacctgccgcattggaagtctaccgatgatcccgctgccaagctgggtgttttctcaaccggacccagcattccgcatacgtttgcaaacgttttaagtgaggttattgggacatttgtccttgtgcttggaattttggcgattggggcaaatgagtttacagagggacttaaccctttaatcgtcggttttctcattgttgctatcggtatttctcttggaggaactacgggatat

SEQ ID NO: 179 - DNA, 504 bp, BACILLUS SP.
attattttcggcggcggcgtttgtgcgggagtcaacttaaagaaatcgctttcataccaatccggatggattgttgttgtatttggctgggggctgggcgtagccatggcggcatatgctgtcggcggcatcagcggcgctcacttgaatccggctttaacgataggccttgcgcttgaaggcagttttccttggaaagacgtccctgtttatattgcgggacagatgatcggagccattatcggtgcggttattgtgtaccttcattatctgccccattggaaggcgacggatgatccggccgctaaactgggcgttttctcaaccggaccgagtattcctcatacatttgggaatatgataagtgaagtcattggtacatttgtgcttgtgctcggcattttggcaatcggagcgaatgaatttacaaaagggctgaacccgctgattgtcggatttctcatcgttgccatcggaatttctcttggaggaacgaccgggtat

SEQ ID NO: 180 - DNA, 504 bp, BACILLUS SP.
attattttcggcggcggcgtttgtgcgggagtcaacttaaagaaatcgctttcataccaatccggatggattgttgttgtatttggctgggggctgggcgtagccatggcggcatatgctgtcggcggcatcagcggcgctcacttgaatccggctttaacgataggccttgcgcttgaaggcagttttccttggaaagacgtccctgtttatattgcgggacagatgatcggagcaattatcggtgcggttattgtgtaccttcattatctgccccattggaaggcgacggatgatccggccgctaaactgggcgttttctcaaccggaccgagtattcctcatacatttgggaatatgattagtgaagtcattggtacatttgtgcttgtgctcggcattttggcaatcggagcgaatgaatttacaaaagggctgaacccgctgattgtcggattcctcatcgttgccatcgggatttctcttggaggaacgaccgggtat

SEQ ID NO: 181 - DNA, 504 bp, BACILLUS SP.
gttatttttggcgcaggtgtttgtgcaggagtcaatttgaagaaatcgctttcatatcaatctggctggattgttgttgtatttggatggggattaggtgtggcaatggccgcttatgcggtcggcggcatcagcggcgcccatctcaatcccgcgttaacgatagcatttgcttttgttggtgattttccgtggaaagaggttccggtttacattgcggcgcaaatgatcggtgcaatgatcggtgcggtgattgtttaccttcattacctgccgcattggaagtctaccgatgatcccgctgccaagctgggtgttttttcaactggacccagcattccgcatacgtttgcaaacgttttaagtgaggttattgggacatttgtccttgtgcttggaattttggcgattggggcaaatgagtttacagagggacttaatcctttaatcgtcggttttctcatcgttgctatcggtatttctcttggaggaaccacgggatat

SEQ ID NO: 182 - DNA, 504 bp, BACILLUS SP.
gttattttcggcggcggcgtttgtgcgggagtcaatttaaagaaatcgctttcataccaatccggatggattgttattgtattcggctggggattgggagtagccatggcggcatatgccgtcggcagcatcagcggcgcccacttgaatccggctttaacgataggccttgcgcttgaaggcagctttccttggaaagaggtccctgtttatattgcaggacagatgatcggagcaatgatcggtgcggttgttgtgttccttcattatctgccgcattggaaggcgacggatgatccggccgctaaactgggcgttttctcgaccggtccgagtattcctcatacatttgggaatatgataagtgaagtcattggtacgtttgtgcttgtgctcggcattttggcaatcggagcgaatgaatttacaaaagggctgaacccgctgattgtcggattcctcatcgttgccatcgggatttctcttggagggacgaccggatat

SEQ ID NO: 183 - DNA, 504 bp, BACILLUS SP.
gttattttcggaggcggcgtttgtgcgggagtcaatttaaagaaatcgctttcataccaatccggatggattgttattgtattcggctggggattgggagtagccatggcggcatatgccgtcggcagcatcagcggcgcccacttgaatccggctttaacgataggccttgcgcttgaaggcagctttccttggaaagaggttcctgtttatattgcgggacagatgatcggagcaatgatcggtgcggttgttgtgttccttcattatctgccgcattggaaggcgacggatgatccggccgctaaactgggcgttttctcgaccggcccgagtattcctcatacatttgggaatatgataagtgaagtcattggtacgtttgtgcttgtgctcggcattttggcaatcggagcgaatgaatttacaaaagggctgaacccgctgattgtcggattcctcatcgttgccatcgggatttctcttggagggacgaccggatat

SEQ ID NO: 184 - DNA, 504 bp, BACILLUS SP.
gttattttcggcggcggcgtttgtgcgggagtcaacttaaagaaatcgctttcataccaatccggatggattgttattgtattcggctggggattgggagtagccatggcggcatatgccgtcggcagcatcagcggcgctcacttgaatccggctttaacgataggccttgcgcttgaaggcagctttccttggaaagacgtccctgtttatattgcgggacagatgatcggagcaatgatcggtgcggttgttgtgtaccttcattatctgccgcattggaaggcgacggatgatccggccgctaaactgggcgttttctcgaccggcccgagtattcctcatacatttgggaatatgataagtgaagtcattggtacgtttgtgcttgtgctcggcattttggcaatcggagcgaatgaatttacaaaagggctgaacccgctgattgtcggattcctcatcgttgccatcgggatttctcttggagggacgaccggatat

SEQ ID NO: 185 - DNA, 504 bp, BACILLUS SP.
atcgtctttggagctggagtttgtgcaggagttaatttgaaaaaatcgctgtcccatcaatccggatggattgtgatcgtcttcggctgggggcttggcgtggccatggctgtatatgccgtcggcggcatcagcggagcgcatttaaatcccgccgttacattagggctggcatttgtcggagattttccttgggaagaagtgccttcctatattttgggacagatgatcggcgcatttttaggagcggtgctcgtttttcttcactacttgccgcactggaaagaaaccgaggatcaaggcgcgaagctgggagtattttcgacaggtccggcgattccaaatacatttgcaaacctgttcagtgaaacattggggacttttattctcgttctcggacttttaacgatcggtgcaaacaagtttactgacggactgaatccgcttgttgtcggatttctgatcgtggcgatcgggatctcgctcggcggaacaacaggctat

SEQ ID NO: 186 - DNA, 504 bp, BACILLUS SP.
atcgtctttggagctggagtttgtgcaggagttaatttgaaaaaatcgctgtcccatcaatccggatggattgtgatcgtcttcggctgggggcttggcgtggccatggctgtatatgccgtcggcggcatcagcggagcgcatttaaatcccgccgttacattggggctggcatttgtcggagattttccttggaaagaagtgccttcctatattttggggcagatgatcggcgcatttttaggggcggtgctcgtttttattcactacttgccgcactggaaagaaaccgaggatcaaggcgcgaagctgggagtattttcgacaggtccggcgattccaaatacatttgcaaacctgttcagtgaaacattggggacttttattctcgttctcggacttttaacgatcggtgcaaacaagtttactgacggactgaatccgcttgttgtcggatttctgatcgtggcgatcgggatctcgctcggcggaacaacaggctat

SEQ ID NO: 187 - DNA, 591 bp, BACILLUS SP.
gtctgtattccgaactgcgacaaaatcacaccgggaatgcttatggcggcaatgcgcatcaacattccgacgatttttgtcagcggcggaccgatggcggcaggaagaacaagtgacgggcgaaaaatctccctttcctcagtattcgaaggggtaggcgcctaccaagcagggaaaatcaacgaaaacgagcttcaagaactagagcagttcggatgcccaacgtgcgggtcttgctcaggcatgtttacggcgaactcaatgaactgtctgtcagaagcacttggtcttgctttgccgggtaatggaaccattctggcaacatctccggaacgcaaagagtttgtgagaaaatcggctgcgcaattaatggaaacgattcgcaaagatatcaaaccgcgtgatattgttacagtaaaagcgattgataacgcgtttgcactcgatatggcgctcggaggttctacaaataccgttcttcatacccttgcccttgcaaacgaagccggcgttgaatactctttagaacgcattaacgaagtcgctgagcgcgtgccgcacttggctaagctggcgcctgcatcggatgtg

SEQ ID NO: 188 - DNA, 591 bp, BACILLUS SP.
gtctgtattccgaactgcgacaaaatcacaccgggaatgcttatggcggcaatgcgcatcaacattccgacgatttttgtcagcggcggaccgatggcggcaggaagaacaagtgacgggcgaaaaatctccctttcctcagtattcgaaggggtaggcgcctaccaagcagggaaaatcaacgaaaacgagcttcaagaactagagcagttcggatgcccaacgtgcgggtcttgctcaggcatgtttacggcgaactcaatgaactgtctgtcagaagcacttggtcttgccttgccgggtaatggaaccattctggcgacatctccggaacgcaaagagtttgtgagaaaatcggctgcgcaattaatggaaacgattcgcaaagatatcaaaccgcgtgatatcgttacagtaaaagcgattgataacgcgtttgcactcgatatggcgctcggaggttctacaaataccgttcttcatacgcttgcccttgcaaacgaagccggcgttgaatactctttagaacgcattaacgaagtcgctgagcgcgtgccgcacttggccaagctggcgcctgcatcggatgtg

SEQ ID NO: 189 - DNA, 591 bp, BACILLUS SP.
gtctgtattccgaactgcgacaaaatcacaccgggaatgcttatggcggcaatgcgcatcaacattccgacgatttttgtcagcggcggaccgatggcggcaggaagaacaagtgacgggcgaaaaatctccctttcctcagtattcgaaggggtaggcgcctaccaagcagggaaaatcaacgaaaacgagcttcaagaactagagcagttcggatgcccaacgtgcgggtcttgctcaggcatgtttacggcgaactcaatgaactgtctgtcagaagcacttggtcttgccttgccgggtaatggaactattctggcgacatctccggaacgcaaagagtttgtgagaaaatcggctgcgcaattaatggaaacgattcgcaaagatatcaaaccgcgtgatatcgttacagtaaaagcgattgataacgcgtttgcactcgatatggcgctcggaggttctacaaataccgttcttcatacgcttgcccttgcaaacgaagccggcgttgaatactctttagaacgcattaacgaagtcgctgagcgcgtgccgcacttggccaagctggctcctgcatcggatgtg

SEQ ID NO: 190 - DNA, 591 bp, BACILLUS SP.
gtctgtattccgaactgcgacaaaatcacgccgggaatgcttatggcggcaatgcgcatcaacattccgacgatttttgtcagcggcgggccgatggcggcaggaagaacaagtgacggacggaaaatctccctttcttcagtattcgaaggggtaggcgcttaccaagcaggaaaaatcaacgaaaacgaacttcaagaattagagcagtttggatgtccaacatgtgggtcttgctctggcatgtttacggcgaattcaatgaactgcctgtccgaagcacttggacttgctttgccgggtaacggaaccattcttgcgacatcaccggaacgcaaagagtttgtcagaaagtcggctgcgcaattaatggaaaccattcgcaaagatatcaaaccgcgtgatattgtcactgtaaaagcgattgataacgcgtttgcactcgatatggcgctcggcggttcaacaaataccgttcttcatacgcttgctcttgcaaacgaggccggcgttgaatactctttagaacgcattaacgaagtcgctgagcgcgtgccgcacttggccaagctggcgcctgcgtctgatgtg

SEQ ID NO: 191 - DNA, 591 bp, BACILLUS SP.
gtctgtattccgaactgcgacaaaatcacgccgggaatgctcatggcggcaatgcgcatcaacattccgacgatttttgtcagcggagggccgatggcggcaggaagaacaagtgacggacggaaaatctccctttcctcagtattcgaaggggtaggcgcttaccaagccggaaaaatcaatgaaaacgaacttcaagaattagagcagttcggatgtccaacatgtgggtcttgctctgggatgtttacggcgaactcaatgaactgcctgtccgaagcacttggacttgctttgccgggtaacgggaccattctcgcgacatcaccggaacgcaaagagtttgtcagaaagtcggctgcgcaattaatggaaaccattcgcaaagatatcaaaccgcgtgatatcgtcactgtaaaagcgattgataacgcgtttgcactcgatatggcgcttggcggttcaacaaataccgttcttcatacgcttgcccttgcaaacgaagccggcgttgaatactctttagaacgcattaacgaagtcgctgagcgcgtgccgcacttggccaagctggctcctgcatctgatgtg

SEQ ID NO: 192 - DNA, 591 bp, BACILLUS SP.
gtctgtattccgaactgtgacaaaatcacgccgggaatgcttatggcggcaatgcgcatcaatattccaacgatttttgtcagcggcgggccgatggcggcaggaagaacaagtgacgggcgcaaaatctccctttcctcagtattcgaaggggtaggcgcttaccaagccggaaaaatcaatgaaaacgagcttcaagaactagagcagttcggatgcccaacgtgcgggtcttgctctggaatgtttacagcaaactcaatgaactgtctgtccgaagcacttggactagctttgccgggtaatggaaccattctcgcgacatcaccggaacgaaaagagtttgtcagaaaatcggctgcgcaattaatggaaacgattcgaaaggatatcaaaccgcgtgatatcgtcacagtaaaagcgattgataacgcctttgcactggatatggcgctcggcggttcaacaaataccgttcttcatacgcttgctctagcaaacgaagccggcgttgaatactctttagaacgcattaacgaagtcgctgagcgtgtgccgcatttggccaagctggctcctgcatcagatgtg

SEQ ID NO: 193 - DNA, 591 bp, BACILLUS SP.
gtctgtattccgaactgtgacaaaatcacgccgggaatgcttatggcggcaatgcgcatcaatattccaacgatttttgtcagcggggggccgatggcggcaggaagaacaagtgacgggcgcaaaatctccctttcctcagtattcgaaggggtaggcgcttaccaagccggaaaaatcaatgaaaacgagcttcaagaactagagcagtttggatgcccaacgtgcgggtcttgctctggaatgtttacagcaaactcaatgaactgtctgtccgaagcacttggactagctttgccgggtaatggaaccattctcgcgacatcaccggaacgaaaagagtttgtcagaaaatcggctgcgcaattaatggaaacaattcgcaaggatatcaaaccgcgtgatatcgtcacagtaaaagcgattgataacgcctttgcactcgatatggcgctcggcggttcaacaaataccgttcttcatacgcttgctctagcaaacgaagccggcgttgaatactctttagaacgcattaacgaagtcgctgagcgcgtgccgcatttggccaagctggctcctgcatcagatgtg

SEQ ID NO: 194 - DNA, 591 bp, BACILLUS SP.
gtttgtattccgaactgtgacaaaatcacgccgggaatgcttatggcagctatgcggatcaacattccaacgatttttgtcagcggagggccgatggcggcaggaagaacaagtgatgggcggaaaatttccctttcctcagtatttgagggggtaggcgcttatcaagcaggaaaaatcaacgagaacgaacttcaagaattagagcagttcggatgcccaacgtgcggatcttgctcaggcatgtttacggcgaactcaatgaattgcctgtctgaagcgcttggtcttgctttgccgggtaacggaaccattctcgcgacagcaccggaacgcaaagagtttgtcagaaaatcggctgcgcaattaatggaaaccattcgcaaagacattaaaccgcgtgatattgtgacagtaaaggcgattgataacgcgtttgcgctcgatatggcgctcggcggttcaacaaataccgttcttcatacgcttgctctcgcaaacgaagccggcgttgactattctttagaacgcattaacgaagtcgctgagcgagtgccgcacttgtccaagctggcgcctgcatcagatgtg

SEQ ID NO: 195 - DNA, 591 bp, BACILLUS SP.
gtctgtattccgaactgtgataaaatcacgccgggaatgcttatggcggctgtgcgcatcaacattccgaccatttttgtcagcgggggaccaatggctgcaggaagaacaagcgacgggcgcaaaatttctctatcttcagtatttgagggagtcggtgcttatcaagccggaaaaatcggcgaaagcgaacttcaggaattagaacagttcggctgcccgacatgcggatcttgctccggaatgtttacagcaaactcaatgaactgtctgtcagaagcacttggattggctttgcctggtaatggaaccattctcgcgacatcgccggaacgaaaagagtttgtcagaaaatcagctaagcaattaatggaaaccattcgcaaagacatcaaaccgagagatatcgtcacagtaaaagcgattgacaatgcgtttgcgcttgacatggcgctcggcggttcaacaaatactgtacttcatacgcttgccctggcaaatgaagctggcgttgaatactctttggaacgcatcaatgaagttgcagagcgcgtgccgcatttgtccaagctggcacctgcatctgatgta

SEQ ID NO: 196 - DNA, 591 bp, BACILLUS SP.
gtctgtatcccgaactgtgacaaaatcacaccgggaatgctaatggccgctatgcgggtcaacattccgactatttttgtcagcggaggtccgatggcggcgggaagaacaagcgacggccgcaaaatttctctctcttctgtatttgaaggtgtcggggcatatcagtccgggaaaatcggtgaaagcgaacttcgggaattagaacagttcggctgtccgacgtgcggatcttgttccggaatgtttacagcaaattcaatgaactgcttatcagaggcgcttggtttggctttgccggggaacggaaccattttggcgacatcacctgcgcgtaaagagtttgtcaagaaatcagccgcgcaattaatggaaaccattcgcaaggatatcaaaccgcgtgacattgtaactgaaaaagcgattgacaatgcgtttgcattggacatggcgcttggcggttcaaccaatacagtgcttcatacgcttgccctcgcaaacgaagcgggagttgagtattctttggaacgcattaatgaagtggctgagagagtgcctcacctaagtaaacttgcaccggcttctgatgtg

SEQ ID NO: 197 - DNA, 591 bp, BACILLUS SP.
gtctgtatcccgaactgtgacaaaatcacaccgggaatgctaatggccgctatgcgggtcaacattccgactatttttgtcagcggaggtccgatggcggcgggaagaacaagcgacggccgcaaaatttctctctcttctgtatttgaaggtgtcggggcatatcagtccgggaaaatcggtgaaagcgaacttcaggaattagaacagttcggctgtccgacgtgcggatcttgttccggaatgtttacagcaaattcaatgaactgcttatcagaggcgcttggcttggctttgccggggaacggaaccattttggcgacatcacctgcgcgtaaagagtttgtcaagaaatcagccacacaattaatggaaaccattcgcaaggatatcaaaccgcgtgacattgtgactgaaaaagcgattgacaatgcgtttgcattggacatggcgcttggcggttcaaccaatacagtgcttcatacgcttgccctcgcaaacgaagcgggagttgagtattctttggaacgcattaatgaagtggctgagagagtgcctcacctaagtaaacttgcaccggcttctgatgtg

SEQ ID NO: 198 - DNA, 591 bp, BACILLUS SP.
gtctgtatcccgaactgtgacaaaatcacaccgggaatgctaatggccgctatgcgggtcaacattccgactatttttgtcagcggaggcccgatggcggcgggaagaacaagcgacggccgcaaaatttctctctcttctgtatttgaaggtgtcggggcatatcagtccgggaaaatcggtgaaagcgaacttcaggaattagaacagttcggctgtccgacgtgcggatcttgttccggaatgtttacagcaaattcaatgaactgcttatcagaggcgcttggcttggctttgccggggaacggaaccattttggcgacatcagctgcgcgtaaagagtttgtcaagaaatcagccgcacaattaatggaaaccattcgcaaggatatcaaaccgcgtgacattgtgactgaaaaagcgattgacaatgcgtttgcattggacatggcgcttggcggttcaaccaatacagtgcttcatacgcttgccctcgcaaacgaagcgggagttgagtattctttggaacgcattaatgaagtggctgagagagtgcctcacctaagtaaacttgcaccggcttctgatgtg

SEQ ID NO: 199 - DNA, 591 bp, BACILLUS SP.
gtctgcattccgaactgcgacaaaattacgccgggaatgctgatggcggctatgcggattaatattccgaccatttttgtcagcggggggccgatggcagccggccgcacaagcgacggccgcaaaatttcgctttcttcagtatttgaaggcgtcggcgcatatcaagccgggaaaatcggtgaaagcgacctgcaggagctggaacagttcggctgcccgacgtgcggatcttgttcgggcatgtttacggcaaactccatgaactgtctgtctgaagcgctcggtctggcgcttccgggcaacggaacaattcttgcaacttcaccggaaagaagagagtttgtgagaaaatcagcggcccaattgatggagaccatcaaaaaagacattaagccgcgcgatatcgtcaccgaaaaagcgattgacaacgcatttgcgttagacatggctctcggcggctcaaccaacacagtgctacatacgcttgcgcttgccaatgaagctggggttgagtactctttagaacgcattaacgaagtggctgaacgagtgcctcacttatcgaaattggcgccggcctcggacgtc

SEQ ID NO: 200 - DNA, 591 bp, BACILLUS SP.
gtatgtattccaaactgcgataaaatcactccgggtatgatcatggcagcaatgcggatcaatattccgaccgtgtttgtcagcggggggccgatggaagcgggaagaacgagcgacggacggaaaatctcgctttcctctgtatttgaaggcgttggcgcttatcaatcaggcaaaatcgatgagaaagggcttgaggagcttgaacagttcggctgtccgacatgcggatcatgctcgggtatgttcacggccaactcgatgaactgcctttctgaagctcttggcatcgccatgccgggcaacggcaccattttggcgacatcgcccgaccgcagggaatttgccaaacagtcggcccgccagctgatggagttgatcaagtcggatattaaaccgcgcgacatcgtgaccgaaaaagcgatcgacaacgcgttcgctttagacatggcgctcggcggatcaacgaatacgatccttcatacgcttgcgatcgccaatgaagcgggggtcgactactcgcttgaacggatcaatgaggtagcggcaagggttccgcatttatcaaagcttgcgccggcttccgatgtg

SEQ ID NO: 201 - DNA, 591 bp, BACILLUS SP.
gtatgcattccaaactgcgataaaatcacgccaggcatgatcatggcagcaatgcggatcaatattccgaccgtgtttgtcagcggggggccgatggaagcgggaagaacgagcgacggacggaaaatctcgctttcctctgtatttgaaggcgttggcgcttatcaatcaggcaaaatcgatgagaaagggcttgaggagcttgaacagttcggctgtccgacatgcggatcatgctcgggtatgttcacggccaactcgatgaactgcctttctgaagctcttggcatcgccatgccgggcaacggcaccattttggcgacatcgcccgaccgcagggaatttgccaaacagtcggcccgccagctgatggagttgatcaagtcggatattaaaccgcgcgacatcgtgaccgaaaaagcgatcgacaacgcgttcgctttagacatggcgctcggcggatcaacgaatacgatccttcatacgcttgcgatcgccaatgaagcgggggtcgactactcgcttgaacggatcaatgaggtagcggcaagggttccgcatttatcaaagcttgcgccggcttccgatgtg

SEQ ID NO: 202 - DNA, 591 bp, BACILLUS SP.
gtatgtattccaaactgtgataaaatcacaccgggcatgatcatggcggcaatgcggatcaacattccgaccgtgtttgtcagcggggggccgatggaagcgggaagaacgagcgacggacgaaaaatctcgctttcctctgtatttgaaggcgttggcgcttatcaatcaggcaaaatcgatgagaaaggactcgaggagcttgaacagttcggctgtccgacttgcggatcatgctcgggcatgtttacggcgaactcgatgaactgtctttctgaagctcttggcatcgccatgccgggcaacggcaccattttggcgacatcgcccgaccgcagggaatttgccaaacagtcggcccgccagctgatggagctgatcaagtcggatatcaaaccgcgcgacatcgtgaccgaaaaagcgatcgacaacgcgttcgctttagacatggcgctcggcggatcaacgaatacgatccttcatacgcttgcgatcgccaatgaagcgggtgtagactattcgcttgaacggatcaatgaggtagcggcaagggttccgcatttatcgaagcttgcaccggcttccgatgtg

SEQ ID NO: 203 - DNA, 591 bp, BACILLUS SP.
gtttgtattccgaactgtgacaaaatcacgccggggatgatcatggcggcaatgaggatcaatattccgaccgtttttgtcagcgggggtccgatggctgcgggaaggacgagtgacgggcgcaaaatctcgctttcctctgtttttgaaggcgtaggcgcctatcaatctggcaaaattgatgaaaaaggattgcaggagcttgaacagtttggctgcccaacctgcggatcatgttccggcatgttcacagccaactcgatgaactgtctatccgaagcgctcggcatcgcgctcccgggcaacggaacgattttggcgacggcgccggaacgaaaggaattcgccaaacaatcggcgcgccagctgatggagctgatcaaatcagatctaaaaccgcgtgacatcgtgacggaaaaggcgattgacaacgcgttcgccttagacatggcgctcggcggatcgacgaatacgatccttcatacactggcaatcgccaatgaagcgggtgttgattattcgcttgagcggattaatgaggttgccgcgagagttcctcatttatcaaagcttgcgcctgcatcagatgtg

SEQ ID NO: 204 - DNA, 591 bp, BACILLUS SP.
gtctgtattccgaactgcgacaaaatcacaccgggaatgcttatggcggcaatgcgcatcaacattccgacgatttttgtcagcggtggaccgatggcggcaggaagaacaagtgacgggcgaaaaatctctctttcctcagtattcgaaggggtaggcgcctaccaagcagggaaaatcaacgaaaacgagcttcaagaactagagcagttcggatgcccaacgtgcgggtcttgctcaggcatgtttacggcgaactcaatgaactgtctgtcagaagcacttggtcttgctttgccgggtaatggaaccattctggcgacatctccggaacgcaaagagtttgtgagaaaatcggctgcgcaattaatggaaacgattcgcaaagatatcaaaccgcgtgatatcgttacagtaaaagcgattgataacgcgtttgcactcgatatggcgctcggaggttctacaaataccgttcttcatacccttgcccttgcaaacgaagccggcgttgaatactctttagaacgcattaacgaagtcgctgagcgcgtgccgcacttggccaagctggcgcctgcatcggatgtg

SEQ ID NO: 205 - DNA, 591 bp, BACILLUS SP.
gtctgtattccgaactgcgacaaaatcacgccgggaatgctcatggcggcaatgcgcatcaacattccgacgatttttgtcagcggagggccgatggcggcaggaagaacaagtgacggacggaaaatctccctttcctcagtattcgaaggggtaggcgcttaccaagccggaaaaatcaatgaaaacgaacttcaagaattagagcagttcggatgtccaacatgtgggtcttgttctgggatgtttacggcgaactcaatgaactgcctgtccgaagcacttggacttgctttgccgggtaacggaaccattctcgcgacatcaccggaacgcaaagagtttgtcagaaagtcggctgcgcaattaatggaaaccattcgcaaagatatcaaaccgcgtgatatcgtcactgtaaaagcgattgataacgcgtttgcactcgatatggcgcttggcggttcaacaaataccgttcttcatacgcttgcccttgcaaacgaagccggcgttgaatactctttagaacgcattaacgaagtcgctgagcgcgtgccgcacttggccaagctggctcctgcatctgatgtg

SEQ ID NO: 206 - DNA, 591 bp, BACILLUS SP.
gtttgtattccgaactgtgacaaaatcacgccgggaatgcttatggcagctatgcggatcaacattccaacgatttttgtcagcggaggaccgatggcggcaggaagaacaagcgatgggcgaaaaatttccctttcctcagtatttgagggggtaggcgcttatcaagcaggaaaaatcaacgagaacgaacttcaagaattagagcagttcggatgcccaacgtgcggatcttgctcaggcatgtttacggcgaactcaatgaattgcctgtctgaagcgcttggccttgctttgccgggtaacggaaccattctcgcgacagcaccggaacgcaaagagtttgtcagaaaatcggctgcgcaattaatggaaaccattcgcaaagacattaaaccgcgtgatattgtgacagtaaaggcgattgataacgcgtttgcgctcgatatggcgctcggcggttcaacaaataccgttcttcatacgcttgctctcgcaaacgaagccggcgttgactattctttagaacgcattaacgaagtcgctgagcgagtgccgcacttgtccaagctggcgcctgcatcagatgtg

SEQ ID NO: 207 - DNA, 591 bp, BACILLUS SP.
gtttgtattccgaactgtgacaaaatcacgccgggaatgcttatggcagctatgcggatcaacattccaacgatttttgtcagcggagggccgatggcggcaggaagaacaagtgatgggcggaaaatttccctttcctcagtatttgagggggtaggcgcttatcaagcaggaaaaatcaacgagaacgaacttcaagaattagagcagttcggatgcccaacgtgcggatcttgctcaggcatgtttacggcgaactcaatgaattgcctgtctgaagcgcttggtcttgctttgccgggtaacggaaccattctcgcaacagcaccggaacgcaaagagtttgtcagaaaatcggctgcgcaattaatggaaaccattcgcaaagacattaaaccgcgtgatattgtgacagtaaaggcgattgataacgcgtttgcgctcgatatggcgctcggcggttcaacaaataccgttcttcatacgcttgctctcgcaaacgaagccggcgttgactattctttagaacgcattaacgaagtcgctgagcgagtgccgcacttgtccaagctggcgcctgcatcagatgtg

SEQ ID NO: 208 - DNA, 591 bp, BACILLUS SP.
gtctgtattccgaactgtgataaaatcacgccgggaatgcttatggcggctatgcgcatcaacattccgaccatttttgtcagcggaggaccgatggcggcaggaagaacaagcgacggccgcaaaatctctctttcttcagtatttgagggagtcggtgcttatcaagctggaaaaatcggcgaaagcgaacttcaagaattagaacagttcggctgcccgacatgcggatcttgctcgggaatgtttacagcaaactcaatgaactgtctgtcagaggcgcttggattggctttgcctggtaatggaaccattctcgcgacatcaccggaacgaaaagagtttgtcagaaaatcagctgcgcaattaatggaaaccattcgcaaagacatcaaaccgagagatatcgttacagtaaaagcgattgataacgcgtttgcgcttgatatggcgctcggcggttcaacaaatactgtacttcatacgcttgcccttgcaaatgaagctggcgttgaatactctttagaacgcatcaatgaagtcgcagagcgcgtgccgcatttggctaagctggcgcctgcatctgatgta

SEQ ID NO: 209 - DNA, 591 bp, BACILLUS SP.
gtctgtattccgaactgtgataaaattacgccgggaatgcttatggcggctgtgcgcatcaacattccgaccatttttgtcagcgggggaccaatggctgcaggaagaacaagcgacgggcgcaaaatttctctatcttcagtatttgagggagtcggtgcttatcaagccggaaaaatcggggaaagcgaacttcaagaattagaacagttcggctgcccgacatgtggatcttgctccggaatgtttacagcaaactcaatgaactgtctgtcagaagcacttggattggctttgcctggtaatggaaccattctcgcgacatccccggatcgaaaagagtttgtcagaaaatcagctaagcaattaatggaaaccattcgcaaagacatcaaaccgagagatatcgtcacggtaaaagcgattgacaatgcgtttgcgcttgacatggcgctcggcggttcaacaaatactgtacttcatacgcttgccctggcaaatgaagctggcgttgaatactctttggaacgcatcaatgaagttgcggagcgcgtgccgcatttgtccaagctggcacctgcatctgatgta

SEQ ID NO: 210 - DNA, 591 bp, BACILLUS SP.
gtctgtattccgaactgtgataaaatcacgccgggaatgcttatggcggctatgcgcatcaacattccgaccatttttgtcagcggaggaccgatggcggcaggaagaacaagcgacggccgcaaaatctctctttcttcagtatttgagggagtcggtgcttatcaagctggaaaaatcggcgaaagcgaacttcaagaattagaacagttcggctgcccgacatgcggatcttgctcgggaatgtttacagcaaactcaatgaactgtctgtcagaagcacttggattggctttgcctggtaatggaaccattctcgcgacatcaccggaacgaaaagagtttgtcagaaaatcagctgcgcaattaatggaaaccattcgcaaagacatcaaaccgagagatatcgttacagtaaaagcgattgataacgcgtttgcgcttgatatggcgctcggcggttcaacaaatactgtacttcatacgcttgcccttgcaaatgaagctggcgttgaatactctttagaacgcatcaatgaagtcgcagagcgcgtgccgcatttggctaagctggcgcctgcatctgatgta

SEQ ID NO: 211 - DNA, 591 bp, BACILLUS SP.
gtctgcattccgaactgcgacaaaattacgccgggaatgctgatggcggctatgcggattaatattccgaccatttttgtcagcggggggccgatggcagccggccgcacaagcgacggccgcaaaatttcgctttcttcagtatttgaaggcgtcggcgcatatcaagccgggaaaatcggtgaaagcgacctgcaggagctggaacagttcggctgcccgacgtgcggatcttgttcgggcatgtttacggcaaactccatgaactgtctgtctgaagcgctcggtctggcgcttccgggcaacggaacaattcttgcaacttcaccggaaagaaaagagtttgtgagaaaatcagcggcccaattgatggagaccatcaaaaaagacattaagccgcgcgatatcgtcaccgaaaaagcgattgacaacgcatttgcgttagacatggctctcggcggctcaaccaacacagtgctacatacgcttgcgcttgccaatgaagctggggttgagtactctttagaacgcattaacgaagtggctgaacgagtgcctcacttatcgaaattggcgccggcctcggacgtc

SEQ ID NO: 212 - DNA, 591 bp, BACILLUS SP.
gtctgcattccgaactgcgacaaaattacgccgggaatgctgatggcggctatgcggattaatattccgaccatttttgtcagcggggggccgatggcagccggccgcacaagcgacggccgcaaaatttcgctttcttcagtatttgaaggcgtcggcgcatatcaagccgggaaaatcggtgaaagcgacctgcaggagctggaacagttcggctgcccgacgtgcggatcttgttcgggcatgtttacggcaaactccatgaactgtctgtctgaagcgctcggtctggcgcttccgggcaacggaacaattcttgcaacttcaccggaaagaagagagtttgtgagaaaatcagcggcccaattgatggagaccatcaaaaaagacattaagccgcgcgatatcgtcaccgaaaaagcgattgacaacgcatttgcgttagacatggctctcggcggctcaaccaacacagtgctacatacgcttgcgcttgccaatgaagctggagttgagtactctttagaacgcattaacgaagtggctgaacgagtgcctcacttatcgaaattggcgccggcctcggacgtc

SEQ ID NO: 213 - DNA, 591 bp, BACILLUS SP.
gtctgcattccgaactgcgacaaaattacgccgggaatgctgatggcggctatgcggattaatattccgaccatttttgtcagcggggggccgatggcagccggccgtacaagcgacggccgcaaaatttcgctttcttcagtatttgaaggcgtcggcgcatatcaagccgggaaaatcggtgaaagcgacctgcaggagctggaacagttcggctgcccgacgtgcggatcttgttcgggcatgtttacggcaaactccatgaactgtctgtctgaagcgctcggtctggcgcttccgggcaacggaacaattcttgcaacttcaccggaaagaagagagtttgtgagaaaatcagcggcccaattgatggagaccatcaaaaaagacattaagccgcgcgatatcgtcaccgaaaaagcgattgacaacgcatttgcgttagacatggctctcggcggctcaaccaacacagtgctacatacgcttgcgcttgccaatgaagctggggttgagtactctttagaacgcattaacgaagtggctgaacgagtgcctcacttatcgaaattggcgccggcctcggacgtc

SEQ ID NO: 214 - DNA, 591 bp, BACILLUS SP.
gtctgcattccgaactgcgacaaaattacgccgggaatgctgatggcggctatgcggattaatattccgaccatttttgtcagcggggggccgatggcagccggccgcacaagcgacggccgcaaaatttcgctttcttcagtatttgaaggcgtcggcgcatatcaagccgggaaaatcggtgaaagcgacctgcaggagctggaacagttcggctgcccgacgtgcggatcttgttcgggcatgtttacggcaaactccatgaactgtctgtctgaagcgctcggcctggcgcttccgggcaacggaacaattctagcaacttcaccggagagaagagagtttgtgagaaaatcagcggcccaattaatggagaccatcaaaaaagacattaagccgcgcgatatcgtcaccgaaaaagcgattgacaacgcatttgcgttagacatggctctcggcggctcaaccaacacagtgcttcatacgcttgcgcttgccaatgaagctggggttgagtactctttagaacgcattaacgaagtggctgaacgagtgcctcacatatcgaaattggcgccggcctcggacgtc

SEQ ID NO: 215 - DNA, 591 bp, BACILLUS SP.
gtttgtattccgaactgtgacaaaatcacgccggggatgattatggcggcaatgaggatcaatattccgaccgtttttgtcagcgggggtccgatggctgcgggaaggacgagtgacgggcgcaaaatctcgctttcctctgtttttgaaggcgtaggcgcctatcaatctggcaaaattgatgaaaaaggattgcaggagcttgaacagtttggctgcccaacctgcggatcatgttccggcatgttcacagccaactcgatgaactgtctatccgaagcgctcggcatcgcgctcccgggcaacggaacgattttggcgacggcgccggaacgaaaggaattcgccaaacaatcggcgcgccagctgatggagctgatcaaatcagatctaaaaccgcgtgacatcgtgacggaaaaggcgattgacaacgcgttcgccttagacatggcgctcggcggatcgacgaatacgatccttcatacactggcaatcgccaatgaagcgggtgttgattattcgcttgagcggattaatgaggttgccgcgagagttcctcatttatcaaagcttgcgcctgcatcagatgtg

SEQ ID NO: 216 - DNA, 591 bp, BACILLUS SP.
gtttgtattccgaactgtgacaaaatcacgccggggatgatcatggcggcaatgaggatcaatattccgaccgtttttgtcagcgggggtccgatggctgcgggaaggacgagtgacgagcgcaaaatctcgctttcctctgtttttgaaggcgtaggcgcctatcaatctggcaaaattgatgaaaaaggattgcaggagcttgaacagtttggctgcccaacctgcggatcatgttccggcatgttcacagccaactcgatgaactgtctatccgaagcgctcggcatcgcgctcccgggcaacggaacgattttggcgacggcgccggaacgaaaggaattcgccaaacaatcggcgcgccagctgatggagctgatcaaatcagatctaaaaccgcgtgacatcgtgacggaaaaggcgattgacaacgcgttcgccttagacatggcgctcggcggatcgacgaatacgatccttcatacactggcaatcgccaatgaagcgggtgttgattattcgcttgagcggattaatgaggttgccgcgagagttcctcatttatcaaagcttgcgcctgcatcagatgtg

SEQ ID NO: 217 - DNA, 591 bp, BACILLUS SP.
gtatgtattccaaactgtgataaaatcacaccgggcatgatcatggcggcaatgcggatcaacattccaaccgtgtttgtcagcgggggaccgatggaagcgggaagaacgagcgacggacgaaaaatctcgctttcctctgtatttgaaggcgttggcgcttatcaatccggcaaaatcgatgagaaaggactcgaggagcttgaacagttcggctgtccgacttgcggatcatgctcgggcatgttcacggccaactcgatgaactgtctttctgaagctcttggcatcgcaatgccgggcaacggcaccattttggcgacatcgcccgaccgcagggaatttgccaaacagtcggcccgccagctgatggagctgatcaaggcggatatcaaaccgcgcgacatcgtgaccgaaaaagcgatcgacaacgctttcgctttagacatggcgctcggcggatcaacgaatacgatccttcatacgcttgcgatcgccaatgaagcgggtgtagagtattcgcttgaacggatcaatgaggtagcggcaagggttccgcatttatcaaagcttgcaccggcttccgatgtg

SEQ ID NO: 218 - DNA, 591 bp, BACILLUS SP.
gtatgtattccaaactgtgataaaatcacaccgggcatgattatggcggcaatgcggatcaacattcccaccgtgtttgtcagcggaggaccgatggaagcgggaagaacgagcgacggacgaaaaatctcgctttcctctgtatttgaaggcgttggcgcttatcaatccggcaaaatcgatgagaaagggctcgaggagcttgaacagttcggctgtccgacttgcggatcatgctcgggtatgttcacggccaactcgatgaactgtctttctgaagctcttggcatcgccatgccgggcaacggcaccattttggcgacatcacccgaccgcagggaatttgccaaacagtcggcccgccagctgatggagctgatcaaggcggatatcaaaccgcgcgacatcgtgaccgaaaaagcgatcgacaacgctttcgcgttagacatggcgctcggcggatcaacgaatacgatccttcatacgcttgcgatcgccaatgaagcgggtgtagactattcgcttgaacggatcaatgaggtagcagcaagggttccgcatttatcaaagcttgcaccggcttccgatgtg

SEQ ID NO: 219 - DNA, 534 bp, BACILLUS SP.
caggctcgtaaagcattattagacgagaactacttcggtacaatgctggtgtataaaggacttgcagacggacttgtaagcggagctgctcactcaactgctgacactgtccgcccggctcttcaaatcatcaaaacaaaagaaggcgtgaaaaagacttcaggcgtgttcatcatggctcgcggagaagagcaatacgtattcgcagattgcgcgatcaacattgcacctgacagccaagatcttgccgagattgcgatcgaaagtgccaatacggcaaaaatgttcgacattgagcctcgcgtggcaatgctcagcttctctacaaaaggctcagcaaaatctgatgaaacagaaaaagtagcggatgcagtgaaaatcgcgaaagaaaaagcgcctgaactgacacttgacggcgaattccaatttgatgctgcatttgttccatctgtagctgagaaaaaagcgccggattccgagatcaaaggggacgctaacgtattcgtattcccaagtcttgaagcaggaaacatc

SEQ ID NO: 220 - DNA, 534 bp, BACILLUS SP.
caggctcgcaaagcgttattagacgagaactacttcggtacaatgctggtgtataaaggacttgcagacggacttgtaagcggagctgctcactcaactgctgacactgtccgcccggctcttcaaatcatcaaaacaaaagaaggcgtgaaaaagacttccggcgtgttcatcatggctcgcggagaagagcaatacgtattcgcagattgcgcgatcaacattgcgcctgacagccaagatcttgccgagattgcgatcgaaagtgccaatacggcaaaaatgttcgacattgagcctcgcgtggcaatgctcagcttctctacaaaaggctcagcaaaatctgatgaaacagaaaaagtagcggatgcagtgaaaatcgcgaaagaaaaagcgcctgaactgacacttgacggcgaattccaatttgatgctgcatttgttccatctgtagctgagaaaaaagcgccggattccgagatcaaaggggacgctaacgtattcgtattcccaagccttgaagcaggaaacatc

SEQ ID NO: 221 - DNA, 534 bp, BACILLUS SP.
caggctcgcaaagcgttattagacgagaactacttcggtacaatgctggtgtataaaggacttgcagacggacttgtaagcggagctgctcactcaactgctgacactgtccgcccggctcttcaaatcatcaaaacaaaagaaggcgtgaaaaagacttccggcgtgttcatcatggctcgcggagaagagcaatacgtattcgcagattgcgcgatcaacattgcgcctgacagccaagatcttgccgagattgcgatcgaaagtgccaatacggcaaaaatgttcgacattgagcctcgcgtggcaatgctcagcttctctacaaaaggctcagcaaaatctgatgaaacagaaaaagtagcggatgcagtgaaaatcgcgaaagaaaaagcgcctgaactgacacttgacggcgaattccaatttgatgctgcatttgttccatctgtagctgagaaaaaagcgccggattccgaaatcaaaggggacgctaacgtattcgtattcccaagccttgaagcaggaaacatc

SEQ ID NO: 222 - DNA, 534 bp, BACILLUS SP.
caggctcgcaaagcgttattagacgagaactacttcggtacaatgctggtgtacaaaggccttgcagacggactcgtaagcggagctgctcactcgacagctgacactgtccgcccggctcttcaaatcatcaaaacaaaagaaggcgtgaaaaagacttcaggcgtgttcatcatggctcgcggagaagagcaatacgtattcgcagattgcgcgatcaacattgcgcctgacagccaagatcttgccgagattgcgatcgaaagtgccaatacggcaaaaatgttcgacattgagcctcgcgtggcaatgctcagcttctctacaaaaggctcagcaaaatctgatgaaacagaaaaagtagcggatgcagtgaaaatcgcgaaagaaaaagcgcctgaactgacacttgacggcgaattccaatttgatgctgcatttgttccatctgtagctgagaaaaaagcgccggattccgagatcaaaggggacgctaacgtattcgtattcccaagccttgaagcaggaaacatc

SEQ ID NO: 223 - DNA, 534 bp, BACILLUS SP.
caggcccgcaaagcgttattagacgagaactacttcggtacaatgctggtgtataaaggccttgcagatggacttgtaagcggagctgctcactcgacagctgacactgtccgcccggctcttcaaatcatcaaaacaaaagaaggcgtgaaaaagacttcaggcgtgttcatcatggctcgcggagaagagcaatacgtattcgcagattgcgcgatcaacattgcgcctgacagccaagaccttgccgagattgcgatcgaaagtgccaatacggcaaaaatgttcgacattgagcctcgcgtggcaatgctcagcttctctacaaaaggctcagcaaaatctgatgaaacagaaaaagtagcggatgcagtgaaaatcgcgaaagaaaaagcgcctgaactgacacttgacggcgaattccaatttgatgctgcatttgttccatctgtagctgagaaaaaagcgccggattccgagatcaaaggggacgctaacgtattcgtattcccaagtcttgaagcaggaaacatc

SEQ ID NO: 224 - DNA, 534 bp, BACILLUS SP.
caggctcgaaaagcgttattagatgagaactacttcggtacaatgctggtgtacaaaggccttgcacacggacttgtaagcggagcggctcattcaacagccgacacagtccgcccggctcttcaaatcatcaaaacaaaagaaggcgtgaaaaagacttccggcgtgttcatcatggctcgtggagaagagcaatatgtattcgcggattgcgcgatcaacattgcgcctgatagccaagatcttgccgagattgcgatcgaaagtgccaatacggcaaaaatgttcgacattgagcctcgcgtggcaatgctcagcttctctacaaagggctcagcaaaatctgatgaaacagaaaaagttgcggatgcggtgaaaattgcgaaagaaaaagcacctgaactgacacttgacggcgaattccaattcgatgctgcatttgttccatctgtagctgagaaaaaagcgccggattccgaaatcaaaggggatgctaacgtattcgtattcccaagccttgaagcaggaaacatc

SEQ ID NO: 225 - DNA, 534 bp, BACILLUS SP.
caggctcgtaaagcgttattagatgagaactacttcggtactatgctggtgtacaaaggccttgcacacggccttgtgagcggagcggctcattcaacagccgatactgtccgccctgctcttcaaatcattaaaacaaaagaaggcgtgaaaaagacttccggcgttttcatcatggctcgcggagaagagcaatatgtattcgccgattgcgcgatcaacattgcgcctgacagccaagatcttgccgagattgcgatcgaaagtgccaatacagcaaaaatgttcgacattgagcctcgcgtggcaatgctcagcttctcgacaaaaggctcagcaaaatctgatgaaacagaaaaagtagctgatgctgtgaaaatcgccaaagaaaaagcgcctgaactgacacttgacggcgaattccaattcgatgctgcatttgttccatctgtagctgagaaaaaagcgccggattccgatatcaaaggggatgctaacgtattcgtattcccaagccttgaagcaggcaacatc

SEQ ID NO: 226 - DNA, 534 bp, BACILLUS SP.
caggctcgtaaagcattattagatgagaactacttcggtacaatgctggtgtacaaaggccttgcacaaggacttgtaagcggagcggctcattcaacagccgacactgtccgcccggctcttcaaatcatcaaaacaaaagaaggcgtgaaaaagacttccggcgtcttcatcatggctcgcggagaagagcaatacgtattcgcagattgcgcgatcaacattgcgcctgacagccaagatcttgccgagattgcgatcgaaagtgccaatacggcaaaaatgttcgacattgagcctcgcgtggcaatgctcagcttctctacaaaaggctcagcaaaatctgatgaaacagaaaaagtagcggatgcggtgaaaatcgcgaaagaaaaagcgcctgaactgacacttgacggcgaattccaattcgatgctgcatttgttccatctgtagctgagaaaaaagcgccggattccgagatcaaaggggatgctaacgtattcgtattcccaagccttgaagcaggaaacatc

SEQ ID NO: 227 - DNA, 534 bp, BACILLUS SP.
caggctcgtaaagcattattagacgagaactacttcggtacaatgctggtgtacaaaggccttgcagacggacttgtaagcggagcggctcattcaacagccgacactgtccgcccggctcttcaaatcatcaaaacaaaagaaggcgtgaaaaagacttccggcgtcttcatcatggctcgcggagaagagcaatacgtgttcgcagattgcgcgatcaacattgcgcctgacagccaagatcttgccgagatcgcgatcgaaagtgccaatacggcaaaaatgttcgacattgagcctcgcgtggcaatgctcagcttctctacaaaaggctcagcaaaatctgacgaaacagaaaaagtagcggatgctgtgaaaatcgcgaaagaaaaagcgcctgaactgacacttgacggagaattccaattcgatgctgcatttgttccatctgtagctgagaaaaaagcgccggattccgagatcaaaggggacgctaacgtattcgtattcccaagccttgaagcaggaaacatc

SEQ ID NO: 228 - DNA, 534 bp, BACILLUS SP.
caagctcgtaaagcattattagacgagaactacttcggtacaatgctggtgtacaaaggccttgcagacggacttgtaagcggagcggctcattcaacagccgacactgtccgcccggctcttcaaatcatcaaaacaaaagaaggcgtgaaaaagacttctggcgtcttcatcatggctcgcggagaagagcaatacgtgttcgcagattgcgcgatcaacattgcgcctgacagccaagatcttgccgagatcgcgatcgaaagtgccaatacggcaaaaatgttcgacattgagcctcgcgtggcaatgctcagcttctctacaaaaggctcagcaaaatctgacgaaacagaaaaagtagcggatgctgtgaaaatcgcgaaagaaaaagcgcctgaactgacacttgacggcgaattccaattcgatgctgcatttgttccatctgtagctgagaaaaaagcgccagattccgagatcaaaggggacgctaacgtattcgtattcccaagccttgaagcaggaaacatc

SEQ ID NO: 229 - DNA, 534 bp, BACILLUS SP.
caggctcgaaaagcgctcttagacgagaactacttcggcacaatgcttgtgtacaaaggcctggctgccggtcttgtgagcggagcggctcattcaactgccgacactgtccgtccggctcttcaaatcatcaaaacaaaagaaggcgtgaaaaagacttccggcgtcttcatcatggcacgcggcgatgagcagtacgtattcgcggattgcgcgatcaacattgcgcctgacagccaagatcttgcggaaatcgcgatcgaaagcgccaatacggcaaaaatgttcgacatcgatcctcgcgtagcaatgctcagcttctctacaaaaggctcagcaaaatctgatgaaacagaaaaagtagcggatgctgtgaaaatcgcgaaagaaaaagcgcctgaattaacgcttgacggcgaattccaatttgatgctgcattcgttccatctgtagctgagaaaaaagcgccggactccgagatcaaaggagacgcgaacgtattcgtattcccaagcttggaagcaggaaacatc

SEQ ID NO: 230 - DNA, 534 bp, BACILLUS SP.
caagctcgtaaagctcttttagacgaaaactacttcggcacgatgcttgtgtacaaaggtcttgcgcacgggcttgtcagcggagcggctcattcaacagcagacacagtgcgccctgctcttcaaattatcaaaacaaaagaaggcgtgaaaaaaacctccggcgtattcatcatggcgcgcggcgacgaacaatatgtatttgcggattgcgcgatcaacattgcacctgacagccaggaccttgctgaaattgcaatcgaaagcgccaatacggctaaaatgttcaacatcgagcctcgggtagcaatgctaagcttctctacaaaaggctcagcgaagtcagatgagactgaaaaagtagcagaagcggtgaaaatcgcaaaagaaaaagcgcctgagcttactcttgacggcgaattccaatttgacgcggcatttgtgccttctgtcgctgagaaaaaagcgccggattccgaaatcaaaggtgatgcgaacgtattcgtattcccaagccttgaagcaggaaacatc

SEQ ID NO: 231 - DNA, 534 bp, BACILLUS SP.
caagctcgtaaagctcttttagacgaaaactacttcggcacgatgcttgtgtacaaaggtcttgcgcacgggcttgtcagcggagcggctcattcaacagcagacacagtgcgccctgctcttcaaattatcaaaacaaaagaaggcgtgaaaaaaacctccggtgtattcatcatggcgcgcggcgacgaacaatatgtatttgcggattgcgcgatcaacattgcacctgacagccaggaccttgctgaaattgcaatcgaaagcgccaatacggctaaaatgtttaacatcgagcctcgggtagcaatgctaagcttctctacaaaaggctcagcgaagtcagatgagactgaaaaagtagcagaagcggtgaaaatcgcaaaagaaaaagcgcctgagcttactcttgacggcgaattccaatttgacgcggcatttgtgccttctgtcgctgagaaaaaagcgccggattccgaaatcaaaggtgatgcgaacgtattcgtattcccaagccttgaagcaggaaacatc

SEQ ID NO: 232 - DNA, 534 bp, BACILLUS SP.
caagctcgtaaagctcttttagacgaaaactacttcggcacgatgcttgtgtacaaaggtcttgcgcacgggcttgtcagcggagcggctcattcaacagcagacacagtgcgccctgctcttcaaattatcaaaacaaaagaaggcgtgaaaaaaacctccggcgtattcatcatggcgcgcggcgacgaacaatatgtatttgcggattgcgcgatcaacattgcacctgacagccaggaccttgctgaaattgcaatcgaaagcgccaatacggctaaaatgttcaacatcgaacctcgggtagcaatgctaagcttctctacaaaaggctcagcgaagtcagatgagactgaaaaagtagcagaagcggtgaaaatcgcaaaagaaaaagcgcctgagcttactcttgacggcgaattccaatttgacgcggcatttgtgccttctgtcgctgagaaaaaagcgccggattccgaaatcaaaggtgatgcgaacgtattcgtattcccaagccttgaagcaggaaacatc

SEQ ID NO: 233 - DNA, 534 bp, BACILLUS SP.
caggcccgcaaagctttgctggacgaaaactatttcggcacgatgcttgtctacaaaggtctcgcagacggacttgtcagcggagctgcacattctactgctgatacggtgcgccctgcactgcaaatcatcaaaacaaaagaaggcgtgaaaaaaacatcaggcgtcttcatcatggcccgcggtgacgagcagtatgtgttcgctgattgcgcgatcaacatttctcctgacagccaggaccttgccgaaatcgccattgaaagcgccaatacggctcaaatgtttgacattgacccgcgcgttgccatgctcagcttctctacaaaaggatcggcgaaatcagacgagacggacaaagtggctgaagccgtgaaaattgcgaaggaaaaagcgcctgagcttacgcttgacggcgaattccaattcgacgctgcatttgtgccgtctgtcgcggagaaaaaagcgccggactccgacattaaaggagacgcgaatgtatttgtattcccaagccttgaagcaggaaacatc

SEQ ID NO: 234 - DNA, 534 bp, BACILLUS SP.
caggcccgccaaatccttttagatgagaactattttggaacgatgcttgtttataaagggcttgccgacggtcttgtcagcggtgcggctcattcaacagcggatacggttcgccctgcgctgcaaattattaaaacaaaagaaggcgtgaagaaaacttccggcgtgtttatcatggcgcgcggcgatgagcagtatgtattcgcagactgtgcgatcaatattgcgcctgacagtcaggaccttgcggaaattgcaatcgaaagtgcgaacacagcgaaaatgttcaacattgaccctcgtgtcgccatgctcagcttttcaacaaaaggctctgcaaaatcagacgaaactgaaaaggtagccaatgcggttgcgatcgccaaagaaaaagcgcctgagcttacactggacggcgaattccaatttgatgcggcattcgttccgtctgtcgccgagaaaaaagcgccggactccgtcattaaaggtgatgcaaacgtatttgttttcccaagccttgaagcggggaacatc

SEQ ID NO: 235 - DNA, 534 bp, BACILLUS SP.
caggcccgccaaatccttttagatgagaactattttggaacgatgcttgtttataaagggcttgccgacggtcttgtcagcggtgcggctcattcaacagcggatacggttcgccctgcgctgcaaattattaaaacaaaagaaggcgtgaagaaaacttccggcgtgtttatcatggcgcgcggcgatgagcagtatgtatttgcagactgtgcgatcaatattgcgcctgacagtcaggaccttgcggaaattgcaatcgaaagtgcgaacacagcgaaaatgttcaacattgaccctcgtgtcgccatgctcagcttttcaacaaaaggctctgcaaaatcagacgaaactgaaaaggtagccaatgcggttgcgatcgccaaagaaaaagcgcctgagcttacactggacggcgaattccaatttgatgcggcattcgttccatctgtcgccgagaaaaaagcgccggactccgtcattaaaggtgatgcaaacgtatttgttttcccaagccttgaagcggggaacatc

SEQ ID NO: 236 - DNA, 534 bp, BACILLUS SP.
caggcccgccaaatccttttagacgaaaactactttggaacgatgcttgtttataaagggcttgccgacggccttgtcagcggtgcggctcattcaacagcggatacggttcgcccggcgctgcaaattattaaaacaaaagaaggcgtgaagaaaacatcaggcgtctttatcatggcgcgcggcgatgagcagtacgtattcgcagactgtgcgatcaacattgcgcctgacagccaggaccttgcggaaatcgcaatcgaaagtgcgaacacagcgaaaatgttcgacatcgaacctcgtgtcgccatgctcagcttttcaacaaaaggttcggcaaaatcagacgaaactgaaaaagtagccaacgcggttgcaatcgcaaaagaaaaagcgcctgagcttacacttgacggcgaattccaatttgacgcggcattcgttccgtctgtcgctgagaaaaaagcgccggactccgtcattaaaggagatgcaaacgtatttgttttcccaagccttgaagcggggaatatc

SEQ ID NO: 237 - DNA, 534 bp, BACILLUS SP.
caggcccgccaaatcctgttggatgaaaactattttggcacaatgcttgtttacaaagggcttgcggacggccttgtcagcggtgcggctcattcaaccgcagacacggttcgcccggcgctgcaaattattaagacaaaagaaggcgtaaaaaaaacatcaggcgtcttcattatggcccgcggcgatgaacagtacgtgttcgctgactgtgcaatcaacattgcgcccgacagccaggacctggcggaaatcgcagtcgaaagcgccaatacggcaaaaatgttcgacatcgagccgcgagtggcgatgctcagcttctcaacaaaaggatctgcaaaatcagatgaaaccgaaaaagtggcaaatgccgttgcaatcgctaaagaaaaagcgcctgagcttacgcttgacggcgaattccaatttgatgccgcatttgttccatctgtcgctgagaaaaaagcgcctggctccgtgattaaaggcgatgcaaacgtatttgttttcccaagccttgaagcggggaacatc

SEQ ID NO: 238 - DNA, 534 bp, BACILLUS SP.
caggctcgcaaagcgttattagacgagaactacttcggtacaatgctggtgtataaaggccttgcagacggactcgtaagcggagctgctcactcgacagctgacactgtccgcccggctcttcaaatcatcaaaacaaaagaaggcgtgaaaaagacttcaggcgtgttcatcatggctcgcggagaagagcaatacgtattcgcagattgcgcgatcaacattgcgcctgacagccaagaccttgccgagattgcgatcgaaagtgccaatacggcaaaaatgttcgacattgagcctcgcgtggcaatgctcagcttctctacaaaaggctcagcaaaatctgatgaaacagaaaaagtagcggatgcagtgaaaatcgcgaaagaaaaagcgcctgaactgacacttgacggcgaattccaatttgatgctgcatttgttccatctgtagctgagaaaaaagcgccggattccgagatcaaaggggacgctaacgtattcgtattcccaagccttgaagcagggaacatc

SEQ ID NO: 239 - DNA, 534 bp, BACILLUS SP.
caggctcgaaaagcgttattagatgagaactacttcggtacaatgctggtgtacaaaggccttgcacacggacttgtaagcggagcggctcattcaacagccgatacagtccgcccggctcttcaaatcatcaaaacaaaagaaggcgtgaaaaagacttccggcgtgttcatcatggctcgtggagaagagcaatatgtattcgcggattgcgcgatcaacattgcgcctgatagccaagatcttgccgagattgcgatcgaaagtgccaatacggcaaaaatgttcgacattgagcctcgcgtggcaatgctcagcttctctacaaagggctcagcaaaatctgatgaaacagaaaaagttgcggatgcggtgaaaattgcgaaagaaaaagcacctgaactgacacttgacggcgaattccaattcgatgctgcatttgttccatctgtagctgagaaaaaagcgccggattccgaaatcaaaggggatgctaacgtattcgtattcccaagccttgaagcaggaaacatc

SEQ ID NO: 240 - DNA, 534 bp, BACILLUS SP.
caggctcgaaaagcgctcttagacgagaactacttcggcacaatgcttgtgtacaaaggccttgcagcgggtcttgtgagcggagcggctcattcaactgccgacactgtccgtccggctcttcaaatcatcaaaacaaaagaaggcgtgaaaaagacttcaggcgtcttcatcatggcacgcggcgatgagcagtacgtattcgcagattgcgcgatcaacattgcaccagacagccaagatcttgcagaaatcgcaatcgaaagcgccaatacggcaaaaatgttcgacattgatcctcgcgtagcaatgctcagcttctctacaaaaggctcagcaaaatctgatgaaacagaaaaagtagcggatgctgtgaaaatcgcgaaagaaaaagcgcctgaattaacgcttgacggcgaattccaatttgacgctgcattcgttccatctgtagctgagaaaaaagcgccggattccgagatcaaaggagacgcgaacgtattcgtattcccaagcttggaagcaggaaacatc

SEQ ID NO: 241 - DNA, 534 bp, BACILLUS SP.
caggctcgaaaagcgctcttagacgagaactacttcggcacaatgcttgtgtacaaaggcctggctgccggtcttgtgagcggagcggctcattcaactgccgacactgtccgtccggctcttcaaatcatcaaaacaaaagaaggcgtgaaaaagacttccggcgtcttcatcatggcacgcggtgatgagcagtacgtattcgcggattgcgcgatcaacattgcaccagacagccaagatcttgcggaaatcgcgatcgaaagcgccaatacggcaaaaatgttcgacatcgatcctcgcgtagcaatgctcagcttctctacaaaaggctcagcaaaatctgatgaaacagaaaaagtagcggatgctgtgaaaatcgcgaaagaaaaagcgcctgaattaacgcttgacggcgaattccaatttgatgctgcattcgttccatctgtagctgagaaaaaagcgccggactccgagatcaaaggagacgcgaacgtattcgtattcccaagcttggaagcaggaaacatc

SEQ ID NO: 242 - DNA, 534 bp, BACILLUS SP.
caggcccgcaaagccttattggacgaaaactatttcggcacaatgcttgtgtacaaaggtctcgcggacggacttgtcagcggagctgcacattctactgccgatacggttcgacctgcactgcaaatcattaaaacaaaagaaggcgtgaaaaaaacatctggtgtcttcatcatggctcgcggtgacgagcaatatgtattcgctgattgcgcgatcaacattgctcctgacagccaggaccttgccgaaatcgccattgaaagcgccaatacggctcaaatgtttgatattgacccgcgcgttgccatgctcagcttctctacaaaaggatcggcgaaatcagacgagacggaaaaagtggccgaagcggtgaaaattgcgaaggaaaaagcgcctgaacttacgcttgacggcgaattccaatttgatgctgcatttgtgccgtctgttgcggagaagaaagcgccggactccgaaattaaaggagacgcgaatgtatttgtattcccaagccttgaagcaggaaacatc

SEQ ID NO: 243 - DNA, 534 bp, BACILLUS SP.
caggcccgcaaagccttattggacgaaaactatttcggcacaatgcttgtgtacaaaggtctcgcggacggacttgtcagcggagctgcacattctactgccgatacggtgcgccctgcactgcaaatcatcaaaacaaaagaaggcgtgaaaaaaacatcaggcgtcttcatcatggctcgcggtgacgagcaatatgtattcgctgattgtgcgatcaacattgctcctgacagccaggaccttgccgaaatcgccattgaaagcgccaatacggctcaaatgtttgatattgacccgcgcgttgctatgctcagcttctctacaaaaggatcggcgaaatcagacgagacggaaaaagtggccgaagcagtgaaaattgcgaaggaaaaagcgcctgaacttacgcttgacggcgaattccaatttgacgctgcatttgtgccgtctgttgcggagaagaaagcgccggactccgaaattaaaggagacgcgaatgtatttgtattcccaagccttgaagcaggaaacatc

SEQ ID NO: 244 - DNA, 534 bp, BACILLUS SP.
caggcccgcaaagccttattggacgaaaactatttcggcacaatgcttgtgtacaaaggtctcgcggacggacttgtcagcggagctgcacattctactgccgatacggttcgccctgcactgcaaatcattaaaacaaaagaaggcgtgaaaaaaacatcaggcgtcttcatcatggctcgcggtgacgagcaatatgtattcgctgattgcgcgatcaacattgctcctgacagccaggaccttgccgaaatcgccattgaaagcgccaatacggctcaaatgtttgatattgacccgcgcgttgccatgctcagcttctctacaaaaggatcggcgaaatcagacgagacggaaaaagtggccgaagcggtgaaaattgcgaaggaaaaagcgcctgaacttacgcttgacggcgaattccaatttgacgctgcatttgtgccgtctgttgcggagaagaaagcgccggactccgaaattaaaggagacgcgaatgtatttgtattcccaagccttgaagcaggaaacatc

SEQ ID NO: 245 - DNA, 534 bp, BACILLUS SP.
caggcccgcaaagctttgctggacgaaaactatttcggcacgatgcttgtgtacaaaggtctcgcagacggacttgtcagcggagctgcacattctactgctgatacggtgcgccctgcactgcaaatcatcaaaacaaaagaaggcgtgaaaaaaacatcaggcgtcttcatcatggcccgcggtgacgagcagtatgtgttcgctgattgcgcgatcaacattgctcctgacagtcaggaccttgccgaaatcgccattgaaagcgccaatacggctcaaatgtttgacattgacccgcgcgttgccatgctcagcttctctacaaaaggatcggcgaaatcagacgagacggacaaagtggctgaagcggtgaaaattgcgaaggaaaaagcgcctgagcttacgcttgacggcgaattccaattcgacgctgcatttgtgccgtctgtcgcggagaaaaaagcgccggactccgacattaaaggagacgcgaatgtatttgtattcccaagccttgaagcaggaaacatc

SEQ ID NO: 246 - DNA, 534 bp, BACILLUS SP.
caggcccgcaaagctttgctggacgaaaactatttcggcacgatgcttgtctacaaaggtctcgcagacggacttgtcagcggagctgcacattctactgctgatacggtgcgccctgcactgcaaatcatcaaaacaaaagaaggcgtgaaaaaaacatcaggcgtcttcatcatggcccgcggtgacgagcagtatgtgttcgctgattgcgcgatcaacatttctcctgacagccaggaccttgccgaaatcgccattgaaagcgccaatacggctcaaatgtttgacattgacccgcgcgttgccatgctcagcttctctacaaaaggatcggcgaaatcagacgagacggacaaagtggctgaagccgtgaaaattgcgaaggaaaaagcgcctgagcttacgcttgacggagaattccaattcgacgctgcatttgtgccgtctgtcgcggagaaaaaagcgccggactccgacattaaaggagacgcgaatgtatttgtattcccaagccttgaagcaggaaacatc

SEQ ID NO: 247 - DNA, 534 bp, BACILLUS SP.
caggcccgcaaagctttgctggacgaaaactatttcggcacgatgcttgtgtacaaaggtctcgcagacggacttgtcagcggagctgcacattctactgctgatacggtgcgccctgcactgcaaatcatcaaaacaaaagaaggcgtgaaaaaaacatcaggcgtcttcatcatggcccgcggtgacgagcagtatgtgttcgctgattgcgcgatcaacattgctcctgacagccaggaccttgccgaaatcgccattgaaagcgccaatacggctcaaatgtttgacattgacccgcgcgttgccatgctcagcttctctacaaaaggatcggcgaaatcagacgagacggacaaagtggctgaagccgtgaaaattgcgaaggaaaaagcgcctgagcttacgcttgacggcgaattccaattcgacgctgcatttgtgccgtctgtcgcggagaaaaaagcgccggactccgacattaaaggagacgcgaatgtatttgtattcccaagccttgaagcaggaaacatc

SEQ ID NO: 248 - DNA, 534 bp, BACILLUS SP.
caggcccgcaaagctttgctggacgaaaactatttcggcacgatgcttgtgtacaaaggtctcgcagacggacttgtcagcggagctgcacattctacagccgatacggtgcgccctgcacttcaaatcattaaaacaaaagaaggcgtgaaaaaaacatcaggcgtcttcatcatggcccgcggtgacgagcagtatgtgttcgctgattgcgcgatcaacattgctcctgacagccaggaccttgccgaaatcgccattgaaagcgccaatacggctcaaatgtttgacattgacccgcgcgttgccatgctcagcttctctacaaaaggatcggcgaaatcagacgagacggacaaagtggctgaagcggtgaaaattgcgaaggaaaaagcgcctgagcttacgcttgacggcgaattccaattcgacgctgcatttgtgccgtctgtcgcggagaaaaaagcgccggactccgacattaaaggagacgcgaatgtatttgtattcccaagccttgaagcaggaaacatc

SEQ ID NO: 249 - DNA, 534 bp, BACILLUS SP.
caggcccgccaaatccttttagacgaaaactactttggaacgatgcttgtttataaagggcttgccgacggccttgtcagcggtgcggctcattcaacagcggatacggttcgcccggcgctgcaaattattaaaacaaaagaaggcgtgaagaaaacgtcaggcgtctttatcatggcgcgcggcgatgagcagtacgtattcgcagactgtgcgatcaacattgcgcctgacagccaggaccttgcggaaatcgcaatcgaaagtgcgaacacagcgaaaatgttcgacatcgaacctcgtgtcgccatgctcagcttttcaacaaaaggttcggcaaaatcagacgaaactgaaaaagtagccaacgcggttgcaatcgcaaaagaaaaagcgcctgagcttacacttgacggcgaattccaatttgacgcggcattcgttccgtctgtcgctgagaaaaaagcgccggactccgtcattaaaggagatgcaaacgtatttgttttcccaagccttgaagcggggaatatc

SEQ ID NO: 250 - DNA, 534 bp, BACILLUS SP.
caggctcgaaaagcgctcttagacgagaactacttcggcacaatgcttgtgtacaaaggccttgcagcgggtcttgtgagcggagcggctcattcaaccgccgacactgtccgcccggctcttcaaatcatcaaaacaaaagaaggcgtgaaaaagacttcaggcgtcttcatcatggcacgcggcgatgagcagtacgtattcgcagattgcgcgattaacattgcaccagacagccaagatcttgcagaaatcgcaatcgaaagcgccaatacggcaaaaatgttcgacattgatcctcgcgtagcaatgctcagcttctctacaaaaggctcagcaaaatctgatgaaacagaaaaagtagcggatgctgtgaaaatcgcaaaagaaaaagcgcctgaattaacgcttgacggcgaattccaatttgacgctgcattcgttccatctgtagctgagaaaaaagcgccggattccgagatcaaaggagacgcgaacgtattcgtattcccaagcttggaagcaggaaacatt

SEQ ID NO: 251 - DNA, 534 bp, BACILLUS SP.
caggcccgcaaagcgttattagacgagaactacttcggtacaatgctggtgtataaaggccttgcagatggacttgtaagcggagctgctcactcgacagctgacactgtccgcccggctcttcaaatcatcaaaacaaaagaaggcgtgaaaaagacttcaggcgtgttcatcatggctcgcggagaagagcaatacgtattcgcagattgcgcgatcaacattgcgcctgacagccaagaccttgccgagattgcgatcgaaagtgccaatacggcaaaaatgttcgacattgagcctcgcgtggcaatgctcagcttctctacaaaaggctcagcaaaatctgatgaaacagaaaaagtagcggatgcagtgaaaatcgcgaaagaaaaagcgcctgaactgacacttgacggcgaattccaatttgatgctgcatttgttccatctgtagctgagaaaaaagcgccggattccgagatcaaaggggacgctaacgtattcgtattcccaagccttgaagcaggaaacatc

SEQ ID NO: 252 - DNA, 534 bp, BACILLUS SP.
caggcccgccaaatcctgttggatgaaaactattttggcacaatgcttgtttacaaagggcttgcggacggccttgtcagcggtgcggctcattcaaccgcagacacggttcgcccggcgctgcaaattattaagacaaaagaaggcgtaaaaaaaacatcaggtgtcttcattatggcccgcggcgatgaacagtacgtgttcgctgactgtgcaatcaacattgcgcccgacagccaggacctggcggaaatcgcagtcgaaagcgccaatacggcaaaaatgttcgacatcgagccgcgagtggcgatgctcagcttctcaacaaaaggatctgcaaaatcagatgaaaccgaaaaagtggcaaatgccgttgcaatcgctaaagaaaaagcgcctgagcttacgcttgacggcgaattccaatttgatgccgcatttgttccatctgtcgctgagaaaaaagcgcctggctccgtgattaaaggcgatgcaaacgtatttgttttcccaagccttgaagcggggaacatc

SEQ ID NO: 253 - DNA, 534 bp, BACILLUS SP.
caggcccgccaaatccttttagacgaaaactactttggaacgatgcttgtttataaagggcttgccgacggtcttgtcagcggtgcggctcattcaacagcagatacggttcgcccggcgctccaaattattaaaacaaaagaaggcgtgaagaaaacatctggcgtctttatcatggcgcgcggcgatgagcagtacgtattcgcagattgtgcgatcaacattgcgcctgacagtcaggaccttgcggaaatcgcaatcgaaagtgcgaacacagcgaaaatgttcaacattgaccctcgtgtcgctatgctcagcttttcaacgaaaggctcggcaaaatcagatgaaactgaaaaggtagccaatgcggttgcgatcgccaaagaaaaagcgcctgagcttacactggacggcgaattccaatttgatgcggcattcgttccgtctgtcgctgagaaaaaagcgccggactccgtcattaaaggtgatgcaaacgtatttgttttcccaagccttgaagcggggaacatc

SEQ ID NO: 254 - DNA, 534 bp, BACILLUS SP.
caggcccgacaaatccttttagacgaaaattactttggaacgatgcttgtttataaaggacttgccgacggtcttgtcagcggtgcggctcattcaacagcggatacggttcgcccggcgctgcaaattattaaaacaaaagaaggcgtgaagaaaacatcgggcgtctttatcatggcgcgtggcgatgagcagtatgtattcgcagactgtgcgatcaacattgcacctgacagtcaggaccttgcggaaatcgcaatcgaaagtgcgaacacagcgaaaatgttcaacattgaccctcgtgtcgccatgctcagcttttcgacaaaaggctcggcaaaatcagatgaaactgaaaaggtagccaatgcggttgcgattgccaaagaaaaagctcctgagcttacactggacggcgaattccaatttgacgcggcattcgttccgtctgtcgctgagaaaaaagcgccggactccgtcattaaaggtgatgcaaacgtatttgttttcccaagccttgaagcggggaacatc

SEQ ID NO: 255 - DNA, 519 bp, BACILLUS SP.
ggagaaaacccgcatcaggaagcaactttctatcaaacagctcttcctgtcaaaggctccattgcgcaagcagaacagcttcacggaaaagagctttcttacaacaacattaaagacgcggatgctgcagttcaaatcgttcgtgaattcactgaaccggctgctgttgctgtgaagcatatgaacccgtgcggcgtgggaacaggaaaaacgatcgcagaagcgtttgacagagcgtttgaagcggataaaacatctatcttcggcggcattatcgcgctgaaccgtgaagtggacaaggcaactgccgaagcgcttcacaacattttcttagaaatcatcattgcgccttcattcagccaagaagcgctcgacgtcctgactgcgaagaaaaatctccgtctgctgacgcttgacgtatccgccgctgttcaaaaggaaaaacagctgacatccgttcaaggcgggctgctgattcaagatttagatatgcacggcttcgatgatgctgagattagc

SEQ ID NO: 256 - DNA, 519 bp, BACILLUS SP.
ggagaaaacccgcatcaggaagcaactttctatcaaacagctcttcctgtcaaaggctccattgcgcaagcagaacagcttcacggaaaagagctttcttacaacaacattaaagacgcggatgctgcagttcaaatcgttcgtgaattcactgaaccggctgctgttgctgtgaagcatatgaacccgtgcggcgtgggaacaggaaaaacgatcgcagaagcgtttgacagagcgtttgaagcggataaaacatctatcttcggcggcattatcgcgctgaaccgtgaagtggacaaggcaactgccgaagcgcttcacaacattttcttagaaatcatcattgcgccttcattcagccaagaagcgctcgacgtcctgactgcgaagaaaaatctccgtctgctgacgcttgacgtatccgccgctgttaaaaaggaaaaacagctgacatccgttcaaggcgggctgctgattcaagatttagatatgcacggcttcgatgatgctgagattagc

SEQ ID NO: 257 - DNA, 519 bp, BACILLUS SP.
ggagaaaacccgcatcaggaagcaactttctatcaaacagctcttcctgtcaaaggctccattgcgcaagcagaacagcttcacggaaaagagctttcttacaacaacattaaagacgcggatgctgcagttcaaatcgttcgtgaattcactgaaccggctgctgttgctgtgaagcatatgaacccgtgcggcgtgggaacaggaaaaacgatcgcagaagcgtttgacagagcgtttgaagcggataaaacatctatcttcggcggcattatcgcgctgaaccgtgaagtggacaaggcaactgccgaagcgcttcacaacattttcttagaaattatcattgcgccttcattcagccaagaagcgctcgacgtcctgactgcgaagaaaaatctccgtctgctgacgcttgacgtatccgtcgctgttcaaaaggaaaaacagttgacatctgttcaaggcgggctgctgattcaagatttagatatgcacggcttcgatgatgctgagattagc

SEQ ID NO: 258 - DNA, 519 bp, BACILLUS SP.
ggagaaaacccgcatcaggaagcaactttctatcaaacagctcttcctgtcaaaggctccattgcgcaagcagaacagcttcacggaaaagagctttcttacaacaacattaaagacacggatgcggcagttcaaatcgttcgtgaattcactgaaccggctgctgttgctgtgaagcacatgaacccgtgcggtgtaggaacaggaaaaacgatcgcagaagcgtttgacagagcgtttgaagcggataaaacatctatcttcggcggcattatcgcgctgaaccgtgaagtggacaaggcaactgccgaagcgcttcacaacattttcttagaaatcatcattgcgccttcattcagccaagaagcgctcgacgtcctgactgcgaagaaaaatctccgtctgctgacgcttgacgtatccgccgctgttcaaaaggaaaaacaactgacatctgttcaaggcgggctgctgattcaagatttagatatgcacggcttcgatgatgctgagattagc

SEQ ID NO: 259 - DNA, 519 bp, BACILLUS SP.
ggagaaaacccgcatcaggaagcaactttctatcaaacagctcttcctgtcaaaggctccattgcgcaagcagaacagcttcacggaaaagagctttcttacaacaacatgaaagacgcggatgcggcagttcaaatcgttcgtgaattcactgaaccggctgctgttgctgtgaagcacatgaacccgtgcggtgtaggaacaggaaaaacgatcgcagaagcgtttgacagagcgtttgaagcggataaaacatctatcttcggcggcattatcgcgctgaaccgtgaagtggacaaggcaactgccgaagcgcttcacaacattttcttagaaatcatcattgcgccttcattcagccaagaagcgctcgacgtcctgactgcgaaaaaaaatctccgtctgctgacgcttgacgtatccgccgctgttcaaaaggaaaaacaactgacatctgttcaaggcgggctgctgattcaagatttagatatgcacggctttgatgatgctgagattagc

SEQ ID NO: 260 - DNA, 519 bp, BACILLUS SP.
ggagaaaacccgcatcaggaagcaactttctatcaaacagctcttcctgtcaaaggctccattgcgcaagcagaacagcttcacggaaaagagctttcttacaacaacatgaaagacgcggatgcggcagttcaaatcgttcgtgaattcactgagccggctgcggttgccgtgaagcatatgaacccgtgcggcgtgggaacaggaaaaacgatcgccgaagcgtttgacagagcgtttgaagcggataaaacatctatcttcggcggcattatcgcgttgaaccgtgaagtggacaagacaactgccgaagcgcttcataacattttcttagaaatcatcattgcgccttcattcagccaagaagcgctcgacgtcctgactgcgaagaaaaatctccgcctgctgacgcttgacgtatctgctgctgttcaaaaggaaaaacagctgacatccgttcaaggcgggctattgattcaagatttagatatgcacggctttgatgatgctgagattagc

SEQ ID NO: 261 - DNA, 519 bp, BACILLUS SP.
ggggaaaacccgcatcaggaagcaacattctatcaaacagcacttcctgttaaaggctccattgcgcaagcggaacagcttcacggcaaagagctttcttacaacaacattaaagacgcagatgcagcagttcaaatcgtccgtgaattcactgagccggctgcggttgccgtgaagcatatgaatccgtgcggcgttggaacaggaaaaacgatttcagaagccttcgacagagcgtttgaagcggataaaacatctatcttcggcggcattatcgctcttaaccgtgaagtggacaaggcaactgccgaagcgcttcacaacattttcttagaaattatcattgcgccttcattcagccaagaagcacttcacatcctgactgcgaagaaaaatcttcgtctgatgacgcttgatgtgacagctaccgttcaaaaagaaaaacagctgacatccgttcaaggcggactgctgattcaagatttagatatgcacggcttcgatgatgccgagatcagc

SEQ ID NO: 262 - DNA, 519 bp, BACILLUS SP.
ggagaaaacccgcatcaggaagcaacattctatcaaacagcacttcctgttaaaggctccattgcgcaagcggaacagctccacggcaaagagctttcttataacaacattaaagatgcggatgcggcagttcaaatcgtccgtgaattcactgagcctgctgcggttgccgtgaagcatatgaatccgtgcggcgttggaacaggggaaacaatctcagaagcgtttgacagagcgtttgaagcggataaaacatctatcttcggcggcattatcgcgctgaaccgtgaagtggacaaggcaactgccgaagcgctccacaacattttcttagaaatcatcattgcgccttcattcagccaagaagcgcttgatatcctgactgcgaagaaaaatcttcgtctgctgacacttgatgtgacagctgccgttcaaaaagaaaaacagttgacatccgttcaaggcggtctgctgattcaagatttagatatgcatggcttcgatgatgctgagatcagc

SEQ ID NO: 263 - DNA, 519 bp, BACILLUS SP.
ggagagaacccgcatcaggaagcaacattctaccaaagcgcgcttcctgttaaaggatctattgcgcaagcagaacagctccacggaaaagagctttcttacaacaacattaaagacgcggatgcagcagttcaaatcgttcgtgaattcactgaaccggctgcggttgccgtaaagcatatgaatccttgcggcgtaggaacaggaaaaacgatcgcagaagcgtttgacagagcgtttgaagcagataaaacatctatcttcggcggcattatcgcgctgaaccgtgaagtggacaaagcaactgccgaagtgcttcacaacattttcttagaaatcatcattgcgccttcattcagtcaagaagcgcttgacatcctgactgcgaagaaaaatcttcgtctgctgacgcttgatgtgacagctgccgttcaaaaagaaaaacagctgacatccgttcaaggcgggctgctgattcaagatttagatatgcacggttttgatgatgctgagatcagc

SEQ ID NO: 264 - DNA, 519 bp, BACILLUS SP.
ggagagaacccgcatcaggaagcaacattctaccaaagcgcgcttcctgttaaaggatctattgcgcaagcagaacagctccacggaaaagagctttcttacaacaacattaaagacgcggatgcagcagttcaaatcgttcgtgaattcactgagccggctgcggttgccgtaaagcatatgaatccttgcggcgtaggaacaggaaaaacgatcgcagaagcgtttgacagagcatttgaagcggataaaacatctatcttcggcggcattatcgcgctgaaccgtgaagtggacaaagcaactgcagaggcgcttcacaacattttcttagaaatcatcattgcgccttcattcagccaagaagcgcttgacatcctgactgcgaagaaaaatcttcgtctgctgacgcttgatgtgacagctgccgttcaaaaagaaaaacagctgacatccgttcaaggcgggctgctgattcaagatttagatatgcacggctttgatgatgctgagatcagc

SEQ ID NO: 265 - DNA, 519 bp, BACILLUS SP.
ggagaaaacccgcatcaggaagcgacattctatcaaacagctcttcctgtcaaaggctccattgcgcaagcagaacagcttcacggcaaagagctttcttacaacaacattaaagacgctgatgcagctgttcaaatcgtccgtgaattcactgagccggctgcggttgctgtgaagcatatgaatccgtgcggcgtcggaacaggaaaaacgatcgtagaagcgtttgacagagcgtttgaagccgatcaaacatcgatcttcggcggcattatcgcgctgaaccgtgaagtggacaaagcaaccgcagaggcgcttcacaacattttcttagaaatcatcattgcgccttcattcagccaagaagcacttgacatcttgactgcgaagaaaaatcttcgtctgctgacgcttgatgtgacagctgccgttcaaaaagaaaaacaactgacatctgttcaaggcgggctgctgattcaagatttagatatgcacggatttgatgatgctgagatcagc

SEQ ID NO: 266 - DNA, 519 bp, BACILLUS SP.
ggagaaaatccgcatcaggaagcaacattctatcaaagcgcgcttcctgtcaaaggttctattgcacaagcagaacagctgcacggcaaagagctttcttacaacaacattaaagatgcggacgcagctgttcagattgttcgtgaactcacggagcctgctgcggttgctgtgaagcatatgaatccatgcggcgtgggaactggcgaaacgatcgcagaagcgtttgacagagcgttcgaagcagataaaacatcgattttcggcggtattatcgcgttgaaccgtgaagtggacaagacaacagccgaagcgcttcataacattttcttagaaatcatcattgcgccttcattcagccaagaagcgcttgatatcctgacagcgaagaaaaatcttcgtctgctgacacttgatgtgacagctgccgtccaaaaagaaaaacagctgacatctgtacaaggcggattgctgattcaagatttagatatgcacggctttaatgatgctgagatcagc

SEQ ID NO: 267 - DNA, 519 bp, BACILLUS SP.
ggtgaaaaccctcaccaagaggctgttttctatcaaagcgcacttcccgtctccggttccatcgcggcggcaaaacagcttcacggcaaagagctttcttacaacaatattaaggacgcagatgcggccgttcaaatcgtccgggaatttacagaacccgcagctgttgccgttaaacatatgaacccgtgcggagtcggtacgggagcttcaattgaggaagcattcaataaagcgtatgaagctgataaaacctccattttcggcggcatcatcgcgctgaaccgtgaagttgatcaggcaacggctgaagcccttcacggcatctttttagaaatcattatcgccccttctttcagtgaagaagcgctgaacgtactgacggcgaagaaaaaccttcgtctgctcacgcttgaagtgtccgcatcagggaagaaagaaaaacagctgacatccgtacagggcggtcttttgattcaagatttagacgtccatggatttgatgacgccgacatcagc

SEQ ID NO: 268 - DNA, 519 bp, BACILLUS SP.
ggagaaaatccgcatcaagaagcgacgttttatcaaagcgcactgccggcaagcggttcaatcgcatcagctgaacagctgcacggcaaagagctttcttataacaacattaaagacgccgatgcggcggtccaaattatacgtgagtttacagagccggcggcagttgcggtcaagcatatgaacccatgcggcgtcggaacaggcaaaacgattgcagaagcgtttaacagagcgtttgcggcagatgaaacgtctattttcggcggcattatcgcgcttaaccgtgaagtggacaaggcaacggcggaagtgctgcataagatctttttagaaatcattattgcgccttcattcagtgaagaagcacttgaggtgttaacgtccaagaaaaacctgcgcctcttaacattagatgtgtcagcttctattaaaaaagagaaacagcttacttccgttcaaggcggactattgtttcaagatttagacatgcacggctttgatgacgcaaaaatcagc

SEQ ID NO: 269 - DNA, 519 bp, BACILLUS SP.
ggagaaaatccgcatcaagaagcgacgttttatcaaagcgcactgccggcaagcggttcaatcgcatcagctgaacagctgcacggcaaagagctttcttataacaacattaaagacgccgatgcggcggtccaaattatacgtgagtttacagagccggcggcagttgcggtcaagcatatgaacccatgcggcgtcggaacaggcaaaacgattgcagaagcgtttaacagagcgtttgcggcagatgaaacgtctattttcggcggcattatcgcgcttaaccgtgaagtggacaaggcaacggcagaagtgctgcataagatctttttagaaatcattattgcgccttcattcagtgcggaagcacttgaggtgttaacgtccaagaaaaacctgcgcctcttaacattagatgtgtcagcttctattaaaaaagagaaacaacttacttccgttcaaggcggactattgattcaagatttagacatgcacggctttgatgacgcaaaaatcagc

SEQ ID NO: 270 - DNA, 519 bp, BACILLUS SP.
ggagaaaatccgcatcaagaagcgacgttttatcaaagcgcactgccggcaagcggttcaatcgcatcagctgaacagctgcacggcaaagagctttcttataacaacattaaagacgccgatgcggcggtccaaattatacgtgagtttacagagccggcggcagttgcggtcaagcatatgaacccatgcggcgttggaacaggcaaaacgattgcagaagcgtttaacagagcgtttgcggcagatgaaacgtctattttcggcggcattatcgcgcttaaccgtgaagtggacaaggcaacggcggaagtgctgcataagatctttttagaaatcattattgcgccttcattctgtgcggaagcacttgaggtgttaacgtccaagaaaaacctgcgcctcttaacattagatgtgtcagcttctattaaaaaagagaaacagcttacttccgttcaaggcggactattgattcaagatttagacatgcacggctttgatgacgcaaaaatcagc

SEQ ID NO: 271 - DNA, 519 bp, BACILLUS SP.
ggagaaaatccgcatcaagaagcgacgttttatcaaagcgcactgccggcaagcggttcaatcgcatcagctgaacagctgcacggcaaagagctttcttataacaacattaaagacgccgatgcggcggtccaaattatacgtgagtttacagagccggcggcagttgcggtcaagcatatgaacccatgcggcgtcggaacaggcaaaacgattgcagaagcgtttaacagagcgtttgcggcagatgaaacgtctattttcggcggcattatcgcgcttaaccgtaaagtggacaaggcaacggcggaagtgctgcataagatctttttagaaatcattattgcgccttcattcagtgcggaagcacttgaggtgttaacgtccaagaaaaacctgcgcctcttaacattagatgtgtcagcttctattaaaaaagagaaacagcttacttccgttcaaggcggactattgattcaagatttagacatgcacggctttgatgacgcaaaaatcagc

SEQ ID NO: 272 - DNA, 519 bp, BACILLUS SP.
ggcgaaaatccgcatcaggaagcagtcttttatcaaagtgcgctccctgtcaaagggtcaatcgcttcagctgaacagctgcacggaaaagagctctcttacaacaatatcaaagacgcggatgcagcacttcaaatcgtccgcgagttcacggagccggctgcggttgccgtcaagcatatgaacccttgcggcgtcgggacgggcaacacgatcgaagaggcgtttaacaaagcatacgccgctgacgaaacgtcaattttcggcggcatcatcgctttgaaccgcgaagtggataaagcgacggctgaaacgcttcacaaaatctttttggaaatcatcatcgcgccagcattcagccaagaagcgcttgatattttaacatccaagaaaaaccttcgcctcttgacccttgatgtcaatcagcctgtgaaaaacgaaaagcagctgacatctgttcaaggcgggcttttgattcaggatctggatatgcacggttttgacgacgccgacatcaca

SEQ ID NO: 273 - DNA, 519 bp, BACILLUS SP.
ggcgaaaatccgcatcaggaagcagtcttttatcaaagtgcgctccctgtcaaagggtcaatcgcttcagctgaacagctgcacggaaaagagctctcttacaacaatatcaaagacgcggatgcagcacttcaaatcgtccgcgagttcacggagccggctgcggttgccgtcaagcatatgaacccttgcggcgtcgggacgggcaacacgatcgaagaggcgtttaacaaagcatacgccgctgacgaaacgtcaattttcggcggcatcatcgctttgaaccgggaagtggataaagcgacggctgaaacgcttcacaaaatctttttggaaatcatcatcgcgccagcattcagccaagaagcgcttgatattttaacatccaagaaaaaccttcgcctcttgacccttgatgtcaatcagcctgtgaaaaacgaaaagcagctgacatctgttcaaggcgggcttttgattcaggatctggatatgcacggttttgacgacgccgacatcaca

SEQ ID NO: 274 - DNA, 519 bp, BACILLUS SP.
ggcgaaaatccgcatcaggaagcagtcttttatcaaagcgcgctccctgtcaaagggtcgatcgcttcagctgaacagctgcacggaaaagagctttcctacaacaacatcaaagacgcggatgcggcacttcaaattgtccgcgagttcaccgagccggctgcggttgccgtcaagcatatgaacccttgcggcgtcgggacgggcaacacgatcgaagaggcgtttaacaaagcatacgccgctgacgaaacatcgattttcggcggcatcatcgccctgaaccgcgaagtggacaaagcgactgctgaaacgcttcacaaaatctttttggaaatcattatcgcgccggcattcagccaagaagcgcttgatattttaacatccaagaaaaaccttcgcctcttgaccctcgatgtcaatcagcctgtgaaaaacgaaaaacagctgacatctgttcaaggcgggctcttgattcaggatctggatatgcacggctttgacgacgctgacattacg

SEQ ID NO: 275 - DNA, 519 bp, BACILLUS SP.
ggggaaaacccgcatcaggaagccgtgttttatcaaagcgccatccctgtgagcggttcgatcgcttcagcgaatcagcttcacggaaaagagctttcctacaataacattaaagatgcggatgccgcagtccaaatcgtccgtgaattcacagagccggcggcggttgcggtcaagcatatgaacccgtgcggcgtcggcacaggcaaaacgattgcggaagcgttcaacaaagcctacgcggctgatcaaacatcgattttcggcggaatcatcgcccttaaccgtgaagtggatcaagcaactgccgaagtccttcatcaaatctttttggaaatcatcatcgcaccttcattcagcacagaagcgcttgagattttaacatccaagaaaaatctccgtctgttgacgcttgatgtcaatcagcctgtcaaaaacgaaaagcagctgacatccgttcaaggcggactcttgattcaggatctggatatgcacggctttgacgacgctgacattacg

SEQ ID NO: 276 - DNA, 519 bp, BACILLUS SP.
ggggaaaacccgcatcaggaagccgtgttttatcaaagcgccatccctgtgagcggttcgatcgcttcagcgaatcagcttcacggaaaagagctttcctacaataacattaaagatgcggatgccgcagtccaaatcgtccgtgaattcacagagccggcggcggttgcggtcaagcatatgaacccgtgcggcgtcggcacaggcaaaacgattgcggaagcgttcaacaaagcctacgcggctgatcaaacatcgattttcggcggaatcatcgcccttaaccgtgaagtggatcaagcaactgccgaagtccttcatcaaatctttttggaaatcatcatcgcaccttcattcagcacagaagcgcttgagattttaacatccaagaaaaatctccgtctgttgacgcttgatgtcaatcagcctgtcaaaaacgaaaagcagctgacatccgttcaaggcggactcttgattcaggatctggatatgcacggctttgatgatgccgacatcacg

SEQ ID NO: 277 - DNA, 519 bp, BACILLUS SP.
ggagaaaacccgcatcaggaagcaactttctatcaaacagctcttcctgtcaaaggctccattgcgcaagcagaacagcttcacggaaaagagctttcttacaacaacattaaagacgcggatgcggcagttcaaatcgttcgtgaattcactgaaccgactgctgttgctgtgaagcacatgaacccgtgcggtgtaggaacaggaaaaacgatcgcagaagcgtttgacagagcgtttgaagcggataaaacatctatcttcggcggcattatcgcgctgaaccgtgaagtggacaaggcaactgccgaagcgcttcacaacattttcttagaaatcatcattgcgccttcattcagccaagaagcgctcgacgtcctgactgcgaaaaaaaatctccgtctgctgacgcttgacgtatccgccgctgttcaaaaggaaaaacaactgacatctgttcaaggcgggctgctgattcaagatttagatatgcacggctttgatgatgctgagattagc

SEQ ID NO: 278 - DNA, 519 bp, BACILLUS SP.
ggagaaaacccgcatcaggaagcaactttctatcaaacagctcttcctgtcaaaggctccattgcgcaagcagaacagcttcacggaaaagagctttcttacaacaacattaaagacgcggatgcggcagttcaaatcgttcgtgaattcactgaaccggctgcggttgccgtgaagcatatgaacccgtgcggcgtgggaacaggaaaaacgatcgccgaagcgtttgacagagcgtttgaagcggataaaacatctatcttcggcggcattatcgcgttgaaccgtgaagtggacaagacaactgccgaagcgcttcataacattttcttagaaatcatcattgcgccttcattcagccaagaagcgctcgacgtcctgactgcgaagaaaaatctccgcctgctgacgcttgacgtatctgctgttgttcaaaaggaaaaacagctgacatccgttcaaggcgggctgctgattcaagatttagatatgcacggcttcgatgatgctgagattagc

SEQ ID NO: 279 - DNA, 519 bp, BACILLUS SP.
ggagaaaacccgcatcaggaagcaacattctatcaaacagcacttcctgttaaaggctccattgcgcaagcggagcagctccacggcaaagagctttcttacaacaacattaaagacgcggatgcggcagttcaaatcgtccgtgaattcactgagcctgctgcggttgccgtgaagcatatgaatccgtgcggcgttggaacaggggaaacaatctcagaagcgtttgacagagcgtttgaagcggataaaacatctatcttcggcggcattatcgcgctgaaccgtgaagtggacaaggcaactgccgaagcactccacaacattttcttagaaatcatcattgcgccttcattcagccaagaagcgcttgatatcctgactgcgaagaaaaatcttcgtctgctgacacttgatgtgacagctgccgttcaaaaagaaaaacagttgacatccgttcaaggcggcctgctgattcaagatttagatatgcatggcttcgatgatgctgagatcagc

SEQ ID NO: 280 - DNA, 519 bp, BACILLUS SP.
ggagaaaacccgcatcaggaagcgacattttatcaaacagctcttcctgtcaaaggctccattgcgcaagcagaacagcttcacggcaaagagctttcttacaacaacattaaagacgctgatgcagctgttcaaatcgtccgtgaattcactgagccggctgcggttgctgtgaagcatatgaatccgtgcggcgtcggaacaggaaaaacgatcgcagaagcgtttgacagagcgtttgaagccgatcaaacatcgatcttcggcggcattatcgcgctgaaccgtgaagtggacaaagcaaccgcagaggcgcttcacaacattttcttagaaatcatcattgcgccttcattcagccaagaagcacttgacatcttgactgcgaagaaaaatcttcgtctgctgacgcttgatgtgacagctgccgttcaaaaagaaaaacaactgacatctgttcaaggcgggctgctgattcaagatttagatatgcacggatttgatgatgctgagatcagc

SEQ ID NO: 281 - DNA, 519 bp, BACILLUS SP.
ggagaaaacccgcatcaggaagcgacattctatcaaacagctcttcctgtcaaaggctccattgcgcaagcagaacagcttcacggcaaagagctttcttacaacaacattaaagacgctgatgcagcagttcaaatcgtccgtgaattcactgagccggctgcggttgctgtgaagcatatgaatccgtgcggcgtcggaacaggaaaaacgatcgcagaagcgtttgacagagcgtttgaagccgatcaaacatcgatcttcggcggcattatcgcgctgaaccgtgaagtggacaaagcaaccgcagaggcgcttcacaacattttcttagaaatcatcattgcgccttcattcagccaagaagcacttgacatcttgactgcgaagaaaaatcttcgtctgctgacgcttgatgtgacagctgccgttcaaaaagaaaaacaactgacatctgttcaaggcgggctgctgattcaagatttagatatgcacggatttgatgatgctgagatcagc

SEQ ID NO: 282 - DNA, 519 bp, BACILLUS SP.
ggggaaaatcctcatcaagaagcaacattctaccaaagcgcgcttcctgtcaaaggatctattgcgcaagcagaacagctgcacggcaaagagctttcttacaacaacattaaagatgcggacgcagctgttcaaattgttcgtgaattcactgagcctgctgcggttgctgtgaagcatatgaatccgtgcggcgtgggaactggcgaaacgatcgcagaagcgtttgacagagcgttcgaagcagataaaacatcgatcttcggcggcattatcgcattgaaccgtgaagtggacaaggcaacagccgaagcgcttcatcatattttcttagaaatcatcattgcgccttcattcagccaagaagcgcttgatatcctgacagcgaagaaaaatctccgtctgctgacacttgatgtgacagctgccgttcaaaaagaaaaacagctgacatctgtacaaggcggtttgctgattcaagatttagatatgcacggctttgctgatgctgagatcagc

SEQ ID NO: 283 - DNA, 519 bp, BACILLUS SP.
ggagaaaatccgcatcaggaagcaacattctatcaaagcgcgcttcctgtcaaaggttctattgcacaagcagaacagctgcacggcaaagagctttcttacaacaacattaaagatgcggacgcagctgttcagattgttcgtgaattcacggagcctgctgcggttgctgtgaagcatatgaatccatgcggcgtgggaactggcgaaacgatcgcagaagcatttgacagagcgttcgaagcagataaaacatcgattttcggcggcattatcgcgttgaaccgtgaagtggacaagccaacagccgaagcgcttcataacattttcttagaaatcatcattgcgccttcattcagccaagaagcgcttgatatcctgacagcgaagaaaaatcttcgtctgctgacacttgatgtgacagctgccgtccaaaaagaaaaacagctgacatctgtacaaggcggattgctgattcaagatttagatatgcacggctttaatgatgctgagatcagc

SEQ ID NO: 284 - DNA, 519 bp, BACILLUS SP.
ggcgaaaaccctcaccaagaggctgttttctatcaaagcgcactgcctgtctccggttccattgcggcggcaaaacagcttcacggcaaagagctttcttacaacaacattaaagacgcggatgcgaccgttcaaatcgtccgggaatttacagaacccgctgctgttgccgttaaacatatgaacccatgcggagtcggtacgggagcttcaattgaggaagcgttcaataaagcatatgaagcagacaaaacgtccattttcggcggcatcatcgcgctgaaccgtgaagttgatcaggcaacggccgaagcccttcacggcatctttttagaaatcataatcgccccttctttcagtgaagaagcgctgaacgtgctgacgtcgaagaaaaatcttcgtctgctcacgcttgacgtgaatgcagcagggaagaaagaaaaacagctgacttccgtacagggcggccttttgattcaagatttagacgtgcacggatttgatgacgcaaaaatcagc

SEQ ID NO: 285 - DNA, 519 bp, BACILLUS SP.
ggcgaaaaccctcaccaagaggctgttttctaccaaagcgcactgcctgtctccggttccattgcggcggcaaaacagcttcacggcaaagagctttcttacaacaacattaaagacgcggatgcggccgttcaaatcgtccgggagtttgcagaacccgcggctgttgccgttaaacatatgaacccgtgcggagtcggtacgggagcttcaattgaggaagcattcaataaagcgtatgaagctgataaaacgtccattttcggcggcatcatcgcgctcaaccgtgaagttgatcaggcaacggccgaagcccttcacggcatctttttagaaatcataatcgccccttctttcagtgaagaagcgctgaatgtgctgacgtcgaagaaaaaccttcgtctgctcacgcttgacgtgaatgcagcagggaagaaagaaaaacagctgacttcggtacagggcggccttttgattcaagatttagacgtgcacggatttgatgacgcaaaaatcagc

SEQ ID NO: 286 - DNA, 519 bp, BACILLUS SP.
ggcgaaaaccctcaccaagaggctgttttctaccaaagcgcactgcctgtctccggttccattgcggcggcaaaacagcttcacggcaaagagctttcttacaacaacattaaagacgcggatgcggccgttcaaatcgtccgggaatttacagaacccgccgctgttgccgttaaacatatgaacccatgcggagtcggtacgggagccacaattgaggaagcgttcaataaagcgtatgaagcggacaaaacgtccattttcggcggcatcatcgcgctcaaccgtgaagttgatcaggcaacggccgaagcccttcacggcatctttttagaaatcatcatcgccccttctttcagtgaagaagcgctgaatgtgctgacgtcgaagaaaaaccttcgtctgctcacgcttgacgtgaatgcagcagggaagaaagaaaaacagctgacttccgtacagggcggccttttgattcaagatttagacgtgcacggatttgatgacgcaaaaatcagc

SEQ ID NO: 287 - DNA, 519 bp, BACILLUS SP.
ggtgaaaaccctcaccaagaggctgttttctatcaaagcgcacttcccgtctccggttccatcgcggcggcaaaacagcttcacggcaaagagctttcttacaacaatattaaggacgcggatgcggccgttcaaatcgtccgggaatttacagaacccgcagctgttgccgttaaacatatgaacccgtgcggagtcggtacgggagcttcaattgaggaagcattcaataaagcgtatgaagctgataaaacctccattttcggcggcatcatcgcgctgaaccgtgaagttgatcaggcaacggctgaagcccttcacggcatctttttagaaatcattatcgccccttctttcagtgaagaagcgctgaacgtactgacggcgaagaaaaaccttcgtctgctcacgcttgacgtgtccgcatcagggaagaaagaaaaacagctgacatccgtacagggcggccttttgattcaagatttagacgtccatggatttgatgacgccgacatcagc

SEQ ID NO: 288 - DNA, 519 bp, BACILLUS SP.
ggtgaaaaccctcaccaagaggctgttttctatcaaagcgcacttcccgtctccggttccatcgcggcggcaaaacagcttcacggcaaagagctttcttacaacaatattaaggacgcagatgcggccgttcaaatcgtccgggaatttacagaacccgcagctgttgccgttaaacatatgaacccgtgcggagtcggtacgggagcttcaattgaggaagcattcaataaagcgtatgaagctgataaaacctccattttcggcggcatcatcgcgctgaaccgtgaagttgatcaggcaacggctgaagcccttcacggcatctttttagaaatcattatcgccccttctttcagtgaagaagcgctgaacgtactgacggcgaagaaaaaccttcgtctgctcacgcttgacgtgtccgcatcagggaagaaagaaaaacagctgacatccgtacagggcggccttttgattcaagatttagacgtccatggatttgatgacgccgacatcagc

SEQ ID NO: 289 - DNA, 519 bp, BACILLUS SP.
ggtgaaaaccctcaccaagaggctgttttctatcaaagcgcacttcccgtctccggttccatcgcggcggcaaaacagcttcacggcaaagagctttcttacaacaatattaaggacgcagatgcggccgttcaaatcgtccgggaatttacagaacccgcagctgttgccgttaaacatatgaacccgtgcggagtcggtacgggagcttcaattgaggaagcattcaataaagcgtatgaagctgataaaacctccattttcggcggcatcatcgcgctgaaccgtgaagttgatcaggcaacggctgaagcccttcacggcatctttttagaaatcattatcgccccttctttcagtgaagaagcgctgaacgtactgacggcgaagaaaaaccttcgtctgctcacgcttgacgtgtccgcatcagggaagaaagaaaaacagctgacatccgtacagggcggccttttgattcaagatttagacgtccatggatttgatgatgccgacatcagc

SEQ ID NO: 290 - DNA, 519 bp, BACILLUS SP.
ggtgaaaaccctcaccaagaggcggttttctaccaaagcgcacttcccgtctccggttccatcgcggcggcaaaacagcttcacggcaaagagctttcttacaacaacattaaggacgcagatgcggccgttcaaatcgtccgggaatttacagaacccgcaactgttgccgttaaacatatgaacccgtgcggagtcggtacgggagcttcaattgaggaagcattcaataaagcgtatgaagctgataaaacctccattttcggcggcatcatcgcgctgaaccgtgaagttgatcaggcaacggctgaagcccttcacggcatctttttagaaatcattatcgccccttctttcagtgaagaagcgctgaacgtactgacggcgaagaaaaaccttcgtctgctcacgcttgacgtgaccgcatcagggaagaaagaaaaacagctgacatccgtacagggcggtcttttgattcaagatttagacgtccatggatttgatgacgccgacatcagc

SEQ ID NO: 291 - DNA, 519 bp, BACILLUS SP.
ggggaaaatcctcatcaagaagccacattctaccaaagcgcgcttcctgtcaaaggatctattgcgcaagcagaacagctgcacggcaaagagctttcttacaacaacattaaagacgcggacgcagctgttcaaattgttcgtgaattcactgagcctgctgcggttgctgtgaagcatatgaatccgtgcggggtgggaattggcgaaacgatcgcagaagcgtttgacagagcgttcgaagcagataaaacatcgatcttcggcggcattatcgcgttgaaccgtgaagtggacaaggcaacagccgaagcgcttcatcatattttcttagaaatcatcattgcgccttcattcagccaagaagcgcttgatatcctgacagcgaagaaaaatctccgtctgctgacacttgatgtgacagctgccgttcaaaaagaaaaacagctgacatctgtacaaggcggtttgctgattcaagatttagatatgcacggctttgctgatgctgagatcagc

SEQ ID NO: 292 - DNA, 519 bp, BACILLUS SP.
ggagaaaacccgcatcaggaagcaactttctatcaaacagctcttcctgtcaaaggctccattgcgcaagcagaacagcttcacggaaaagagctttcttacaacaacattaaagacgcggatgcggcagttcaaatcgttcgtgaattcactgaaccggctgctgttgctgtgaagcacatgaacccgtgcggtgtaggaacaggaaaaacgatcgcagaagcgtttgacagagcgtttgaagcggataaaacatctatcttcggcggcattatcgcgctgaaccgtgaagtggacaaggcaactgccgaagcgcttcacaacattttcttagaaatcatcattgcgccttcattcagccaagaagcgctcgacgtcctgactgcgaaaaaaaatctccgtctgctgacgcttgacgtatccgccgctgttcaaaaggaaaaacaactgacatctgttcaaggcgggctgctgattcaagatttagatatgcacggctttgatgatgctgagattagc

SEQ ID NO: 293 - DNA, 519 bp, BACILLUS SP.
ggtgaaaatccgcatcaggaagcagtcttttatcaaagcgcgctccctgtcaaagggtcaatcgcttcagctgaacagctgcacggaaaagagctctcctacaacaacatcaaagacgcggatgcagcactacaaatcgtccgcgagttcaccgagccggctgcggttgccgtcaagcatatgaacccttgcggcgtcgggacgggcaacacgatcgaagaggcgtttaacaaagcatacgccgctgacgaaacgtcgattttcggcggcatcatcgctctgaatcgtgaagtggacaaagcgacggctgaaacgcttcacaaaatctttttggaaatcattatcgcaccatcattcagccaagaagcgcttgatattttaacatccaagaaaaaccttcgtctcttgacgcttgatgtcaatcagcttgtgaaaaacgaaaagcagctgacatctgttcaaggcgggctcttgattcaggatctggatatgcacggctttgacgacgctgacattacg

SEQ ID NO: 294 - DNA, 519 bp, BACILLUS SP.
ggcgaaaatccgcatcaggaagcagtcttttatcaaagcgcgctccctgtcaaagggtcaatcgcttcaactgaacagctgcacggaaaagagctctcctacaacaacatcaaagatgcggatgcagcactacaaatcgtccgcgagttcaccgagccggctgcggttgccgtcaagcatatgaacccttgcggcgtcgggacgggcaacacgatcgaagaggcgtttaacaaagcatacgccgctgacgaaacgtcgattttcggcggcatcattgccctgaatcgtgaagtggacaaagcgacggctgaaacgcttcacaaaatctttttggaaatcattatcgcaccatcattcagccaagaagcgcttgatattttaacatccaagaaaaaccttcgtctcttgaccctcgatgtcaatcagcctgtgaaaaacgaaaagcagctgacatctgttcaaggcgggctcttgattcaggatctggatatgcacggctttgacgacgctgacattacg

SEQ ID NO: 295 - DNA, 519 bp, BACILLUS SP.
gaaaaattaattgagaatccgaaacatattgaggttcaggtcattggagacaagcagggcaatgtcgtccatctttttgagagggattgctccgttcaaagacgccatcaaaaagtcattgaagtggcgccgagtgtctcgctgtcacctgaattaagggaccaaatttgtgaggctgcagttgcgcttgccaaaaatgtaaactatataaatgcggggacggtcgaattccttgttgcaaacaacgagttctactttattgaagtaaatcctcgcgtacaagttgaacacacgataacagaaatgattactggtgtcgatattgttcaaactcagatccttgttgcccaagggcacagccttcacagcaaaaaagtaaatattcctgagcaaaaggacatttttacaatcggctatgccattcagtcacgggttacgactgaggatccgcaaaatgatttcatgcctgatacaggaaaaatcatggcttaccgctcaggcggcggttttggtgtccgt

SEQ ID NO: 296 - DNA, 519 bp, BACILLUS SP.
gaaaaattaattgagaatccgaaacatattgaggttcaggtcattggagacaagcagggaaatgtcgtccatctttttgagagggattgctccgttcaaagacgccatcaaaaggtcattgaagtggcgccgagtgtctcgctgtcacctgaattaagggaccaaatttgtgaggcggcagttgcgcttgccaaaaatgtaaactatataaatgcggggacggtcgaattccttgttgcaaacaacgagttctactttattgaagtaaaccctcgcgtacaagttgaacacacgataacagaaatgattactggtgtcgatattgttcaaactcagatccttgttgcccaagggcacagccttcacagcaaaaaagtaaatattcctgagcaaaaggacatttttacaatcggctatgccattcagtcacgggttacgactgaggatccgcaaaatgatttcatgcctgatacaggaaaaatcatggcttaccgctcaggcggcggttttggtgtccgt

SEQ ID NO: 297 - DNA, 519 bp, BACILLUS SP.
gaaaaattaattgagaatccgaaacatattgaggttcaggtcattggagacaagcagggaaatgtcgtccatctttttgagagggattgttccgttcaaagacgccaccaaaaggtcattgaagtggcgccgagtgtctcgctgtcacctgaattaagggatcaaatttgtgaggcggcagttgcgcttgctaaaaatgtaaactatataaatgcggggacggtcgaattccttgttgcaaacaacgagttctactttattgaagtaaatcctcgcgtacaagttgaacacacgataacagaaatgattactggtgtcgatattgttcaaactcagatccttgttgcccaagggcatagccttcacagcaaaaaagtaaatattcctgagcaaaaggacatttttacaatcggctatgccattcagtcacgggttacgactgaggatccgcaaaatgacttcatgcctgatacaggaaaaatcatggcttaccgctcaggcggcggttttggtgtccgt

SEQ ID NO: 298 - DNA, 519 bp, BACILLUS SP.
gaaaaattaattgagaatccgaaacatattgaggttcaggtcattggagacaagcagggaaatgtcgtccatctttttgagagggattgttccgttcaaagacgccatcaaaaggtcattgaagtggcgccgagtgtctcgctgtcacctgaattaagggatcaaatttgtgaggcggcagttgcgcttgctaaaaatgtaaactatataaatgcggggacggtagaattccttgttgcaaacaacgagttctactttattgaagtaaatcctcgcgtacaagttgaacacacgataacagaaatgattactggtgtcgatattgttcaaactcagatccttgttgcccaagggcacagccttcacagcaaaaaagtaaatattcctgagcaaaaggacatttttacaatcggctatgccattcagtcacgggttacgactgaggatccgcaaaatgatttcatgcctgatacaggaaaaatcatggcttaccgctcaggcggcggttttggtgtccgt

SEQ ID NO: 299 - DNA, 519 bp, BACILLUS SP.
gaaaaattgattgaaaatccgaaacatattgaggttcaggtgattggtgacaagcagggcaatgtcgttcacctttttgagagggactgttctgttcaaagacgccaccaaaaagtcattgaagtggcgccgagtgtctctctgtcacctgaattaagaaaccaaatttgtgaagcggcagttgcgcttgcgaaaaatgtaaactatataaacgcgggtacggtagagttccttgttgcaaacaacgaattttactttattgaagtaaaccctcgcgtacaagttgaacatacgattacagaaatgattacaggtgtcgatattgttcaaacacagatccttgttgctcaagggcacacccttcacagcaaaaaagtaaatattcctgagcaaaaggacatttctacaatcggctatgccattcagtcacgggttacgactgaggacccgcaaaatgatttcatgcctgatacaggaaaaatcatggcttaccgctcaggcggcggttttggcgtccgt

SEQ ID NO: 300 - DNA, 519 bp, BACILLUS SP.
gaaaaattgattgaaaatccgaaacatattgaggttcaggtgattggtgacaagcagggcaatgtcgtccacctttttgagagggactgttctgttcaaagacgccatcaaaaagtcattgaagtggcgccgagtgtctccctgtcacctgaattaagaaaccaaatttgtgaagcggcagttgcgcttgcgaaaaatgtaaactatataaacgctggtacggtagagtttcttgttgcaaacaacgaattctactttattgaagtaaaccctcgcgtacaagttgaacacacgattacagaaatgattacaggtgtcgatatcgttcaaacacagatccttgttgcccaaggacacagccttcacagcaaaaaagtaaatattcctgagcaaaaggacatttctacaatcggctatgccattcagtcacgggttacgactgaggatccgcaaaatgatttcatgcctgatacaggaaaaatcatggcttaccgctcaggcggcggttttggtgtccgt

SEQ ID NO: 301 - DNA, 519 bp, BACILLUS SP.
gaaaaattgattgaaaatccgaaacatattgaggttcaggtgattggagacaagcagggcaatgtcgtccacctttttgagagggactgttctgttcaaagacgccaccaaaaagtcattgaagtggcaccgagtgtctcgctgtcacctgaattgagagatcagatttgtgaagcagcagttgcgcttgcgaaaaatgtaaactatataaacgcaggtacggttgaattccttgttgcaaacaacgagttctactttattgaagtaaaccctcgcgtacaagttgaacatacgattacagaaatgattacaggtgtcgacattgttcaaacacagatccttgttgcccaaggacacagccttcacagcaaaaaagtaaatattcctgagcaaaaggacatttctacaaacggctatgccattcagtcacgggttacgactgaggatccgcaaaatgatttcatgcctgatactggaaaaatcatggcttaccgctcaggcggcggttttggtgtccgt

SEQ ID NO: 302 - DNA, 519 bp, BACILLUS SP.
gaaaaattgattgaaaatccgaaacatattgaggttcaggtgattggagacaagcagggcaatgtcgtccacctttttgagagggactgttctgttcaaagacgccaccaaaaagtcattgaagtggcaccgagtgtctcgctgtcacctgaattgagagatcagatttgtgaagcggcagttgcgcttgcgaaaaatgtaaactatataaacgcaggtacggttgaattccttgttgcaaacaacgagttctactttattgaagtaaaccctcgcgtacaagttgaacatacgattacagaaatgattacaggtgtcgacattgttcaaacacagatccttgttgcccaagggcacagccttcacagcaaaaaagtaaatattcctgagcaaaaggacatttctacaaacggctatgccattcagtcacgggttacgactgaggatccgcaaaatgatttcatgcctgatacaggaaaaataatggcttaccgctcaggcggcggttttggtgtccgt

SEQ ID NO: 303 - DNA, 519 bp, BACILLUS SP.
gagaaactaattgaaaaccctaaacatattgaagttcaagtcattggagacaagcagggcaacgtggttcatctttttgaaagggattgttccgttcaaaggcgccatcaaaaagtaatcgaagtagcgccgagtgtctcgctgtcatctgaattgagagatcaaatttgtgaagcggcagttgcgcttgctaaaaatgtggattacataaacgcaggtacggttgagttccttgtcgcgaacaatgagttctattttattgaagtaaatccgcgcgtacaagttgaacatacaattacggaaatgattaccggtgtcgatattgtacaatcacagattcttgttgcccaagggcacagtcttcacagcaaaaaagtaaatatcccgcagcaaaaggacatttctacaatcggttatgcaattcagtcgcgggtaacgactgaagatccgcagaatgatttcatgcctgatacaggaaaaattatggcttaccgctctggcggcggctttggtgtccgg

SEQ ID NO: 304 - DNA, 519 bp, BACILLUS SP.
gagaaactgattgaaaaccctaagcatattgaggttcaggtgattggagacaatcagggcaacgtgattcatctttttgaacgggactgttctgttcaaagacgccaccaaaaggtgattgaagtagcgccgagcgtttcgctgtcaactgaattgagagatcaaatttgtgaagcggcggttgcgcttgctaaaaacgtgaactatataaatgctggtacggttgaattccttgtcgcgaacaacgagttttattttatagaagtaaatccgcgtgttcaagttgagcatacaattacggaaatgattaccggtgttgatattgtgcagacacagatccttgttgcccaaggccacagccttcacagcaaaaaagtcaacattccggagcagaaggacatttttacaatcggctatgcgattcagtcaagggtaacgactgaggatccgcaaaatgattttatgcctgacacagggaaaatcatggcttaccgctcgggtggaggatttggtgttcgt

SEQ ID NO: 305 - DNA, 519 bp, BACILLUS SP.
gagaaactgatcgaaaacccaaaacatattgaagtccaggtgattggggataaaaacggcaatgtcgttcatctctatgaaagagactgctccgtccaaagacgccatcaaaaagtcatcgaggtagcaccaagtgtgtcgctatcaaatgaattgagagatcagatttgcgaagctgcagtggcgctcgctaaaaatgttcaatacataaatgcagggacagtagaatttctggttgctaacgacgagtttttcttcattgaagtaaacccgcgagtacaggtcgagcatacgattacagagatgattacaggagtcgatattgttcaaacgcagattctagttgcacaagggtacgaactgcacagccgtgaagtaaacattcctgagcaaaaggatattttcacaatcggtttcgccatccagtctcgtgtcacaacggaagatccgctgaacgattttatgcctgatacagggaaaatcatggcttaccgctcaggcggcggcttcggtgtccgc

SEQ ID NO: 306 - DNA, 519 bp, BACILLUS SP.
gagaaactgatcgaaaacccaaaacatattgaagtccaggtgattggggataaaaacggcaatgtcgttcatctctatgaaagagactgctccgttcaaagacgccatcaaaaagtcatcgaggtagcaccaagtgtgtcgctatcaaatgaattgagagatcagatttgcgaagctgcagtggcgctcgctaaaaatgttcaatacataaatgcagggacagtagaatttctggttgctaacgacgagtttttcttcattgaagtaaacccgcgtgtacaggtcgagcatacgattacagagatgattacaggagttgatattgttcaaacgcagattctagttgcacaagggtacgaactgcacagccgtgaagtaaacattcctgagcaaaaggatattttcacaatcggtttcgccatccagtctcgtgtcacaacggaagatccgctgaacgattttatgcctgatacagggaaaatcatggcttaccgctcaggcggcggcttcggtgtccgc

SEQ ID NO: 307 - DNA, 519 bp, BACILLUS SP.
gagaaactgatcgaaaacccaaaacatattgaagtccaggtgattggggataaaaacggcaatgtcgttcatctctatgaaagagactgctccgttcaaagacgccatcaaaaagtcatcgaggtagcaccaagtgtgtcgctatcaaatgaattgagagatcagatttgcgaagctgcagtggcgctcgctaaaaatgttcaatacataaatgcagggacagtagaatttctggttgctaacgacgagtttttcttcattgaagtaaacccgcgtgtacaggtcgagcatacgattacagaaatgattacaggagtcgatattgttcaaacgcagattctagttgcacaagggtacgaactgcacagccgtgaagtaaacattcctgagcaaaaggatattttcacaatcggtttcgccatccagtctcgtgtcacaacggaagatccgctgaacgattttatgcctgatacagggaaaatcatggcttaccgctcaggcggcggcttcggtgtccgc

SEQ ID NO: 308 - DNA, 519 bp, BACILLUS SP.
gagaaactgatcgaaaacccaaaacatattgaagtccaggtgattggggataaaaacggcaatgtcgttcatctctatgaaagagactgctccgttcaaagacgccatcaaaaagtcatcgaggtagcaccaagtgtgtcgctatcaaatgaattgagagatcagatttgcgaagctgcagtggcgctcgctaaaaatgttcaatacataaatgcagggacagtagaatttctggttgctaacgacgagtttttcttcattgaagtaaacccgcgtgtacaggttgagcatacgattacagaaatgattacaggagtcgatattgttcaaacgcagattctagttgcacaagggtacgaactgcacagccgtgaagtaaacattcctgagcaaaaggatattttcacaatcggtttcgccatccagtctcgtgtcacaacggaagatccgctgaacgattttatgcctgatacagggaaaatcatggcttaccgctcaggcggcggcttcggtgtccgc

SEQ ID NO: 309 - DNA, 519 bp, BACILLUS SP.
gaaaaactgatcgaaaatcctaagcatattgaagttcaggtgatcggagataaagaaggcaatgtcgtccatctgtatgagcgggactgttccgttcagagacgccatcaaaaggtcattgaggtggcgcctagtgtatctttgcagcctgaattaagggacgaaatctgtgaagcggctgtggcgcttgcgaaaaatgtcggctatatcaacgcagggaccgtggagtttctcgtagcgggcggtgaattttactttattgaagtgaacccgcgcgtgcaagtggagcatacgattacggaaatgataacgggggttgatatcgttcagacgcaaatcctcgtggcgcagggccacggactgcacagccgtgccgtcaatatccctgagcagaaagacatttttacaaacgggtatgcgatacaatcacgtgtgaccactgaagatccgctgaatgactttatgcctgacacgggtaaaattatggcttaccgctcaggcggcggcttcggcgtccgc

SEQ ID NO: 310 - DNA, 519 bp, BACILLUS SP.
gaaaagctgatcgaaaagccgaagcacattgaagttcaagtcatcggagacaaagaaggtaacgtggttcacctttacgaccgtgactgctctgtgcaaaggcgtcatcaaaaggttatcgaaatagcgccgagcgtttcgctttctgagtccctccgggaaaagatttgcgatgctgccgttaagcttgcgaagaatgttgattatgtcaatgccggtacggtcgaatttttagttgcgaacgatgagtttttctttattgaagtcaacccgcgtgttcaggtggagcacaccattacggaaatggtgacgggtgtcgatatcgttcagacacaaatcctgattgctgccggactcagtctgggcagcagcgaaatcagcattccgaaccaggatgccatcacgctgcacggatatgcgatccagtcaagggttacgactgaagacccgtcaaacaatttcatgcctgacacaggcaaaatcatggcatatcgctcaggcggcggtttcggggtgagg

SEQ ID NO: 311 - DNA, 519 bp, BACILLUS SP.
gaaaagctgattgaaaagccgaagcacattgaagttcaagtcatcggagataaagaagggaacgtggttcacctttacgaccgcgactgctctgtgcaaaggcgtcatcaaaaggtcatcgaagtggcgccgagcgtttcgctttctgaatccctccgggaaaagatttgcgatgctgccgttaagcttgcgaagaatgttgaatatgtcaatgccggtacagtcgaatttttagttgcgaacgatgagtttttctttattgaagtgaacccgcgtgttcaggtggagcataccattacggaaatggtaacgggcgtcgatatcgttcagacgcaaatcctgatcgctgccggtctcagtctggacagcagcgaaatcagcattccgaaccaggatgcgatcacgctgcacggatatgcgatccagtcaagggttacgactgaagatccgtcaaacaatttcatgcctgacacaggcaaaatcatggcatatcgctcaggcggcggtttcggggtgagg

SEQ ID NO: 312 - DNA, 519 bp, BACILLUS SP.
gaaaagctgatcgaaaagccgaagcatattgaggttcagattatcggagacaaggaagggaacgttgttcatctgtatgaccgggactgctccgtgcaaaggcgtcatcaaaaagtcattgaaatcgcaccaagcgtttctctttccgagtcgctgagggaaaaaatctgcgatgccgccgtcgcacttgcgaaaaacgtcaattatatcaatgccggaacggtcgaatttttagtcgcgaacgatgaatttttctttattgaagtcaatccgcgcgttcaggtcgaacacaccatcacagaaatggtgacaggcgtcgatatcgtccagacgcagattatgatcgccggggggctgagcctgaagagcaaagaagtgaacatcccgtcacaggatgcggtcactcttcacggcttcgcgatccagtcacgggtaacgacagaagacccgtcaaacaacttcatgcctgatacaggcaagattatggcctaccggtctggcgggggattcggggtccgg

SEQ ID NO: 313 - DNA, 519 bp, BACILLUS SP.
gaaaagctgatcgaaaagccgaagcatattgaggttcagattatcggagacaaggaagggaacgttgttcatctgtatgaccgggactgctccgtgcaaaggcgtcatcaaaaagtcattgaaatcgcaccaagcgtttctctttccgagtcgctgagggaaaaaatctgcgatgccgccgtcgcacttgcgaaaaacgtcaattatatcaatgccggaacggtcgaatttttagtcgcgaacgatgaatttttctttattgaagtcaatccgcgcgttcaggtcgaacacaccatcacagaaatggtgacaggcgtcgatatcgtccagacgcagattatgatcgccggggggctgagcctgaagagcaaagaagtgaacatcccgtcacaggatgcggtcactcttcacggcttcgcgatccagtcacgggtaacgacagaggacccgtcaaacaacttcatgcctgatacaggcaagattatggcctaccggtctggcgggggattcggggtccgg

SEQ ID NO: 314 - DNA, 519 bp, BACILLUS SP.
gaaaaattaattgagaatccgaaacatattgaggttcaggtcattggagacaagcagggaaatgtcgtccatctttttgagagggattgctccgttcaaagacgccatcaaaaggtcattgaagtggcgccgagtgtctcgctgtcacctgaattaagggaccaaatttgtgaggcggcagttgcgcttgccaaaaatgtaaactatataaatgcggggacggtcgaattccttgttgcaaacaacgagttctactttattgaagtaaaccctcgcgtacaagttgaacacacgataacagaaatgattactggtgtcgatattgttcaaactcagatccttgttgctcaagggcacagccttcacagcaaaaaagtaaatattccggagcaaaaggacatttttacaatcggctatgccattcagtcacgggttacgactgaggatccgcaaaatgatttcatgcctgatacaggaaaaatcatggcttaccgctcaggcggcggttttggtgtccgt

SEQ ID NO: 315 - DNA, 519 bp, BACILLUS SP.
gaaaaattgattgaaaatccgaaacatattgaggttcaggtgattggtgacaagcagggcaatgtcgtccacctttttgagagggactgttctgttcaaagacgccatcaaaaagtcattgaagtggcgccgagtgtctccctgtcacctgaattaagaaaccaaatttgtgaagcggcagttgcgcttgcgaaaaatgtaaactatataaacgctggtacggtagagtttcttgttgcaaacaacgaattctactttattgaagtaaaccctcgcgtacaagttgaacacacgattacagaaatgattacaggtgtcgatatcgttcaaacacagatccttgttgcccaagggcacagccttcacagcaaaaaagtaaatattcctgagcaaaaggacatttctacaatcggctatgccattcagtcacgggttacgactgaggatccgcaaaatgatttcatgcctgatacaggaaaaatcatggcttaccgctcaggcggcggttttggtgtccgt

SEQ ID NO: 316 - DNA, 519 bp, BACILLUS SP.
gagaaactaattgaaaaccctaaacatattgaagttcaagtcattggagacaagcagggcaacgtggttcatctttttgaaagggattgttccgttcaaaggcgccatcaaaaagtaatcgaagtagcgccgagtgtctcgctgtcatctgaattgagagatcaaatttgtgaagcggcagttgcgcttgctaaaaatgtggattacataaacgcaggtacggttgagttccttgtcgcgaacaacgagttctattttattgaagtaaatccgcgcgtacaagttgaacatacaattacggaaatgattaccggtgtcgatattgtacaatcacagattcttgttgcccaagggcacagtcttcacagcaaaaaagtaaatatcccgcagcaaaaggacatttctacaatcggttatgcaattcagtcgcgggtaacgactgaagatccgcagaatgatttcatgcctgatacaggaaaaattatggcttatcgctctggcggcggctttggtgtccgg

SEQ ID NO: 317 - DNA, 519 bp, BACILLUS SP.
gagaaactgattgaaaaccctaagcatattgaggtccaggttatcggggacaatcagggaaacgtgattcatctatttgaacgggattgttctgttcaaagacgccaccaaaaggtaatcgaagtagcgccgagcgtttcgctgtcaactgaattgagagaccaaatttgtgaagctgcggttgcgcttgctaaaaacgtgaactatataaatgctggtacggttgaattccttgtcgcgaacaacgagttttattttatagaagtaaatccgcgtgttcaagttgaacatacaattacggaaatgattaccggtgttgatattgtgcagacacagatacttgttgcccaaggccacagcctacatagcaaaaaagtcaacattccggagcagaaggacatttttacaatcggctatgcgattcagtcacgggtaacgactgaagatccgcaaaatgattttatgcctgacacagggaaaatcatggcttaccgttcaggcggaggctttggtgttcgt

SEQ ID NO: 318 - DNA, 519 bp, BACILLUS SP.
gagaaactgattgaaaaccctaagcatattgaggttcaggtgattggagacaatcagggcaacgtgattcatctttttgaacgggactgttctgttcaaagacgccaccaaaaggtgattgaagtagcgccgagcgtttcgctgtcaactgaattgagagatcaaatttgtgaagcggcggttgcgcttgctaaaaacgtgaactatataaatgctggtacggttgaattccttgtcgcgaacaacgagttttattttatagaagtaaatccgcgtgttcaagttgagcatacaattacggaaatgattaccggtgttgatattgtgcagacacagatccttgttgcccaaggccacagccttcacagcaaaaaagtcaacattccggagcagaaggacatttttacaatcggctatgcgattcagtcacgggtaacgactgaagatccgcaaaatgattttatgcctgacacagggaaaatcatggcttaccgctcgggtggaggctttggtgttcgt

SEQ ID NO: 319 - DNA, 519 bp, BACILLUS SP.
gagaaactgattgaaaaccctaagcatattgaggtccaggttattggagacaatcagggaaacgtgattcatctatttgaacgggattgttctgttcaaagacgccaccaaaaggtaatcgaagtagcgccgagcgtttcgctgtcaactgaattgagagaccaaatttgtgaagctgcggttgcgcttgctaaaaacgtgaactatataaatgctggtacggttgaattccttgtcgcgaacaacgagttttattttatagaagtaaatccgcgtgtccaagttgagcatacaattacggaaatgattaccggtgttgatattgtgcagacacagatacttgttgcccaaggccacagcctacatagcaaaaaagtcaacattccggagcagaaggacatttttacaatcggctatgcgattcagtcacgggtaacgactgaagatccgcaaaatgattttatgcctgatacagggaaaatcatggcttaccgttcaggcggaggctttggtgttcgt

SEQ ID NO: 320 - DNA, 519 bp, BACILLUS SP.
gaaaaattaattgagaatccgaaacatattgaggttcaggtcattggagacaagcagggaaatgtcgtccatctttttgagagggattgttccgttcaaagacgccatcaaaaggtcattgaagtggcgccgagtgtctcgctgtcacctgaattaagggatcaaatttgtgaggcggcagttgcgcttgctaaaaatgtaaactatataaatgcggggacggtcgaattccttgttgcaaacaacgagttctactttattgaagtaaatcctcgcgtacaagttgaacacacgataacagaaatgattactggtgtcgatattgttcaaactcagatccttgttgcccaagggcatagccttcacagcaaaaaagtaaatattcctgagcaaaaggacatttttacaatcggctatgccattcagtcacgggttacgactgaggatccgcaaaatgacttcatgcctgatacaggaaaaatcatggcttaccgctcaggcggcggttttggtgtccgt

SEQ ID NO: 321 - DNA, 519 bp, BACILLUS SP.
gaaaagctgattgaaaagccgaagcacattgaagttcaagtcatcggagataaagaagggaacgtggttcacctttacgaccgcgactgctctgtgcaaaggcgacatcaaaaggtcatcgaagtggcgccgagcgtttcgctttctgaatccctccgggaaaagatttgcgatgctgccgttaagcttgcgaagaatgttgactatgtcaatgccggtacagtcgaatttttagttgcgaacgatgagtttttctttattgaagtgaacccgcgtgttcaggtggagcataccattacggaaatggtaacgggcgtcgatatcgttcagacgcaaatcctgatcgctgccggtctcagtctggacagcagcgaaatcagcattccgaaccaggatgcgatcacgctgcacggatatgcgatccagtcaagggttacgactgaagatccgtcaaacaatttcatgcctgacacaggcaaaatcatggcatatcgctcaggcggcggtttcggggtgagg

SEQ ID NO: 322 - DNA, 519 bp, BACILLUS SP.
gaaaaactgatcgaaaatcctaagcatattgaagttcaggtgatcggagataaagaaggcaatgtcgtccatctgtatgagcgggactgttccgttcagagacgccatcaaaaggtcattgaggtggcgccgagtgtatctttgcagcctgaattaagggacgaaatctgtgaagcggctgtggcgcttgcgaaaaatgtcggctatatcaacgcggggaccgtggaatttctcgtagcgggcggtgaattttactttattgaagtgaacccgcgcgtgcaagtggagcatacgattacggaaatgataacgggggttgatatcgttcagacgcaaatcctcgtcgctcagggctacggactccacagccgtgccgtcaatatccctgagcagaaagacatttttacaaacgggtatgcgatacaatcacgtgtgaccactgaagatcctctgaatgactttatgcctgacacgggtaaaattatggcttaccgctcaggcggcggcttcggcgtccgc

SEQ ID NO: 323 - DNA, 519 bp, BACILLUS SP.
gaaaaactgatcgaaaatcctaagcatattgaagttcaggtgatcggagataaagaaggcaatgtcgtccatctgtatgagcgggactgttccgttcagagacgccatcaaaaagtcattgaggtggcgccgagtgtatctttgcagcctgaattaagggacgaaatctgtcaagcggctgtggcgcttgcgaaaaatgtcggctatatcaacgcggggaccgtggaatttctcgtagcgggcggtgaattttactttattgaagtgaacccgcgcgtgcaagtggagcatacgattacggaaatgataacgggggttgatatcgttcagacgcaaatcctcgtcgcgcagggccacggactgcacagccgtgccgtcaatatccctgagcagaaagacatttttacaaacgggtatgcgatacaatcacgtgtgaccactgaagatccgctgaatgactttatgcctgacacgggtaaaattatggcttaccgctcaggcggcggcttcggcgtccgc

SEQ ID NO: 324 - DNA, 519 bp, BACILLUS SP.
gaaaaactgatcgaaaatcctaagcatattgaagttcaggtgatcggagataaagaaggcaatgtcgtccatctgtatgagcgggactgttccgttcagagacgccatcaaaaggtcattgaggtggcgccgagtgtatctttgcagcctgaattaagggacgaaatctgtgaagcggctgtggcgcttgcgaaaaatgtcggctatatcaacgcggggaccgtggaatttctcgtagcgggcggtgaattttactttattgaagtgaacccgcgcgtgcaagtggagcatacgattacggaaatgataacgggggttgatatcgttcagacgcaaatcctcgtcgcgcagggccacggactgcacagccgtgccgtcaatatccctgagcagaaagacatttttacaaacgggtatgcgatacaatcacgtgtgaccactgaagatccgctgaatgactttatgcctgacacgggtaaaattatggcttaccgctcaggcggcggcttcggcgtccgc

SEQ ID NO: 325 - DNA, 519 bp, BACILLUS SP.
gaaaagctgatcgaaaagccgaagcatattgaggttcagattatcggagacaaggaagggaacgttgttcatctgtatgaccgggactgctccgtgcaaaggcgtcatcaaaaagtcattgaaatcgcaccaagcgtttctctttccgagtcgctgagggaaaaaatctgcgatgccgccgtcgcacttgcgaaaaacgtcaattatatcaatgccggaacggtcgaatttttagtcgcgaacgatgaatttttctttattgaagtcaatccgcgcgttcaggtcgaacacaccatcacagaaatggtgacaggcgtcgatatcgtccagacgcagattatgatcgccggggggctgagcctgaagagcaaagaagtgaacatcccgtcacaggatgcggtcactcttcacggcttcgcgatccagtcacgggtaacgacagaggacccgtcaaacaacttcatgcctgatacaggcaagattatggcctaccggtctggtgggggattcggggtccgg

SEQ ID NO: 326 - DNA, 519 bp, BACILLUS SP.
gaaaagctgatcgaaaagccgaagcacattgaagttcaagtcattggagataaagaagggaacgtggttcacctttacgaccgcgactgctctgtgcaaaggcgtcatcaaaaggtcatcgaagtggcgccgagcgtttcgctttctgaatccctccgggaaaagatttgcgatgccgccgttaagcttgcgaagaatgttgactatgtcaatgccggtacggtcgaatttttagttgcgaacgatgagtttttctttattgaagtcaacccgcgtgttcaggtggagcacaccattacggaaatggtgacgggtgtcgatatcgttcagacgcaaatcctgattgctgccggattcagtctggacagcagcgaaatcagcattccgaaccaggatgccatcacgctgcacggatatgcgatccagtcaagggttacgactgaagacccgtcaaacaatttcatgcctgacacaggcaaaatcatggcatatcgctcaggcggcggtttcggggtgagg

SEQ ID NO: 327 - DNA, 519 bp, BACILLUS SP.
gaaaagctgatcgaaaagccgaagcacattgaagttcaagtcattggagataaagaagggaacgttgttcacctttacgaccgcgactgctctgtgcaaaggcgtcatcaaaaggtcatcgaagtggcgccgagcgtttcgctttcagaatccctccgggaagagatttgcgaagccgccgttaagcttgcgaagaatgttgactatgtcaatgccggtacggtcgaatttttagtcgcgaacgatgagtttttctttattgaagtcaacccgcgtgttcaggtggagcacaccattacggaaatggtgacgggtgtcgatatcgtacagacgcaaatcctgattgctgccggattcagtctggacagcagcgaaatcagcattccgaaccaggatgccatcacgctgcacggatatgcgatccagtcaagggttacgactgaagacccgtcaaacaatttcatgcctgacacaggcaaaatcatggcatatcgctcaggcggcggtttcggggtgagg

SEQ ID NO: 328 - DNA, 504 bp, BACILLUS SP.
cgcggtatgctgttccttgatctgatccaggaaggaaacatgggcctgatgaaagccgttgaaaaatttgattatcgcaaaggttataaattcagtacgtatgctacgtggtggatcagacaggcgattacacgcgccattgccgatcaggcgagaacgatccggattcccgttcatatggttgaaaccattaataaattaatccgtgtgcagcgtcaattactgcaagacttaggcagagaaccaacacctgaagaaattgcggaagatatggatttaacgcctgaaaaagtacgcgaaatcttaaagattgctcaagagccggtatctctggaaacaccgatcggtgaagaggatgactcgcaccttggtgatttcattgaagaccaagaagcaacttcaccttctgaccacgccgcatacgagctattgaaagagcagctggaagatgtgcttgatacgttaactgatcgtgaagaaaatgtattgcgtcttcgattcggt

SEQ ID NO: 329 - DNA, 504 bp, BACILLUS SP.
cgcggtatgctgttccttgatctgatccaggaaggaaacatgggtctgatgaaagccgttgaaaaatttgattatcgcaaaggttataaattcagtacgtatgctacgtggtggatcagacaggcgattacacgcgccattgccgatcaggcgagaacgatccggattcccgttcatatggttgaaaccattaataaattaatccgtgtgcagcgtcaattactgcaagacttaggcagagaaccaacacctgaagaaattgcggaagatatggatttaacgcctgaaaaagtacgcgaaatcttaaagattgctcaagagccggtatctctggaaacaccgatcggtgaagaggatgactcgcaccttggtgatttcattgaagaccaagaagcaacttcaccttctgaccacgccgcatacgagctattgaaagagcagctggaagatgtgcttgatacgttaactgatcgtgaagaaaatgtattgcgtcttcgattcggt

SEQ ID NO: 330 - DNA, 504 bp, BACILLUS SP.
cgcggtatgctgttccttgatctgatccaggaaggaaacatgggtctgatgaaagccgttgaaaaatttgattatcgcaaaggttataaattcagtacgtatgctacgtggtggatcagacaggcgattacacgtgccattgccgatcaggcgagaacgatccggattcccgttcatatggttgaaaccattaataaattaatccgtgtgcagcgtcaattactgcaagacttaggcagagaaccaacacctgaagaaattgcggaagatatggatttaacgcctgaaaaagtacgcgaaatcttaaagattgctcaagagccggtatctctggaaacaccgatcggtgaagaggatgactcgcaccttggtgatttcattgaagaccaagaagcaacttcaccttctgaccacgccgcatacgagctattgaaagagcagctggaagatgtgcttgatacgttaactgatcgtgaagaaaatgtattgcgtcttcgattcggt

SEQ ID NO: 331 - DNA, 504 bp, BACILLUS SP.
cgcggaatgctgtttcttgacctgatccaggaaggcaacatgggtctgatgaaagccgttgaaaaatttgattatcgcaaaggttataaattcagtacgtatgctacgtggtggatcagacaggcgattactcgcgccattgccgatcaggcgagaacgatccggattcccgttcatatggttgaaaccattaataaattaatccgtgtgcagcgtcaactactgcaagatttaggcagagaaccgacacctgaagaaattgcggaagatatggatttaacacctgaaaaagtacgcgaaatcttaaagattgcacaagagccggtatcgctggaaacaccgatcggtgaagaggatgattcgcatcttggtgatttcattgaagaccaggaagccacttcaccttcagaccacgccgcatatgagctattgaaagagcaactggaagatgtacttgatacgttaactgatcgtgaagaaaatgtattgcgtcttcgattcgga

SEQ ID NO: 332 - DNA, 504 bp, BACILLUS SP.
cgcggaatgctgttccttgacctgatccaggaaggtaacatgggtctgatgaaagccgttgaaaaatttgattaccgcaaaggttataaattcagtacgtatgctacgtggtggatcagacaggcaattactcgcgccattgccgatcaggcgagaacgatccggattcccgttcatatggttgaaaccattaataaattaatccgtgtgcagcgtcaattactgcaagatttaggcagagaaccaacacctgaagaaattgcggaagatatggatttaacacctgaaaaagtacgcgaaatcttaaagattgcacaagagccggtatcgctggaaacaccgatcggtgaagaggatgattcgcatcttggtgatttcattgaagaccaggaagcaacttcaccttcagaccacgccgcatatgagctattgaaagagcagctggaagatgtacttgatacgttaactgatcgtgaagaaaatgtattgcgtcttcgattcggt

SEQ ID NO: 333 - DNA, 504 bp, BACILLUS SP.
cgcggaatgctgttccttgacctgattcaggaaggcaacatgggtctgatgaaagccgttgaaaaatttgattaccgcaaaggttataaattcagtacgtatgctacgtggtggatcagacaggcgattactcgcgccattgccgatcaggcgagaacgatccggattcccgttcatatggttgaaaccattaataaattaatccgtgtgcaacgtcaattactgcaagatttaggcagagaaccaacacctgaagaaattgcggaagatatggatttaacacctgaaaaagtacgcgaaatcttaaagattgcacaagagccggtatcgctggaaacaccgattggtgaagaggatgactcgcatcttggtgatttcattgaagaccaagaagcaacttcaccttcagaccacgccgcatatgagctattgaaagagcagctggaagatgtgcttgatacgttaactgatcgtgaagaaaatgtattgcgtcttcgattcggt

SEQ ID NO: 334 - DNA, 504 bp, BACILLUS SP.
cgcggaatgctgttccttgacctgattcaggaaggcaacatgggtctgatgaaagccgttgaaaaatttgattaccgcaaaggttataaattcagtacgtatgctacgtggtggatcagacaggcgattactcgcgccattgccgatcaggcgagaacgatccggattcccgttcatatggttgagaccattaataaattaatccgtgtgcagcgtcaattactgcaagatttaggcagagaaccaacacccgaagaaattgcggaagatatggatttaacacctgaaaaagtacgcgaaatcttaaagattgcacaagagccggtatcgctggaaacaccgattggtgaagaggatgactcgcatcttggtgatttcattgaagatcaagaagcaacttcaccttcagaccacgccgcatatgagctattgaaagagcagctggaagatgtgcttgatacgttaactgatcgtgaagaaaatgtattgcgtcttcgattcggt

SEQ ID NO: 335 - DNA, 504 bp, BACILLUS SP.
cgcggaatgcttttccttgacttaattcaggaaggcaatatgggtctgatgaaagccgttgaaaaatttgattaccgcaaaggttataaattcagtacgtatgctacgtggtggatcagacaggcgattacacgcgccattgccgaccaggcgagaacgatccggattcccgttcatatggttgaaaccattaataaattaatccgtgtgcagcgtcaattactgcaagatttaggcagagaaccaacacctgaagaaattgcggaagatatggatttaacacctgaaaaagtacgcgaaatcttaaagatcgcacaagagccggtatcgctggaaacaccgatcggtgaagaggatgactcacatcttggtgattttattgaagaccaagaagcaacttcaccttcagaccatgccgcatatgagctgttgaaagagcagctggaagatgtgcttgatacgttaactgatcgtgaagaaaatgtattgcgtcttcgattcggt

SEQ ID NO: 336 - DNA, 504 bp, BACILLUS SP.
cgcggaatgctgttccttgatctgatccaagaaggtaacatgggtctgatgaaagccgttgagaaatttgattatcgtaagggttataaattcagtacgtatgctacgtggtggatcagacaggcgattacacgcgccattgccgatcaggcgagaacgatccggatccccgttcatatggttgaaaccattaataaattaatccgcgtacagcgtcagctattgcaagacttaggcagagagccaacacctgaagaaattgcggaagacatggatctaacacctgaaaaagtacgtgaaatcttaaaaatagcacaagagccagtgtctcttgaaacaccgatcggtgaagaggatgattcccatcttggtgatttcatcgaagatcaagaggctacttcaccttcagatcatgcggcatacgagctattgaaagagcagcttgaagacgtgcttgatacgttaacagatcgtgaagaaaatgtattgcggcttcgattcggt

SEQ ID NO: 337 - DNA, 504 bp, BACILLUS SP.
cgcggaatgcttttccttgatctgatccaagaaggcaatatgggtctgatgaaagccgttgaaaagttcgactatcgtaaaggatataaattcagtacgtatgctacgtggtggatcaggcaggcaattacacgcgctatcgccgatcaggcaagaacgattcgtatccctgttcatatggttgaaaccatcaataaattaatccgtgtgcagcgccagctcctgcaagacttaggaagagagccgacaccggaagaaatcgcagaagacatggatcttacacctgaaaaagtgcgtgaaatcttaaaaatagctcaagagccggtatctttagagacaccgatcggtgaggaagatgactctcatcttggcgatttcatcgaagatcaggaggcgacttcaccttcagatcatgcggcgtatgagcttttaaaagaacagctggaagatgtgctggatacgttaacggatcgtgaagaaaatgtattgcgccttcgtttcggg

SEQ ID NO: 338 - DNA, 504 bp, BACILLUS SP.
cgcggaatgcttttccttgatctgatccaagaaggcaatatgggtctgatgaaagccgttgaaaagttcgactatcgtaaaggatataaattcagtacgtatgctacgtggtggatcaggcaggcaattacacgcgctatcgccgatcaggcaagaacgattcgtatccctgttcatatggttgaaaccatcaataaattaatccgagtacagcgccagctcctgcaagacttaggaagagagccgacaccggaagaaatcgcagaagacatggatcttacacctgaaaaagtgcgtgaaatcttaaaaatagctcaagagccggtatctttagagacaccgatcggtgaggaagatgactctcatcttggcgatttcatcgaagatcaggaggcgacttcaccttcagatcatgcggcgtatgagcttttaaaagaacagctggaagatgtgctggatacgttaacggatcgtgaagaaaatgtattgcgccttcgtttcggg

SEQ ID NO: 339 - DNA, 504 bp, BACILLUS SP.
cgcggaatgcttttccttgatctgatccaagaaggcaatatgggtctgatgaaagccgttgaaaagttcgactatcgtaaaggatataaattcagtacgtatgctacgtggtggatcaggcaggcaattacacgcgctatcgccgatcaggcaagaacgattcgtatccctgttcatatggttgaaaccatcaataaattaatccgtgtgcagcgccagctcctgcaagacttaggaagagagccgacaccggaagaaatcgcagaagacatggatcttacacctgaaaaagtgcgtgaaatcttaaaaatagctcaagagccggtatctttagagacaccgatcggtgaggaagatgactctcatcttggcgatttcatcgaagatcaggaggctacttcaccttcagatcatgcggcgtatgagcttttaaaagaacagctggaagatgtgctggatacgttaacggatcgtgaagaaaatgtattgcgccttcgtttcggg

SEQ ID NO: 340 - DNA, 504 bp, BACILLUS SP.
cgcggcatgcttttcttagacttgatccaagaaggaaacatgggtctcatgaaagccgtcgagaagtttgactaccgtaaaggatataaattcagtacgtatgcgacttggtggatcagacaggcgattacacgcgccatcgctgaccaggcgagaacaatccgtatcccggttcacatggtggaaaccatcaacaaattaatccgggtgcagcgtcagcttcttcaggatcttggcagagagccgacaccggaagagattgctgaagacatggatcttacgcctgaaaaagtccgtgaaatattaaaaattgctcaagagcctgtttctttagaaacgccgatcggtgaagaggacgactcacatctcggtgatttcatcgaagaccaggaagcgacatctccgtcggatcatgcggcatatgagcttttaaaagaacagctggaagatgtgctggatacgttgacagatcgtgaagaaaacgtgctgcgtctccgtttcggt

SEQ ID NO: 341 - DNA, 504 bp, BACILLUS SP.
cgcggaatgcttttccttgaccttattcaggaaggaaacatgggcctgatgaaggcggtggaaaagtttgactaccgcaaaggatataagttcagcacatacgcgacatggtggatcagacaggcgattacaagggcgattgccgaccaggcgagaacgatcaggattcctgttcatatggttgaaaccatcaacaagctgattcgtgttcaaaggcagctccttcaggatctcggccgcgagccaacgcctgaagagattgcggaagatatggatctgacgcctgagaaagtccgtgaaatcctaaagatcgcccaggaaccggtttcactcgagactccgatcggtgaagaggacgattcacatctgggagacttcattgaggatcaggaagcgacttctccatcagatcatgcagcatatgaattgctgaaagaacagcttgaagatgtgctggatacattgacagaccgtgaagaaaatgtgctccgccttcgcttcggc

SEQ ID NO: 342 - DNA, 504 bp, BACILLUS SP.
cgcggaatgcttttccttgaccttattcaggaaggaaacatgggcctgatgaaggcggtagaaaagtttgactaccgcaaaggatacaaattcagcacatacgcgacatggtggatcagacaggcgattacaagagcgattgccgaccaggcgagaacgatcaggattcctgttcatatggttgaaaccatcaacaagctgattcgtgttcaaaggcagctccttcaggatctcggccgcgaaccaacgcctgaagagattgcggaagatatggatctgacgcctgaaaaagtccgcgaaatcctaaagatcgcccaggaaccggtttcactcgagactccgatcggtgaagaggacgattcacatctgggagacttcattgaggatcaggaagcgacttctccgtcagatcatgcagcatatgaattgctgaaagaacagcttgaagatgtactggatacattgacagaccgtgaagaaaatgtgctccgccttcgcttcggc

SEQ ID NO: 343 - DNA, 504 bp, BACILLUS SP.
cgcggaatgcttttccttgaccttattcaggaaggaaacatgggcctgatgaaggcggtagaaaagtttgactaccgcaaaggatacaaattcagcacatacgcgacatggtggatcagacaggcgattacaagagcgattgccgaccaggcgagaacgatcaggattcctgttcatatggttgaaaccatcaacaaactgattcgtgttcaaaggcagctccttcaggatctcggccgcgagccaacgcctgaagagattgcggaagatatggatctgacgcctgaaaaagtccgtgaaattctaaagatcgcccaggaaccggtttcactcgagactccgatcggtgaagaggacgattcacatctgggagacttcattgaggatcaggaagcgacttctccgtcagatcatgcagcatatgaattgctgaaagaacagcttgaagatgtactggatacattgacagaccgtgaagaaaatgtgctccgccttcgcttcggc

SEQ ID NO: 344 - DNA, 504 bp, BACILLUS SP.
cgcggcatgcttttcctagacctgatccaggaaggaaacatgggtctgatgaaggcggttgaaaagtttgactaccgtaaaggatacaaattcagtacgtatgcaacatggtggatcagacaggcgattacaagagcaatagccgaccaggcaagaacgatccggattcctgtgcatatggtggaaacgatcaataagctgatccgcgtccaaagacagctccttcaggatctcggccgggagccaactcctgaagaaattgcagaagatatggatttgacgccggaaaaagtccgtgaaattctaaagattgcccaagaaccggtttcacttgaaaccccgataggagaagaggatgattcacatcttggagatttcatcgaagaccaggaagcgacttctccgtcagatcacgcggcatatgaacttctgaaagaacagcttgaagatgtattagatacactgacagaccgcgaagaaaatgtattgcgccttcgtttcggc

SEQ ID NO: 345 - DNA, 504 bp, BACILLUS SP.
cgcggaatgcttttccttgatttaattcaggaaggcaacatgggtctgatgaaagccgttgaaaaatttgattaccgcaaaggttataaattcagtacgtatgctacgtggtggatcagacaggcgattacacgcgccattgccgaccaggcgagaacgatccggattcccgttcatatggttgaaaccattaataaattaatccgtgtgcagcgtcaattactgcaagatttaggcagagaaccaacacctgaagaaattgcggaagatatggatttaacacctgaaaaagtacgcgaaatcttaaagatcgcacaagagccggtatcgctggaaacaccgatcggtgaagaggatgactcacatcttggtgattttattgaagaccaagaagcaacttcaccttcagaccatgccgcatatgagctgttgaaagagcagctggaagatgtgcttgatacgttaactgatcgtgaagaaaatgtattgcgtcttcgattcggt

SEQ ID NO: 346 - DNA, 504 bp, BACILLUS SP.
cgcggcatgcttttcttagacttaatccaagaaggaaacatgggtctcatgaaagccgtcgaaaagtttgactaccgtaaaggatataaattcagtacgtatgcaacttggtggatcagacaggcgattacacgcgccatcgctgaccaggcgagaacgattcgtatccctgttcacatggtggaaaccatcaacaaattaatccgtgtgcagcgtcagcttcttcaggatctgggcagagagccgacaccggaagagattgctgaagacatggatcttacgcctgaaaaagtccgtgaaatattaaaaattgcccaagagcctgtttctttagaaacgccgatcggtgaggaagacgattcacatctcggtgatttcatcgaagaccaggaagcgacgtctccgtcggatcatgcggcatatgagcttttgaaagaacagctggaagacgtgctggatacgttgacggatcgtgaagaaaacgtgctgcgtctccgtttcggt

SEQ ID NO: 347 - DNA, 504 bp, BACILLUS SP.
cgcggcatgcttttcttagacttaatccaagaaggaaacatgggtcttatgaaagccgtcgaaaagtttgactaccgtaaaggatataaattcagtacgtatgcaacttggtggatcagacaggcgattacacgcgccatcgctgaccaggcgagaacgatccgtatccctgttcacatggtggaaaccatcaacaaattaatccgtgtgcagcgtcagcttcttcaggatctgggcagagagccgacaccggaagagattgctgaagacatggatcttacgcctgaaaaagtccgtgaaatattaaaaattgcccaagagcctgtttctttagaaacgccgatcggtgaggaagacgactcacatctcggtgatttcatcgaagaccaggaagcgacgtctccgtcggatcatgcggcatatgagcttttgaaagaacagctggaagacgtgctggatacgttgacggatcgtgaagaaaacgtgctgcgtctccgtttcggt

SEQ ID NO: 348 - DNA, 504 bp, BACILLUS SP.
cgcggaatgctgttccttgacctgatccaagaaggtaacatgggtctgatgaaagccgttgagaaatttgattaccgtaagggttataaattcagtacgtatgctacgtggtggatcagacaggcgattacacgcgccattgccgatcaggcgagaacgatccggatccccgttcatatggttgaaaccattaataaattaatccgcgtacagcgtcagctattgcaagacttaggcagagagccaacacctgaagaaattgcggaagacatggatttaacacctgaaaaagtacgtgaaatcttaaaaatagcacaagagccagtgtctcttgaaacaccgatcggtgaagaggatgattcccatcttggtgatttcatcgaagatcaagaggctacttcaccttcagatcacgcggcatacgagctattgaaagagcagcttgaagacgtgcttgatacgttaacagatcgtgaagaaaatgtattgcggcttcgattcggt

SEQ ID NO: 349 - DNA, 504 bp, BACILLUS SP.
cgcggaatgctgttccttgatctgattcaagaaggtaacatgggtctgatgaaagccgttgagaaatttgattatcgtaagggttataaattcagtacgtatgctacgtggtggatcagacaggcgattacacgcgccattgccgatcaggcgagaacgatccggatccccgttcatatggttgaaaccattaataaattaatccgcgtacagcgtcagctattgcaagacttaggcagagagccaacacctgaagaaattgcggaagacatggatctaacacctgaaaaagtacgtgaaatcttaaaaatagcacaagagccagtgtctcttgaaacaccgatcggtgaagaggatgattcccatcttggtgatttcatcgaagatcaagaggctacttcaccttcagatcatgcggcatacgagctattgaaagagcagcttgaagacgtgcttgatacgttaacagatcgtgaagaaaatgtattgcggcttcgattcggt

SEQ ID NO: 350 - DNA, 504 bp, BACILLUS SP.
cgcggaatgctgttcctcgacctgatccaagaaggtaacatgggtctgatgaaagccgttgagaaatttgattaccgtaagggttataaattcagtacgtatgctacgtggtggatcagacaggcgattacacgcgccattgccgatcaggcgagaacgatccggatccccgttcatatggttgaaaccattaataaattaatccgcgtacagcgtcagctattgcaagacttaggcagagagccaacacctgaagaaattgcggaagacatggatttaacacctgaaaaagtacgtgaaatcttaaaaatagcacaagagccagtgtcccttgaaacaccgatcggtgaagaggatgattcccatcttggtgatttcatcgaagatcaagaggctacttcaccttcagatcacgcggcatacgagctattgaaagagcagcttgaagacgtgcttgatacgttaacagatcgtgaagaaaatgtattgcggcttcgattcggt

SEQ ID NO: 351 - DNA, 504 bp, BACILLUS SP.
cgcggcatgcttttcctagacctgatccaggaaggaaacatgggtctgatgaaggcggttgaaaagtttgactaccgtaaaggatacaaattcagtacgtatgcaacatggtggatcagacaggcgattacaagagcaatagccgaccaggcaagaacgatccggattcctgtgcatatggtggaaacgatcaataagctgatccgcgtccaaagacagctccttcaggatctcggccgggagccaactcctgaagaaattgctgaagatatggatttgacgccggaaaaagtccgtgaaattctaaagattgcccaagaaccggtttcacttgaaaccccgataggagaagaggatgattcacatcttggagatttcatcgaagaccaggaagcgacttctccgtcagatcacgcggcatatgaacttctgaaagaacagcttgaagatgtattagatacactgacagaccgcgaagaaaatgtattgcgccttcgtttcggc

SEQ ID NO: 352 - DNA, 504 bp, BACILLUS SP.
cgcggcatgcttttcttagacttgatccaagaaggaaacatgggtctcatgaaagccgtcgagaagtttgactaccgtaaaggatataaattcagtacgtatgcgacttggtggatcagacaggcgattacacgcgccatcgctgaccaggcgagaacaatccgtatcccggttcacatggtggaaaccatcaacaaattaatccgggtgcagcgtcagcttcttcaggatcttggcagagagccgacaccggaagagattgctgaagacatggatcttacacctgaaaaagtccgtgaaatattaaaaattgctcaagagcctgtttctttagaaacgccgatcggtgaagaagacgactcacatctcggtgacttcatcgaagaccaggaagcgacatctccgtcggatcatgcggcatatgagcttttaaaagaacagctggaagatgtgctggatacgttgacagatcgtgaagaaaacgtgctgcgtctccgtttcggt

SEQ ID NO: 353 - DNA, 504 bp, BACILLUS SP.
cgcggcatgcttttcttagacttgatccaagaaggaaacatgggtctcatgaaagcagtcgagaagtttgactaccgtaaaggatataaattcagtacgtatgcgacttggtggatcagacaggcgattacacgcgccatcgctgaccaggcgagaacaatccgtatcccggttcacatggtggaaaccatcaacaaattaatccgggtgcagcgtcagcttcttcaggatcttggcagagagccgacaccggaagagattgctgaagacatggatcttacacctgaaaaagtccgtgaaatattaaaaattgctcaagagcctgtttctttagaaacgccgatcggtgaagaagacgactcacatctcggtgacttcatcgaagaccaggaagcgacatctccgtcggatcatgcggcatatgagcttttaaaagaacagctggaagatgtgctggatacgttgacagatcgtgaagaaaacgtgctgcgtctccgtttcggt

SEQ ID NO: 354 - DNA, 504 bp, BACILLUS SP.
cgcggaatgcttttccttgaccttattcaggaaggaaacatgggcctgatgaaggcggtagaaaagtttgactaccgcaaaggatacaaattcagcacatacgcgacatggtggatcagacaggcgattacaagggcgattgccgaccaggcgagaacgatcaggattcctgttcatatggttgaaaccatcaacaagctgattcgtgttcaaaggcagctccttcaggatctcggccgcgaaccaacgcctgaagagattgcggaagatatggatctgacgcctgaaaaagtccgtgaaatcctaaagatcgctcaggaaccggtttcacttgaaactccgatcggtgaagaggacgattcacatctgggagactttattgaggatcaggaagcgacttctccgtcagatcatgcagcatatgaattgctgaaagaacagcttgaagatgtactggatacattgacagaccgtgaagaaaatgtgctccgccttcgcttcggc

SEQ ID NO: 355 - DNA, 504 bp, BACILLUS SP.
cgcggaatgcttttccttgaccttattcaggaaggaaacatgggcctgatgaaggcggtagaaaagtttgactaccgcaaaggatacaaattcagcacatacgcgacatggtggatcagacaggcgattacaagggcgattgccgaccaggcgagaacgatcaggattcctgttcatatggttgaaaccatcaacaagctgattcgtgttcaaaggcagctccttcaggatctcggccgcgaaccaacgcctgaagagattgcggaagatatggacctgacgcctgaaaaagtccgtgaaatcctaaagatcgctcaggaaccggtttcactcgaaactccgatcggtgaagaggacgattcacatctgggagactttattgaggatcaggaagcgacttctccgtcagatcatgcagcatatgaattgctgaaagaacagcttgaagatgtactggatacattgacagaccgtgaagaaaatgtgctccgccttcgcttcggc

SEQ ID NO: 356 - DNA, 540 bp, BACILLUS SP.
gcagcagacaaagctgaagctgttgtttgcgcgccagcgcttttcttagaaaagcttgcttctgctgtgaaaggcactgacctaaaagtcggcgctcaaaacatgcacttcgaagaaagcggcgcgttcacaggcgaaatcagcccggttgctctgaaagaccttggcgttgactactgcgtcatcggccactctgagcgccgtgaaatgttcgctgaaactgatgaaacagttaacaaaaaagcacacgcagctttcaaacacggcattgtgccgatcatctgtgtaggtgaaacgcttgaagagcgtgaagccggcaaaacaaatgatcttgttgctgaccaagtgaaaaaaggccttgctggtctttctgaagaacaagttgctgcttctgttattgcgtatgagccaatctgggcaatcggaacaggcaaatcttctacagcgaaagatgcgaatgacgtgtgtgcgcatatccgtaaaaccgtcgctgaaagcttcagccaagaagctgcagacaagcttcgcatccaatatggc

SEQ ID NO: 357 - DNA, 540 bp, BACILLUS SP.
gcagcagacaaagctgaagctgttgtttgcgcgccagcgcttttcttagaaaagcttgcttctgctgtgaaaggcactgacctaaaagtcggcgctcaaaacatgcacttcgaagaaagcggcgcgttcacaggcgaaatcagcccggttgctctgaaagaccttggcgttgactactgcgtcattggccactctgagcgccgtgaaatgttcgctgaaactgatgaaacagttaacaaaaaagcacacgcagctttcaaacacggcattgtgccgatcatctgtgtaggtgaaacgcttgaagagcgtgaagccggcaaaacaaatgatcttgttgctgaccaagtgaaaaaaggccttgctggtctttctgaagaacaagttgctgcttctgttattgcgtatgagccaatctgggcaatcggaacaggcaaatcttctacagcgaaagatgcgaatgacgtgtgtgcgcatatccgtaaaaccgtcgctgaaagcttcagccaagaagctgcagacaagcttcgcatccaatatggc

SEQ ID NO: 358 - DNA, 540 bp, BACILLUS SP.
gcagcagacaaagctgaagctgttgtttgcgcgccagcgcttttcttagaaaagcttgcttctgctgttaaaggcactgacctaaaagtcggcgctcaaaacatgcacttcgaagaaagcggcgcgttcacaggcgaaatcagcccggttgctctgaaagaccttggcgttgactactgcgtcatcggccactctgagcgccgtgaaatgttcgctgaaactgatgaaacagttaacaaaaaagcacacgcagctttcaaacacggcattgtgccgatcatctgtgtaggtgaaacgcttgaagagcgtgaagccggcaaaacaaatgatcttgttgctgaccaagtgaaaaaaggccttgctggtctttctgaagaacaagttgctgcttctgttattgcgtatgagccaatctgggcaatcggaacaggcaaatcttctacagcgaaagatgcgaatgacgtgtgtgcgcatatccgtaaaaccgtcgctgaaagcttcagccaagaagctgcagacaagcttcgcatccaatatggc

SEQ ID NO: 359 - DNA, 540 bp, BACILLUS SP.
gcagcagacaaagctgaagctgttgtttgcgcgccagcgcttttcttagaaaagcttgcttctgctgtgaaaggcactgacctaaaagtcggcgctcaaaacatgcacttcgaagaaagcggcgcgttcacaggcgaaatcagcccggttgctctgaaagaccttggcgttgactactgcgtcatcggccactctgagcgccgtgaaatgttcgctgaaactgatgaaacagttaacaaaaaagcacatgcagctttcaaacacggcattgtgccgatcatctgtgtaggtgaaacgcttgaagagcgtgaagccggcaaaacaaatgatcttgttgctgaccaagtgaaaaaaggccttgctggtctttctgaagaacaagttgctgcttctgttattgcgtatgagccaatctgggcaatcggaacaggcaaatcttctacagcgaaagatgcgaatgacgtgtgtgcgcatatccgtaaaaccgtcgctgaaagcttcagccaagaagctgcagacaagcttcgcatccaatatggc

SEQ ID NO: 360 - DNA, 540 bp, BACILLUS SP.
gcagcagacaaagcggaagctgttgtttgcgcgccagcacttttcttagaaaagcttacttctgctgttaaaggcactgatctaaaagtcggcgcacaaaacatgcacttcgaagaaagcggcgcgttcacaggcgaaatcagcccggttgctcttaaagaccttggcgttgactactgcgtcattggccactctgagcgccgtgaaatgttcgctgaaactgatgaaacagttaacaaaaaagcacatgctgctttcaaacacggcattgtgccgatcatctgtgtaggtgaaacgcttgaagagcgcgaagccggtaaaacaaatgatcttgttgctgaccaagtaaaaaaaggccttgctggtctttctgaagaacaagttgctgcttctgttattgcgtatgagccaatctgggcaatcggaacaggcaaatcttctacagcaaaagatgcgaacgacgtgtgtgcgcatatccgtaaaaccgtcgctgaaagcttcagccaagaagttgcagacaagcttcgcatccaatatggc

SEQ ID NO: 361 - DNA, 540 bp, BACILLUS SP.
gcagcagacaaagcggaagctgttgtttgcgcgccagcacttttcttagaaaagcttacttctgctgtgaaaggcactgaccttaaagtcggcgcacaaaacatgcacttcgaagaaagcggcgcgttcacaggcgaaatcagtccggttgctcttaacgaccttggcgttgactactgcgtcatcggccactctgagcgccgtgaaatgttcgccgaaacggatgaaacagtcaataaaaaagcacatgctgctttcaaacacggcattgtgccgatcatctgtgtaggtgaaacgcttgaagagcgcgaagccggtaaaacaaatgatcttgttgctgatcaagtaaaaaaaggccttgctggtctttctgaagaacaagttgctgcttctgttattgcgtatgagccaatctgggcaatcggaacaggcaaatcttctacagcaaaagatgcgaacgacgtgtgtgcgcatatccgtaaaaccgtcgctgaaagcttcagtcaagaagttgcagacaagcttcgcattcaatatggc

SEQ ID NO: 362 - DNA, 540 bp, BACILLUS SP.
gcagcagacaaagcggaagctgttgtttgcgcgccagcacttttcttagaaaagcttacttctgctgttaaaggcactgacctaaaagtcggcgcacaaaacatgcacttcgaagaaagcggcgcgttcacaggcgaaatcagcccggttgctcttaacgaccttggcgttgactactgcgtcatcggccactctgagcgccgtgaaatgttcgctgaaactgatgaaacagttaacaaaaaagcacacgctgctttcaaacacggcattgtgccgatcatctgtgtaggtgaaacgcttgaagagcgcgaagccggcaaaacaaatgatcttgttgctgaccaagtgaaaaaaggccttgctggtctttctgaagaacaagttgctgcttctgttattgcgtatgagccaatctgggcaatcggaacaggcaaatcttctacagcaaaagatgcgaacgacgtgtgtgcgcatatccgtaaaaccgtcgctgaaagcttcagccaagaagttgcagacaagcttcgcatccaatatggc

SEQ ID NO: 363 - DNA, 540 bp, BACILLUS SP.
gcagcagacaaagcggaagctgttgtttgcgcgccagcacttttcttagaaaagcttacttctgctgttaaaggcactgacctaaaagtcggcgctcaaaacatgcacttcgaagaaagcggcgcgttcacaggcgaaatcagcccggttgcactgaaagaccttggcgttgactactgcgtcatcggccactctgagcgccgtgaaatgttcgctgaaactgatgaaacagttaacaaaaaagcacatgctgctttcaaacacggcatcgtgccgatcatctgtgtgggtgaaacgcttgaagagcgcgaagccggtaaaacaaatgatcttgttgctgaccaagtgaaaaaaggccttgctggtctttctgaagaacaagttgctgcttctgttattgcgtatgagccaatctgggcaatcggaacaggcaaatcttctacagcgaaagatgcgaacgacgtgtgtgcgcatatccgtaaaaccgtcgctgaaagcttcagccaagaaactgcagacaagcttcgcatccaatatggc

SEQ ID NO: 364 - DNA, 540 bp, BACILLUS SP.
gcagcagacaaagcggaagctgttgtttgcgcgccagcacttttcttagaaaagcttacttctgctgttaaaggcactgacctaaaagtcggcgctcaaaacatgcacttcgaagaaagcggcgcgttcacaggcgaaatcagcccggttgcactgaaagaccttggcgttgactactgcgtcatcggccactctgagcgccgtgaaatgttcgctgaaactgatgaaacagttaacaaaaaagcacatgctgctttcaaacacggcattgtgccgatcatctgtgtgggtgaaacgcttgaagagcgcgaagccggcaaaacaaatgatcttgttgctgaccaagtgaaaaaaggccttgctggtctttctgaagaacaagttgctgcttctgttattgcgtatgagccaatctgggcaatcggaacaggcaaatcttctacagcgaaagatgcgaacgacgtgtgtgcgcatatccgtaaaaccgtcgctgaaagcttcagccaagaaactgcagacaagcttcgcatccaatatggc

SEQ ID NO: 365 - DNA, 540 bp, BACILLUS SP.
gcagcagacaaagcggaagctgttgtttgcgcgccagcgcttttcttagaaaagctgacttctgctgtgaaaggcactgaccttaaagtcggcgctcaaaacatgcactttgaagaaagcggcgcgttcacaggcgaaatcagcccggttgctctgaaagaccttggcgttgactactgcgtcatcggccactctgagcgccgtgaaatgttcgctgaaactgatgaaacagttaacaaaaaagcacatgctgctttcaaacacggtattgtgccgatcatctgtgttggtgaaacgcttgaagagcgtgaagccggcaaaacaaatgatcttgtggctgaccaagtgaaaaaaggcctagcgggtctttctgaagaacaagttgcagcttctgttattgcttatgagccaatctgggcaatcggaacaggcaaatcttctacatctaaagatgcgaacgacgtgtgtgcgcatatccgtaaaaccgtcgctgaaagcttcggccaagaagcggcagacaagcttcgcattcaatatggc

SEQ ID NO: 366 - DNA, 540 bp, BACILLUS SP.
gcagcagacaaagcggaagccgttgtttgcgcaccagctcttttcttagaaaagctgacttctgctgttaaaggaactgacctgaaagtcggcgcccaaaacatgcattttgaagaaaacggtgcgttcacaggtgaaatcagcccggtcgctctgaaagaccttggcgtggattactgcgtcatcggccactctgagcgccgtgaaatgttcgctgaaactgatgaaacagttaacaaaaaagcgcatgccgctttcaagcacggcattgtgccgatcatttgcgtaggtgaaacgcttgaagagcgcgaagccggaaaaacaaatgatcttgttgccgatcaagtgaaaaaaggtcttgcgggtctttctgaagagcaagttgccgcttccgttattgcgtacgagccaatctgggcaatcggaacaggcaaatcttctacagcgaaagatgctaacgacgtctgcgcgcatatccgtaaagtcgttgctgaaagcttcagccaggaaactgctgacaagctccgcattcaatacggc

SEQ ID NO: 367 - DNA, 540 bp, BACILLUS SP.
gcagcagacaaagctgaagctgtagtttgcgcaccagcgcttttcttagaaaaactggctacagctgtgaaaggcactgaccttaaagtcggcgcccaaaacatgcactttgaagaaaacggcgcgttcacaggcgaaatcagcccggtagcattgaaagaccttggcgttgaatactgcgtcatcggccactcagagcgccgcgaaatgttcgctgaaacggacgaaacggttaacaaaaaagcacacgctgctttcaaacacggcattgtgccgattatctgtgtaggcgaaacgcttgaagagcgtgaagcaggcaaaacaaacgatcttgttgcagatcaagtgaaaaaaggccttgccggactttctcaagatcaagtcgcagaatctgttattgcttacgagcctatttgggcaatcggaacaggcaaatcttctacatctcaagatgcaaatgacgtgtgcgcgcacatccgtcaaacagttgctggcgaattcggccaagaaactgctgacaaactgcgcattcaatacggc

SEQ ID NO: 368 - DNA, 540 bp, BACILLUS SP.
gcagcagacaaagctgaagctgtagtttgcgcaccagcgcttttcttagaaaaactggctacagctgtgaaaggcactgatcttaaagtcggcgcccaaaacatgcactttgaagaaaacggcgcgttcacaggcgaaatcagcccggtagcattgaaagaccttggcgttgaatactgcgtcatcggccactcagagcgccgcgaaatgttcgctgaaacggacgaaacggttaacaaaaaagcacacgctgctttcaaacacggcattgtgccgattatctgtgtaggcgaaacgcttgaagagcgtgaagcaggcaaaacaaacgatcttgttgcagatcaagtgaaaaaaggccttgccggactttctcaagatcaagtcgcagaatctgttattgcttacgagcctatttgggcaatcggaacaggcaaatcttctacatctcaagatgcaaatgacgtgtgcgcgcacatccgtcaaacagttgctggcgaattcggccaagaaactgctgacaaactgcgcattcaatacggc

SEQ ID NO: 369 - DNA, 540 bp, BACILLUS SP.
gcagcagataaagcagaagctgttgtctgtgcgcctgcactgttccttgaaaagcttgcatcagctgttaaaggtacagatctgaaagtcggcgcgcaaaacatgcactttgaagaaagcggtgctttcacgggtgaaatcagccctgcggctttgaaagacctcggagttgaatactgcgtcatcggccactccgagcgccgcgaaatgtttgctgaaacggatgaaacggtcaacaaaaaagcgcatgctgcattcaaatatggcatcgtgccgatcatctgcgtaggtgaaacgcttgaagagcgcgaagcaaacaaaacaaacgagcttgttgcagaccaagtgaaaaaagcacttgcaggtttaacaactgaacaggtagctgcttccgtgatcgcttatgagccgatctgggcgattggaaccggcaaatcttcaactgcacaagatgcaaacgaagtatgcgcgcacatccgcaaaaccgttgcatcagaattcggacaagctgcagcagacagcgtccgcattcagtacggt

SEQ ID NO: 370 - DNA, 540 bp, BACILLUS SP.
gcagcagataaagcagaagctgttgtctgtgcgcctgcactgttccttgaaaagcttgcatcagctgttaaaggtacagatctgaaagtcggcgcgcaaaacatgcactttgaagaaagcggtgctttcacgggtgaaatcagccctgcggctttgaaagacctcggagttgaatactgcgtcatcggccactccgagcgccgcgaaatgtttgctgaaacggatgaaacggtcaacaaaaaagcgcatgctgcattcaaatatggcatcgtgccgatcatctgcgtaggtgaaacgcttgaagagcgcgaagcaaacaaaacaaacgagcttgttgcagaccaagtgaaaaaagcacttgcaggtttaacaactgaacaggtagctgcttccgtgatcgcttatgagccgatctgggcgattggaaccggcaaatcttcaactgcacaagatgcaaacgaagtatgcgcgcacatccgcaaaaccgttgcatcagaattcggacaagctgcagcagacagcgtccgcattcagtacggc

SEQ ID NO: 371 - DNA, 540 bp, BACILLUS SP.
gcagcagataaagcagaagctgttgtctgtgcgcctgcactgttccttgaaaagctcacatcagctgtaaaaggcacagatctgaaagtcggcgcgcaaaacatgcattttgaagaaagcggagctttcacgggtgaaatcagccctgcggctttgaaagaccttggagttgaatactgcgtcatcggccactccgagcgccgcgaaatgtttgctgaaacggatgaaacggtcaacaaaaaagcgcatgctgcatttaaatacggcatcgttccgatcatctgcgtaggtgaaacgcttgaagagcgcgaagcaaacaaaacaaacgagcttgttgcagaccaagtgaaaaaagcacttgcaggtttaacaactgaacaggtagctgcttccgtgatcgcttatgagccgatctgggcgattggaaccggcaagtcttcaactgcacaagatgcaaacgaagtatgcgcgcacatccgcaaaaccgtggcatcagagttcggacaaactgcagcagacagcgtccgcattcagtacggc

SEQ ID NO: 372 - DNA, 540 bp, BACILLUS SP.
gcagcagataaagcagaagctgttgtctgcgcgccagcgctgttccttgaaaagctgacttcagctgtcaaaggcactgatcttaaagtcggcgcgcaaaacatgcacttcgaagaaagcggcgctttcactggagaaatcagccctgccgctctgaaagatctaggggttgaatattgcgtgattggccactctgagcgccgcgaaatgtttgccgaaacggacgagaccgtaaacaaaaaagcgcatgccgctttcaaatacgggattgtgccgatcatctgcgtaggtgaaacgcttgaagaacgtgaagcaaacaaaacaaatgagcttgtagccgatcaggtgaaaaaagctcttgcaggcctgacagcagagcaagtatctgcttctgtcatcgcctatgagccaatctgggcgatcggaacaggcaaatcttcaacagctcaagatgcaaatgaagtgtgcgcacacatccgccaaactgtctccgctgaattcggacaagcggctgcggacagcatccgcatccagtacggc

SEQ ID NO: 373 - DNA, 540 bp, BACILLUS SP.
gcagcagataaagcagaagctgttgtctgcgcgccagcgctgttccttgaaaagctgacttcagctgtcaaaggcactgatcttaaagtcggcgcgcaaaacatgcactttgaagaaagcggcgctttcactggagaaatcagccctgccgctctgaaagatctaggggttgaatattgcgtgattggccactctgagcgccgcgaaatgtttgccgaaacggacgagaccgtaaacaaaaaagcgcatgccgctttcaaatacgggattgtgccgatcatctgcgtaggtgaaacgcttgaagaacgtgaagcaaacaaaacaaatgagcttgtagccgatcaggtgaaaaaagctcttgcaggcctgacagcagagcaagtatctgcttctgtcatcgcctatgagccaatctgggcgatcggaacaggcaaatcttcaacagctcaagatgcaaatgaagtgtgcgcacacatccgccaaactgtctccgctgaattcggacaagcggctgcggacagcatccgcatccagtacggc

SEQ ID NO: 374 - DNA, 540 bp, BACILLUS SP.
gcagcagacaaagcggaagccgttgtttgcgcaccagctcttttcttagaaaagctgacttctgctgttaaaggaactgacttaaaagtcggcgcccaaaacatgcattttgaagaaaacggtgcgttcacaggcgaaatcagcccggtcgctctgaaagaccttggcgtggattactgcgtcatcggccactctgagcgccgtgaaatgttcgctgaaactgatgaaacagttaacaaaaaagcgcatgccgctttcaaacacggcattgtgccgatcatttgcgtaggtgaaacgcttgaagagcgcgaagccggaaaaacaaatgatcttgttgccgatcaagtgaaaaaaggtcttgcgggtctttctgaagagcaagttgcagcttccgttattgcgtacgagccaatctgggcaatcggaacaggcaaatcttctacagcgaaagatgcaaacgacgtctgcgcgcatatccgcaaagttgttgctgaaagcttcagccaagaagctgctgacaagctccgcattcaatacggc

SEQ ID NO: 375 - DNA, 540 bp, BACILLUS SP.
gcagcagacaaagcggaagccgttgtttgcgcaccagctcttttcttagaaaagctgacttctgctgttaaaggaactgacttaaaagtcggcgcccaaaacatgcattttgaagaaaacggtgcgttcacaggcgaaatcagcccggtcgctctgaaagaccttggcgtggattactgcgtcatcggccactctgagcgccgtgaaatgttcgctgaaactgatgaaacagttaacaaaaaagcgcatgccgctttcaaacacggtattgtgccgatcatttgcgtaggtgaaacgcttgaagagcgcgaagccggaaaaacaaatgatcttgttgccgatcaagtgaaaaaaggtcttgcgggtctttctgaagagcaagttgccgcttctgttattgcgtacgagccaatctgggcaatcggaacaggcaaatcttctacagcgaaagatgcaaacgacgtctgcgcgcatatccgcaaagttgttgctgaaagcttcagccaagaagctgctgacaagctccgcattcaatacggc

SEQ ID NO: 376 - DNA, 540 bp, BACILLUS SP.
gcagcagacaaagcggaagctgttgtttgcgcgccagcacttttcttagaaaagcttacttctgctgttaaaggcactgacctaaaagtcggcgcacaaaacatgcacttcgaagaaagcggcgcgttcacaggcgaaatcagcccggttgctcttaacgaccttggcgttgactactgcgtcatcggccactctgagcgccgtgaaatgttcgctgaaactgatgaaacagttaacaaaaaagcacacgctgctttcaaacacggcattgtgccgatcatctgtgtaggtgaaacgcttgaagagcgcgaagccggcaaaacaaatgatcttgttgctgaccaagtgaaaaaaggccttgctggtctttctgaaaaacaagttgctgcttctgttattgcgtatgagccaatctgggcaatcggaacaggcaaatcttctacagcaaaagatgcgaacgacgtgtgtgcgcatatccgtaaaaccgtcgctgaaagcttcagccaagaaactgcagacaagcttcgcatccaatatggc

SEQ ID NO: 377 - DNA, 540 bp, BACILLUS SP.
gcagcagacaaagcggaagctgttgtttgcgcgccagcgcttttcctagaaaagctgacttctgctgttaaaggcactgaccttaaagtcggcgcccaaaacatgcacttcgaagaaagcggcgcgttcacaggcgaaatcagcccggttgctctgaaagaccttggcgttgactactgcgtcatcggccactctgagcgccgtgaaatgttcgctgaaactgatgaaacagttaacaaaaaagcacatgctgctttcaaacacggcattgtgccgatcatctgtgttggagaaacgcttgaagagcgcgaagccggcaaaacaaatgatcttgtggctgatcaagtgaaaaaaggccttgctggtctttctgaagaacaagttgcagcttctgttattgcttatgagccaatctgggcaatcggaacaggcaaatcttctacatctaaagatgcgaacgacgtgtgtgcgcatatccgtaaaaccgtcgctgaaagcttcggccaagaagcggcagataagcttcgcattcaatatggc

SEQ ID NO: 378 - DNA, 540 bp, BACILLUS SP.
gcagcagacaaagcggaagctgttgtttgcgcgccagcacttttcttagaaaagcttacttctgctgttaaaggcactgaccttaaagtcggcgcacaaaacatgcacttcgaagaaagcggcgcgttcacaggcgaaatcagtccggttgctcttaacgaccttggcgttgactactgcgtcatcggccactctgagcgccgtgaaatgttcgccgaaacggatgaaacagtcaacaaaaaagcacatgctgctttcaaacacggcattgtgccgatcatctgtgtaggtgaaacgcttgaagagcgcgaagccggtaaaacaaatgatcttgttgctgaccaagtgaaaaaaggacttgctggtctttctgaagaacaagttgctgcttctgttattgcgtatgagccaatctgggcaatcggaacaggcaaatcttctacagcaaaagatgcgaacgacgtgtgtgcgcatatccgtaaaaccgtcgctgaaagcttcagtcaagaagttgcagacaagcttcgcattcaatatggc

SEQ ID NO: 379 - DNA, 540 bp, BACILLUS SP.
gcagcagacaaagcggaagctgttgtttgcgcgccagcacttttcttagaaaagcttacttctgctgtgaaaggcactgaccttaaagtcggcgcacaaaacatgcacttcgaagaaagcggcgcgttcacaggcgaaatcagtccggttgctcttaacgaccttggcgttgactactgcgtcatcggccactctgagcgccgtgaaatgttcgccgaaacggatgaaacagtcaataaaaaagcacatgctgctttcaaacacggcattgtgccgatcatctgtgtaggtgaaacgcttgaagagcgcgaagccggtaaaacaaatgatcttgttgctgaccaagtaaaaaaaggccttgctggtctttctgaagaacaagttgctgcttctgttattgcgtatgagccaatctgggcaatcggaacaggcaaatcttctacagcaaaagatgcgaacgacgtgtgtgcgcatatccgtaaaaccgtcgctgaaagcttcagtcaagaagttgcagacaagcttcgcattcaatatggc

SEQ ID NO: 380 - DNA, 540 bp, BACILLUS SP.
gcagcagacaaagcggaagctgttgtttgcgcgccagcgcttttcctagaaaagctgacttctgctgttaaaggcactgaccttaaagtcggcgcccaaaacatgcacttcgaagaaagcggcgcgttcacaggcgaaatcagcccggttgctctgaaagaccttggcgttgactactgcgtcatcggccactctgagcgccgtgaaatgttcgctgaaactgatgaaacagttaacaaaaaagcacatgctgctttcaaacacggcattgtgccgatcatctgtgttggtgaaacgcttgaagagcgcgaagccggcaaaacaaatgatcttgtggctgaccaagtgaaaaaaggccttgctggtctttctgaagaacaagttgcagcttctgttattgcttatgagccaatctgggcaatcggaacaggcaaatcttctacatctaaagatgcgaacgacgtgtgtgcacatatccgtaaaaccgtcgctgaaagcttcggccaagaagcggcagacaagcttcgcattcaatatggc

SEQ ID NO: 381 - DNA, 540 bp, BACILLUS SP.
gcagcagacaaagcggaagccgttgtttgcgcaccagctcttttcttagaaaagctgacttctgctgttaaaggaactgacctgaaagtcggcgcccaaaacatgcattttgaagaaaacggtgcgttcacaggtgaaatcagcccggtcgctctgaaagaccttggcgtggattactgcgtcatcggccactctgagcgccgtgaaatgttcgctgaaactgatgaaacagttaacaaaaaagcgcatgccgctttcaaacacggcattgtgccgatcatttgcgtaggtgaaacgcttgaagagcgcgaagccggaaaaacaaatgatcttgttgccgatcaagtgaaaaaaggtcttgcgggtctttctgaagagcaagttgctgcttctgttattgcgtacgagccaatctgggcaatcggaacaggcaaatcttctacagcgaaagatgctaacgacgtctgcgcgcatatccgtaaagtcgttgctgaaagcttcagccaggaaactgctgacaagctccgcattcaatacggc

SEQ ID NO: 382 - DNA, 540 bp, BACILLUS SP.
gcagcagacaaagcggaagccgttgtttgcgcaccagctcttttcttagaaaagctgacttctgctgttaaaggaactgacctgaaagtcggcgcccaaaacatgcattttgaagaaaacggtgcgttcacaggtgaaatcagcccggtcgctctgaaagaccttggcgtggattactgcgtcatcggccactctgagcgccgtgaaatgttcgctgaaactgatgaaacagttaacaaaaaagcgcatgccgctttcaaacacggcattgtgccgatcatttgcgtaggtgaaacgcttgaagagcgcgaagccggaaaaacaaatgatcttgttgccgatcaagtgaaaaaaggtcttgcgggtctttctgcagagcaagttgccgcttccgttattgcgtacgagccaatctgggcaatcggaacaggcaaatcttctacagcgaaagatgctaacgacgtctgcgcgcatatccgtaaagtcgttgctgaaagcttcagccaggaaactgctgacaagctccgcattcaatacggc

SEQ ID NO: 383 - DNA, 540 bp, BACILLUS SP.
gcagcagacaaagcggaagccgttgtttgcgcaccagctcttttcttagaaaagctgacttctgctgttaaaggaactgacctgaaagtcggcgcccaaaacatgcattttgaagaaaacggtgcgttcacaggtgaaatcagcccggtcgctctgaaagaccttggcgtggattactgcgtcatcggccactctgagcgccgtgaaatgttcgctgaaactgatgaaacagttaacaaaaaagcgcatgccgctttcaaacacggcattgtgccgatcatttgcgtaggtgaaacgcttgaagagcgcgaagccggaaaaacaaatgatcttgttgccgatcaagtgaaaaaaggtcttgcgggtctttctgaagagcaagttgccgcttccgttattgcgtacgagccaatctgggcaatcggaacaggcaaatcttctacagcgaaagatgctaacgacgtctgcgcgcatatccgtaaagtcgttgctgaaagcttcagccaggaaactgctgacaagctccgcattcaatacggc

SEQ ID NO: 384 - DNA, 540 bp, BACILLUS SP.
gcagcagacaaagcggaagccgttgtttgcgcaccagctcttttcttagaaaagctgacttctgctgttaaaggaactgacctgaaagtcggcgcccaaaacatgcattttgaagaaaacggtgcgttcacaggtgaaataagcccggtcgctctgaaagaccttggcgtggattactgcgtcatcggccactctgagcgccgtgaaatgttcgctgaaactgatgaaacagttaacaaaaaagcgcatgccgctttcaaacacggcattgtgccgatcatttgcgtaggtgaaacgcttgaagagcgcgaagccggaaaaacaaatgatcttgttgccgatcaagtgaaaaaaggtcttgcgggtctttctgaagagcaagttgccgcttccgttattgcgtacgagccaatctgggcaatcggaacaggcaaatcttctacagcgaaagatgctaacgacgtctgcgcgcatatccgtaaagtcgttgctgaaagcttcagccaggaaactgctgacaagctccgcattcaatacggc

SEQ ID NO: 385 - DNA, 540 bp, BACILLUS SP.
gcagcagataaagcagaagctgttgtctgtgcgcctgcactgttccttgaaaagcttacatcagctgtaaaaggcacagatctgaaagtcggcgcgcaaaacatgcactttgaagaaagcggtgctttcacgggtgaaatcagccctgcggctttgaaagaccttggagttgaatactgcgtcatcggccactctgagcgccgcgaaatgtttgctgaaacggatgaaacggtcaacaaaaaagcgcatgctgcattcaaatacggcatcgtgccgatcatctgcgtaggtgaaacgcttgaagagcgcgaagcaaacaaaacaaacgagcttgttgccgaccaagtgaaaaaagcacttgcaggcttaacaactgaacaggtagctgcttccgtgatcgcttatgagccgatctgggcgattggaaccggcaagtcttcaactgcacaagatgcaaacgaagtatgcgcgcacatccgcaaaaccgttgcatcagaatttggacaaactgcagcagacagcgtccgcattcagtacggc

SEQ ID NO: 386 - DNA, 540 bp, BACILLUS SP.
gcagcagataaagcagaagctgttgtctgtgcgcctgcactgttccttgaaaagcttacatcagctgtaaaaggcacagatctgaaagtcggcgcgcaaaacatgcactttgaagaaagcggtgctttcacgggtgaaatcagccctgcggctttgaaagaccttggagttgaatactgcgtcatcggccactccgagcgccgcgaaatgtttgctgaaacggatgaaacggtcaacaaaaaagcgcatgctgcattcaaatacggcatcgtgccgatcatctgcgtaggtgaaacgcttgaagagcgcgaagcaaacaaaacaaacgagcttgttgcagaccaagtgaaaaaagcacttgcaggcttaacaattgaacaggtagctgcttccgtgatcgcttatgagccgatctgggcgattggaaccggcaagtcttcaactgcacaagatgcaaacgaagtatgcgcgcacatccgcaaaaccgttgcatcagaattcggacaaactgcagcagacagcgtccgcattcagtacggc